(19) United States
(12) Patent Application Publication
(10) Pub. No.: US 2009/0238808 A1
(43) Pub. Date: Sep. 24, 2009

(54) Process for the identification of novel enzyme interacting compounds

(76) Inventors: Gerard Drewes, Heidelburg (DE); Bernhard Kuester, Heidelburg (DE); Ulrich Kruse, Dossenheim (DE); Carsten Hopf, Mannheim (DE); Dirk Eberhard, Mauer (DE); Marcus Bantscheff, Heidelburg (DE); Valerie Reader, Linton (GB); Manfred Raida, Singapore (SG); David Middlemiss, Bishops Stortford (GB)

Correspondence Address: CLARK & ELBING LLP, 101 FEDERAL STREET, BOSTON, MA, 02110, US

(21) Appl. No.: 12/002,222

(22) Filed: Dec. 14, 2007

Related U.S. Application Data
(63) Continuation-in-part of application No. PCT/EP2006/062984, filed on Jun. 7, 2006.
(60) Provisional application No. 60/711,399, filed on Aug. 25, 2005.
(60) Provisional application No. 60/782,170, filed on Mar. 14, 2006.

(30) Foreign Application Priority Data
Jun. 14, 2005 (EP) EP 05012722.4

(51) Int. Cl. A61K 38/43; G01N 33/573

(52) U.S. Cl. 424/94.1; 435/7.4

(57) ABSTRACT

The present invention relates to methods for the characterization of enzymes or of enzyme-compound complexes, wherein the enzyme is obtained from a protein preparation with the help of at least one broad spectrum ligand immobilized on a solid support and wherein the enzyme is characterized by mass spectrometry. These methods are useful for the screening of non-immobilized compound libraries, selectivity profiling of lead compounds and mechanism of action studies in living cells.

### CROSS-REFERENCE TO RELATED APPLICATIONS

This application is a continuation-in-part of International Application No. PCT/EP2006/062984, filed Jun. 7, 2006, which claims priority to U.S. Provisional Application Nos. 60/711,399, filed Aug. 25, 2005, and 60/782,170, filed Mar. 14, 2006, and European Application No. 05012722.4, filed Jun. 14, 2005, each of which is hereby incorporated by reference.

### REFERENCE TO TABLES SUBMITTED ON COMPACT DISC

Supplementary Tables 1 and 2 are submitted on duplicate compact discs, which are hereby incorporated by reference. Each disc contains the files Supplemental_Table_{—}1_{—}1.txt, 10,779 kB and Supplemental_Table_{—}1_{—}2.txt, 10,817 kB, both created Dec. 13, 2007, and Supplemental_Table_{—}2.txt, 3,513 kB, created Dec. 12, 2007. Supplementary Table 1 is found in files Supplemental_Table_{—}1_{—}1.txt and Supplemental_Table_{—}1_{—}2.txt, and Supplementary Table 2 is found in Supplemental_Table_{—}2.txt.

LENGTHY TABLES
The patent application contains a lengthy table section. A copy of the table is available in electronic form from the USPTO web site (http://seqdata.uspto.gov/?pageRequest=docDetail&DocID=US20090238808A1). An electronic copy of the table will also be available from the USPTO upon request and payment of the fee set forth in 37 CFR 1.19(b)(3).

### BACKGROUND OF THE INVENTION

The present invention relates to methods for the characterization of enzymes or enzyme-compound complexes using enzyme ligands bound to solid supports.

The goal of drug discovery is to develop effective and safe medicines. In order to achieve this goal, pharmaceutical research aims at identifying preferably small molecule drugs directed at drug targets that are known to be causative for the disease of interest.

Traditionally, the majority of small molecule drugs are directed against receptor proteins (cell membrane receptors such as G protein coupled receptors (GPCRs) or nuclear hormone receptors), ion channels and enzymes. Of the enzymes, of particular interest are e.g. proteases, phosphodiesterases and kinases (Review: Drews, 2000, “Drug discovery: a historical perspective”, Science 287, 1960-1964).

Proteases are considered as tractable drug targets as demonstrated by the effective management of AIDS with HIV protease inhibitors or the use of angiotensin-converting enzyme inhibitors to treat hypertension. For the treatment of cancer protease inhibitors directed against matrix metalloproteinases and caspases are under development (Docherty et al., 2003, “Proteases as drug targets”, Biochemical Society Symposia 70, 147-161).

Phosphodiesterases (PDEs) comprise a family of enzymes that catalyse the hydrolysis of cAMP or cGMP and are implicated in various diseases. The PDE5 inhibitor sildenafil (Viagra) provides an effective treatment for erectile dysfunction. Currently PDE4 inhibitors (e.g. cilomast and roflumast) are in clinical testing as anti-inflammatory therapeutics. A major challenge in this field is the development of PDE isotype specific inhibitors in order to avoid cross-reactivity that is responsible for side effects (Card et al., 2004, “Structural basis for the activity of drugs that inhibit phosphodiesterases”, Structure 12, 2233-2247).

Kinases catalyse the phosphorylation of proteins, lipids, sugars, nucleosides and other cellular metabolites and play key roles in all aspects of eukaryotic cell physiology. Phosphorylation of proteins is a common posttranslational modification of proteins and affects protein structure and function in numerous ways.

One kinase class that has become a recent focus of drug discovery comprises the protein kinases because they were shown to play important roles in the initiation and progression of tumors through dysregulation of signal transduction pathways (EGF receptor in lung cancer; overexpression of the ErbB2/Her-2 receptor in breast cancer; BCR-ABL fusion protein in leukemia; Review: Blume-Jensen and Hunter, 2001, “Oncogenic kinase signaling”, Nature 411, 355-365).

The complement of protein kinases encoded in the human genome comprises 518 family members (kinome) which can be grouped into several subfamilies according to sequence similarity (Review: Manning et al., 2002, The Protein Kinase Complement of the Human Genome, Science 298, 1912-1934). In any given cell or tissue only a subset of the kinome is expressed. Kinases transfer phosphate groups from ATP to substrate molecules and thereby influence the stability, activity and function of their targets. The ATP binding pocket of different kinases is structurally similar and therefore it is considered difficult to develop selective ATP-competitive inhibitors.

The kinase family is a very large enzyme family (compared to other enzyme classes relevant as drug targets, e.g. phosphodiesterases) providing multiple opportunities for drug discovery but also unique challenges (large size of family; structural similarity of the ATP binding pockets; high intracellular ATP concentration) (Review: Cohen, P., 2002, Protein kinases—the major drug targets of the twenty-first century? Nature Reviews Drug Discovery, volume 1, 309-315).

Another kinase class of interest are lipid kinases. Lipid kinases catalyse the transfer of gamma-phosphate groups from nucleoside triphosphates to lipid substrates.

Lipid kinases such as the phosphoinositide 3-kinase (PI3K) family members are known to be modulators of the cellular response to growth factors, hormones and neurotransmitters and are involved in cancer, diabetes and other diseases (Fruman et al., 1998. Phosphoinositide kinases. Annual Review Biochemistry 67, 481-507; Cantley, L. C., 2002, Science 296, 1655-1657).

One prerequiste for the identification of compounds interacting with proteins, e.g. enzymes, is the provision of protein preparations containing as many proteins as possible of one class in a great purity. Especially, the provision of many proteins of one class (e.g. kinases) is important since this enables the screening of potentially pharmaceutically interesting compounds against many members of the protein family (so called hit identification). Other feasible uses of such protein preparations include the testing of chemically optimized compounds (lead optimization), the determination of the selectivity of a given compound (selectivity profiling) as well as the confirmation of the mode of action of a given compound.

In the art, several strategies have been proposed to assess this issue.

One approach to enrich ATP-binding proteins such as kinases and other nucleotide-binding proteins from cell extracts relies on immobilized ATP as affinity reagent, i.e. on the use of a ligand binding potentially all ATP-binding enzymes. In this case ATP is covalently immobilized by coupling the gamma-phosphate group through a linker to a resin (Graves et al., 2002, Molecular Pharmacology 62, No. 6 1364-1372; U.S. Pat. No. 5,536,822). This approach was further extended to coupling single compounds of combinatorial compound libraries (WO00/63694).

One disadvantages of immobilized ATP is that the affinity for kinases is rather low leading to inefficient capturing of kinases or rapid elution due to high off-rates. Another disadvantage is that kinases are not preferentially captured, but also other classes of ATP-binding proteins which can be expressed at much higher levels in the cell. The more abundant ATP-binding proteins can cause inefficient capturing due to competition or can lead to problems during the mass spectrometry analysis of the bound proteins if the analytical depth is not sufficient.

Another approach described in the art is the use of ligands specific for an individual enzyme, namely high affinity and highly selective kinase inhibitors or close derivatives (e.g. optimized drugs). These are used to enrich kinases from cell lysates and the same non-modified compound is used for specific elution in order to identify the cellular drug target or targets (Godl et al., Proc. Natl. Acad. Sci. 100, 15434-15439; WO 2004/013633). This approach is only successful if the structure-affinity-relationship (SAR) is not destroyed through the chemical modification of the drug but fails if the SAR is impaired. In addition, it is difficult to identify targets mediating unwanted side effects because the SAR of the cognate drug target and the side-effect-target can be different and the latter SAR is usually not known.

Another strategy is the in vitro expression of enzymes of a given class, e.g. kinases. Fabian and colleagues (Fabian et al., 2005, Nature Biotechnology 23(3), 329-336; WO 03084981) describe a kinase profiling method that does not rely on capturing the endogenous kinases contained in cell lysates but uses kinases displayed on bacteriophage T7. The kinases (or kinase domains) used in this assay are fusion proteins that are tagged in order to allow expression, purification and detection. In the competition binding assay these phage-tagged kinases are bound to an immobilized kinase inhibitor, treated with a non-immobilized test compound and the bound tagged kinases are quantified by real-time PCR using the phage DNA as a template. A disadvantage of this method is that the kinases need to be cloned and only a fraction of the phage-tagged kinases are folded in the correct native state. Furthermore, such protein preparations do not reflect at all the natural situation in a cell.

Yet another approach uses active-site directed probes (socalled activity-based probes) that form covalent links with target enzymes. This method was used to profile the expression of serine hydrolases with highly selective probes (Liu et al., 1999, Proc. Natl. Acad. Sci. 96, 14694-14699, WO 01/77668) and further expanded to other enzyme families by using more promiscuous probes consisting of non-directed activity-based probe libraries of rhodamine- and biotin-tagged fluorescent sulfonate esters (Adam et al., 2002, Nature Biotechnology 20, 805-809, WO 01/77684, WO 03/047509). However, it remains unclear what structural and/or catalytic properties are shared by these sulfonate-targeted enzymes. Another limitation of this approach is the difficulty to distinguish specific interactions with enzymes and non-specific interactions caused by the intrinsic reactivity of the probes.

Finally, it was tried to enrich proteins phosphorylated on tyrosines by tyrosine specific antibodies (Blogoev et al., 2004, Nature Biotechnology 9, 1139-1145). Blogoev and colleagues describe a method that can be used to study the effect of compounds such as epidermal growth factor (EGF) on phosphotyrosine-dependent signal transduction pathways. After lysis of the EGF-stimulated cells phosphotyrosine-containing proteins are enriched by immunoprecipitation with antibodies directed against phosphotyrosine. In the second step these enriched proteins are analysed and identified by mass spectrometric analysis. One major limitation of this approach is that only proteins phosphorylated on tyrosine can be captured.

In view of this, there is a need for improved methods for the characterization of those enzymes of a given class which are expressed in a cell. Furthermore, there is a need for improved methods for the identification of enzymes being binding partners of a given compound.

### SUMMARY OF THE INVENTION

The present invention satisfies these needs. In the context of the present invention, it has been surprisingly found that the use of at least one broad spectrum enzyme ligand immobilized on a solid support enables the effective isolation of enzymes out of a protein preparation, preferably a cell lysate. After this isolation, effective methods can be applied either for the characterization of the enzyme bound to the broad spectrum enzyme ligand or for the identification of compound enzyme interactions. The enzyme is preferably identified by mass spectrometry. Therefore, the present invention provides effective methods for either the characterization of enzymes or the identification of binding partners to a given compound.

In a first aspect, the present invention provides a method for the characterization of at least one enzyme, comprising the steps of:
a) providing a protein preparation containing the enzyme, preferably by harvesting at least one cell containing the enzyme and lysing the cell,
b) contacting the protein preparation under essentially physiological conditions with at least one broad spectrum enzyme ligand immobilized on a solid support under conditions allowing the binding of the enzyme to said broad spectrum enzyme ligand,
c) eluting the enzyme, and
d) characterizing the eluted enzyme by mass spectrometry.
In a second aspect, the present invention provides a method for the characterization of at least one enzyme, comprising the steps of:
a) providing two aliquots comprising each at least one cell containing the enzyme,
b) incubating one aliquot with a given compound,
c) harvesting the cells,
d) lysing the cells,
e) contacting the protein preparation under essentially physiological conditions with at least one broad spectrum enzyme ligand immobilized on a solid support under conditions allowing the binding of the enzyme to said broad spectrum enzyme ligand,
f) eluting the enzyme or enzymes, and
g) characterizing the eluted enzyme or enzymes by mass spectrometry.

According to a third aspect of the invention, the invention provides a method for the characterization of at least one enzyme, comprising the steps of:
a) providing two aliquots of a protein preparation containing the enzyme, preferably by harvesting at least one cell containing the enzyme and lysing the cell,
b) contacting one aliquot under essentially physiological conditions with at least one broad spectrum enzyme ligand immobilized on a solid support under conditions allowing the binding of the enzyme to said broad spectrum enzyme ligand,
c) contacting the other aliquot under essentially physiological conditions with at least one broad spectrum enzyme ligand immobilized on a solid support under conditions allowing the binding of the enzyme to said broad spectrum enzyme ligand and with a given compound,
d) eluting the enzyme or enzymes, and
e) characterizing the eluted enzyme or enzymes by mass spectrometry.

According to a fourth aspect of the present invention, a method for the characterization of an enzyme-compound complex is provided, comprising the steps of:
a) providing a protein preparation containing the enzyme, preferably by harvesting at least one cell containing the enzyme and lysing the cell,
b) contacting the protein preparation under essentially physiological conditions with at least one broad spectrum enzyme ligand immobilized on a solid support under conditions allowing the binding of the enzyme to said broad spectrum enzyme ligand,
c) contacting the bound enzymes with a compound to release at least one bound enzyme, and
d) characterizing the released enzyme or enzymes by mass spectrometry, or
e) eluting the enzyme or enzymes from the ligand and characterizing the enzyme or enzymes by mass spectrometry, thereby identifying one or more binding partners of the compound.

The approaches as mentioned above, especially the method of the invention according to the 4^{th} aspect, have the following advantages:
No in vitro expression of enzymes is necessary, but endogenous enzymes from cell lysates are used.
Surprisingly, it was found that broad specificity kinase ligands capture kinases very efficiently.
The test compounds do not need to be linked or immobilized (label-free assay method).

The competition binding or elution is possible with any compound of interest (non-modified, non-immobilized).
No enzyme substrates are necessary (as in biochemical enzyme assays).
It is possible to characterize the in vivo effect of the compound on a signal transduction pathway (see 2^{nd} aspect).
The identification of direct and indirect targets is possible.

Throughout the invention, the term “enzyme” includes also every protein or peptide in the cell being able to bind a ligand such as transporters, ion channels and proteins that interact with enzymes such as adapter proteins containing peptide interaction domains (e.g. SH2, SH3, and PDZ domains).

Preferably, however, the term “enzyme” is interpreted in its usual way as being a biocatalysator in a cell.

Throughout the invention, the term “broad spectrum enzyme ligand” refers to a ligand which is able to bind some, but not all enzymes present in a protein preparation.

The present invention preferably relates to methods for the characterization of enzymes or the identification of binding partners to a given compound, wherein the enzyme or the binding partners are included in a cell lysate. However, the methods of the present invention can also be performed with any protein preparation as a starting material, as long as the protein(s) is/are solubilized in the preparation. Examples include a liquid mixture of several proteins, a partial cell lysate which contains not all proteins present in the original cell or a combination of several cell lysates.

Partial cell lysates can be obtained by isolating cell organelles (e.g. nucleus, mitochondria, ribosomes, golgi etc.) first and then prepare protein preparations derived from these organelles. Methods for the isolation of cell organelles are known in the art (Chapter 4.2 Purification of Organelles from Mammalian Cells in “Current Protocols in Protein Science”, Editors: John. E. Coligan, Ben M. Dunn, Hidde L. Ploegh, David W. Speicher, Paul T. Wingfield; Wiley, ISBN: 0-471-14098-8).

In addition, protein samples can be prepared by fractionation of cell extracts thereby enriching specific types of proteins such as cytoplasmic or membrane proteins (Chapter 4.3 Subcellular Fractionation of Tissue Culture Cells in “Current Protocols in Protein Science”, Editors: John. E. Coligan, Ben M. Dunn, Hidde L. Ploegh, David W. Speicher, Paul T. Wingfield; Wiley, ISBN: 0-471-14098-8).

Furthermore protein preparations from body fluids can be used (e.g. blood, cerebrospinal fluid, peritoneal fluid and urine).

Throughout the invention, the term “solid support” relates to every undissolved support being able to immobilize said broad spectrum enzyme ligand on its surface.

The method of the invention according to the second aspect encompasses as initial steps a provision of two aliquots comprising each at least one cell containing the enzyme and incubating one aliquot with a given compound. In a preferred embodiment, the at least one cell is part of the cell culture system which is divided into at least two aliquots. One aliquot of cells is then incubated with the given compound. Methods for the incubation of cell culture systems with compounds are known in the art (Giuliano et al., 2004, “High-content screening with siRNA optimizes a cell biological approach to drug discovery: defining the role of p53 activation in the cellular response to anticancer drugs”. Journal of Biomolecular Screening 9(7), 557-568).

However, it is also included within the present invention that the at least one cell of each aliquot is part of an in vivo system, e.g. a mouse system or a lower vertebrate system.

For example whole embryo lysates derived from defined development stages or adult stages of model organisms such as C. elegans can be used. In addition, whole organs such as heart dissected from mice can be the source of protein preparations. These organs can also be perfused in vitro and so be treated with the test compound or drug of interest.

All methods of the present invention include at least in a preferred embodiment the steps of harvesting at least one cell containing the enzyme and lysing the cell.

In a preferred embodiment, the cell is part of a cell culture system and methods for the harvest of a cell out of a cell culture system are known in the art (literature supra).

The choice of the cell will mainly depend on the class of enzymes supposed to be analyzed, since it has to be ensured that the class of enzyme is principally present in the cell of choice. In order to determine whether a given cell is a suitable starting system for the methods of the invention, methods like Westernblot, PCR-based nucleic acids detection methods, Northernblots and DNA-microarray methods (“DNA chips”) might be suitable in order to determine whether a given class of enzymes is present in the cell.

The choice of the cell will also be influenced by the purpose of the study. If the in vivo target for a given drug needs to be identified then cells or tissues will be selected in which the desired therapeutic effect occurs (e.g. breast cancer tissue for anticancer drugs). By contrast, for the elucidation of protein targets mediating unwanted side effects the cell or tissue will be analysed in which the side effect is observed (e.g. brain tissue for CNS side effects).

Furthermore, it is envisaged within the present invention that the cell containing the enzyme may be obtained from an organism, e.g. by biopsy. Corresponding methods are known in the art. For example, a biopsy is a diagnostic procedure used to obtain a small amount of tissue, which can then be examined miscroscopically or with biochemical methods. Biopsies are important to diagnose, classify and stage a disease, but also to evaluate and monitor drug treatment. Breast cancer biopsies were previously performed as surgical procedures, but today needle biopsies are preferred (Oyama et al., 2004, Breast Cancer 11(4), 339-342).

The described methods of the invention allow to profile the tissue samples for the presence of enzyme classes. For example, mutated enzymes causative for the disease can be identified (e.g. point mutations that activate oncogenic kinases). In addition, mutated enzymes that arise during treatment and are responsible for treatment resistance can be elucidated (e.g. EGF-receptor mutations causing resistance to anti-cancer drugs).

Liver biopsy is used for example to diagnose the cause of chronic liver disease that results in an enlarged liver or abnormal liver test results caused by elevated liver enzyme activities (Rocken et al., 2001, Liver 21(6), 391-396).

It is encompassed within the present invention that by the harvest of the at least one cell, the lysis is performed simultaneously. However, it is equally preferred that the cell is first harvested and then separately lysed.

Methods for the lysis of cells are known in the art (Karwa and Mitra: Sample preparation for the extraction, isolation, and purification of Nuclei Acids; chapter 8 in “Sample Preparation Techniques in Analytical Chemistry”, Wiley 2003, Editor: Somenath Mitra, print ISBN: 0471328456; online ISBN: 0471457817). Lysis of different cell types and tissues can be achieved by homogenizers (e.g. Potter-homogenizer), ultrasonic desintegrators, enzymatic lysis, detergents (e.g. NP-40, Triton X-100, CHAPS, SDS), osmotic shock, repeated freezing and thawing, or a combination of these methods.

Furthermore, all methods of the invention contain the step of contacting the cell preparation or cell lysate under essentially physiological conditions with at least one broad spectrum enzyme ligand immobilized on a solid support under conditions allowing the binding of the enzyme to said broad spectrum enzyme ligand.

The contacting under essentially physiological conditions has the advantage that the interactions between the ligand, the cell preparation (i.e. the enzyme to be characterized) and optionally the compound reflect as much as possible the natural conditions. “Essentially physiological conditions” are inter alia those conditions which are present in the original, unprocessed sample material. They include the physiological protein concentration, pH, salt concentration, buffer capacity and post-translational modifications of the proteins involved. The term “essentially physiological conditions” does not require conditions identical to those in the original living organism, wherefrom the sample is derived, but essentially cell-like conditions or conditions close to cellular conditions. The person skilled in the art will, of course, realize that certain constraints may arise due to the experimental set-up which will eventually lead to less cell-like conditions. For example, the eventually necessary disruption of cell walls or cell membranes when taking and processing a sample from a living organism may require conditions which are not identical to the physiological conditions found in the organism. Suitable variations of physiological conditions for practicing the methods of the invention will be apparent to those skilled in the art and are encompassed by the term “essentially physiological conditions” as used herein. In summary, it is to be understood that the term “essentially physiological conditions” relates to conditions close to physiological conditions, as e.g. found in natural cells, but does not necessarily require that these conditions are identical.

Preferably, “essentially physiological conditions” may comprise 50-200 mM NaCl or KCl, pH 6.5-8.5, 20-45° C., and 0.001-10 mM divalent cation (e.g. Mg++, Ca++,); more preferably about 150 m NaCl or KCl, pH7.2 to 7.6, 5 mM divalent cation and often include 0.01-1.0 percent non-specific protein (e.g. BSA). A non-ionic detergent (Tween, NP40, Triton-X100) can often be present, usually at about 0.001 to 2%, typically 0.05-0.2% (volume/volume). For general guidance, the following buffered aequous conditions may be applicable: 10-250 mM NaCl, 5-50 mM Tris HCl, pH5-8, with optional addition of divalent cation(s) and/or metal chelators and/or non-ionic detergents.

Preferably, “essentially physiological conditions” mean a pH of from 6.5 to 7.5, preferably from 7.0 to 7.5, and/or a buffer concentration of from 10 to 50 mM, preferably from 25 to 50 mM, and/or a concentration of monovalent salts (e.g. Na or K) of from 120 to 170 mM, preferably 150 mM. Divalent salts (e.g. Mg or Ca) may further be present at a concentration of from 1 to 5 mM, preferably 1 to 2 mM, wherein more preferably the buffer is selected from the group consisting of Tris-HCl or HEPES.

In the context of the present invention, the term “under conditions allowing the binding of the enzyme to said broad spectrum enzyme ligand” includes all conditions under which such binding is possible. This includes the possibility of having the solid support on an immobilized phase and pouring the lysate onto it. In another preferred embodiment, it is also included that the solid support is in a particulate form and mixed with the cell lysate.

In a preferred embodiment, the binding between ligand and enzyme is a non covalent, reversible binding, e.g. via salt bridges, hydrogen bonds, hydrophobic interactions or a combination thereof.

Additionally, the methods of the invention include the step of eluting the enzyme or enzymes from the ligand immobilized on the solid support.

Such methods are principally known in the art and depend on the nature of the ligand enzyme interaction. Principally, change of ionic strength, the pH value, the temperature or incubation with detergents are suitable methods to dissociate the target enzymes from the immobilized ligand. The application of an elution buffer can dissociate binding partners by extremes of pH value (high or low pH; e.g. lowering pH by using 0.1 M citrate, pH2-3), change of ionic strength (e.g. high salt concentration using NaI, KI, MgCl2, or KCl), polarity reducing agents which disrupt hydrophobic interactions (e.g. dioxane or ethylene glycol), or denaturing agents (chaotropic. salts or detergents such as Sodium-docedyl-sulfate, SDS; Review: Subramanian A., 2002, Immunoaffinity chromatography. Mol. Biotechnol. 20(1), 41-47).

With these rather non-specific methods most or all bound proteins will be released and then need to be analysed by mass spectrometry (or alternatively by detection with antibodies, see below).

The method according to the 4^{th} aspect of the present invention further includes the step of contacting the bound enzymes with a compound to release at least one bound enzyme. This contacting preferably also occurs under essentially physiological conditions.

One advantage of using a compound of interest for elution instead of the non-specific reagents described above is that not all bound proteins are released but only a subfraction, preferably the enzyme class of interest. Consequently fewer proteins need to be identified by mass spectrometry resulting in faster analysis and more analytical depth (sensitivity) for the enzyme class of interest.

The skilled person will appreciate that between the individual steps of the methods of the invention, washing steps may be necessary. Such washing is part of the knowledge of the person skilled in the art. The washing serves to remove non-bound components of the cell lysate from the solid support. Nonspecific (e.g. simple ionic) binding interactions can be minimized by adding low levels of detergent or by moderate adjustments to salt concentrations in the wash buffer.

After the elution or contacting, in some cases the solid support has preferably to be separated from the released material. The individual methods for this depend on the nature of the solid support and are known in the art. If the support material is contained within a column the released material can be collected as column flowthrough. In case the support material is mixed with the lysate components (so called batch procedure) an additional separation step such as gentle centrifugation may be necessary and the released material is collected as supernatant. Alternatively magnetic beads can be used as solid support so that the beads can be eliminated from the sample by using a magnetic device.

According to the present invention, the eluted enzyme or enzymes or coeluted binding partners (see below) as well as the released enzymes according to the method of the fourth aspect of the invention are preferably characterized by mass spectrometry. Alternatively, it is throughout the invention also possible to perform this characterization with specific antibodies directed against the respective enzyme or coeluted binding partner.

The identification of proteins with mass spectrometric analysis (mass spectrometry) is known in the art (Shevchenko et al., 1996, Analytical Chemistry 68: 850-858, (Mann et al., 2001, Analysis of proteins and proteomes by mass spectrometry, Annual Review of Biochemistry 70, 437-473) and is further illustrated in the example section.

As an alternative to mass spectrometry analysis, the eluted enzyme or enzymes (including coeluted binding partners, for example enzyme subunits or scaffold proteins), can be detected by using specific antibodies directed against a protein of interest.

Furthermore, in another preferred embodiment, once the identity of the eluted enzyme or enzymes has been established by mass spectrometry analysis, each enzyme of interest can be detected with specific antibodies directed against this enzyme.

Suitable antibody-based assays include but are not limited to Western blots, ELISA assays, sandwich ELISA assays and antibody arrays or a combination thereof. The establishment of such assays is known in the art (Chapter 11, Immunology, pages 11-1 to 11-30 in: Short Protocols in Molecular Biology. Fourth Edition, Edited by F. M. Ausubel et al., Wiley, New York, 1999).

Multiple assays can be performed using several antibodies in parallel, for example directed against many members of an enzyme family (Pelch et al., Kinetworks Protein Kinase Multiblot analysis. Chapter 8, pages 99-112 in: Cancer Cell Signalling. Methods and Protocols. Editor: David M. Terrian. Humana Press, Totowa, USA, 2002).

These assays can not only be configured in a way to detect and quantify an enzyme of interest, but also to analyse posttranslational modification patterns such as phosphorylation. For example, the activation state of a kinase can be determined by probing its phosphorylation status with specific anti-phosphotyrosine, anti-phosphoserine or anti-phosphothreonine antibodies. It is known in the art how to select and use such anti-phospho antibodies (Zhang et al., 2002. Journal of Biological Chemistry 277, 43648-43658).

According to a preferred embodiment of the method of the invention according to the 2^{nd} aspect, by characterizing the enzyme, it is determined whether the administration of the compound results in a differential expression or activation state of the enzyme. Therefore, by administration of the compound, either the expression of the enzyme may be changed or the activation state of the enzyme may be changed.

In this context, a change in the expression of the enzyme may preferably either mean that more or that less enzyme is produced in the cell.

By change of the activation state, it is preferably meant that either the enzyme is more active after administration of the compound or less active after the administration of the compound. It can also mean that the affinity of the enzyme for the immobilized ligand is increased or decreased (e.g. change of the activation state of a kinase through phosphorylation by an upstream kinase; or binding of the compound to an allosteric regulatory side of the enzyme and thereby altering the conformation of the ATP-binding pocket of an ATP-binding enzyme).

According to a preferred embodiment of the method of the invention according to the 1^{st} aspect, the protein preparation is incubated, preferably under essentially physiological conditions, with a compound as defined below. In consequence, only enzymes not binding to the compound are subsequently bound to the ligand, eluted and characterized.

According to a preferred embodiment of the method according to the 3^{rd} aspect of the invention, in step c) the aliquot is contacted, preferably under essentially physiological conditions, with the compound before the incubation with the ligand. In consequence, only enzymes not binding to the compound are subsequently bound to the ligand, eluted and characterized.

In a preferred embodiment of the method of the invention according to the third aspect, a reduced detection of the enzyme in the aliquot incubated with the compound indicates that the enzyme is a direct target of the compound. This results from the fact that in step c) of this method of the invention, the compound competes with the ligand for the binding of the enzyme. If less enzyme can be detected in the aliquot incubated with the compound, this means preferably that the compound has competed with the inhibitor for the interaction with the enzyme and is, therefore, a direct target of the enzyme and vice versa.

According to a preferred embodiment of the method of the invention according to the fourth aspect, this method is performed as a medium or high throughput screening. Such assays are known to the person skilled in the art (Mallari et al., 2003, A generic high-throughput screeing assay for kinases: protein kinase A as an example, Journal of Biomolecular Screening 8, 198-204; Rodems et al., 2002, A FRET-based assay platform for ultra-high density screening of protein kinases and phosphatases, Assay and Drug Development Technologies 1 (1PT1), 9-19).

Essential to the methods according to the second, third and fourth aspect of the invention is the provision of a compound which is supposed to interact with the enzyme. Principally, according to the present invention, such a compound can be every molecule which is able to interact with the enzymes. Preferably, the compound has an effect on the enzyme, e.g. a stimulatory or inhibitory effect.

Preferably, said compound is selected from the group consisting of synthetic or naturally occurring chemical compounds or organic synthetic drugs, more preferably small molecules, organic drugs or natural small molecule compounds. Preferably, said compound is identified starting from a library containing such compounds. Then, in the course of the present invention, such a library is screened.

Such small molecules are preferably not proteins or nucleic acids. Preferably, small molecules exhibit a molecular weight of less than 5000 Da, more preferred less than 2000 Da, even more preferred less than 1000 Da and most preferred less than 500 Da.

A “library” according to the present invention relates to a (mostly large) collection of (numerous) different chemical entities that are provided in a sorted manner that enables both a fast functional analysis (screening) of the different individual entities, and at the same time provide for a rapid identification of the individual entities that form the library. Examples are collections of tubes or wells or spots on surfaces that contain chemical compounds that can be added into reactions with one or more defined potentially interacting partners in a high-throughput fashion. After the identification of a desired “positive” interaction of both partners, the respective compound can be rapidly identified due to the library construction. Libraries of synthetic and natural origins can either be purchased or designed by the skilled artisan.

Examples of the construction of libraries are provided in, for example, Breinbauer R, Manger M, Scheck M, Waldmann H. Natural product guided compound library development. Curr Med. Chem. 2002 December; 9(23):2129-45, wherein natural products are described that are biologically validated starting points for the design of combinatorial libraries, as they have a proven record of biological relevance. This special role of natural products in medicinal chemistry and chemical biology can be interpreted in the light of new insights about the domain architecture of proteins gained by structural biology and bioinformatics. In order to fulfil the specific requirements of the individual binding pocket within a domain family it may be necessary to optimise the natural product structure by chemical variation. Solid-phase chemistry is said to become an efficient tool for this optimisation process, and recent advances in this field are highlighted in this review article. Other related references include Edwards P J, Morrell A I. Solid-phase compound library synthesis in drug design and development. Curr Opin Drug Discov Devel. 2002 July; 5(4):594-605; Merlot C, Domine D, Church D J. Fragment analysis in small molecule discovery. Curr Opin Drug Discov Devel. 2002 May; 5(3):391-9. Review; Goodnow R A Jr. Current practices in generation of small molecule new leads. J Cell Biochem Suppl. 2001; Suppl 37:13-21; which describes that the current drug discovery processes in many pharmaceutical companies require large and growing collections of high quality lead structures for use in high throughput screening assays. Collections of small molecules with diverse structures and “drug-like” properties have, in the past, been acquired by several means: by archive of previous internal lead optimisation efforts, by purchase from compound vendors, and by union of separate collections following company mergers. Although high throughput/combinatorial chemistry is described as being an important component in the process of new lead generation, the selection of library designs for synthesis and the subsequent design of library members has evolved to a new level of challenge and importance. The potential benefits of screening multiple small molecule compound library designs against multiple biological targets offers substantial opportunity to discover new lead structures.

The test compounds that can elute target enzymes from the immobilized ligands (4^{th} aspect of the invention) may be tested in conventional enzyme assays. In the following, exemplary assays will be described that can be used to further characterize these compounds. It is not intended that the description of these assays limits the scope of the present invention.

### Protease Assay

An exemplary protease assay can be carried out by contacting a protease with a double labeled peptide substrate with fluor (e.g. EDANS) and quencher chromophores (e.g. DABCYL) under appropriate conditions and detecting the increase of the fluorescence after cleavage.

The substrate contains a fluorescent donor near one end of the peptide and an acceptor group near the other end. The fluorescence of this type of substrate is initially quenched through intramolecular fluorescence resonance energy transfer (FRET) between the donor and acceptor. When the protease cleaves the substrates the products are released from quenching and the fluorescence of the donor becomes apparent. The increase of the fluorescence signal is directly proportional to the amount of substrate hydrolysed (Taliani, M. et al, 1996, Methods 240: 60-7).

### Phosphodiesterase Assay

A cell lysate of human leukocytes (U937) cells may be prepared in a suitable buffer and serves as source of the PDE enzyme. After 20 minutes of incubation at 25° C. with [3H]cAMP as substrate in incubation buffer (50 mM Tris-HCl, ph 7.5, 5 mM MgCl2) the [3H]Adenosine is quantified (Cortijo et al., 1993, British Journal of Pharmacology 108, 562-568).

### In Vitro Enzyme Activity Assay for Protein Kinases

Briefly, a fluorescein-labeled peptide substrate may be incubated with the tyrosine kinase (e.g. Lck), ATP and an anti-phosphotyrosine antibody. As the reaction proceeds, the phosphorylated peptide binds to the anti-phosphotyrosine antibody, resulting in an increase in the polarization signal. Compounds that inhibit the kinase result in a low polarization signal.

Alternatively, the assay can be configured in a modified indirect format. A fluorescent phosphopeptide is used as a tracer for complex formation with the anti-phospho-tyrosine antibody yielding a high polarization signal. When unlabeled substrate is phosphorylated by the kinase, the product competes with the fluorescent phosphorylated peptide for the antibody. The fluorescent peptide is then released from the antibody into solution resulting in a loss of polarization signal. Both the direct and indirect assays can be used to identify inhibitors of protein tyrosine kinase activity (Seethala, 2000, Methods 22, 61-70; Seethala and Menzel, 1997, Anal. Biochem. 253, 210-218; Seethala and Menzel, 1998, Anal. Biochem. 255, 257-262).

This fluorescence polarization assay can be adapted for the use with protein serine/threonine kinases by replacing the antiphophotyrosine antibody with an anti-phosphoserine or anti-phosphothreonine antibody (Turek et al., 2001, Anal. Biochem. 299, 45-53, PMID 11726183; Wu et al., 2000, J. Biomol. Screen. 5, 23-30, PMID 10841597).

The compounds identified in the method according to the 4^{th} aspect of the present invention may further be optimized (lead optimisation). This subsequent optimisation of such compounds is often accelerated because of the structure-activity relationship (SAR) information encoded in these lead generation libraries. Lead optimisation is often facilitated due to the ready applicability of high-throughput chemistry (HTC) methods for follow-up synthesis.

Preferably, lead optimisation is supported with a method according to the 2^{nd}, 3^{rd} and 4^{th} aspect of the present invention, more preferably with a method according to the 4^{th} aspect. The results of these methods may provide guidance to medicinal chemists or to another person skilled in the art how to further optimize compounds with respect to e.g. selectivity.

One use of such a library is finally described in, for example, Wakeling A E, Barker A J, Davies D H, Brown D S, Green L R, Cartlidge S A, Woodburn J R. Specific inhibition of epidermal growth factor receptor tyrosine kinase by 4-anilinoquinazolines. Breast Cancer Res Treat. 1996; 38(1):67-73.

The enzyme which may be characterized according to the present invention, is preferably selected from the group consisting of a kinase, a phosphatase, a protease, a phosphodiesterase, a hydrogenase, a dehydrogenase, a ligase, an isomerase, a transferase, an acetylase, a deacetylase, a GTPase, a polymerase, a nuclease and a helicase.

Preferably, the protein is a kinase, and more preferably a protein kinase. Equally preferred, the protein is a lipid kinase.

As already indicated above, it is essential to the present invention that the ligand is a broad spectrum ligand which is able to bind various, but not all enzymes of a given class of enzymes. Preferably, the ligand binds to 10 to 50%, more preferably to 30 to 50% of the enzymes of a given class of enzymes.

Preferably, the ligand is an inhibitor of the enzyme.

In a more preferred embodiment, the enzyme is a kinase and the ligand is a kinase inhibitor.

Preferably, this kinase inhibitor is selected from the group consisting of Bisindolylmaleimide VIII, Purvalanol B, CZC00007324 (linkable PD173955), CZC00008004.

Further ligands include indol ligand 91, quinazoline ligand 32 and a modified Staurosporine (see Example 5 to 7).

The structure of indol ligand 91 (5-[5-Fluoro-2-oxo-1,2-dihydro-indol-(3Z)-ylidenemethyl]-2,4-dimethyl-1H-pyrrole-3-carboxylic acid (3-amino-propyl)-amide) is given in FIG. 3. This compound is a molecule structurally similar to the kinase inhibitor Sutent (SU11248; Sun et al., 2003. J. Med. Chem. 46, 1116-1119). Indol ligand 91 can be covalently coupled to a suitable solid support material via the primary amino group and be used for the isolation of binding proteins. The synthesis of indol ligand 91 is described in Example 1. According to the invention, the expression “indol ligand 91” also includes compounds comprising the identical core but which have another linker, preferably coupled to the NH group not being part of the cyclic structures, for linkage to the solid support. Typically linkers have backbone of 8, 9 or 10 atoms. The linkers contain either a carboxy- or amino-active group.

According to a further preferred embodiment, the characterization of the enzyme is performed by characterizing co-eluted binding partners of the enzyme, enzyme subunits or post-translational modifications of the enzyme.

The basis of this preferred embodiment is that due to the use of essentially physiological conditions during the binding between the ligand and the enzyme, it is preferably possible to preserve the natural condition of the enzyme which includes the existence of binding partners, enzyme subunits or post-translational modifications. With the help of mass spectrometry (MS), it is possible not only to identify the enzyme, but also the co-eluted binding partners, enzyme subunits or said post-translational modifications.

According to a further preferred embodiment of the present invention, the characterization by mass spectrometry (MS) is performed by the identification of proteotypic peptides of the enzyme or of the binding partner of the enzyme. The concept of proteotypic peptides is described in detail in the example section. The idea is that the eluted enzyme or binding partner is digested with proteases and the resulting peptides are determined by MS. As a result, peptide frequencies for peptides from the same source protein differ by a great degree, the most frequently observed peptides that “typically” contribute to the identification of this protein being termed “proteotypic peptide”. Therefore, a proteotypic peptide as used in the present invention is an experimentally well observable peptide that uniquely identifies a specific protein or protein isoform.

According to a preferred embodiment, the characterization is performed by comparing the proteotypic peptides obtained for the enzyme or the binding partner with known proteotypic peptides. Since, when using fragments prepared by protease digestion for the identification of a protein in MS, usually the same proteotypic peptides are observed for a given enzyme, it is possible to compare the proteotypic peptides obtained for a given sample with the proteotypic peptides already known for enzymes of a given class of enzymes and thereby identifying the enzyme being present in the sample.

Preferably, the mass spectrometry analysis is performed in a quantitative manner, for example by using iTRAQ technology (isobaric tags for relative and absolute quantification) or cICAT (cleavable isotope-coded affinity tags) (Wu et al., 2006. J. Proteome Res. 5, 651-658).

According to a further preferred embodiment, the solid support is selected from the group consisting of agarose, modified agarose, sepharose beads (e.g. NHS-activated sepharose), latex, cellulose, and ferri- or ferromagnetic particles.

The broad spectrum enzyme ligand may be coupled to the solid support either covalently or non-covalently. Non-covalent binding includes binding via biotin affinity ligands binding to steptavidin matrices.

Preferably, the broad spectrum ligand is covalently coupled to the solid support.

Before the coupling, the matrixes can contain active groups such as NHS, Carbodimide etc. to enable the coupling reaction with compounds. The compounds can be coupled to the solid support by direct coupling (e.g. using functional groups such as amino-, sulfhydryl-, carboxyl-, hydroxyl-, aldehyde-, and ketone groups) and by indirect coupling (e.g. via biotin, biotin being covalently attached to the compound and non-covalent binding of biotin to streptavidin which is bound to solid support directly).

The linkage to the solid support material may involve cleavable and non-cleavable linkers. The cleavage may be achieved by enzymatic cleavage or treatment with suitable chemical methods.

Preferred binding interfaces for binding the compound of interest to solid support material are linkers with a C-atom backbone. Typically linkers have backbone of 8, 9 or 10 atoms. The linkers contain either, depending on the compound to be coupled, a carboxy- or amino-active group.

More complete coverage of an enzyme class can be achieved by using combinations of broad spectrum ligands.

Preferably, 1 to 10 more preferred 1 to 6, even more preferred 1 to 4 different ligands are used. Most preferred, 3 or 4 different ligands are used

In case that more than one ligand is used, each ligand is preferably on a different support.

However, it is equally preferred that when more than one ligand is used, at least two or all different ligands are present on one solid support.

In case that more than one ligand is used, it is preferred that the spectrum which each individual ligand can bind is different so that maximum coverage of the enzyme class can be achieved.

Preferably, each ligand binds to 10 to 50%, more preferably to 30 to 50% of the enzymes of a given class of enzymes.

According to a further preferred embodiment, by characterizing the enzyme or the compound enzyme complex, the identity of all or several of the members of an enzyme class in the cell is determined. This is due to the fact that by incubating the ligand with the cell lysate, potentially all enzymes being capable of binding to the ligand are isolated and later on characterized. Depending on the expression profile of the enzymes, the ligand is able to bind to all or some of the members of an enzyme class, which can thus be identified. In the case of kinases, the methods of the present invention enable the skilled person to identify and characterize the kinome expressed in a given cell.

Throughout the invention, it is preferred that the compound is different from the ligand, although identity is not excluded.

The invention further relates to a method for the production of a pharmaceutical composition, comprising the steps of:
a) identifying an enzyme compound complex according to the method of the fourth aspect of the present invention, and
b) formulating the compound to a pharmaceutical composition.

Therefore, the invention provides a method for the preparation of pharmaceutical compositions, which may be administered to a subject in an effective amount. In a preferred aspect, the therapeutic is substantially purified. The subject to be treated is preferably an animal including, but not limited to animals such as cows, pigs, horses, chickens, cats, dogs, etc., and is preferably a mammal, and most preferably human. In a specific embodiment, a non-human mammal is the subject.

In general, the pharmaceutical compositions of the present invention comprise a therapeutically effective amount of a therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The therapeutics of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

The amount of the therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. In general, suppositories may contain active ingredient in the range of 0.5% to 10% by weight; oral formulations preferably contain 10% to 95% active ingredient.

Various delivery systems are known and can be used to administer a therapeutic of the invention, e.g., encapsulation in liposomes, microparticles, and microcapsules: use of recombinant cells capable of expressing the therapeutic, use of receptor-mediated endocytosis (e.g., Wu and Wu, 1987, J. Biol. Chem. 262:4429-4432); construction of a therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion, by bolus injection, by absorption through epithelial or mucocutaneous linings (e.g., oral, rectal and intestinal mucosa, etc.), and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the therapeutic can be delivered in a vesicle, in particular a liposome (Langer, 1990, Science 249:1527-1533; Treat et al., 1989, In: Liposomes in the Therapy of Infectious Disease and Cancer, Lopez-Berestein and Fidler, eds., Liss, New York, pp. 353-365; Lopez-Berestein, ibid., pp. 317-327; see generally ibid.)

In yet another embodiment, the therapeutic can be delivered via a controlled release system. In one embodiment, a pump may be used (Langer, supra; Sefton, 1987, CRC Crit. Ref. Biomed. Eng. 14:201-240; Buchwald et al., 1980, Surgery 88:507-516; Saudek et al., 1989, N. Engl. J. Med. 321:574-579). In another embodiment, polymeric materials can be used (Medical Applications of Controlled Release, Langer and Wise, eds., CRC Press, Boca Raton, Fla., 1974; Controlled Drug Bioavailability, Drug Product Design and Performance, Smolen and Ball, eds., Wiley, New York, 1984; Ranger and Peppas, 1983, Macromol. Sci. Rev. Macromol. Chem. 23:61; Levy et al., 1985, Science 228:190-192; During et al., 1989, Ann. Neurol. 25:351-356; Howard et al., 1989, J. Neurosurg. 71:858-863). In yet another embodiment, a controlled release system can be placed in proximity of the therapeutic target, i.e., the brain, thus requiring only a fraction of the systemic dose (e.g., Goodson, 1984, In: Medical Applications of Controlled Release, supra, Vol. 2, pp. 115-138). Other controlled release systems are discussed in the review by Langer (1990, Science 249:1527-1533).

In a preferred embodiment, the method further comprises the step of modulating the binding affinity of the compound to the enzyme. This can be accomplished by methods known to the person skilled in the art, e.g. by a chemical modification of various residues of the compound and subsequent analysis of the binding affinity of the compound to the enzyme.

The invention further relates to the use of at least one broad spectrum enzyme ligand immobilized on a solid support for the characterization of at least one enzyme or of an enzyme-compound complex. With respect to this use of the invention, all embodiments as described above for the methods of the invention also apply.

The invention is further illustrated by the following figures and examples, which are not considered as being limiting for the scope of protection conferred by the claims of the present application.

### SHORT DESCRIPTION OF THE FIGURES

FIG. 1: Structures of kinobead ligands. The source and synthetic routes of the ligands are described in Example 1.

FIG. 1a: Kinobead ligand I (Bisindolylmaleimide VIII)

FIG. 1b: Kinobead ligand 2 (Purvalanol B)

FIG. 1c: Kinobead ligand 3 (CZC00007324, (7-(4-Aminomethyl-phenylamino)-3-(2,6-dichloro-phenyl)-1-methyl-1H-[1,6]naphthyridin-2-one))

FIG. 1d: Kinobead ligand 4 (CZC00008004, 2-(4′-aminomethyl phenylamine)-5-fluoro-pyrimidin-4-yl)-phenyl-amine)

FIG. 2: Signalokinome (see Example 2).
This figure shows a comparison of Drug pulldowns using kinobeads (left circle) and conventional immunoprecipitations with anti-phosphotyrosine antibody beads (right circle). In the Kinobeads experiment a total of 626 proteins were identified, of these are 100 kinases. In the immunoprecipitation (IP) experiment a total of 503 proteins were identified, 12 of these were kinases. Kinases identified with both experiments (common kinases) are listed in the overlap area. The result shows that with the kinobeads significantly more kinases were identified (100 kinases) compared to the anti-phosphotyrosine antibody beads (12 kinases).

FIG. 3: Structures of kinobeads ligands 5, 6 and 7. The synthetic routes of the ligands are described in Example 5, 6 and 7.

FIG. 3a: Structure of kinobead ligand 5 (indol ligand 91)

FIG. 3b: Structure kinobead ligand 6 (quinazoline ligand 32)

FIG. 3c: Structure kinobead ligand 7 (modified Staurosporine)

FIG. 4: Quantitative protein affinity profile (PAP).

The figure shows in lysate competition with test compound Bis VIII and detection of proteins with Western blot analysis. The pulldown experiment was performed as described in Example 8 with Jurkat cell lysate samples containing 10 mg of protein. Input lysate (lane L; 50 μg protein) and SDS-eluates from kinobeads (lanes 1 to 7) were separated on a SDS-polyacrylamide gel, transferred to a membrane and probed with antibodies.

FIG. 4A: The Western blot was probed first with an antibody directed against GSK3beta. Secondary detection antibodies labeled with peroxidase were used for chemiluminescent detection. As a loading control a blot was probed with an anti-ITK antibody. Lane L: 50 μg of Jurkat lysate; lane 1: 6.0 μM BisVIII; lane 2: 2.0 μM BisVIII; lane 3: 0.67 μM BisVIII; lane 4: 0.22 μM BisVIII; lane 5: 0.074 μM BisVIII; lane 6: 0.025 μM BisVIII; lane 7: 0.5% DMSO (solvent control).

FIG. 4B: Concentration dependent competition of GSK3 beta binding to kinobeads by Bis VIII. The GSK3beta bands on the Western blot shown in FIG. 2 A were quantified and plotted against the concentration of Bis VIII added to the lysate.

FIG. 5: Quantitative protein affinity profile for kinases

The results of the quantitative affinity profile experiment of example 8 are displayed for four kinases. Relative Intensity (RI) values are plotted against compound concentration (Bis VIII). The RI50 value represents the compound concentration at which the relative intensity of the MS signal for a given kinase is 50% compared to the DMSO control.

FIG. 5A: Curve for Glycogen Synthase Kinase 3 alpha (GSKa; RI50=72.7 nM)

FIG. 5A: Curve for Glycogen Synthase Kinase 3 beta (GSKb; RI50=95 nM)

FIG. 5C: Curve for protein kinase C alpha (PKCa; RI50=12.2 nM)

FIG. 5D: Curve for protein kinase C beta (PKCb; RI50=21.5 nM)

FIG. 6 Kinobeads use immobilized kinase inhibitors as affinity reagents.

Kinase profile of mixed kinase inhibitor beads (kinome beads or kinobeads). Seven broad selectivity kinase inhibitors were immobilized and simultaneously exposed to lysates of human cell lines and primary tissue. Bound proteins were identified by mass spectrometry. The number of spectrum-to-sequence matches was translated into a heat map as a semi-quantitative indicator of the amount of protein captured.

FIG. 7 Kinobeads coverage of the kinome.

Mass spectrometric analysis of kinobeads purifications from 14 human and rodent cell lines and tissues (human HEK 293, HeLa, Jurkat, K562, Ramos, THP-1, kidney, placenta; mouse heart, liver, brain, muscle, kidney; and rat RBL-2H3) led to the identification of 307 kinases (269 human and 196 rodent) across all branches of the phylogenetic tree. Kinases that were found both in human and rodent samples are shown as green dots, while the ones specific for either human or rodent are shown in blue or red respectively. Kinase tree adapted with permission from Cell Signaling Inc. (www.cellsignal.com).

FIG. 8 Proteomic profiling of drugs in cell lysate by a kinobeads competition assay.

(a) Schematic overview of the kinobeads assay. Either lysates or cells are treated with vehicle and with compound over a range of concentrations (upper panel). Subsequently, proteins are captured on kinobeads. The ‘free’ inhibitor competes with the immobilized ligands for ATP-binding or related ligand-binding sites of its targets (middle panels). Bound proteins are digested with trypsin and each peptide pool is labeled with iTRAQ reagent (not shown). All four samples are combined and analyzed by mass spectrometry. Each peptide gives rise to four characteristic iTRAQ reporter signals (scaled to 100%) indicative of the inhibitor concentration used (bottom left panel). For each peptide detected, the decrease of signal intensity compared to the vehicle control reflects competition by the ‘free’ compound for its target (bottom right panel).

(b) Examples of competition binding curves calculated from iTRAQ reporter signals. Binding of several known and novel targets to kinobeads is shown as dependent on the addition of imatinib (blue), dasatinib (green), and bosutinib (red) to K562 cell lysate. For each compound, three independent quadruplexed experiments (vehicle plus three compound concentrations each) were performed in duplicates, and iTRAQ reporter signal data were combined to display the dose response over 9 different concentrations.

(c) Kinase binding profiles of the ABL inhibitors imatinib (upper panel), dasatinib (middle panel), and bosutinib (bottom panel) across a set of protein kinases simultaneously identified from K562 cells. The bars indicate the IC50 values, defined as the concentration of drug at which half-maximal competition of kinobeads binding is observed.

FIG. 9 Targets of imatinib.

(a) Western blot analysis of proteins captured on kinobeads. Top panel: Imatinib treatment of K562 lysate reduces the amount of DDR1 captured on kinobeads. Second panel: Imatinib treatment of K562 cells in culture similarly reduces the amount of DDR1 captured on kinobeads. Using a phosphorylation-specific Y703P-KIT antibody (third panel) and a general KIT antibody (bottom panel) shows that only the Y703P-KIT species are affected by imatinib. The different mobilities of the Y703P-KIT bands may reflect differential phosphorylation and/or ubiquitination.

(b) Inhibition of DDR1 autophosphorylation in K562 cells by imatinib. Cells were treated with pervanadate to induce tyrosine autophosphorylation of DDR1. DDR1 was analyzed by immunoprecipitation and western blotting with anti-phosphotyrosine antibodies (4G 10, upper panel) and DDR1 antibodies (middle panel). Pre-incubation of the cells with imatinib (lane 4) reduces the pervanadate-induced tyrosine phosphorylation and phosphorylation-mediated degradation of DDR1 (lane 3).

(c) Imatinib is a potent inhibitor of the tyrosine receptor kinases DDR1 and DDR2. The enzymatic activity of a purified recombinant fragment of human DDR1 containing the cytoplasmic kinase domain was measured in radiometric assays of DDR1 autophosphorylation (triangles, IC₅₀₌₂₂ nM) and the activity towards a substrate peptide (squares, IC50=31 nM). Imatinib also inhibits the only human DDR1 paralogue, DDR2 (circles, IC50=112 nM).

(d) Imatinib is a potent competitive inhibitor of the oxidoreductase NQO2. Recombinant human NQO2 was assayed spectrophotometrically in a coupled redox reaction using menadione as substrate, the nicotinamide analogue CMCDP as co-substrate, and MTT as indicator. Competitive inhibition is demonstrated by determining apparent Kₘ values for the co-substrate at different imatinib concentrations (Kᵢ=39 nM, see inset).

FIG. 10 Phosphorylation analysis of kinobeads-captured proteins to assess targets and downstream effects of imatinib.

(a) Dose-dependent reduction of regulatory phosphorylation sites in imatinib-treated K562 cells (triangles) or lysates (squares) of regulatory sites on Csk (upper left panel) and RSK2 (bottom left panel).

(b) Schematic representation of the proposed mechanism of action of imatinib in K562 chronic myelogenous leukemia cells. Direct targets (blue symbols) bind directly to the drug, or are associated in a complex with proteins directly binding and hence exhibit decreased binding to kinobeads in the presence of the drug. Indirect targets (white symbols) represent substrates of the direct targets. They do not bind directly to the drug and hence their binding to kinobeads is not affected, but they do exhibit reduced phosphorylation of potential or known regulatory sites. Imatinib binds to its direct target, which appears to be a BCR-ABL/GRB2/SHC/SHIP2/STS-1 complex, since all of these proteins are competed by imatinib (and also by dasatinib and bosutinib) with similar characteristic potencies. Additional direct imatinib targets are the kinases Arg, DDR1, and KIT, and the oxidoreductase NQO2. Inhibition of the constitutively active BCR-ABL kinase leads to down-regulation of the MAP kinase pathway and subsequent prevention of nuclear entry and transcriptional activation of RSK kinases.

FIG. 11 Kinase binding profiles of individual immobilized tool compounds and drugs. The research tool inhibitors Bis (III) indolyl maleimide (protein kinase C inhibitor), purvalanol B (cyclin-dependent kinase inhibitor), CZC8004 and staurosporine (pan-kinase inhibitors); and the drugs or drug candidates PD173955 (Src kinases), vandetanib (VEGFR, EGFR), sunitinib (VEGFR, PDGFR, Flt3, KIT), Ro 320-1195 (p38 MAP kinase), imatinib (ABL, PDGFR, KIT), gefitinib (EGFR), pelitinib (EGFR), and lapatinib (EGFR, Her-2) were immobilized and exposed to lysates from HeLa or K562 cells. Bound proteins were identified by mass spectrometry. The number of spectrum-to-sequence matches was translated into a heat map as a semi-quantitative indicator of the amount of protein captured.

FIG. 12 iTRAQ-based quantification of the proteins captured on kinobeads.

Distribution of iTRAQ areas for all proteins identified on kinobeads. Gray bars represent kinases, white bars represent non-kinases. According to Table 23, 13% of all proteins identified on kinobeads are protein kinases. However, when using the total iTRAQ area as a measure of protein quantity, it is interesting to note that 79% of the total protein is represented by protein kinases (gray bars) compared to 21% for other proteins.

FIG. 13 Examples of competition binding curves calculated from iTRAQ reporter signals.

Binding of several known and novel targets to kinobeads is shown as dependent on the addition of irnatinib (triangles), dasatinib (diamonds), or bosutinib (squares) to K562 cell lysate. Competition binding data were recorded from duplicate experiments (defined as two parallel compound treatments, carried out using the same batch of K562 cell lysate used throughout this study) over 6 different concentrations. In this figure, all replicated experiment are shown as separate points; curves were fitted to the averaged value of each duplicate, while the top of the curve was fixed to 1 (vehicle control).

FIG. 13A: Binding curves for imatinib (triangles)

FIG. 13B: Binding curves for dasatinib (diamonds)

FIG. 13C: Binding curves for bosutinib (squares)

FIG. 14 Focal adhesion kinase (FAK/PTK2) binds dasatinib only in an activated conformation.

The graphs show the dose-dependent reduction of regulatory phosphorylation sites in dasatinib-treated K562 cells (triangles) or lysates (squares) of a double-phosphorylated regulatory site on focal adhesion kinase (FAK). Whereas the FAK total protein level is only affected at high compound concentrations, a subset of FAK represented by phosphorylation on Y598/599 is affected when dasatinib was added to the lysate (gray squares), and even more strongly affected when dasatinib was added to the cultured cells (gray triangles).

FIG. 15 Proteomic target profiling of drugs in cultured cells by a kinobeads competition assay.

FIG. 15A: Examples of competition binding curves calculated from iTRAQ reporter signals. Binding of selected known and novel targets to kinobeads is shown as dependent on the treatment of K562 cells with imatinib (triangles), dasatinib (diamonds), and bosutinib (squares) in culture, before cells are lysed. For each compound, three independent quadruplexed experiments (vehicle plus three compound concentrations each) were performed in duplicates, and iTRAQ reporter signal data were combined to display the dose response over 9 different concentrations.

FIG. 15B: Kinase binding profiles of the ABL kinase inhibitors imatinib (right panel), dasatinib (middle panel), and bosutinib (left panel) across a set of protein kinases simultaneously identified from K562 cells treated with the drugs in culture. The bars indicate the IC50 values, defined as the concentration of drug at which half-maximal competition of kinobeads binding is observed.

FIG. 16 Fraction of initial protein captured on beads.

FIG. 17 Compounds for immobilization to beads in target profiling with immobilized kinase inhibitors

FIG. 18 Kinase binding profiles of individual immobilized tool compounds and drugs

FIG. 19 Protein binding profiles of mixed kinase inhibitor beads (kinobeads)

FIG. 20 Kinobeads competition data calculated from iTRAQ reporter signals for bosutinib, dasatinib, and imatinib in K562 cell lysate

FIG. 21 Kinobeads competition data calculated from iTRAQ reporter signals for compounds added to K562 cells in culture

FIG. 22 Phosphopeptides identified from Kinobeads samples

FIG. 23 Dose-dependent reduction of regulatory phosphorylation sites in drug-treated cells

FIG. 24 Layout of quantitative Kinobeads experiments

FIG. 25 Overview of individual experiments performed in Example 9

FIGS. 17-25 and Supplementary Tables 1 and 2 were published as supplmentary material to Bantscheff et al. Nature Biotechnology 2007, 25:1035-1044, which is hereby incorporated by reference.

### EXAMPLES

### Example 1

### Preparation of Kinobeads

This example illustrates the preparation of kinobeads with 4 different ligands. These kinobeads were later used in example 2 and example 3.

Broad spectrum capturing ligands were covalently immobilized on a solid support through covalent linkage using suitable functional groups (e.g. amino or carboxyl or groups). Compounds that do not contain a suitable functional group were modified in order to introduce such a group. The necessary chemical methods are known in the medicinal chemistry literature and illustrated below.

### 1. Selection and Synthesis of Ligands

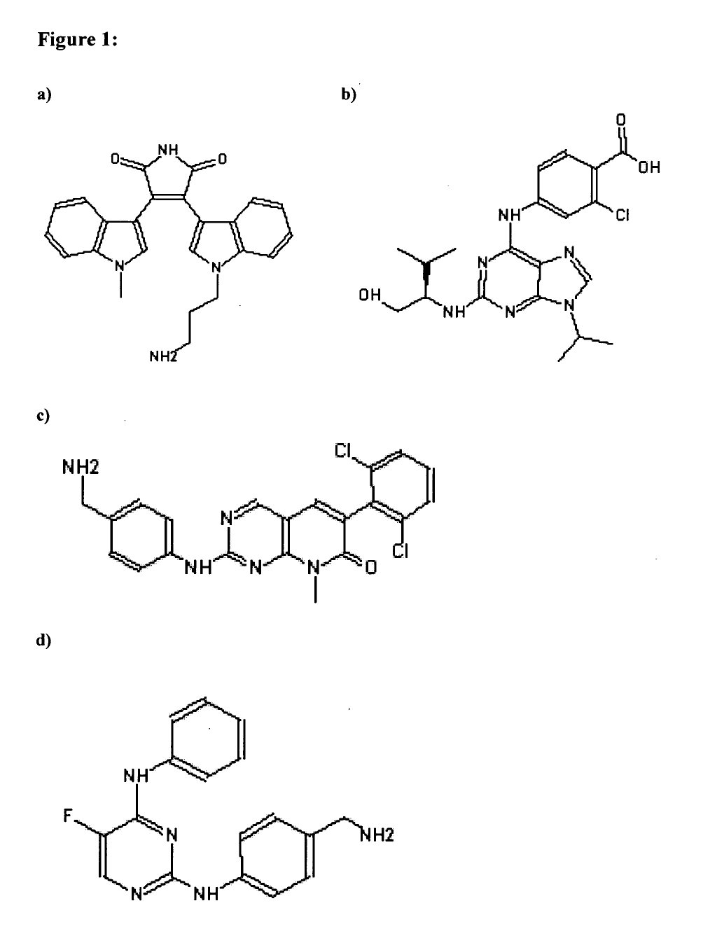

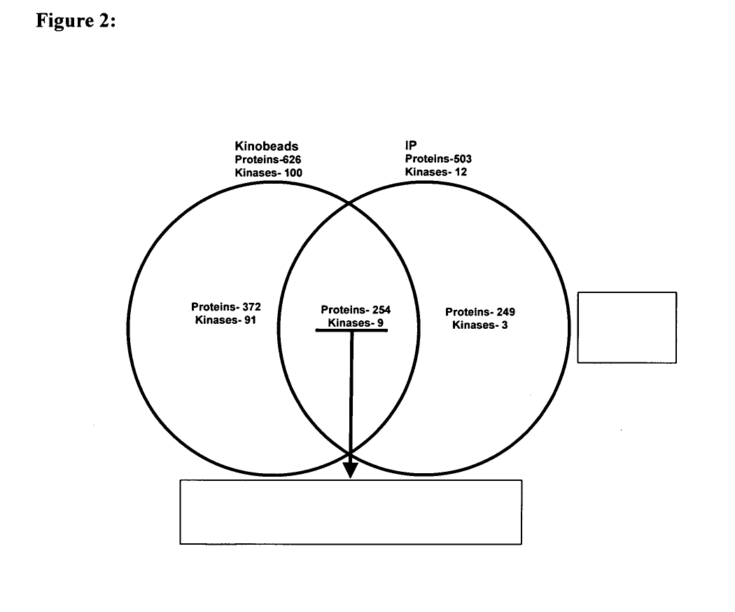

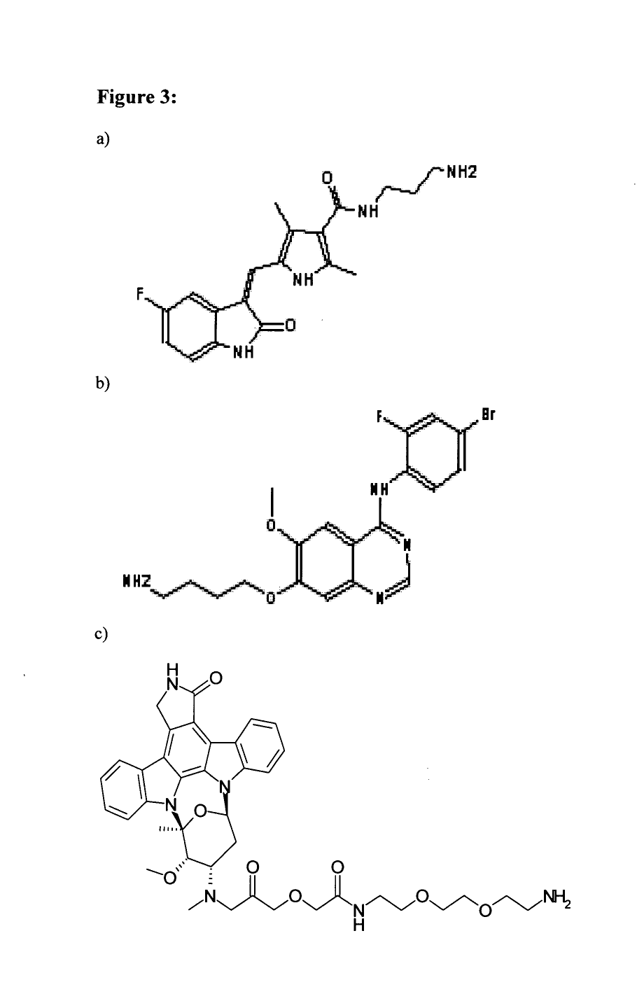

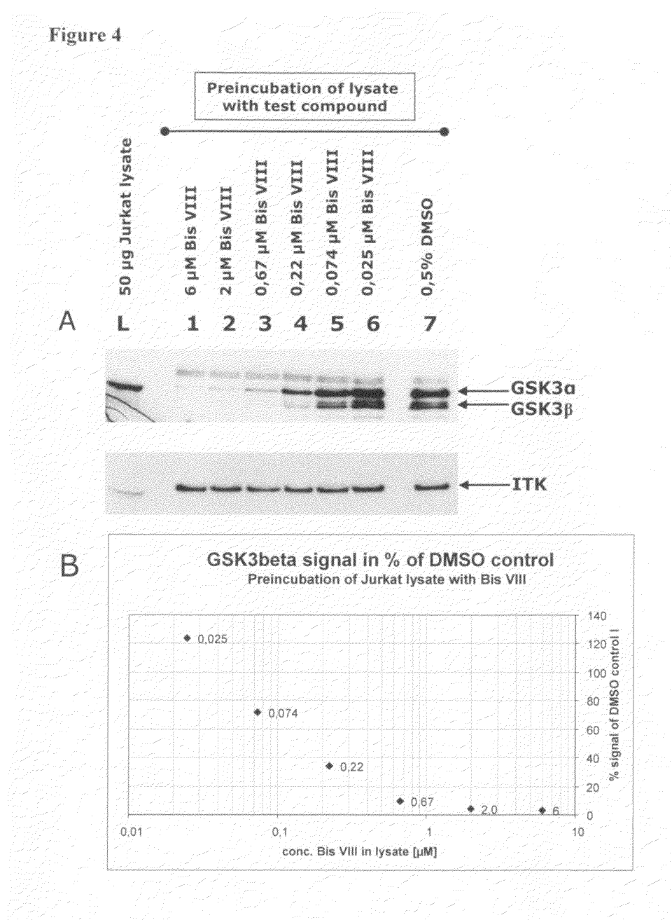

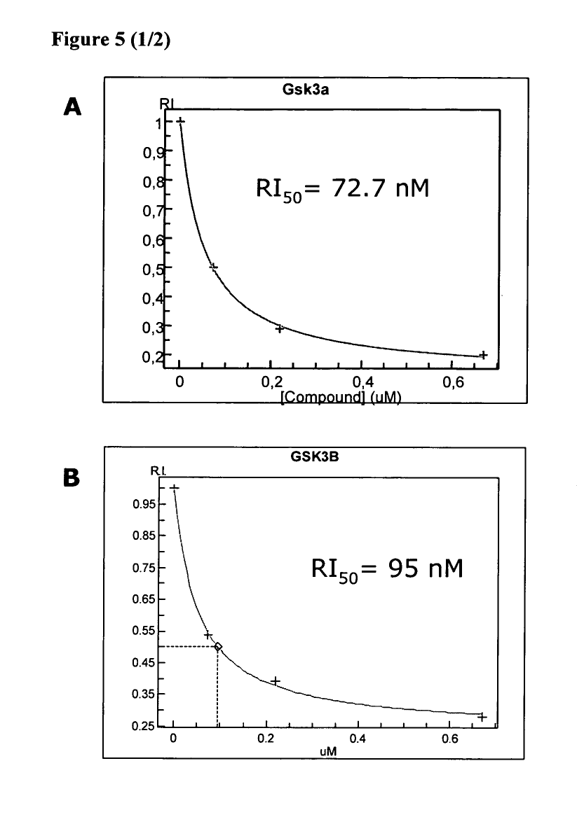

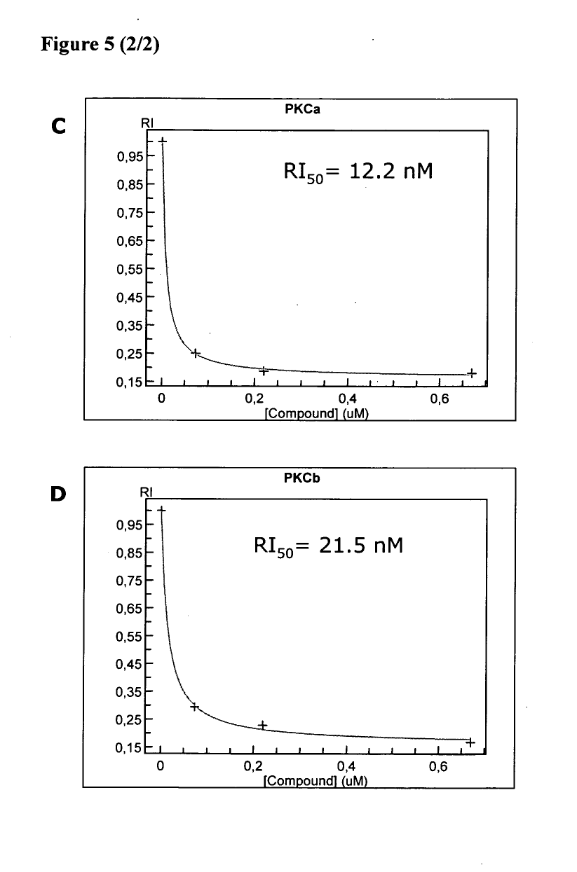

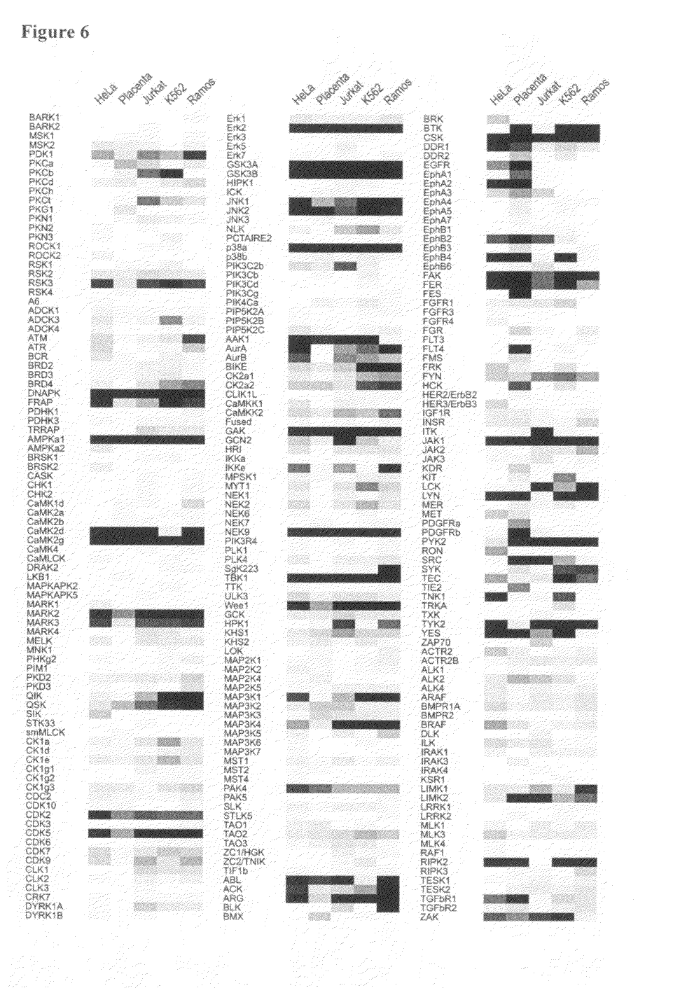

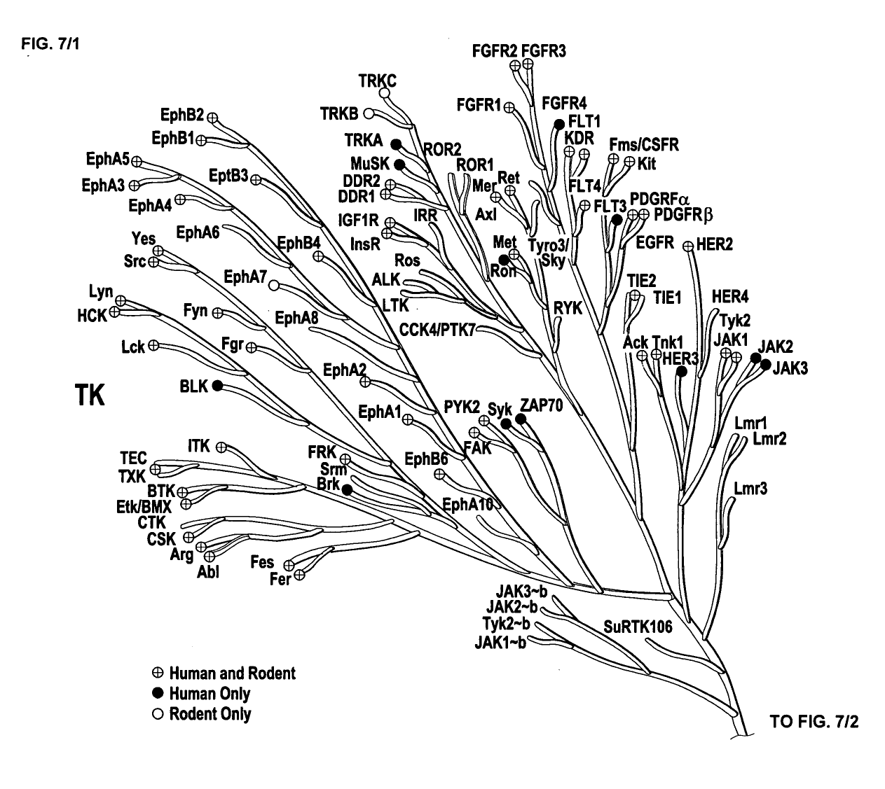

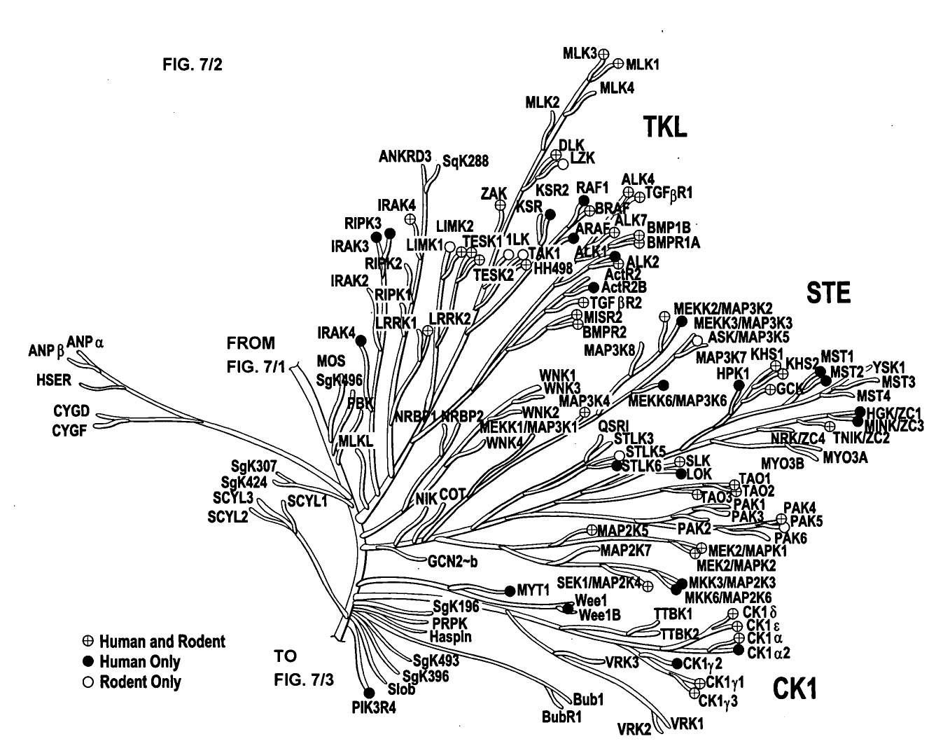

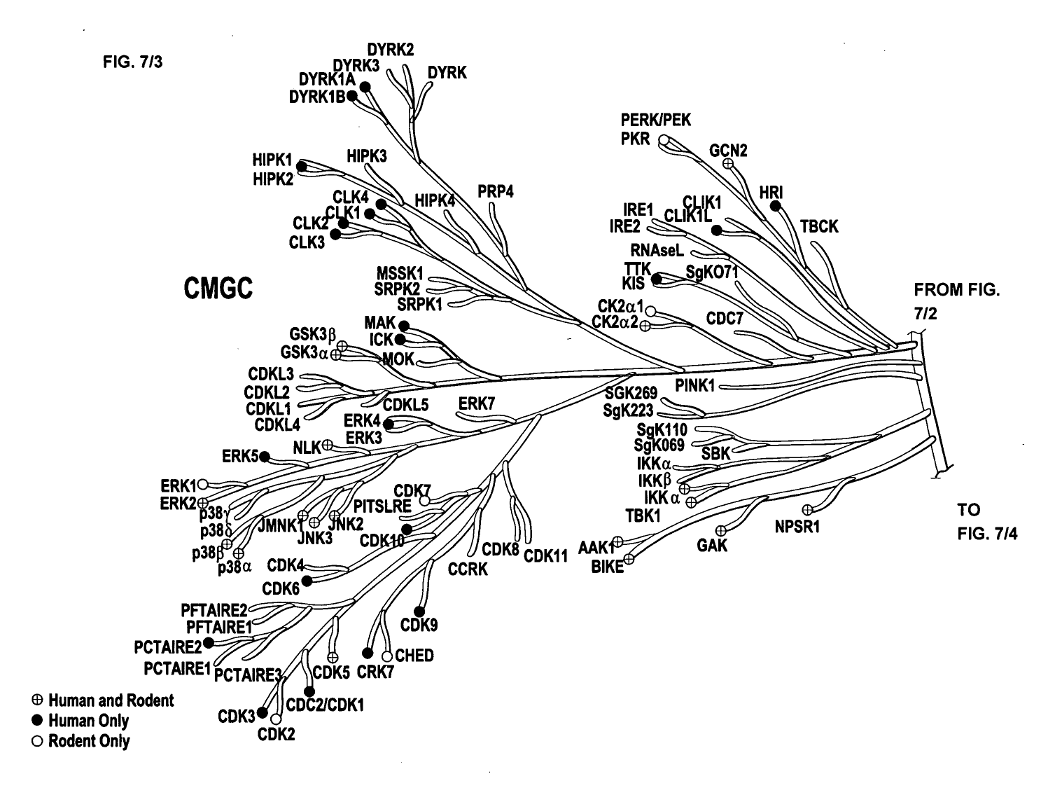

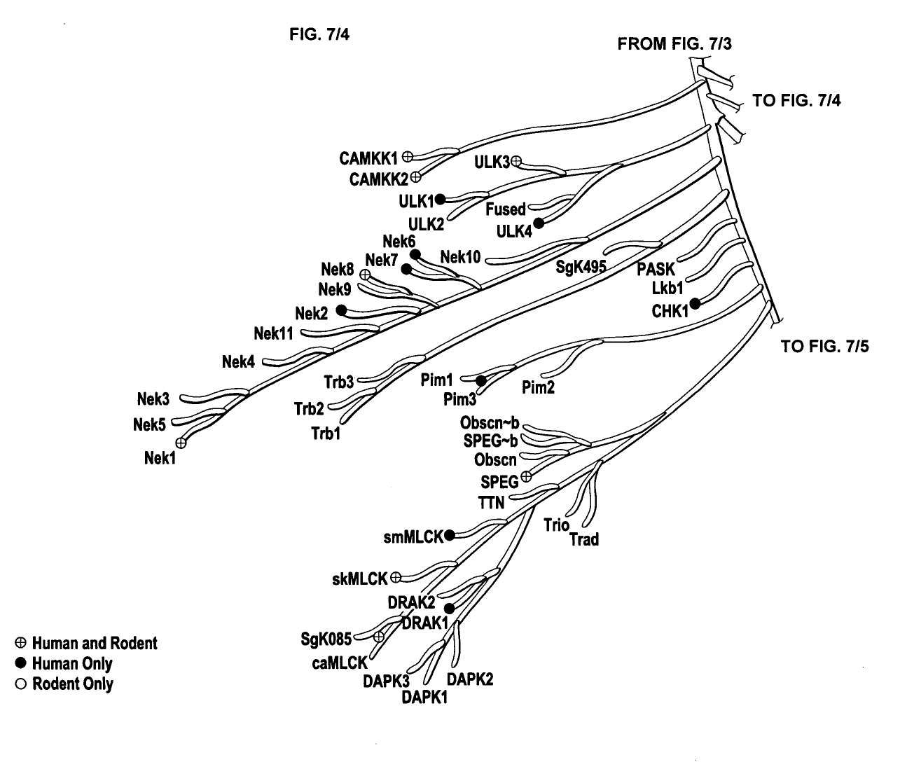

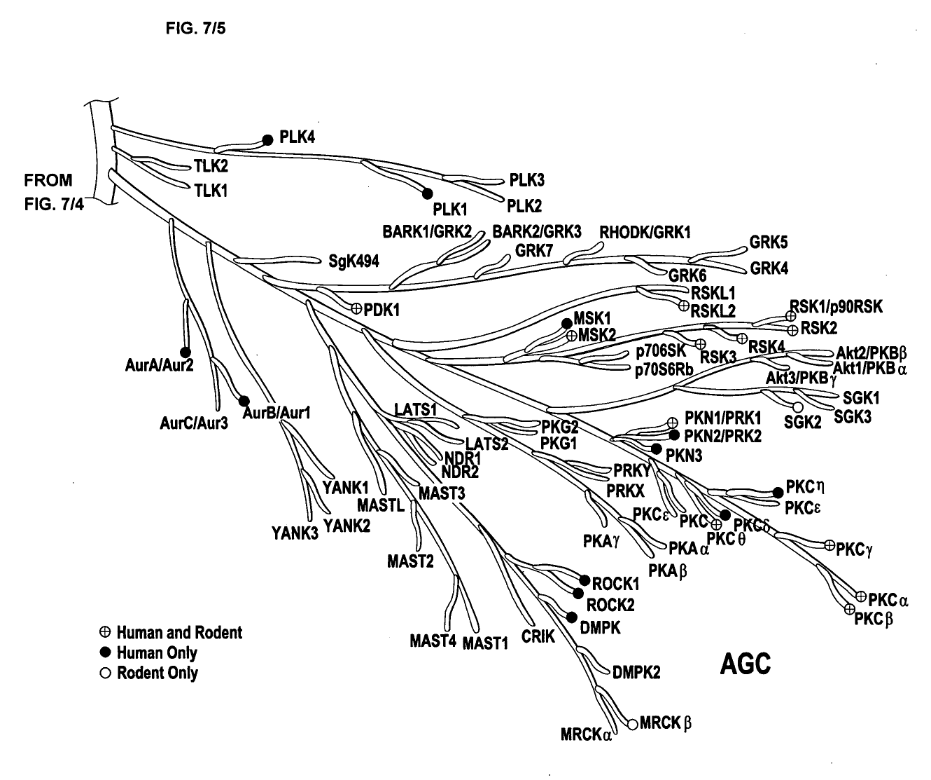

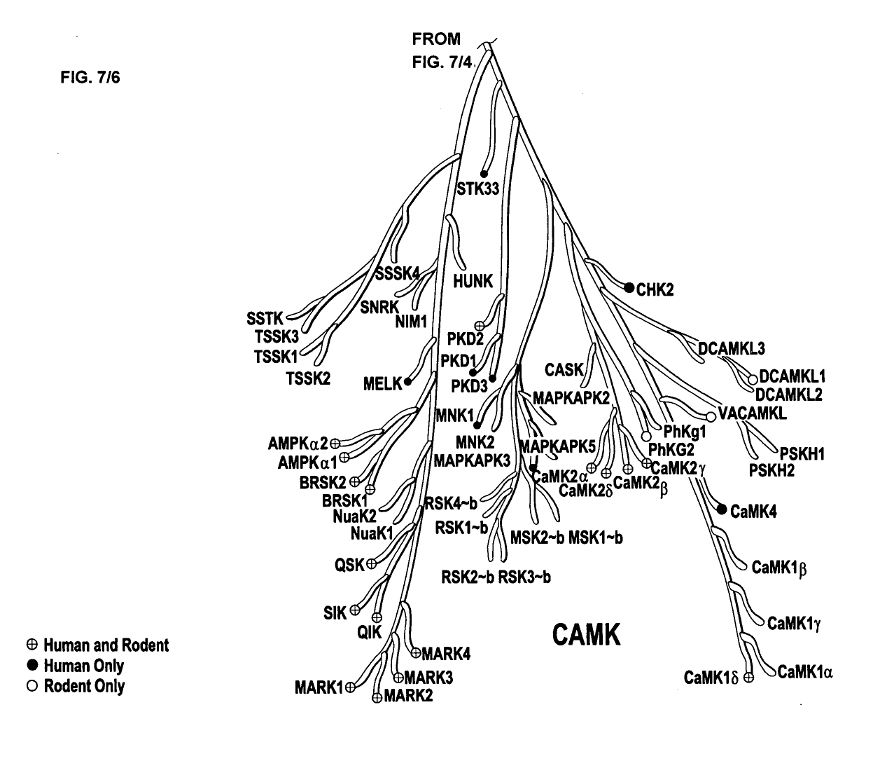

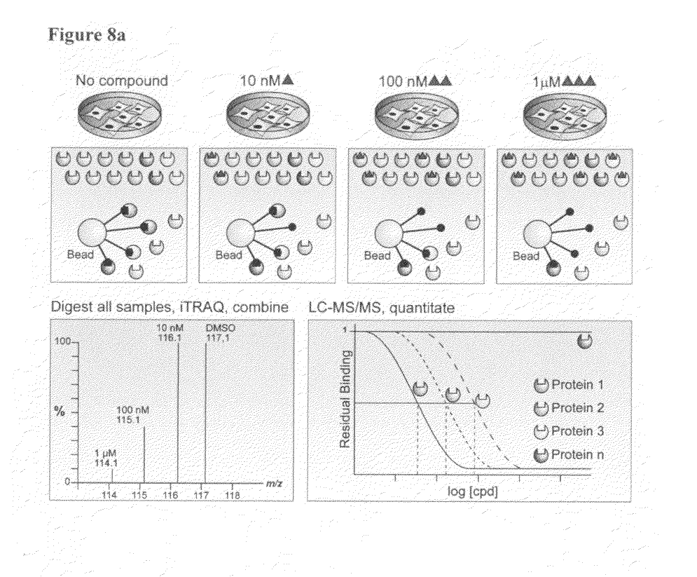

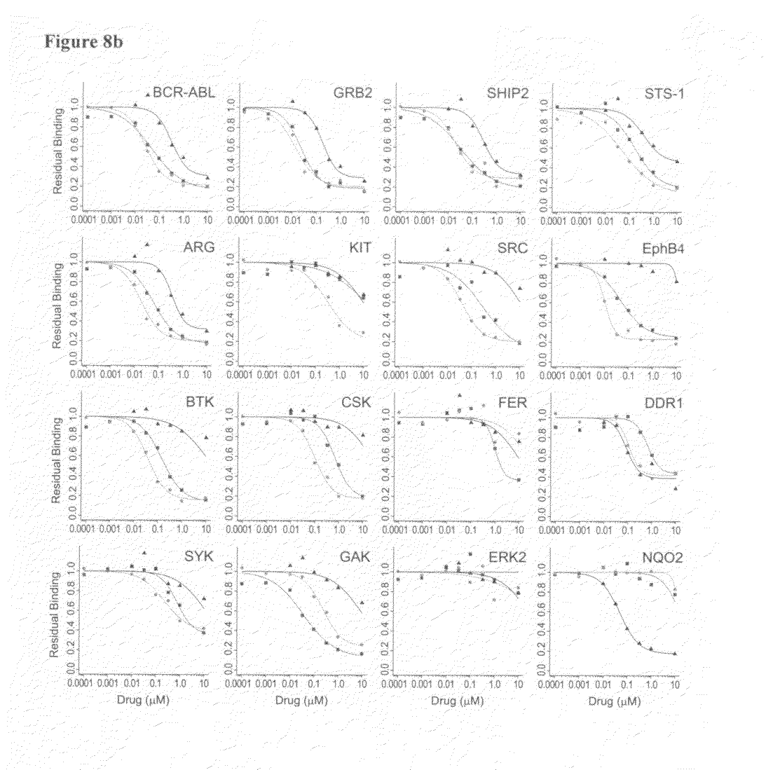

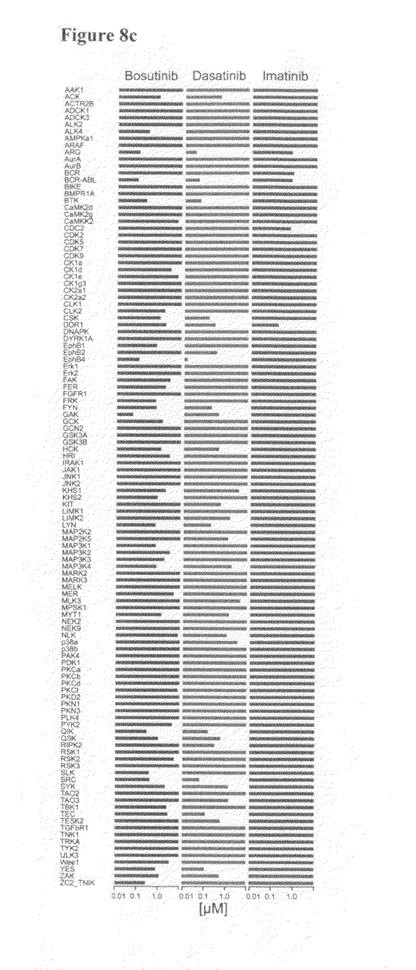

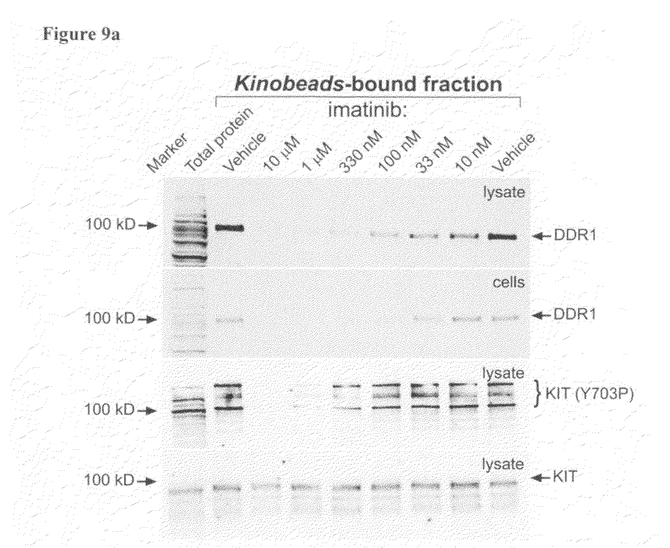

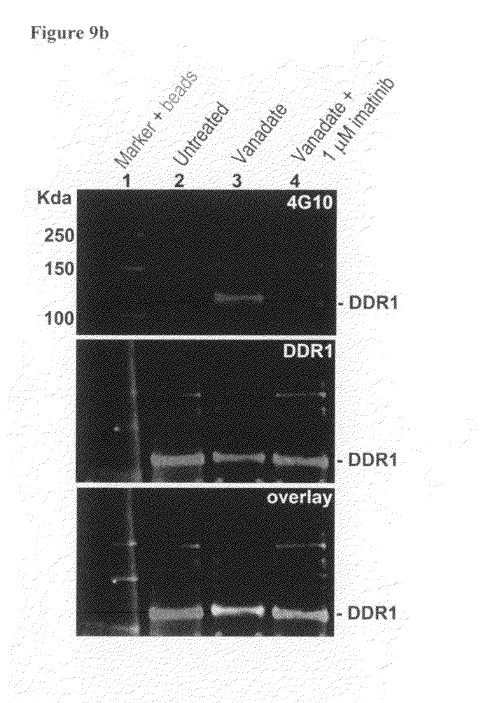

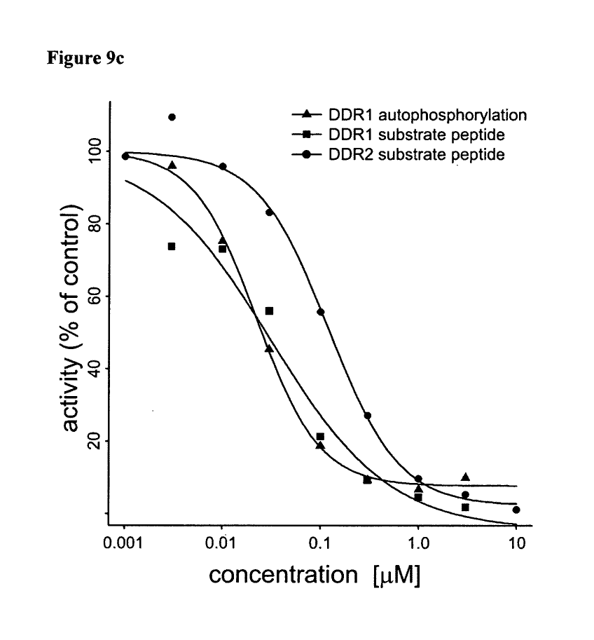

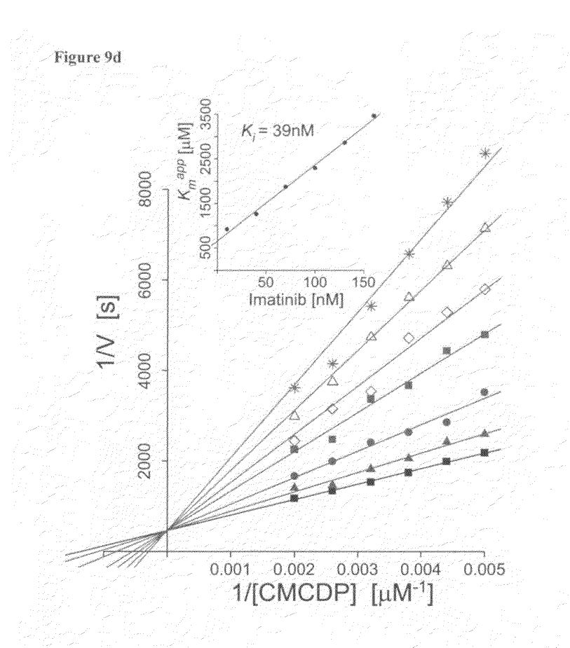

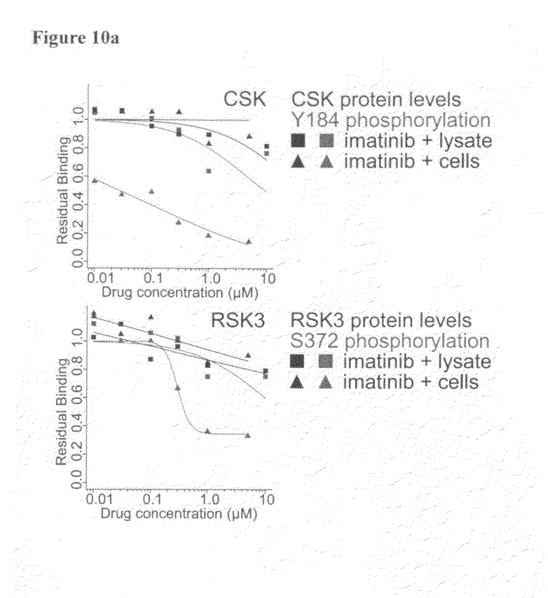

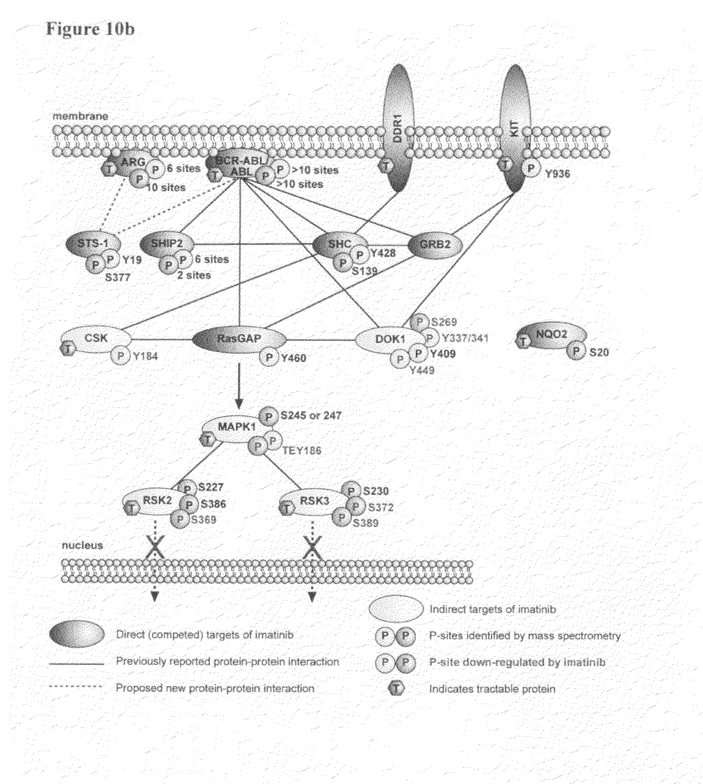

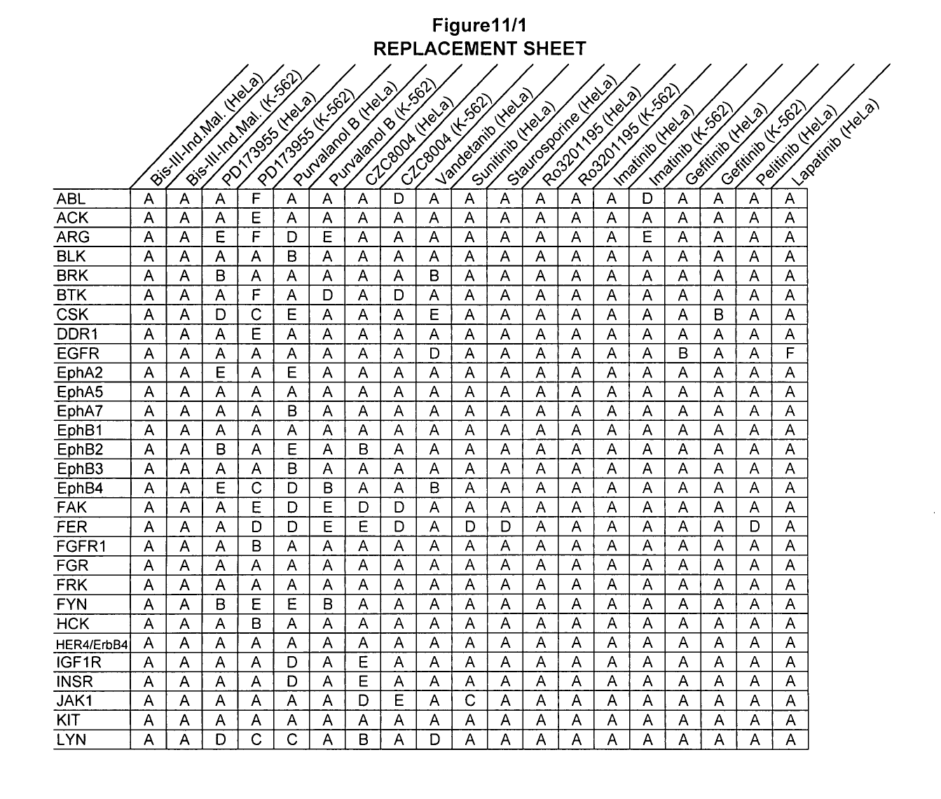

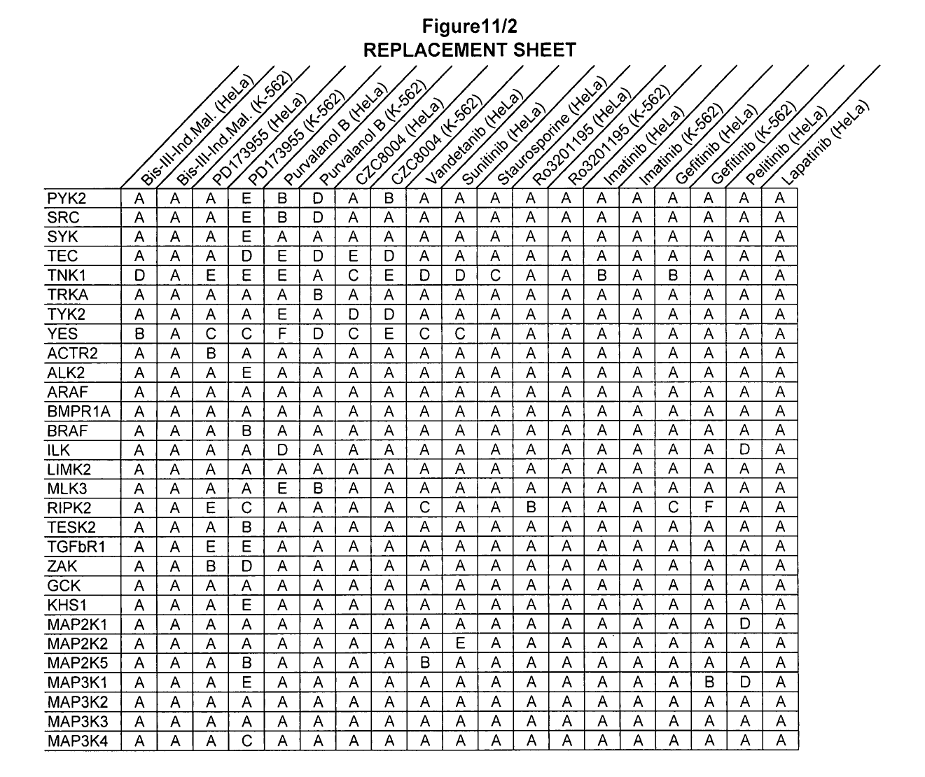

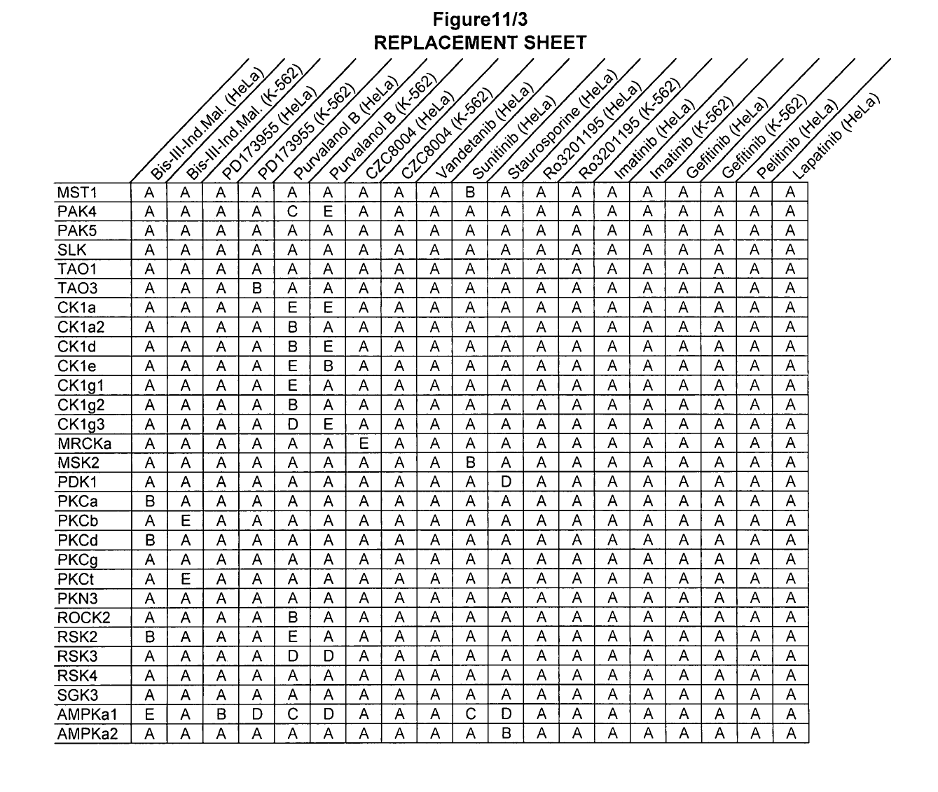

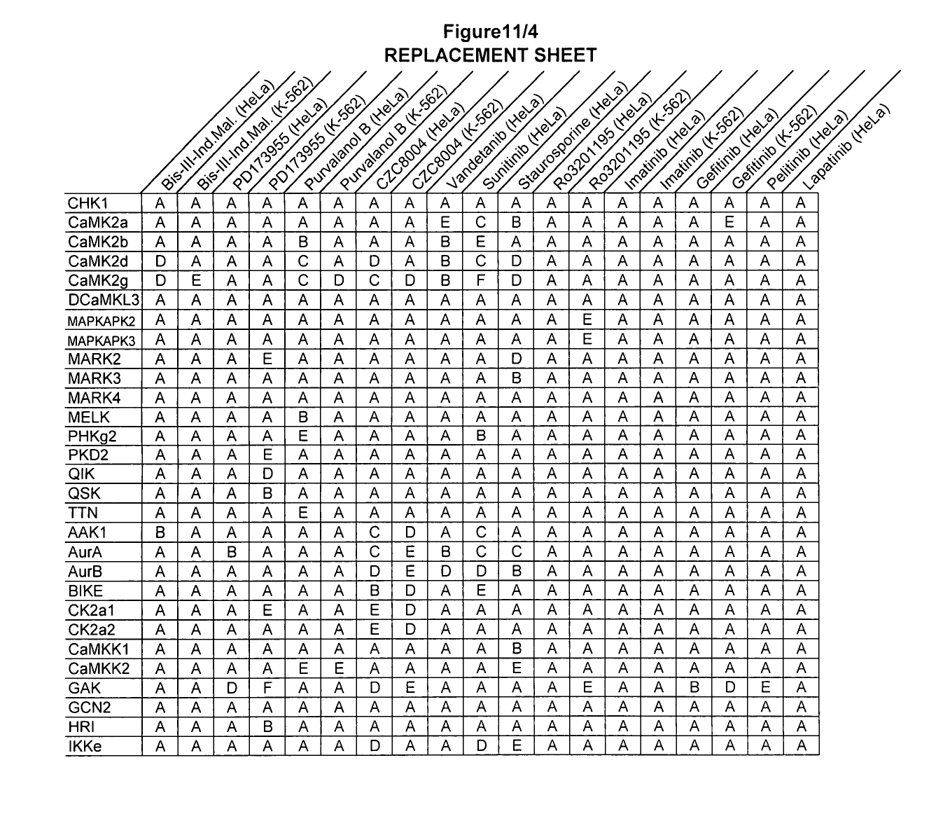

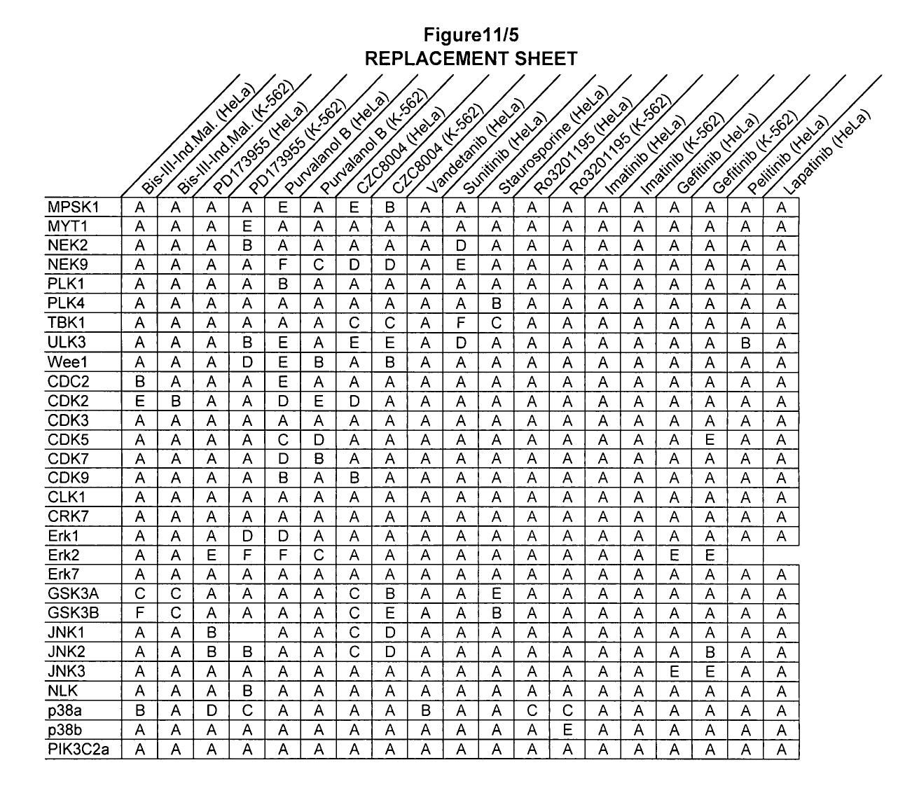

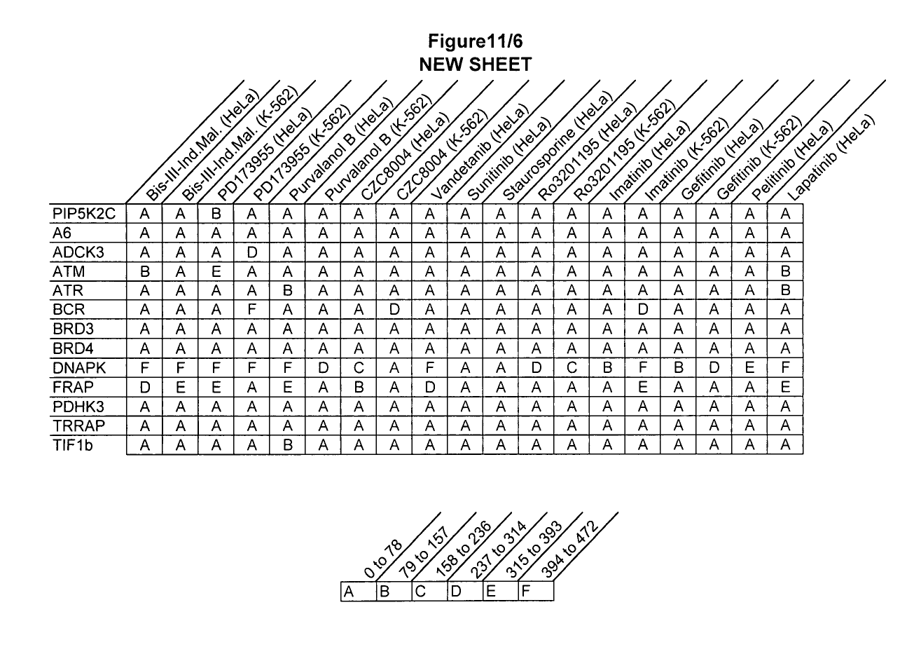

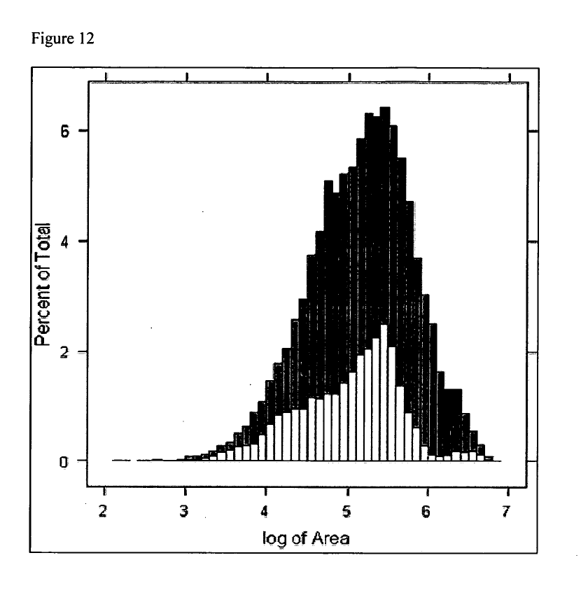

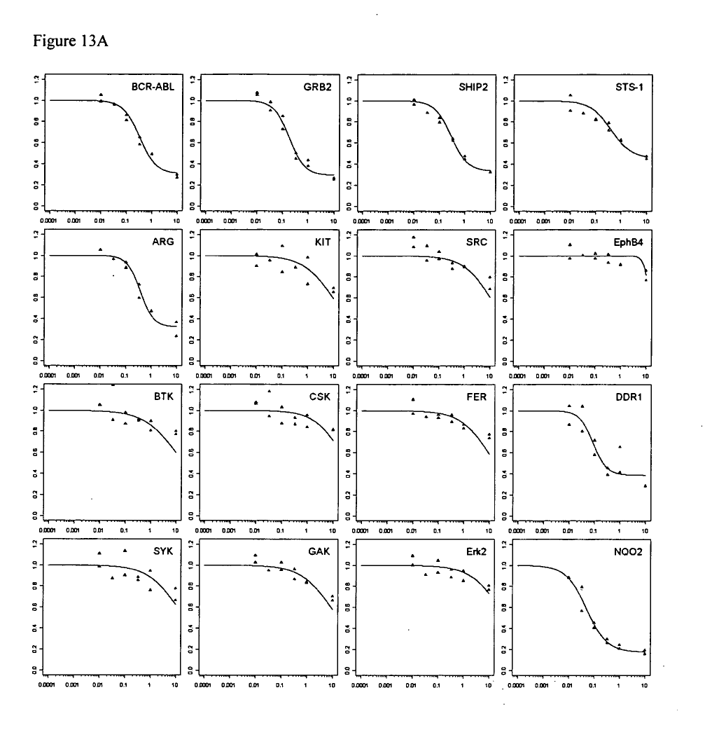

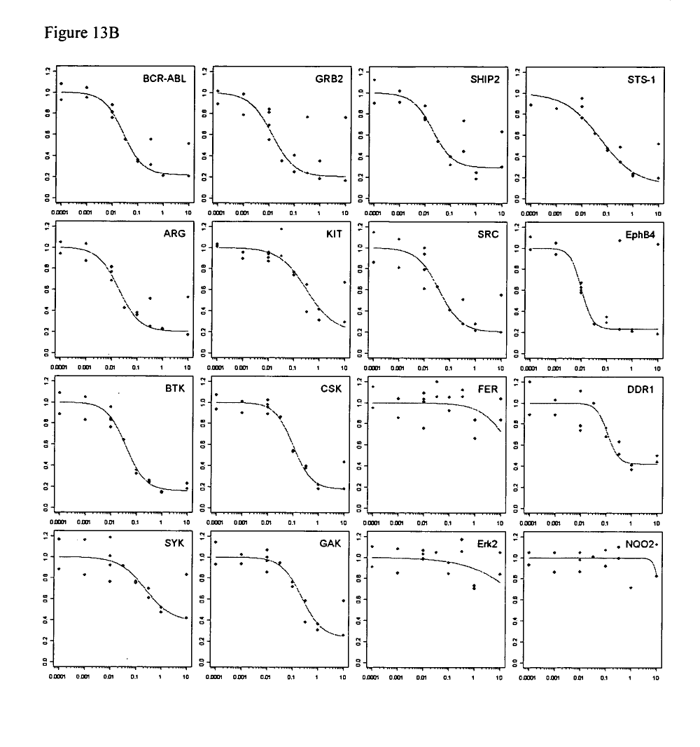

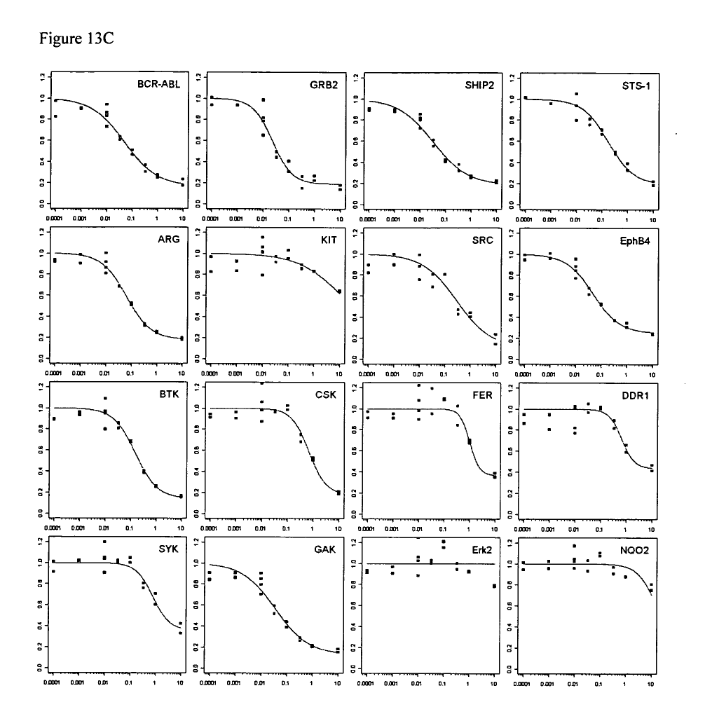

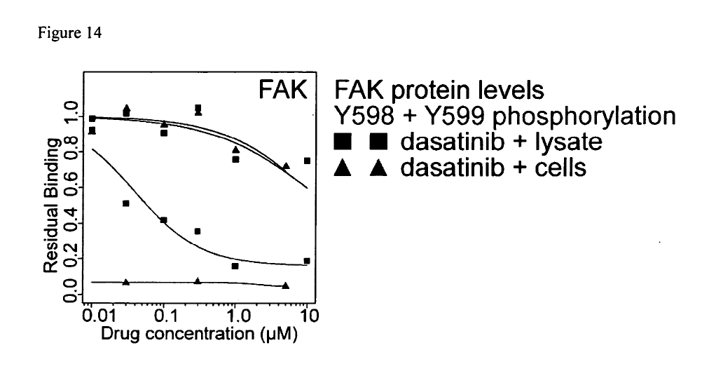

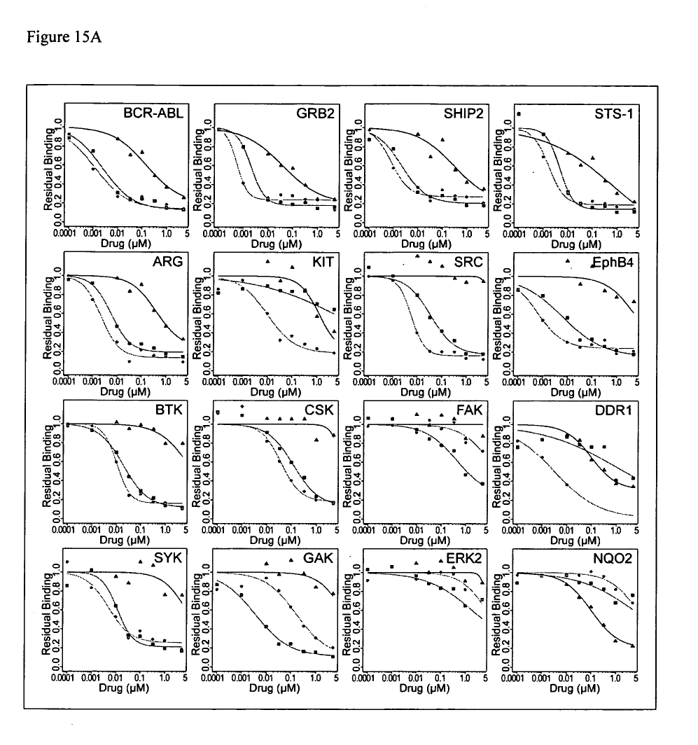

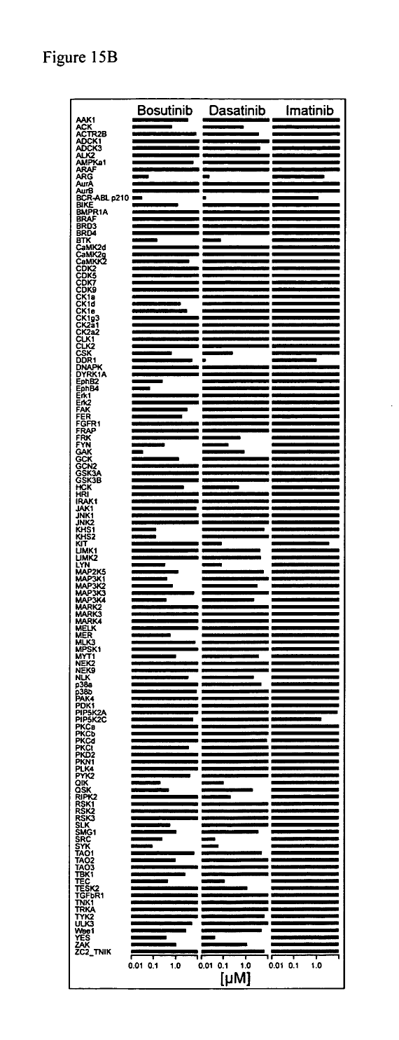

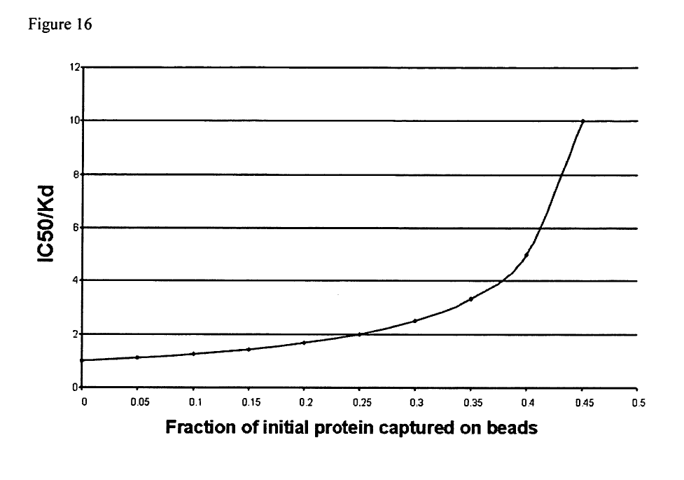

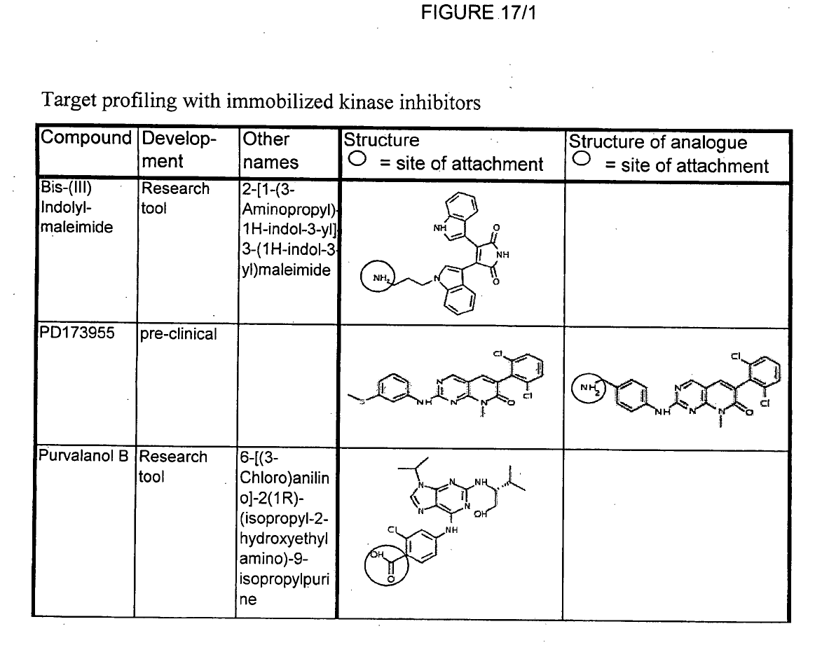

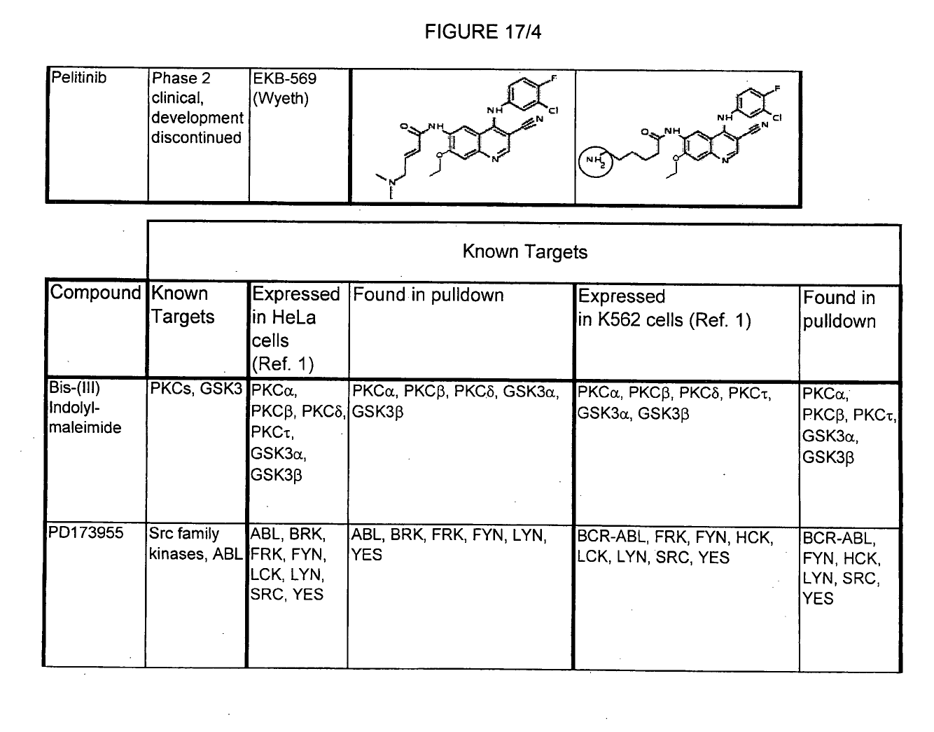

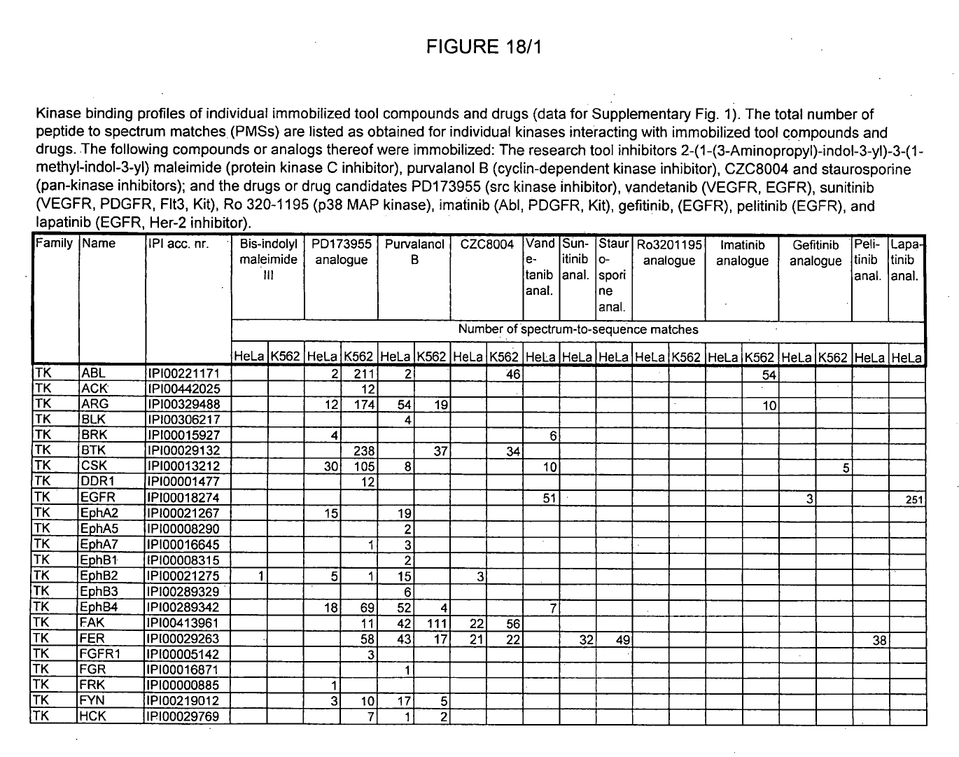

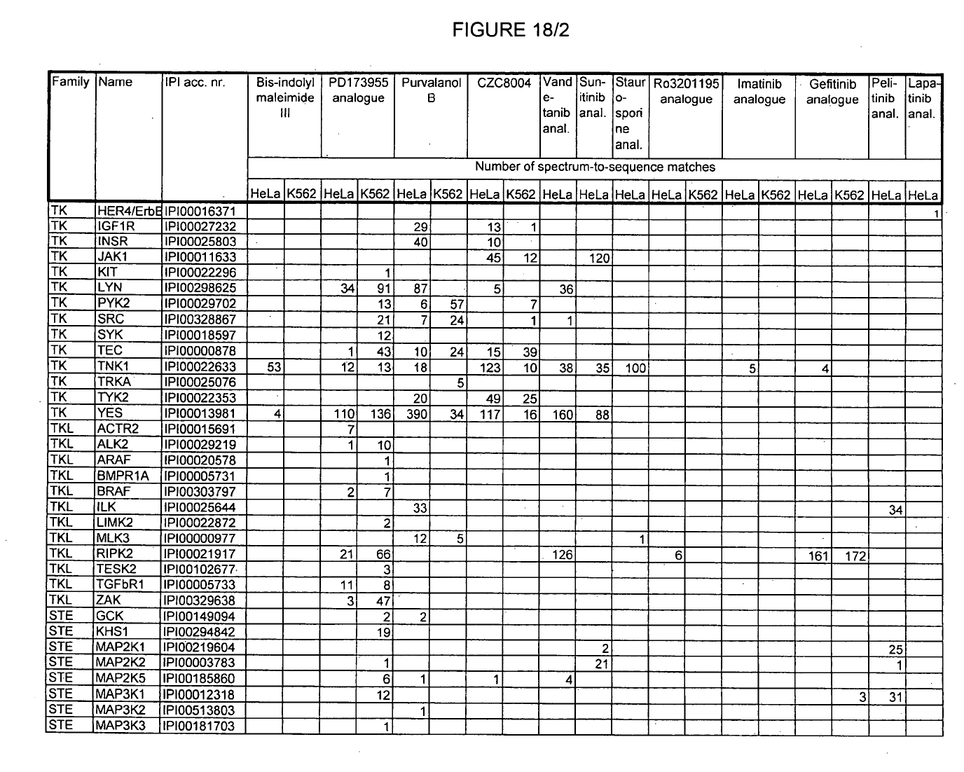

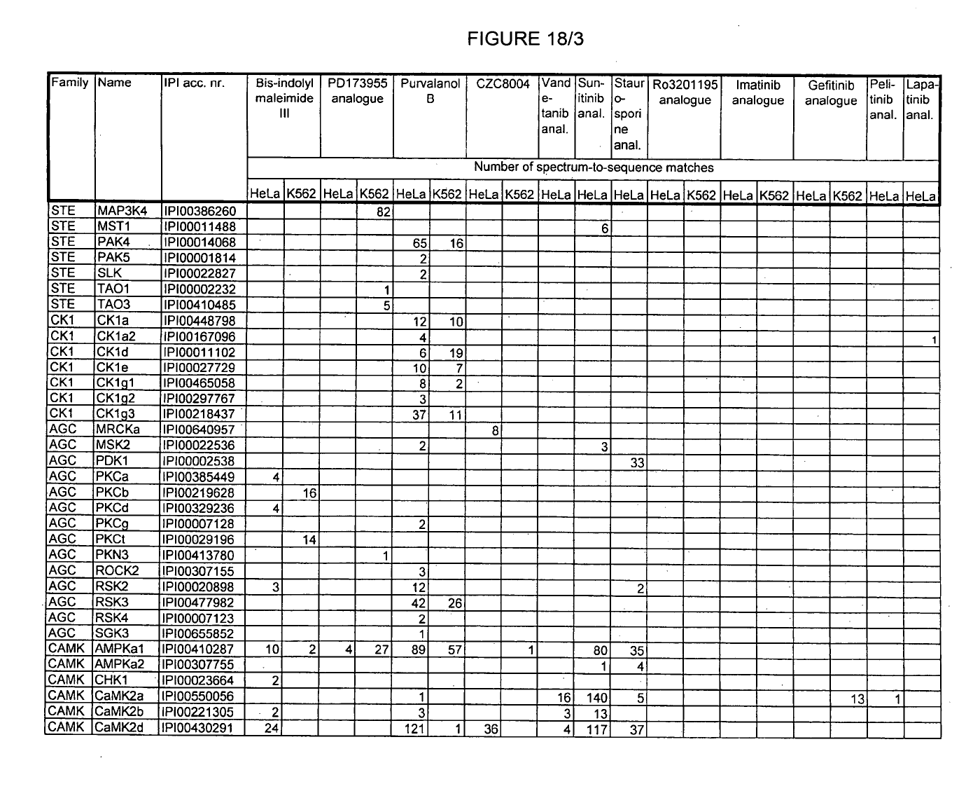

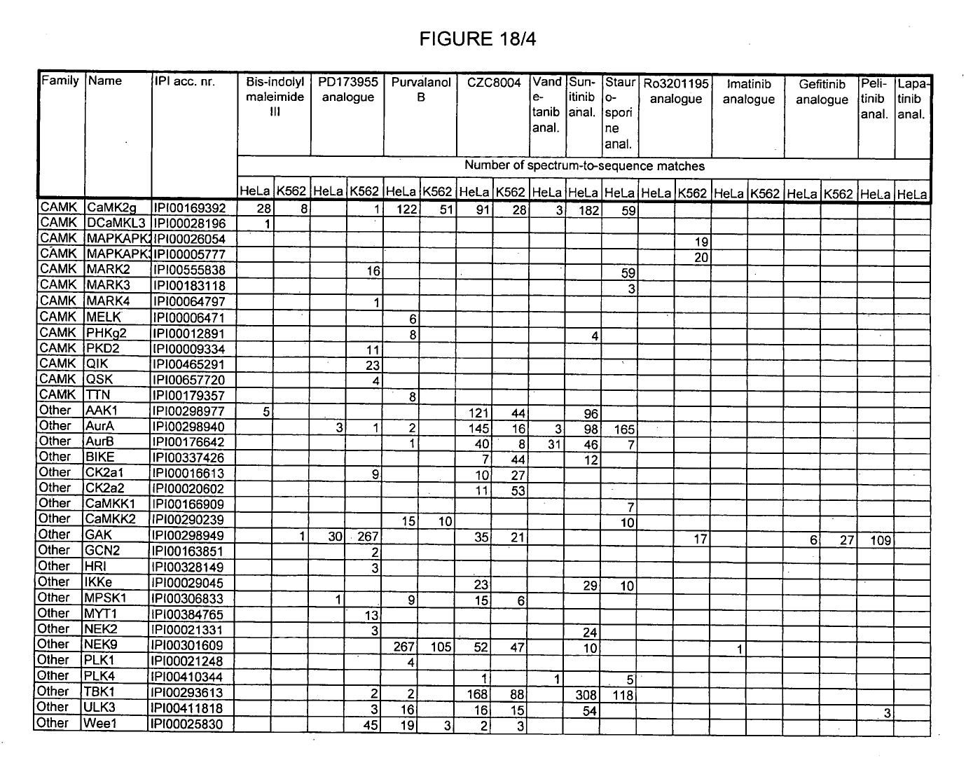

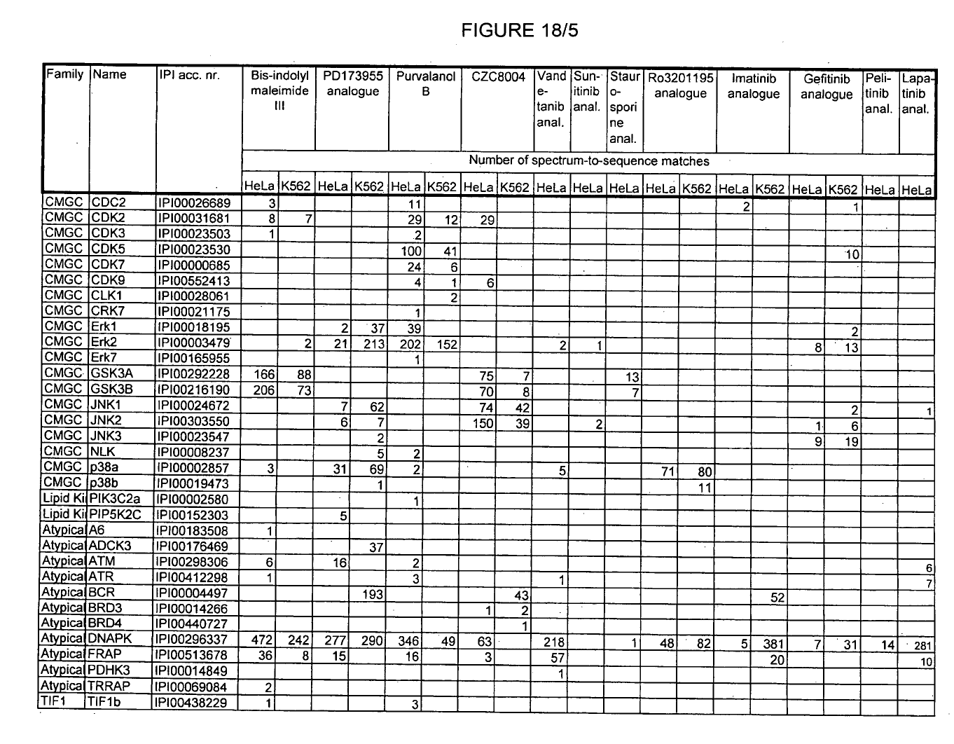

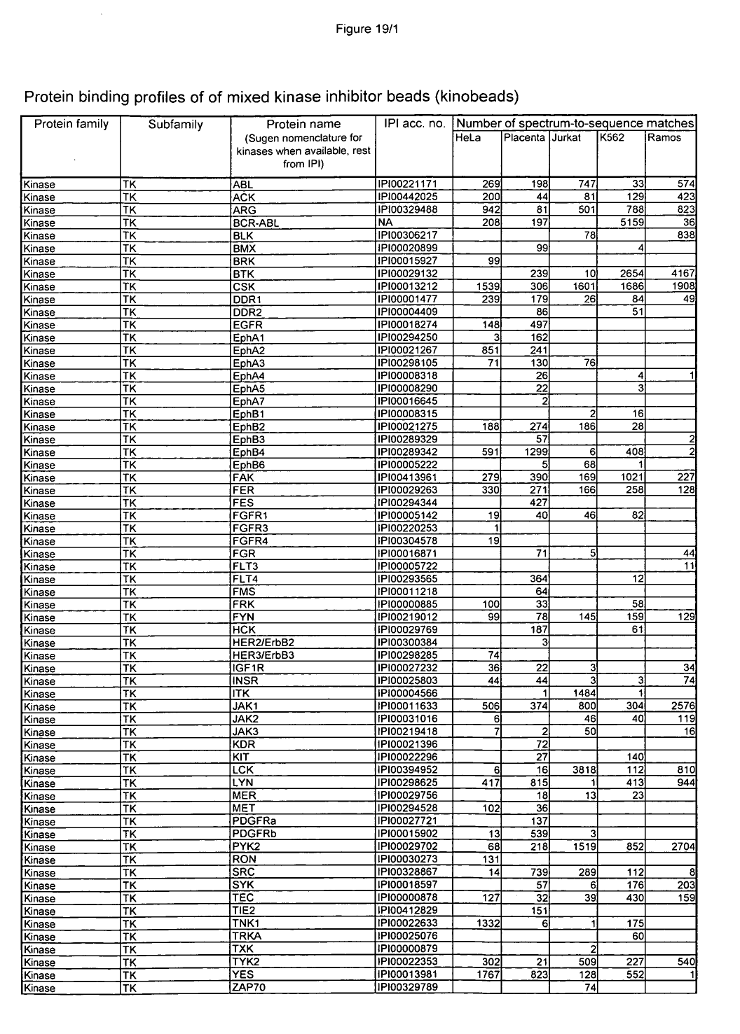

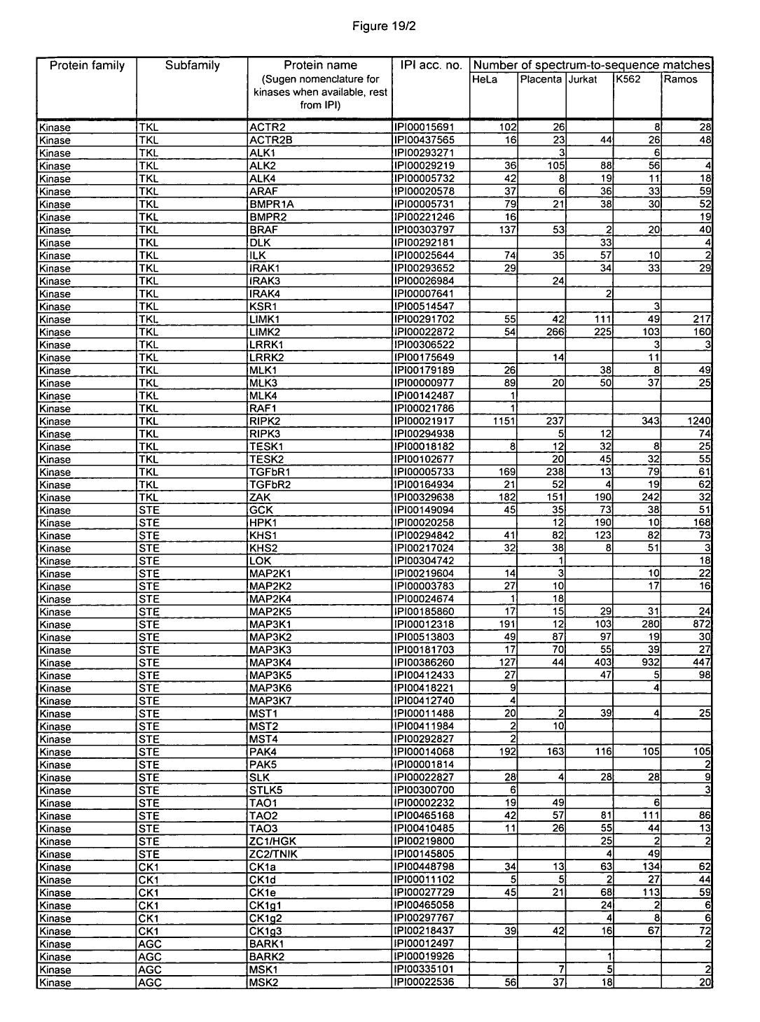

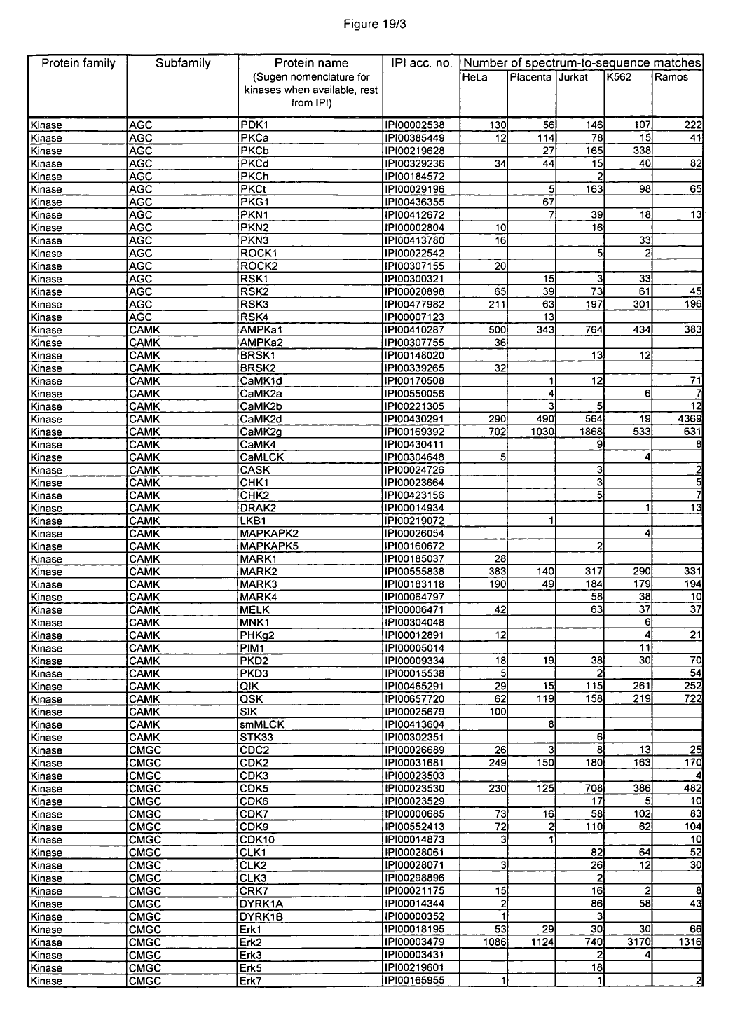

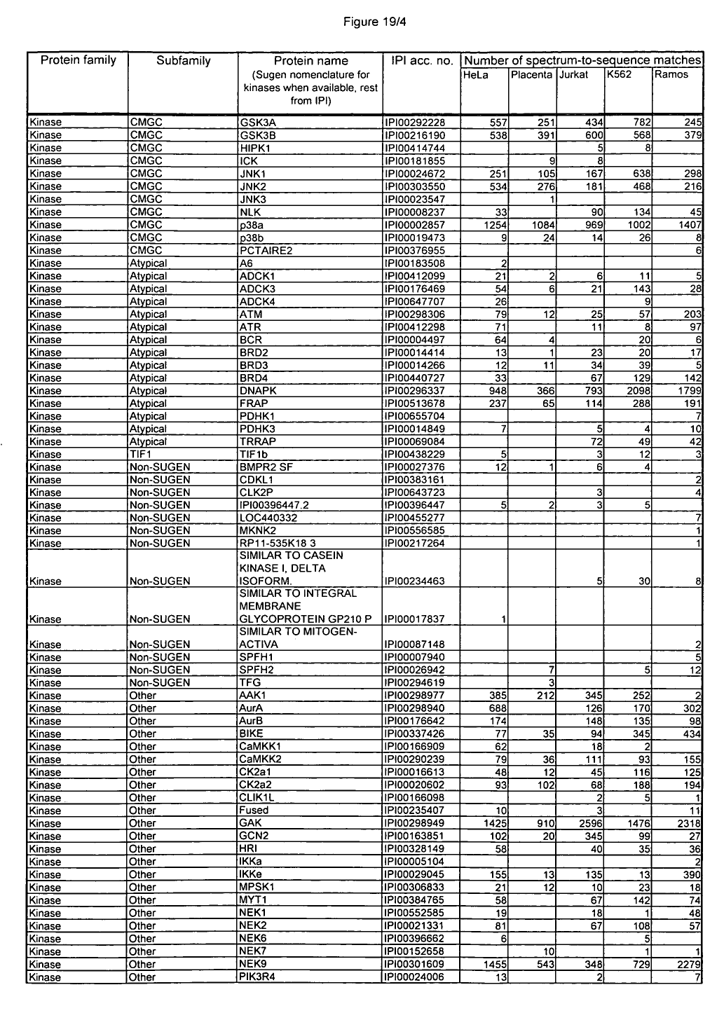

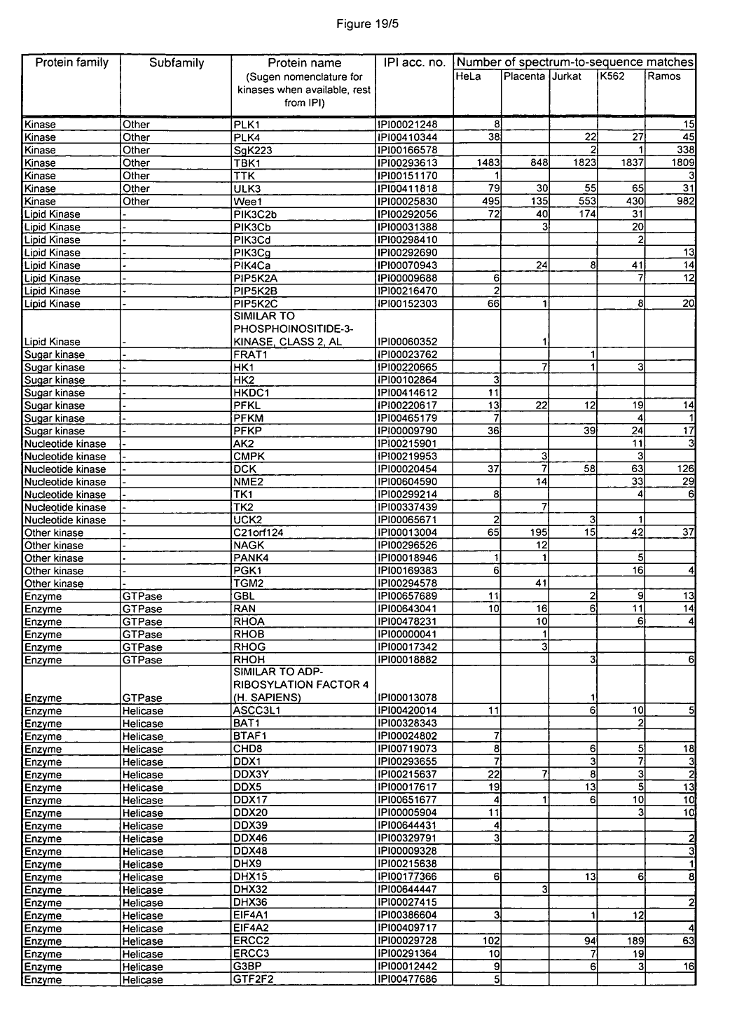

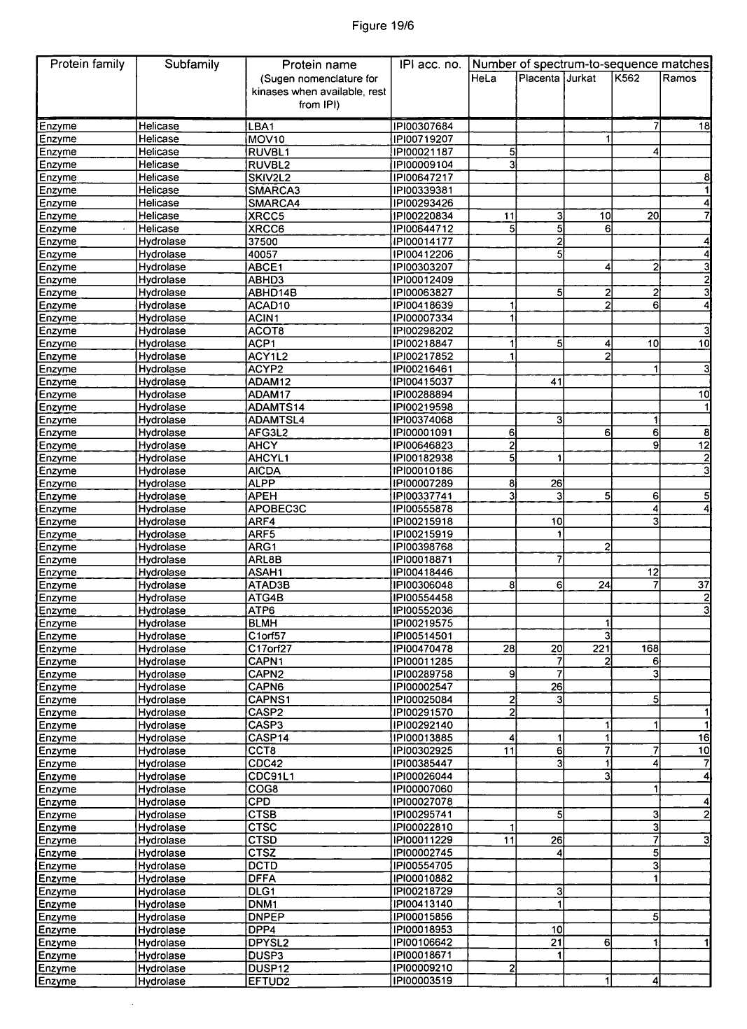

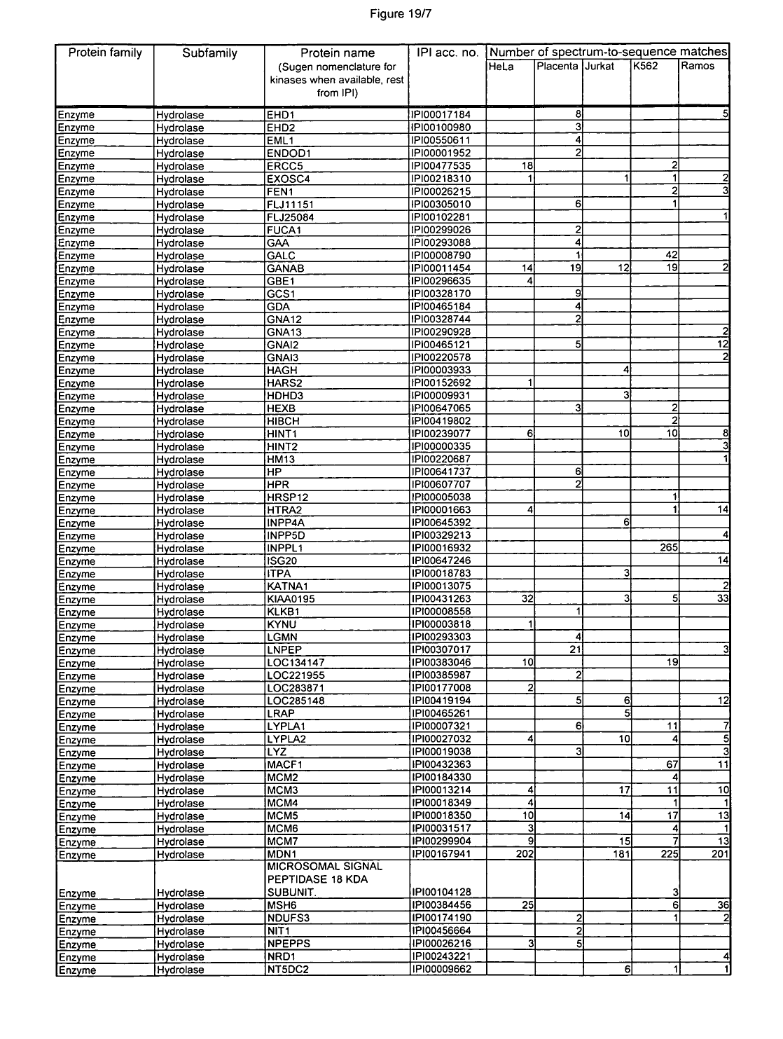

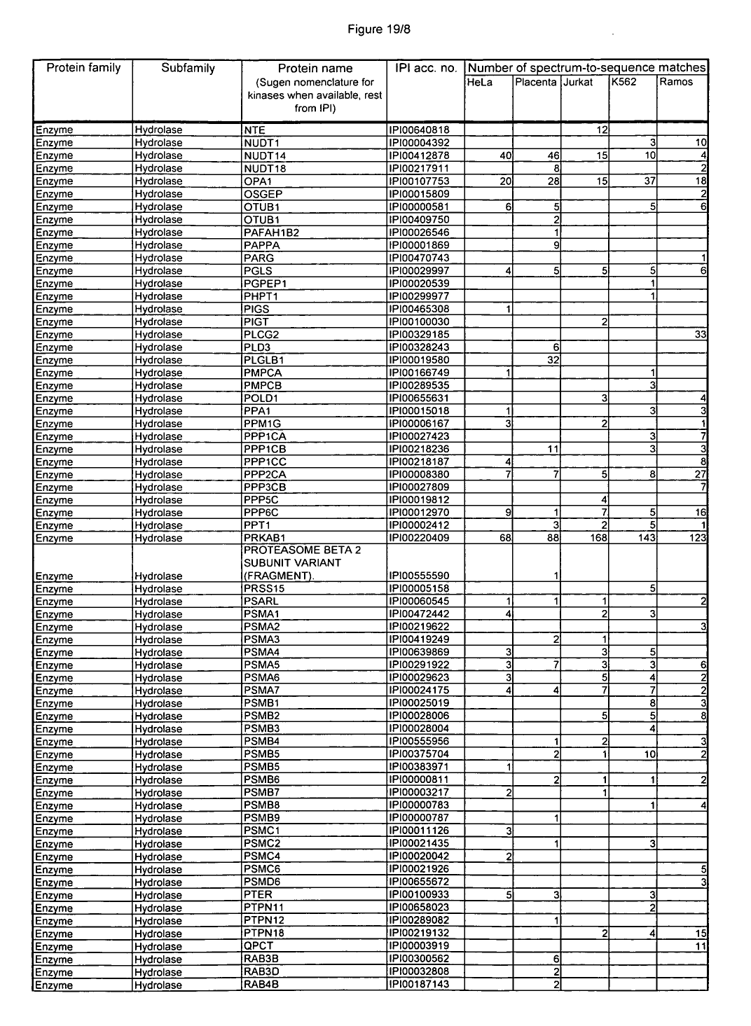

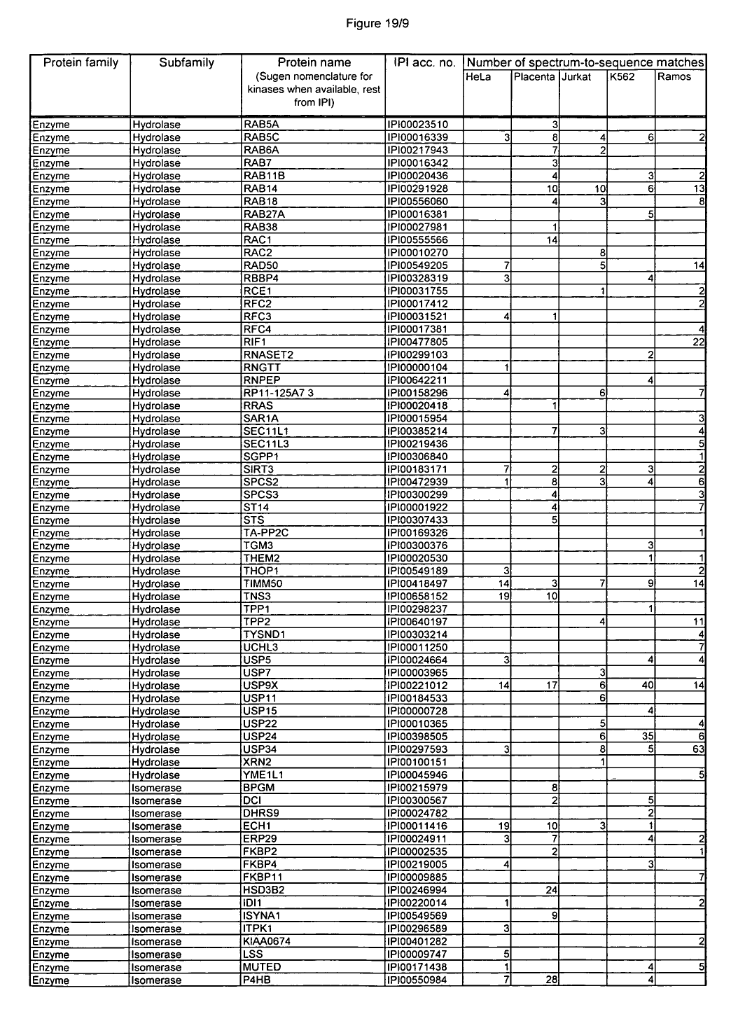

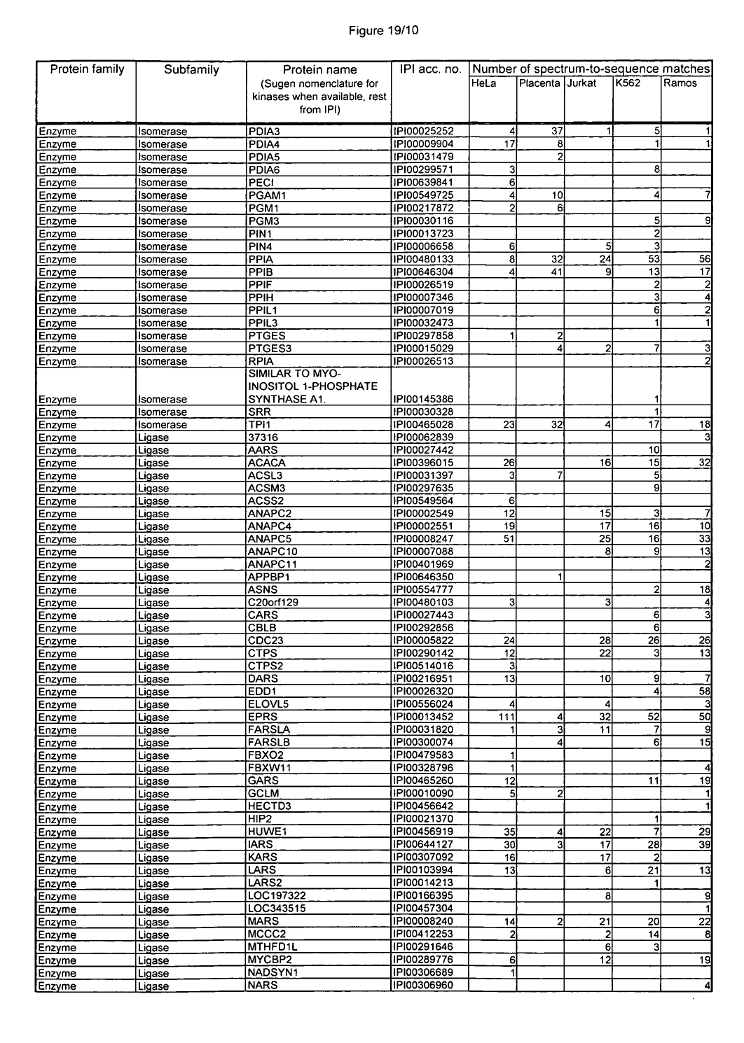

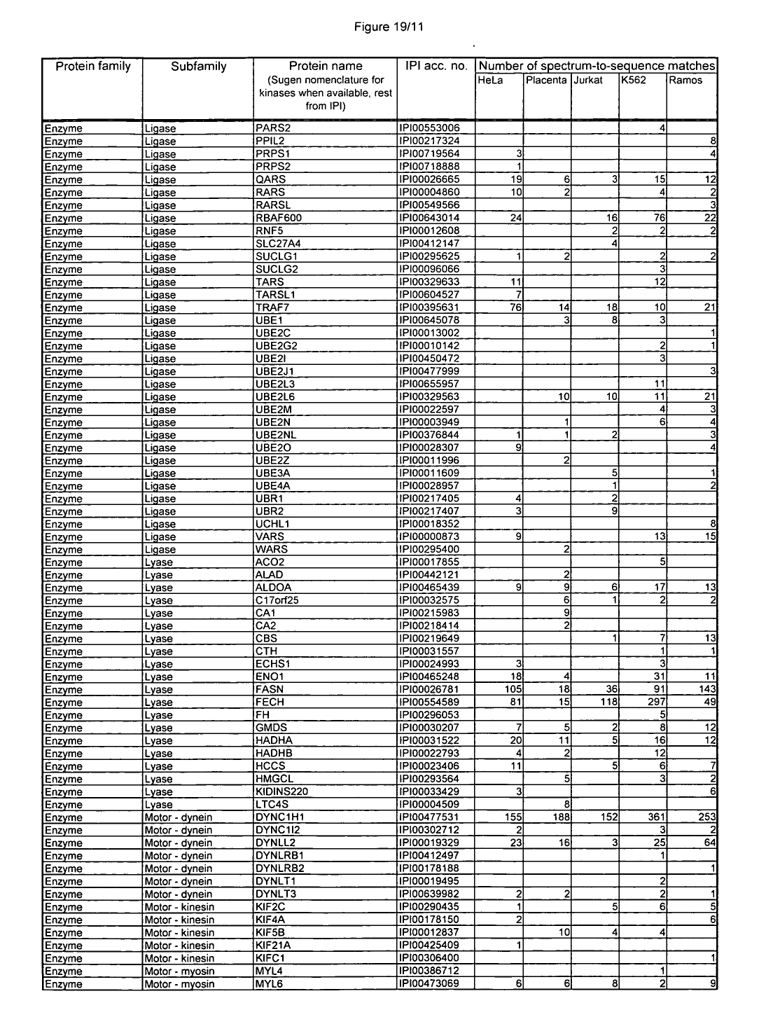

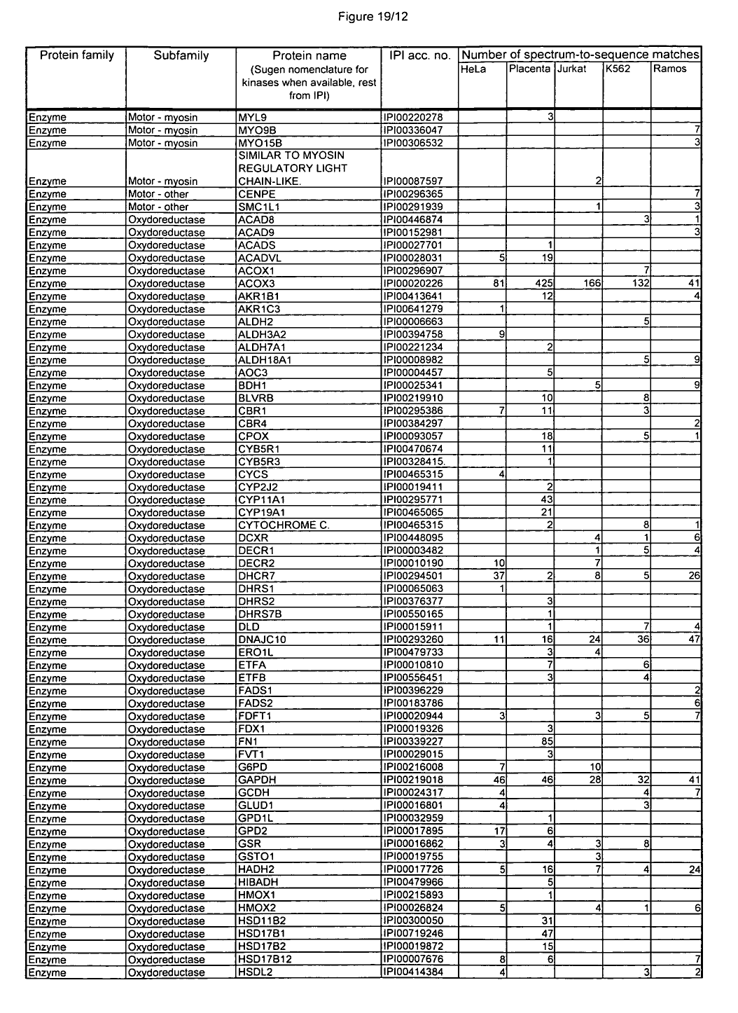

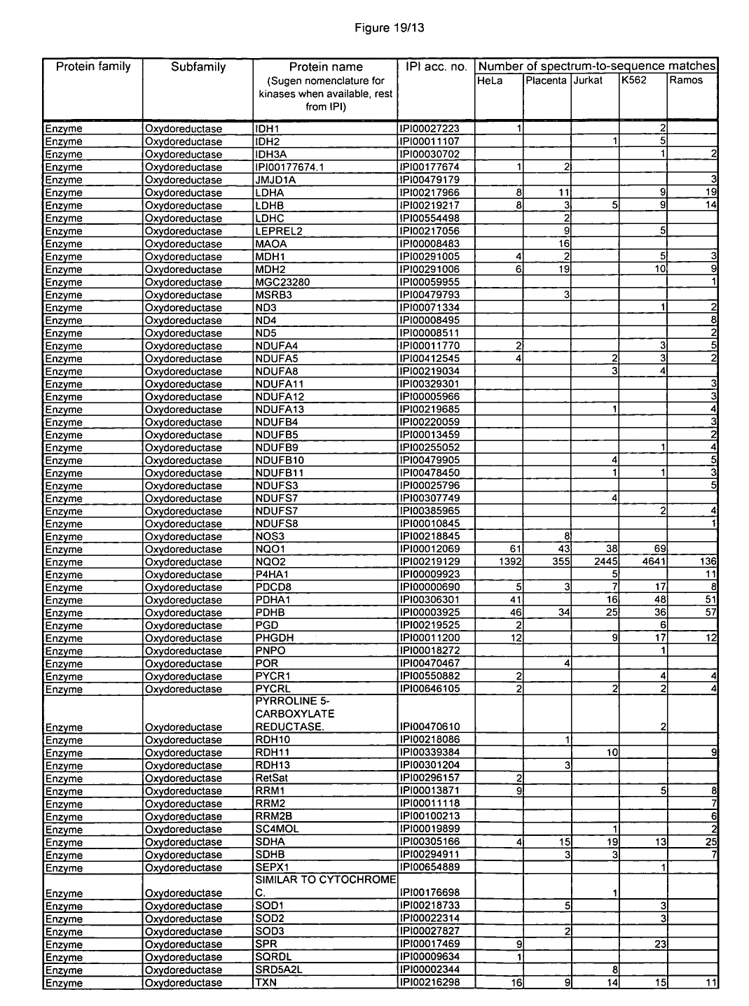

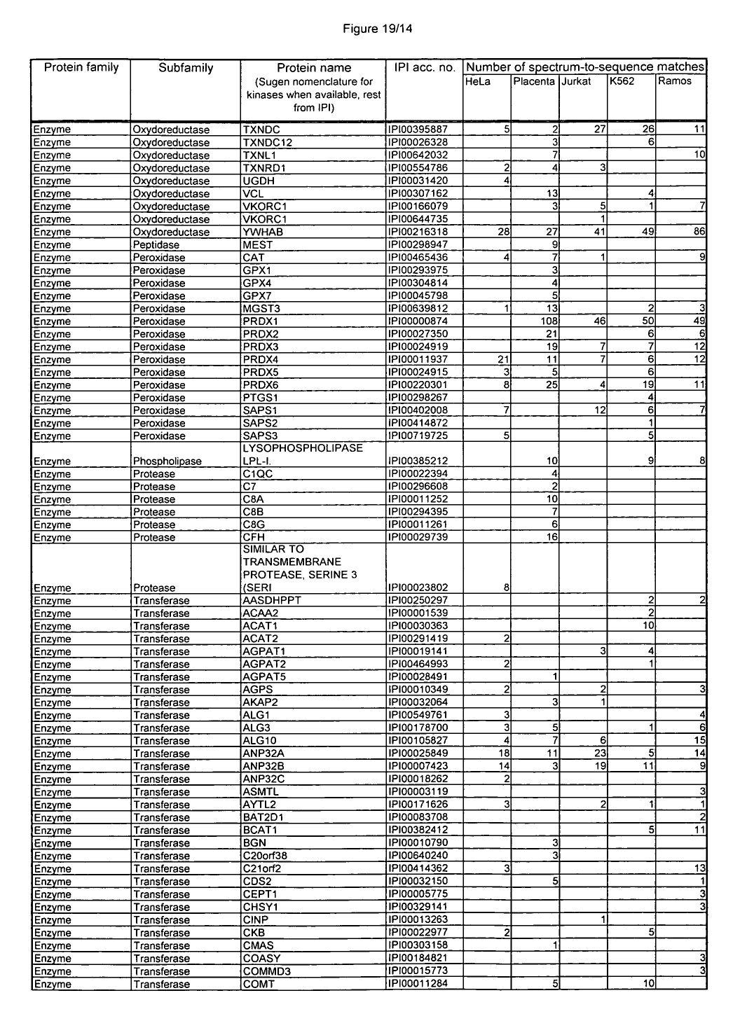

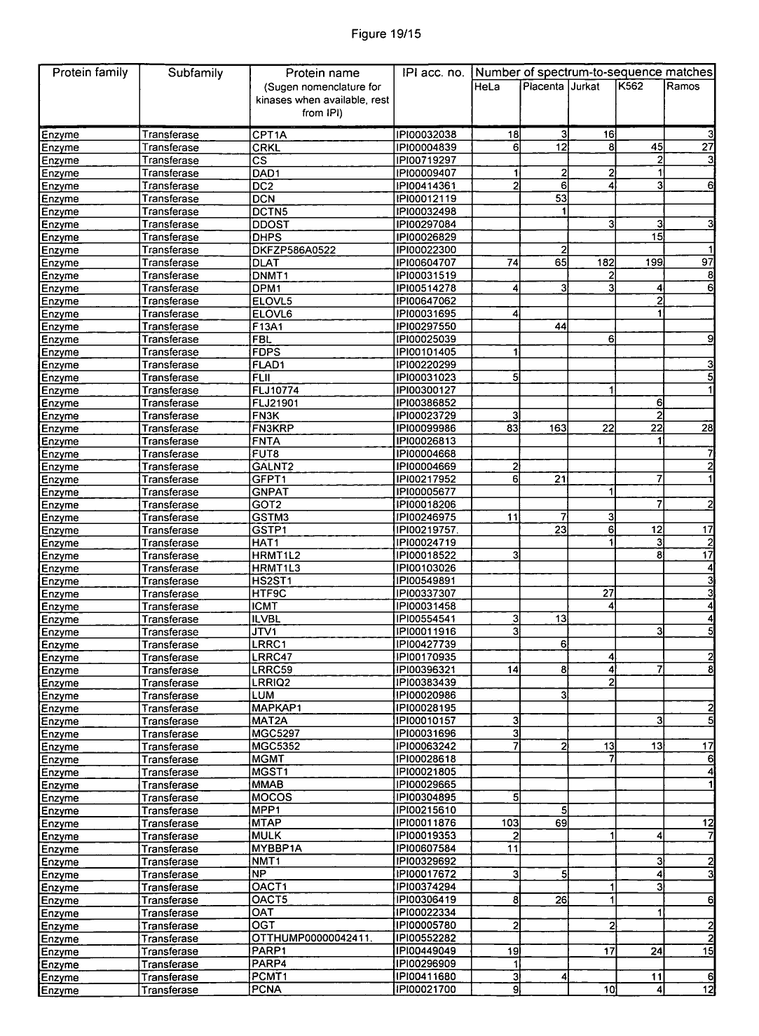

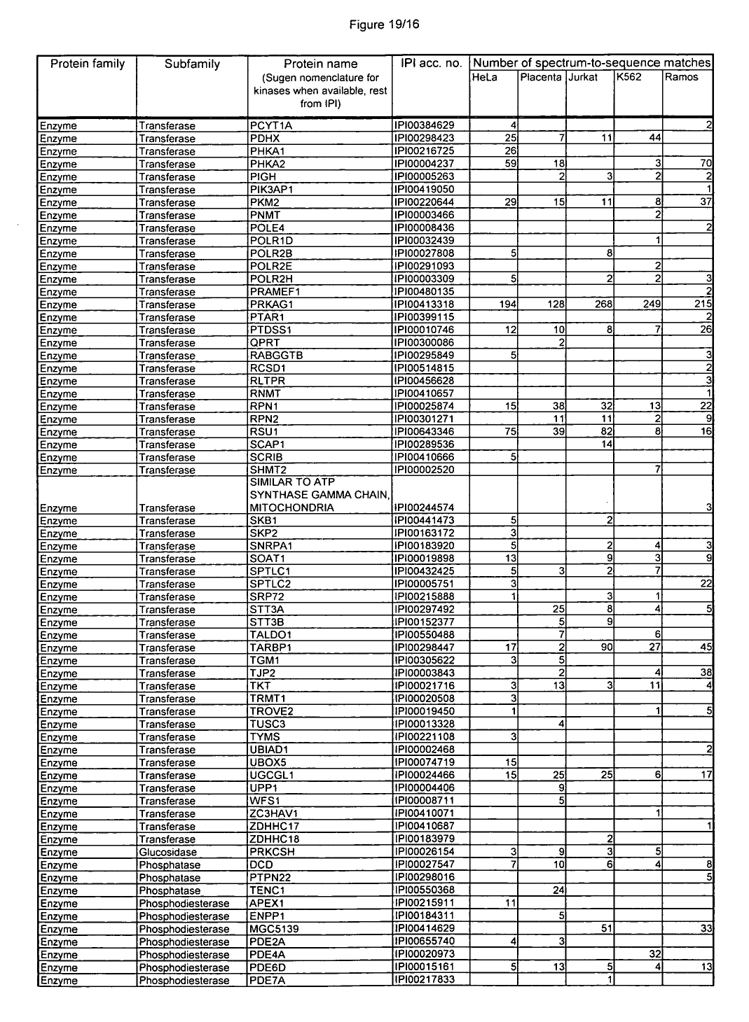

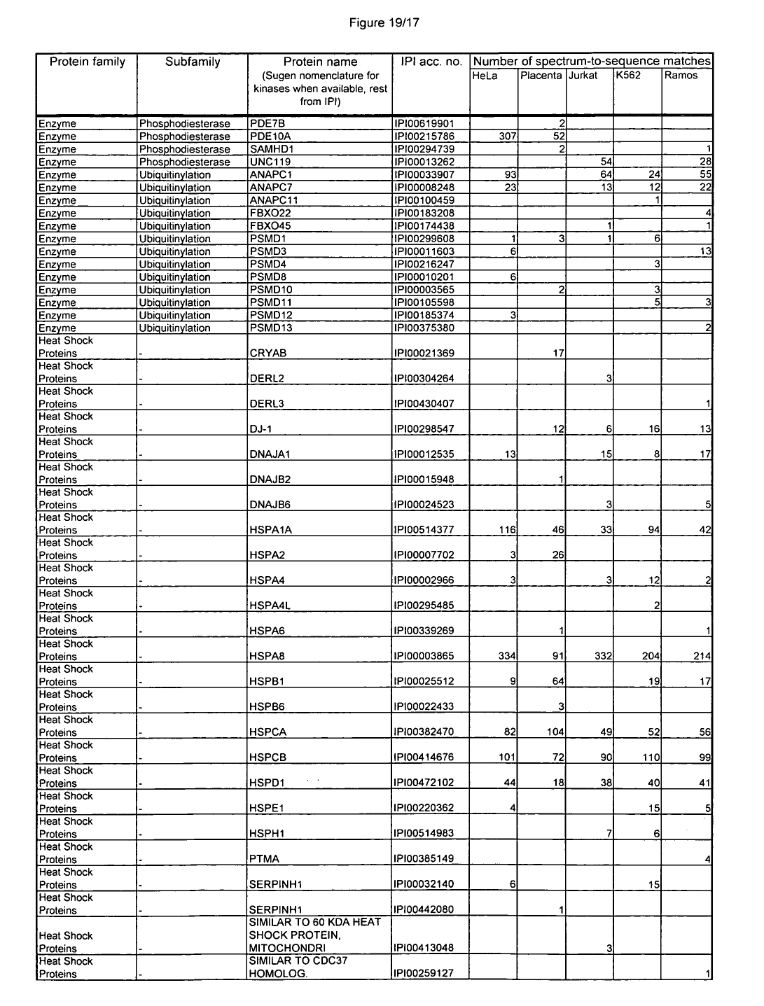

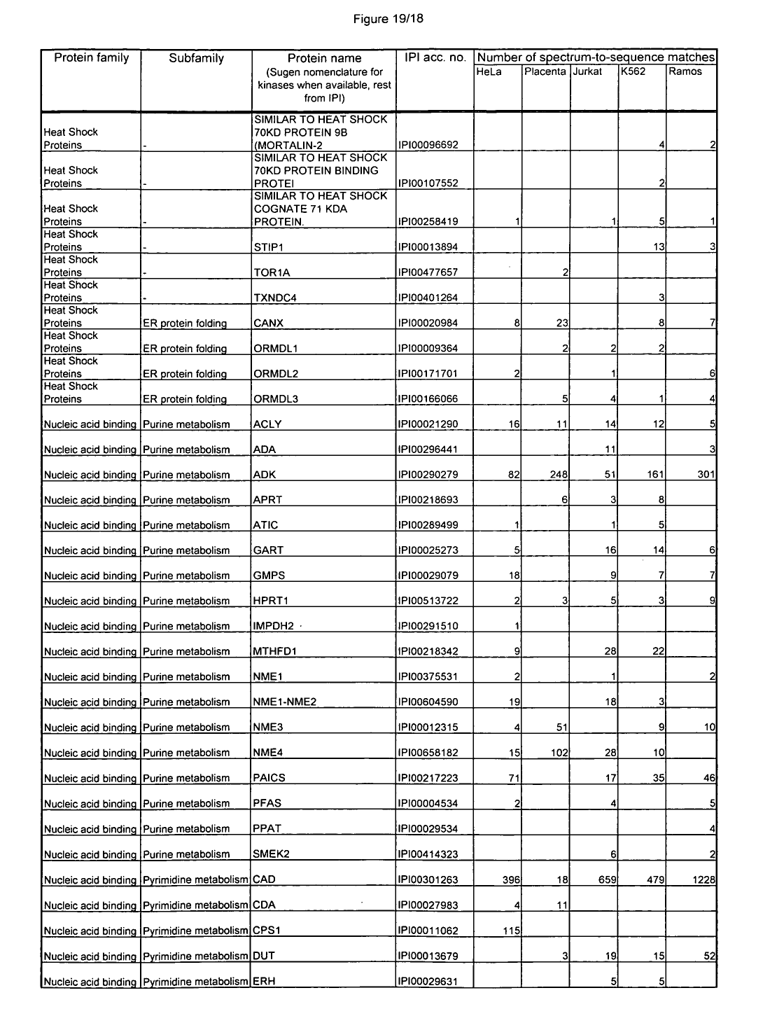

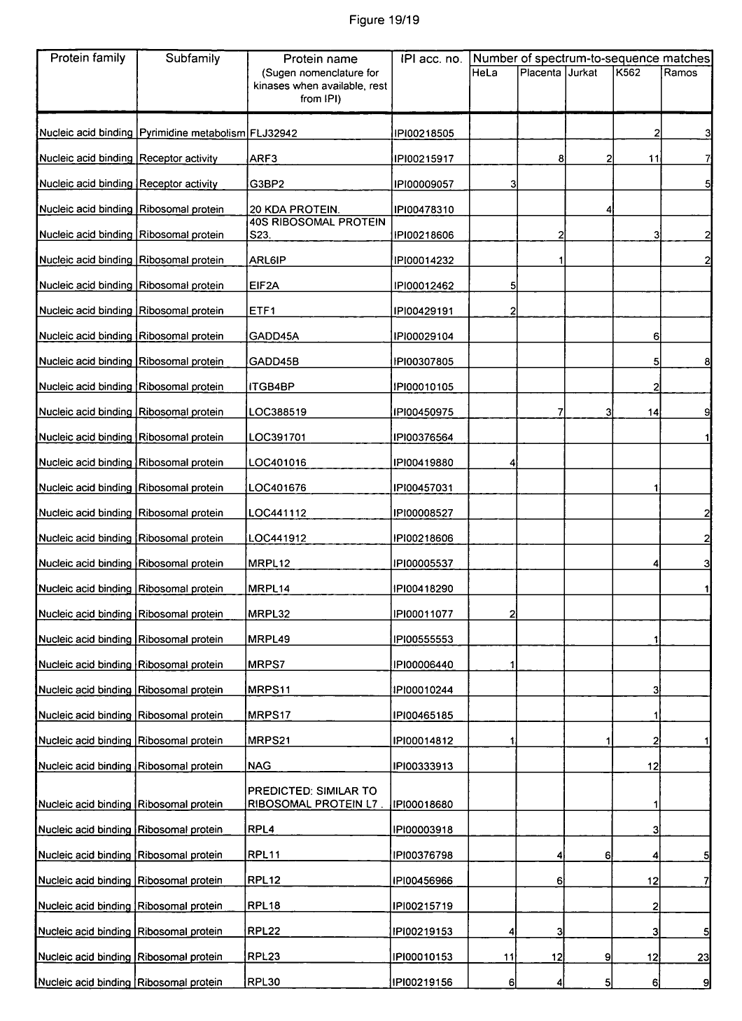

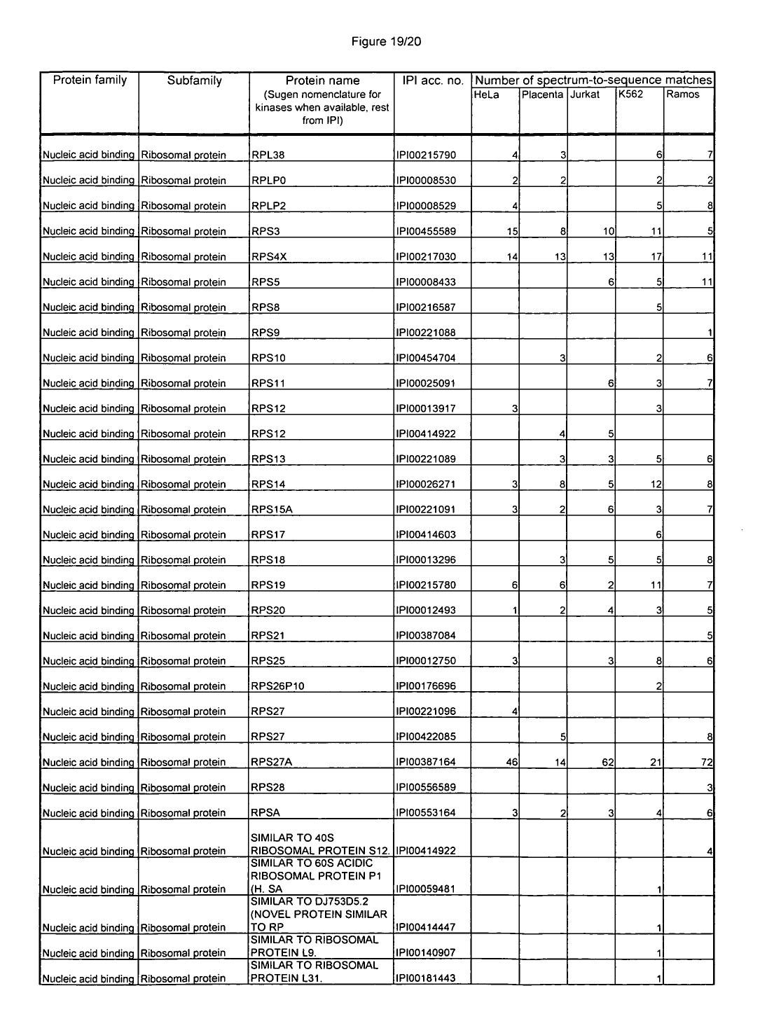

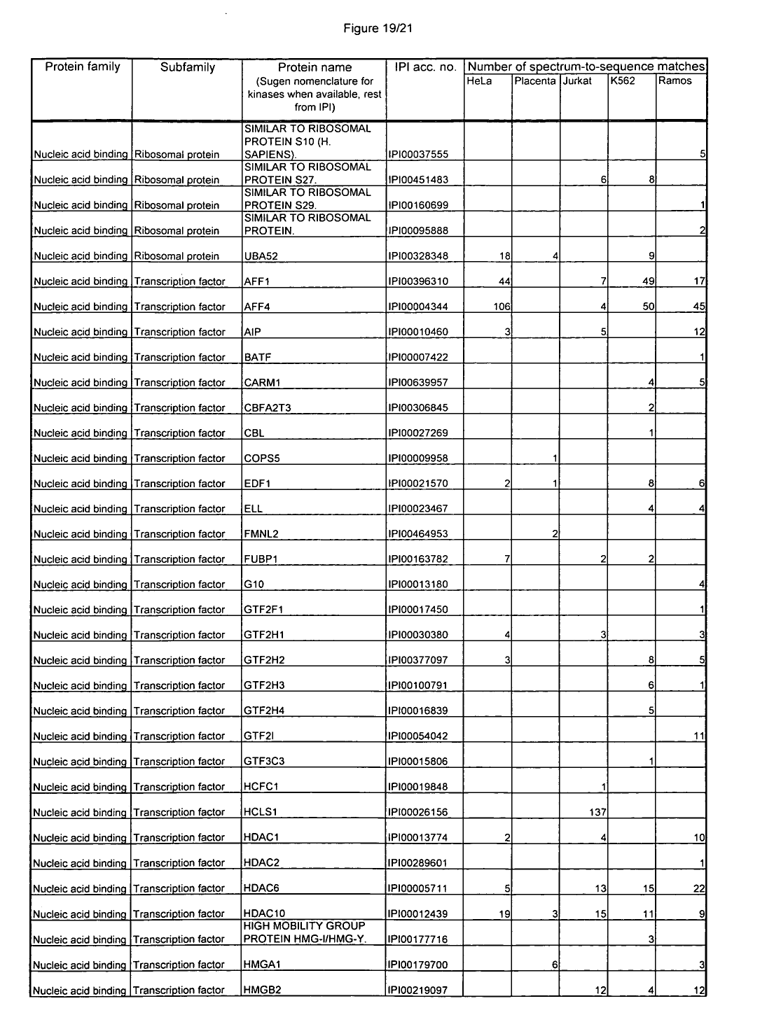

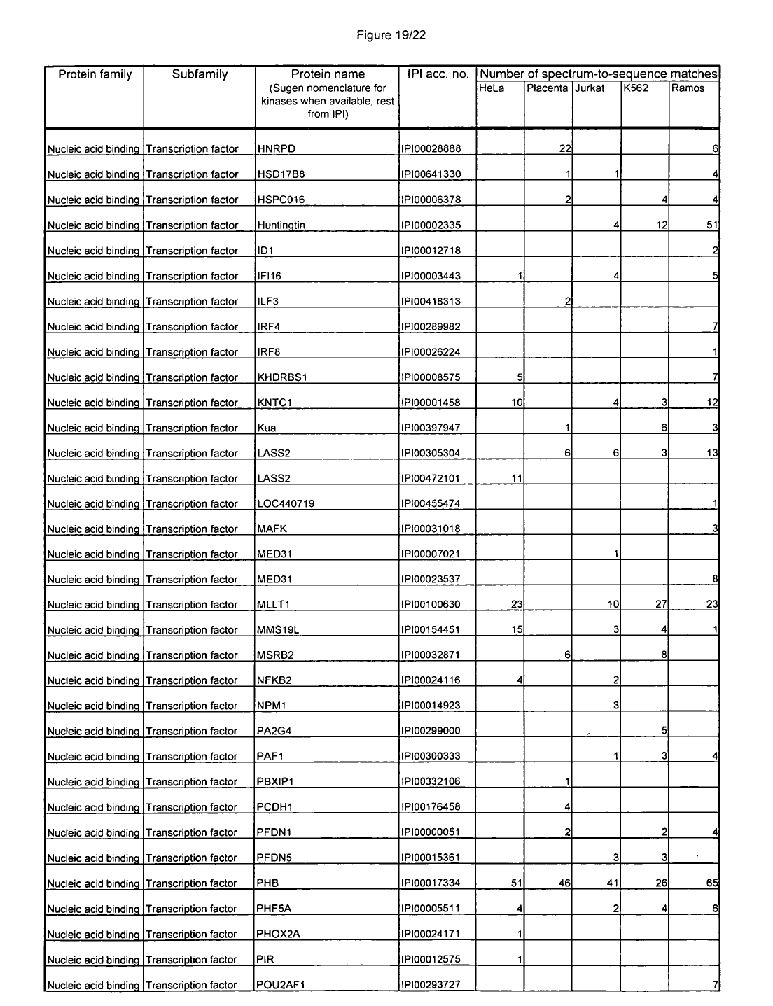

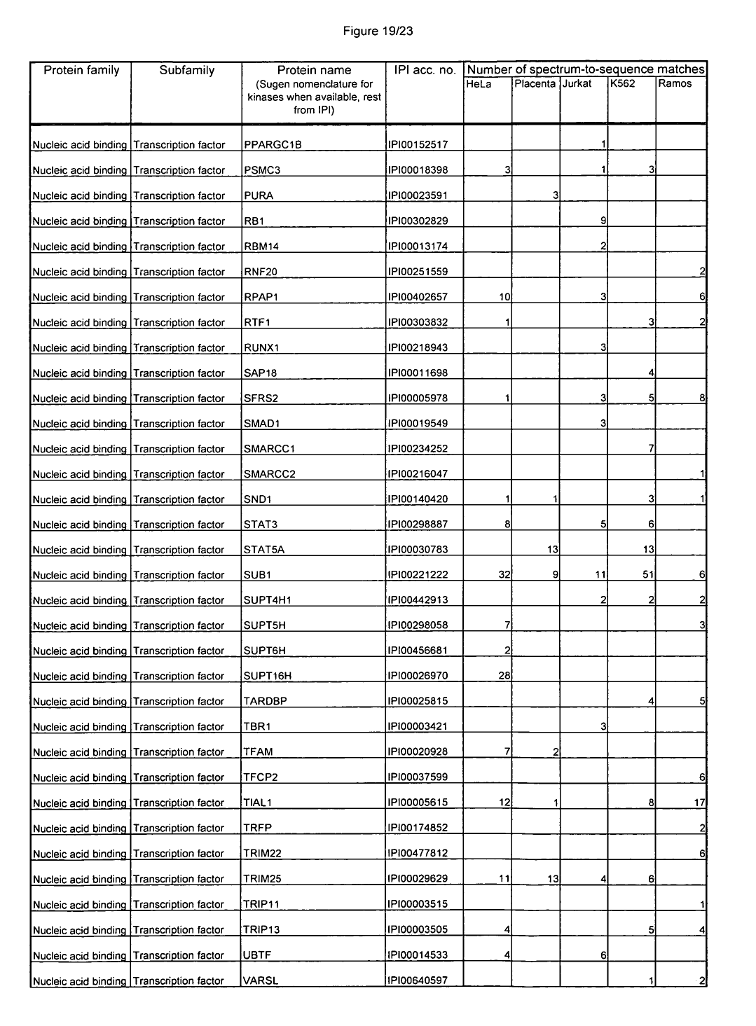

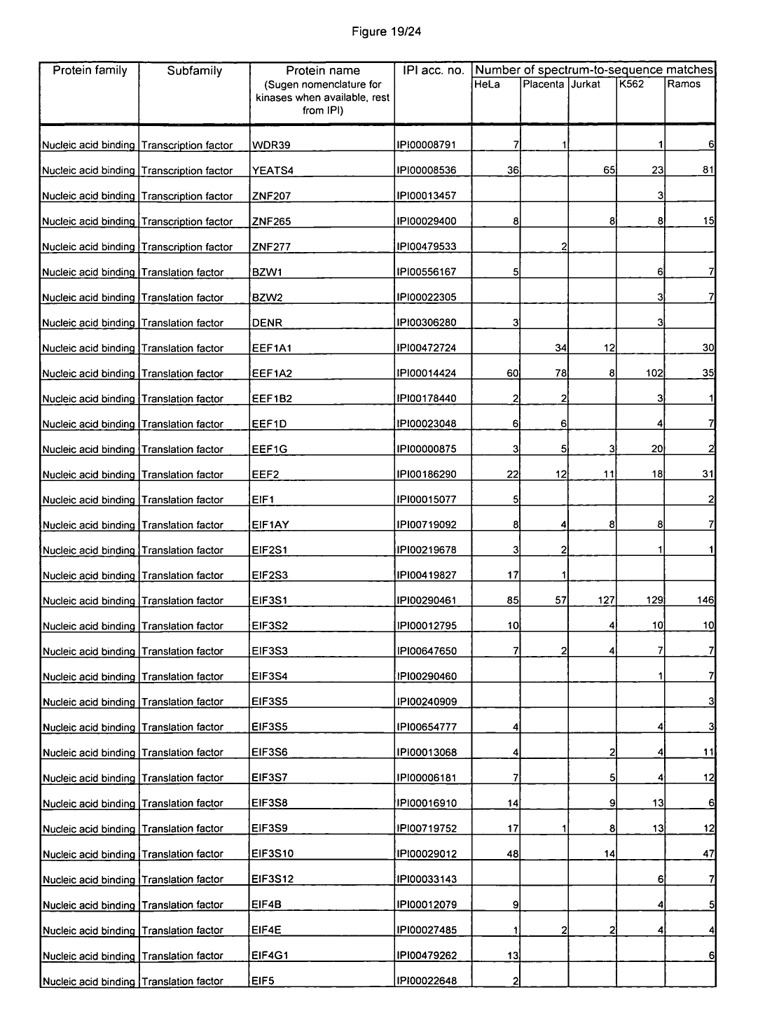

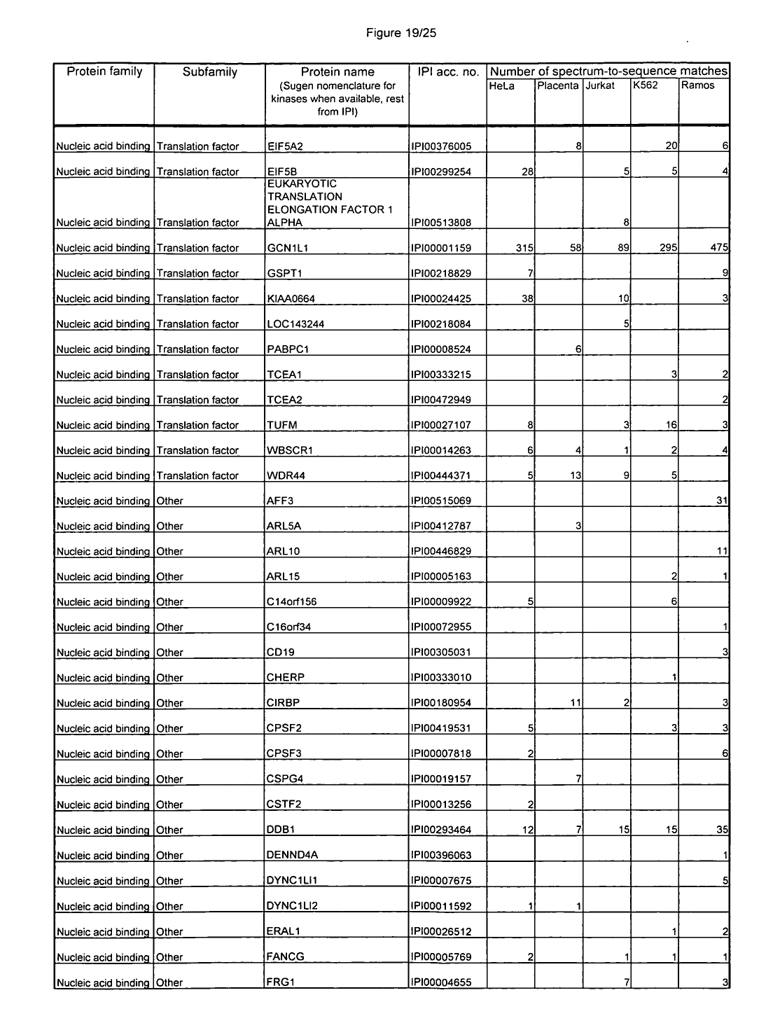

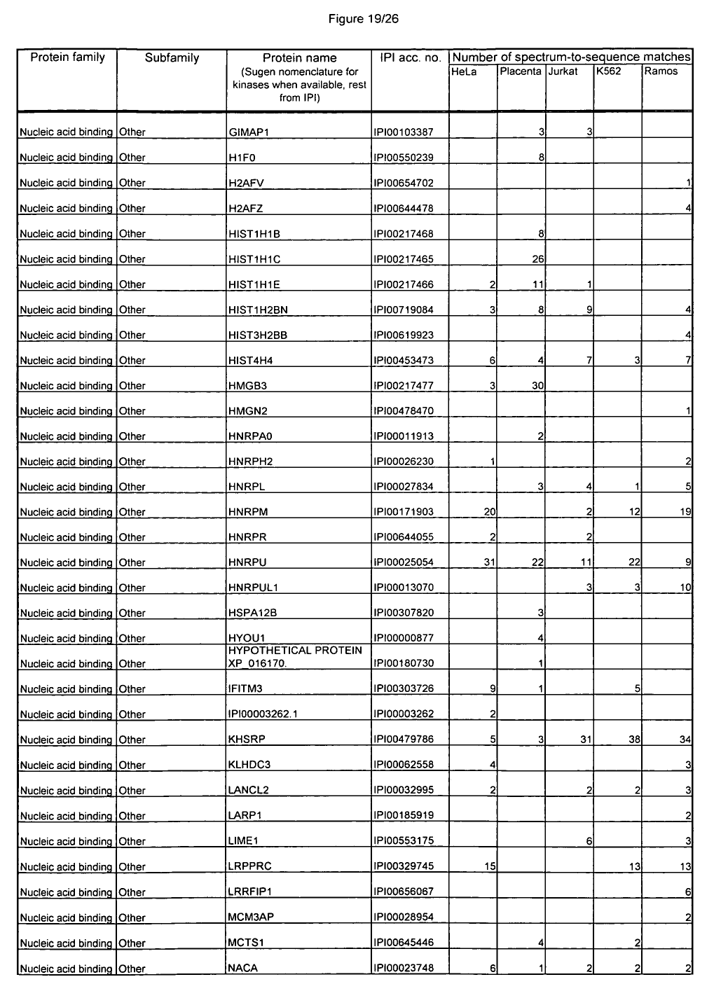

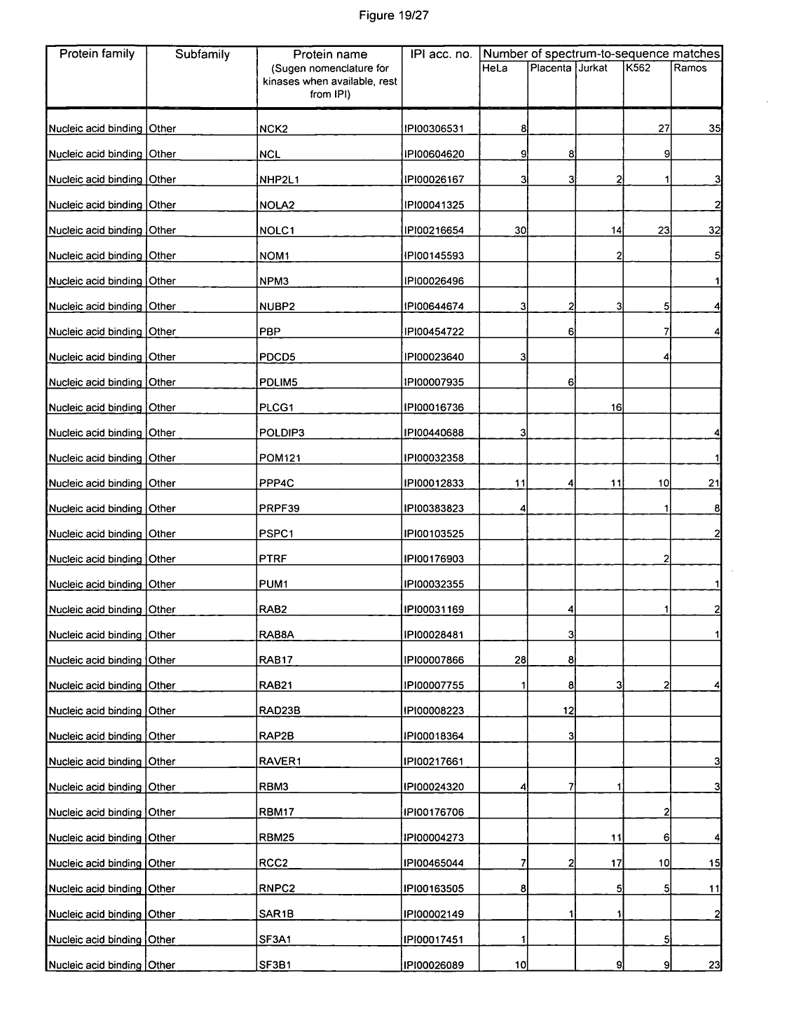

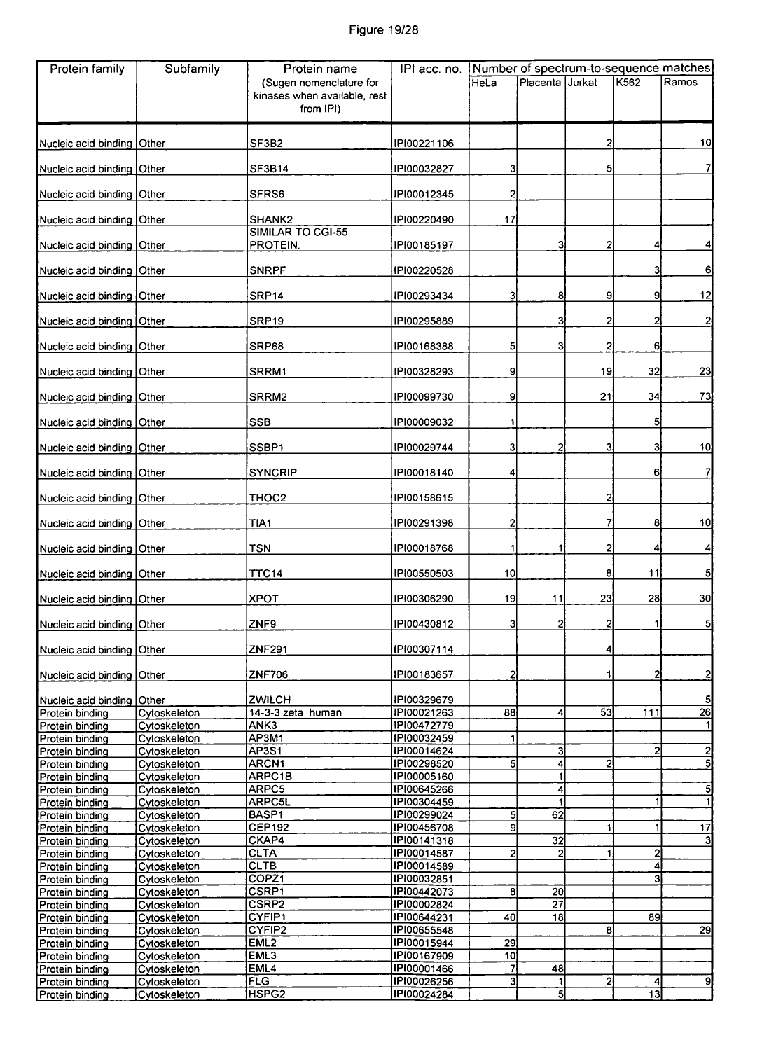

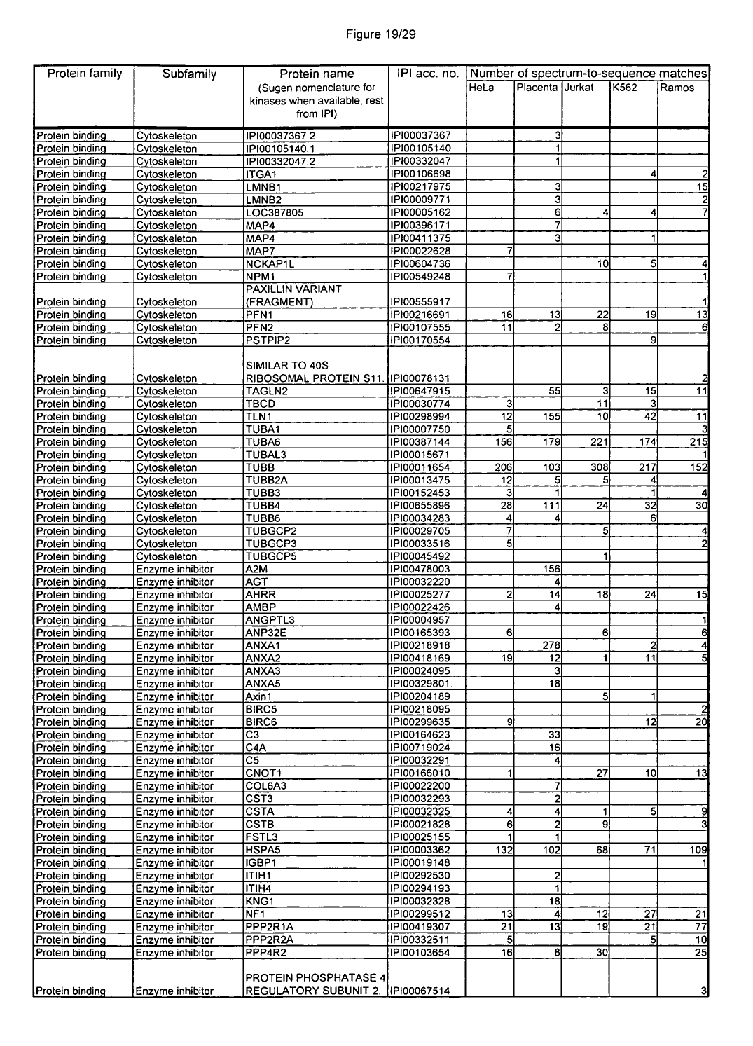

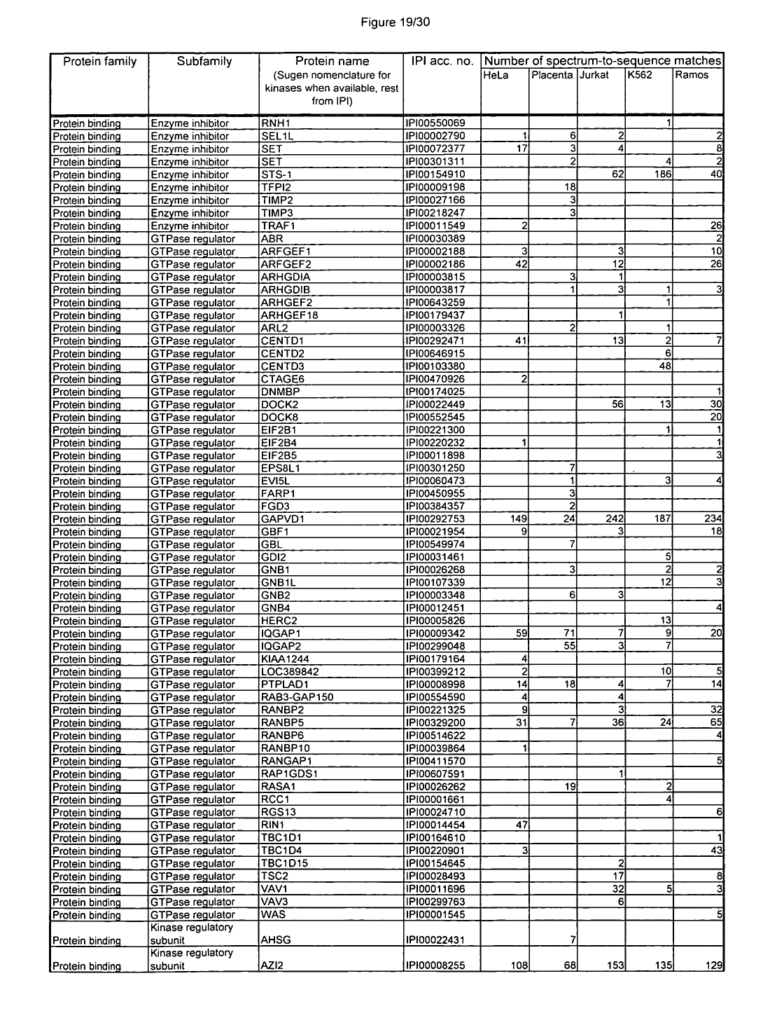

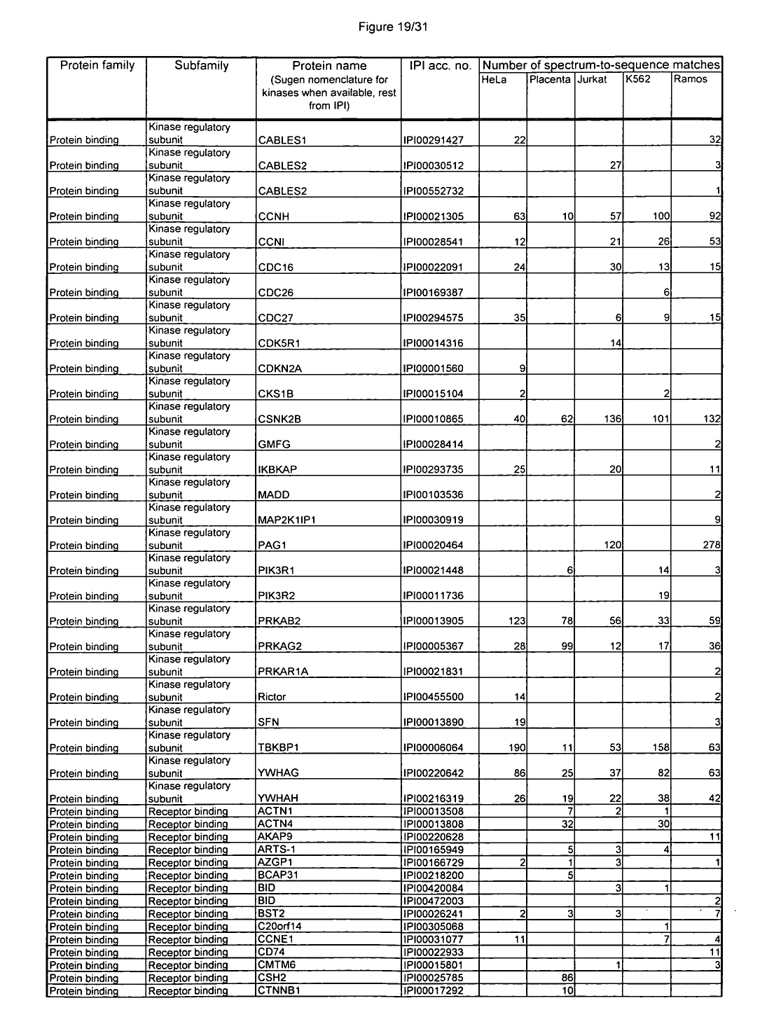

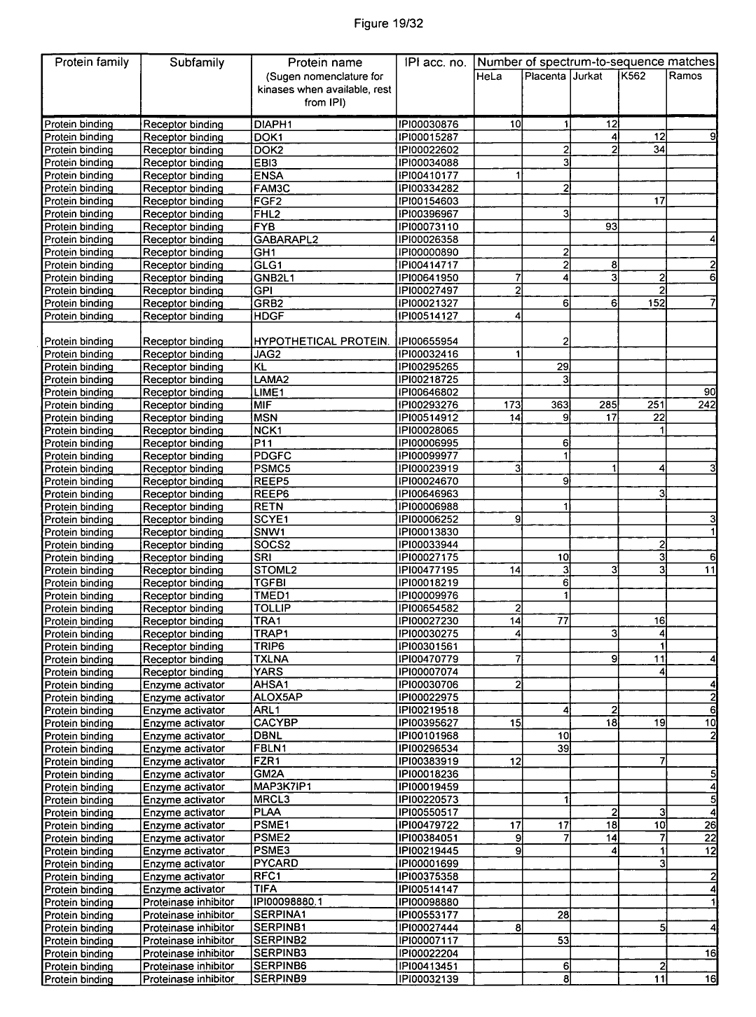

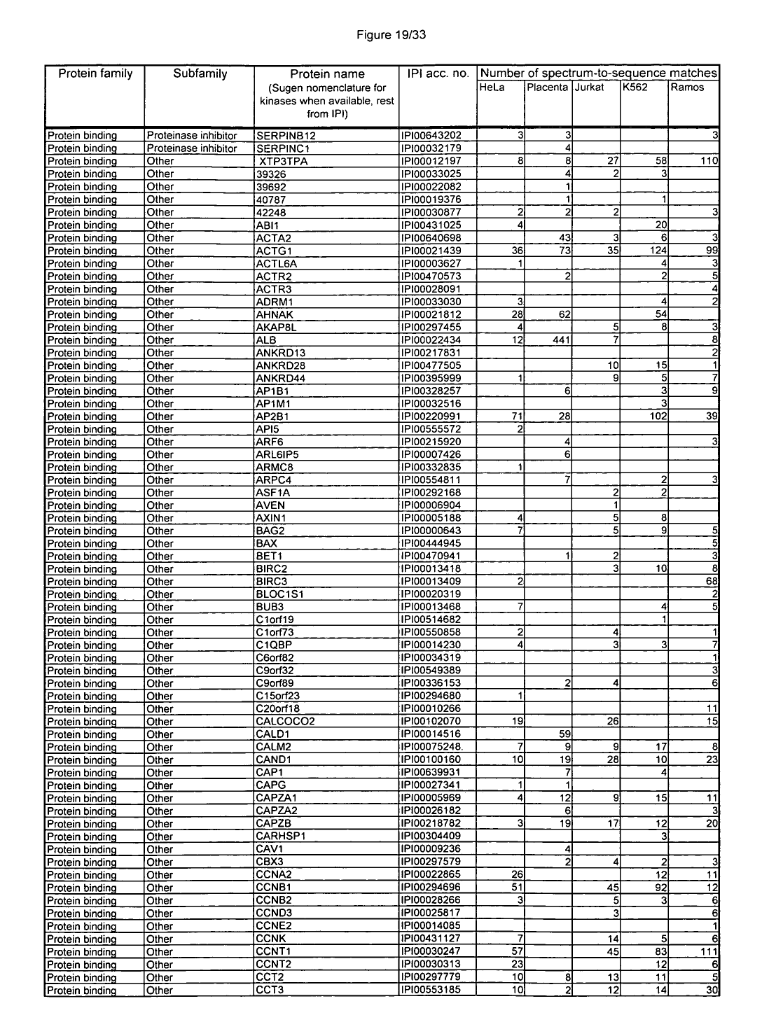

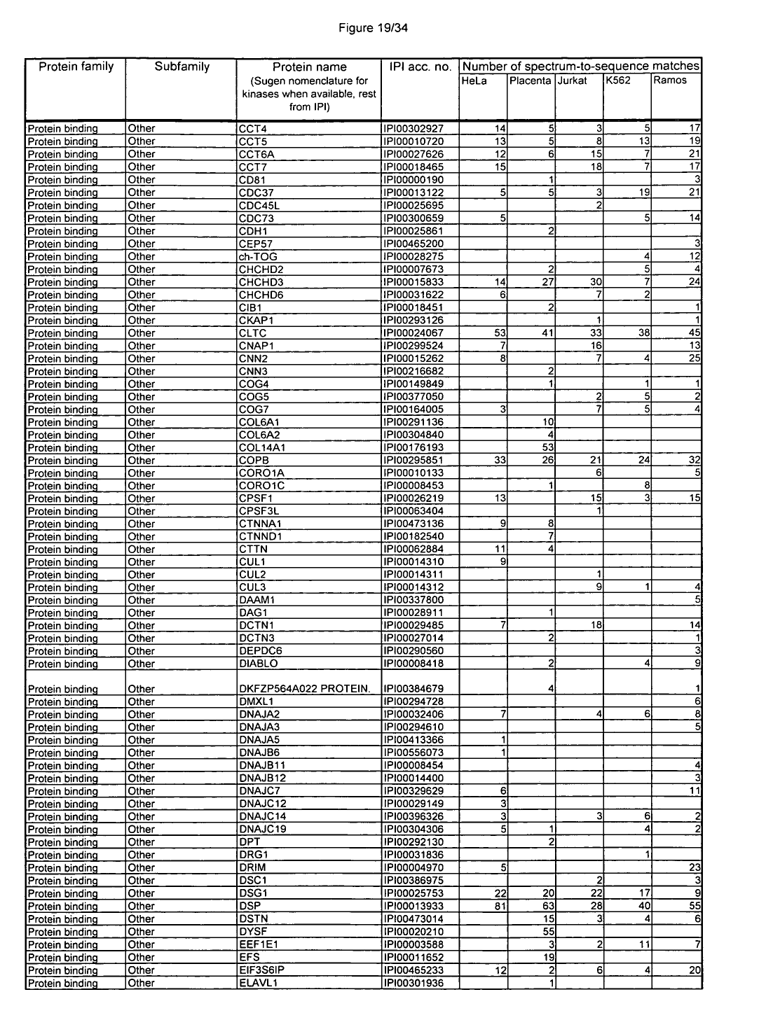

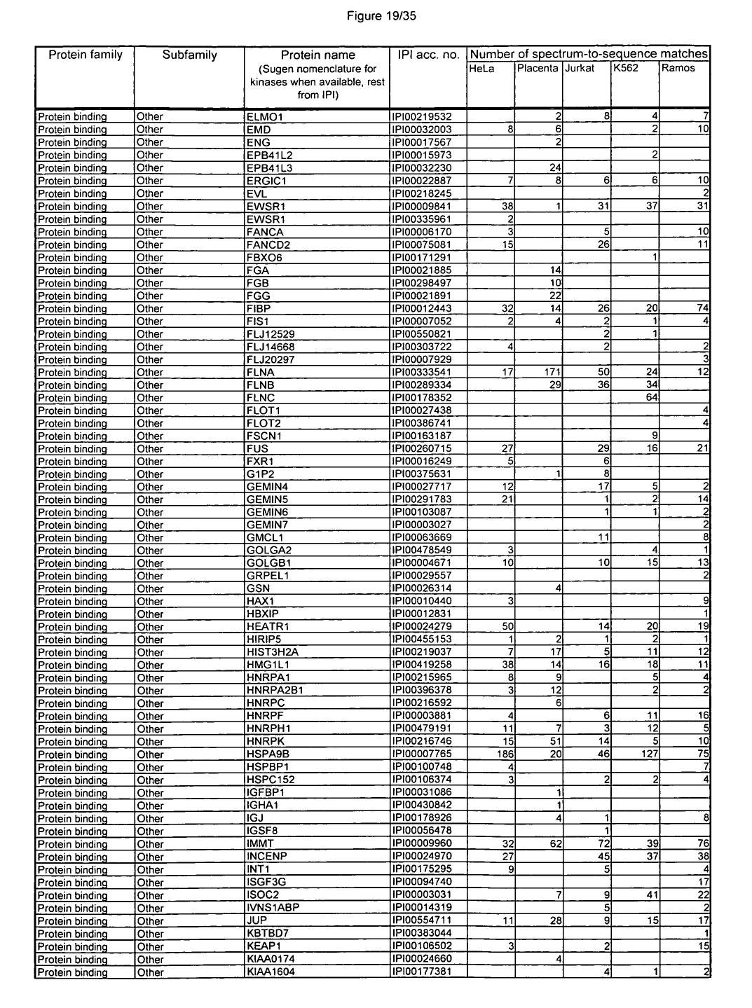

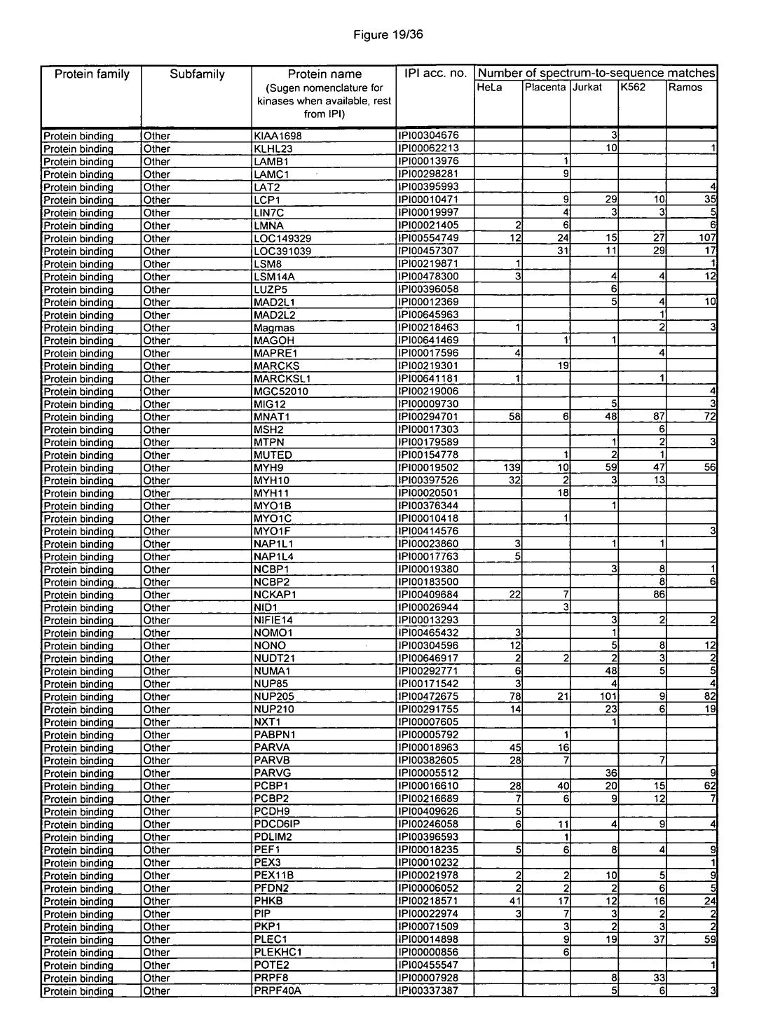

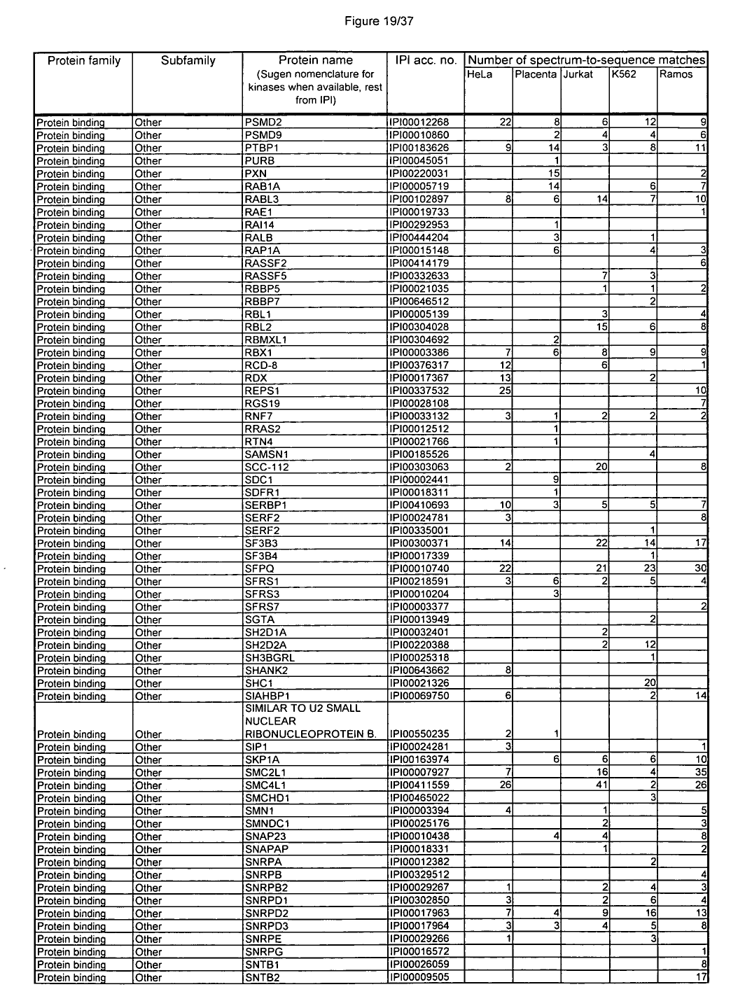

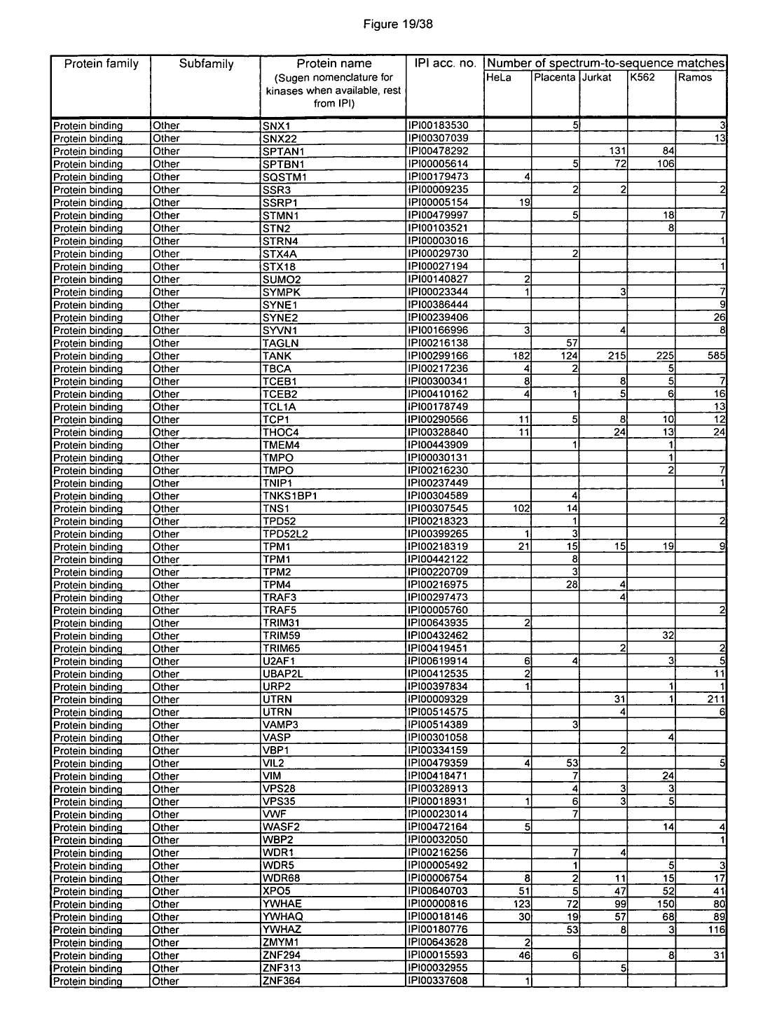

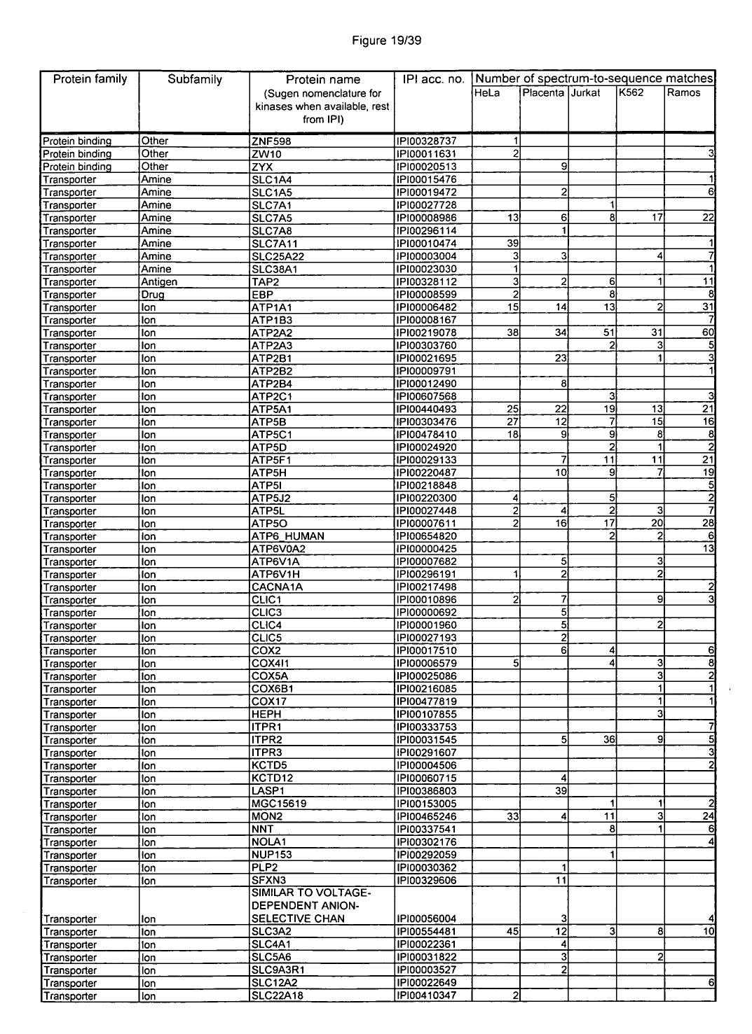

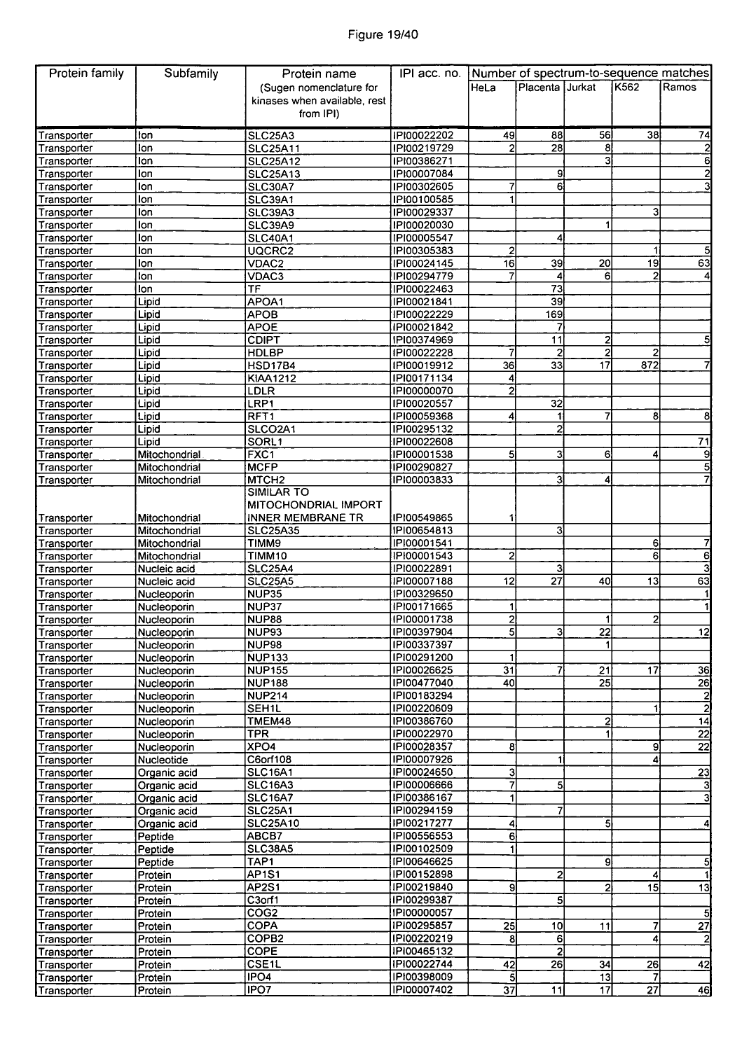

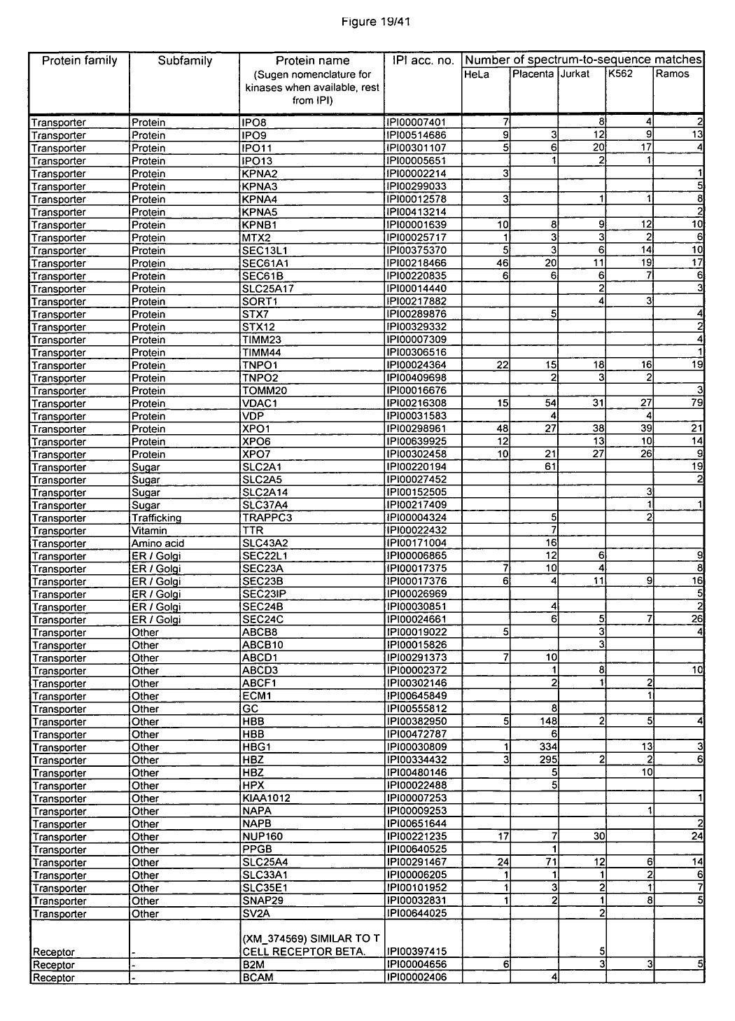

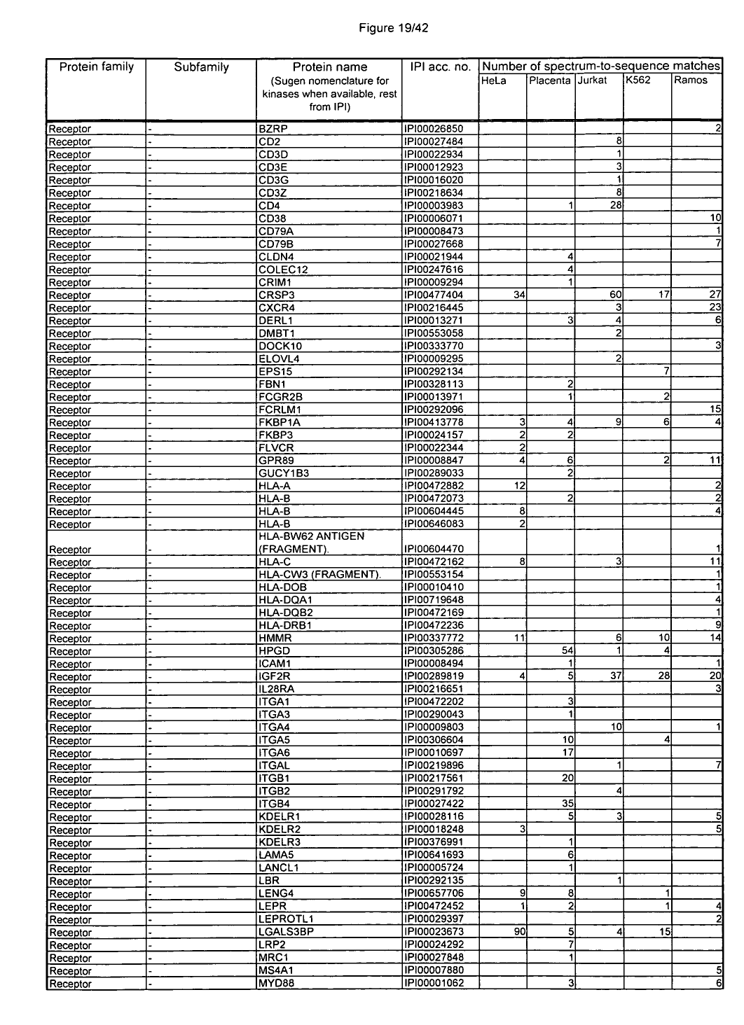

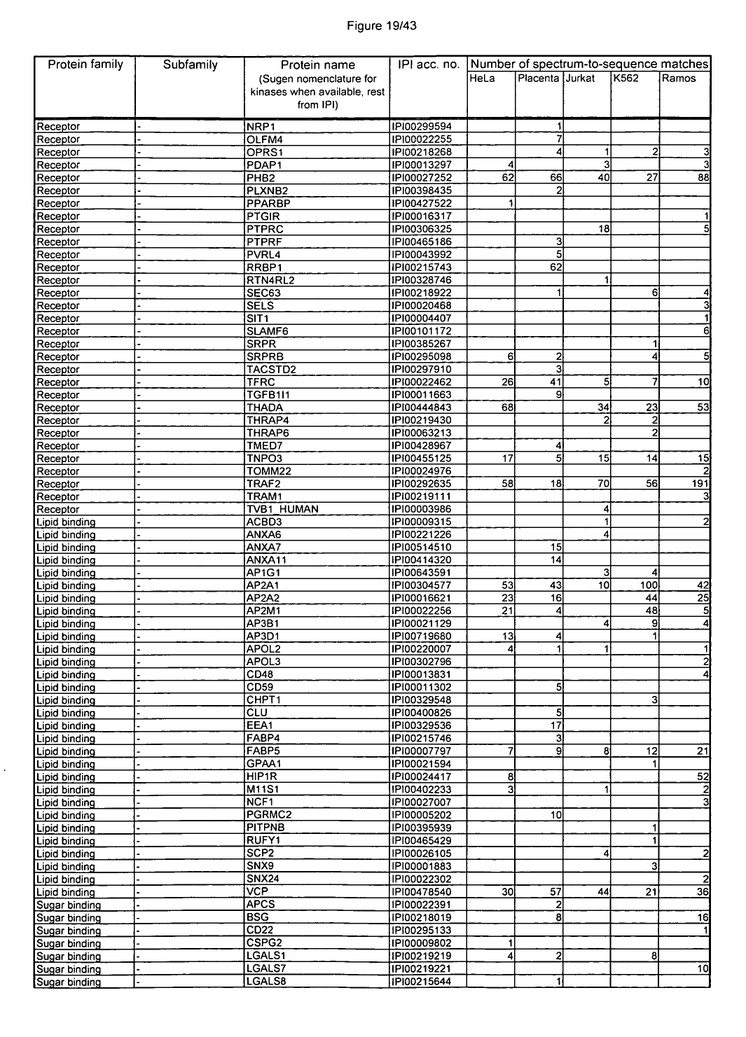

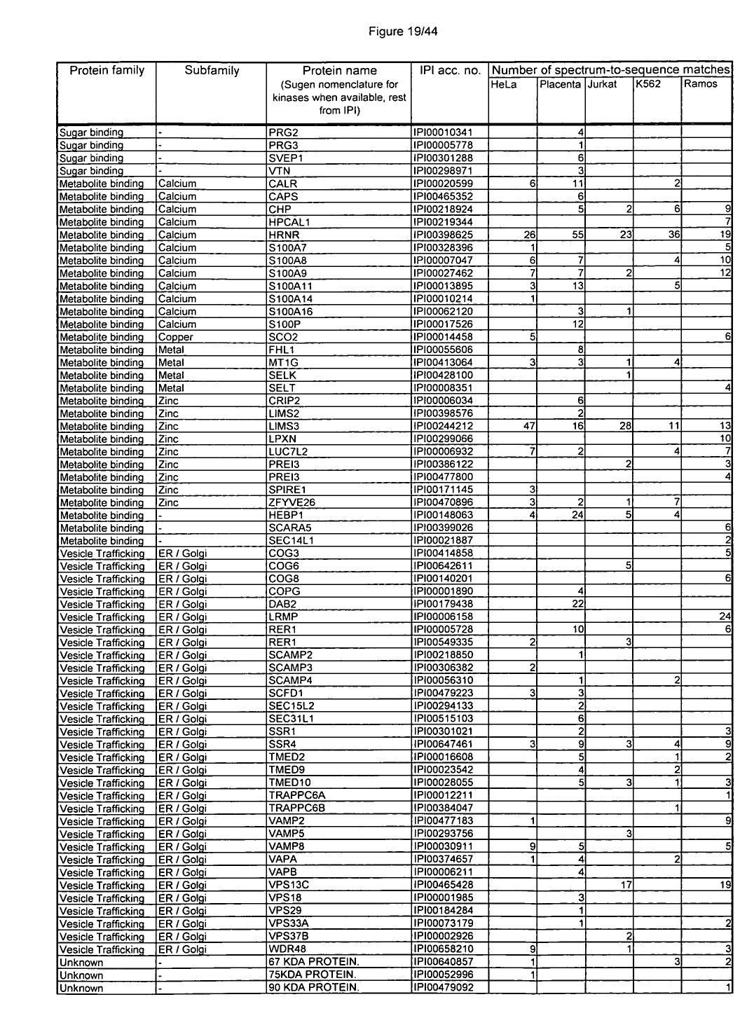

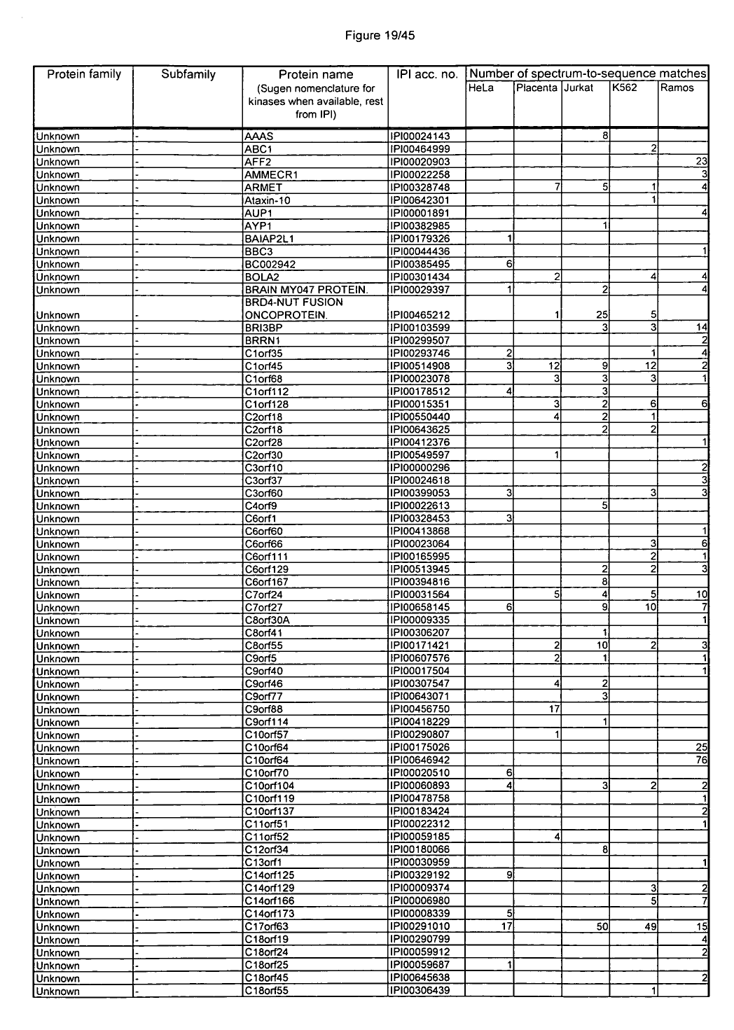

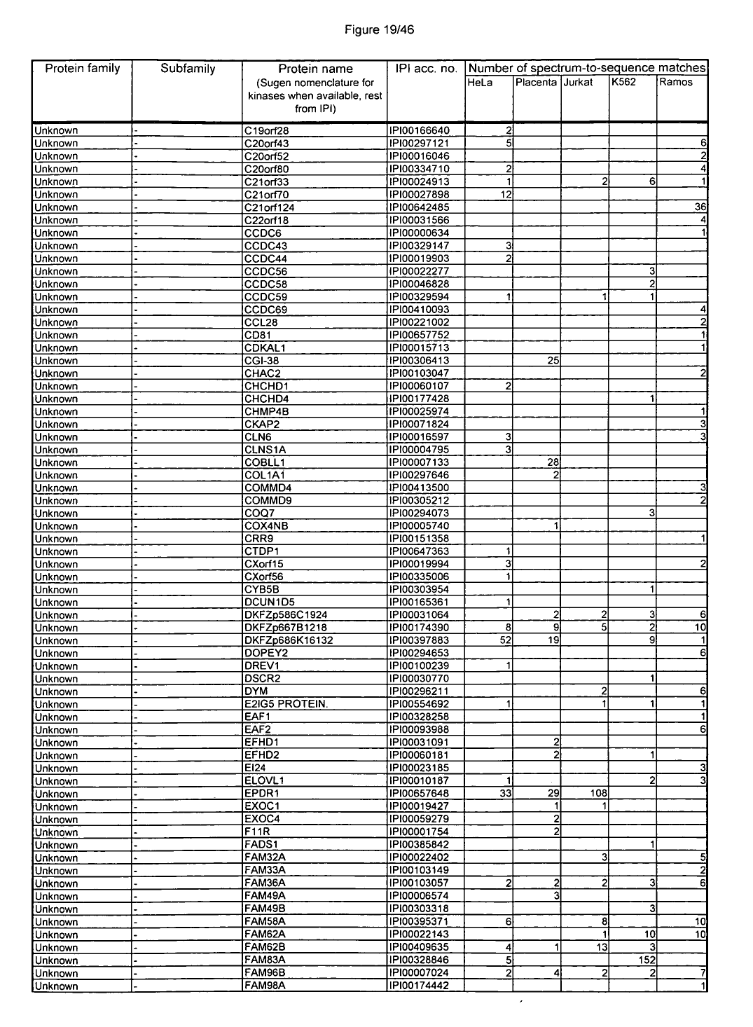

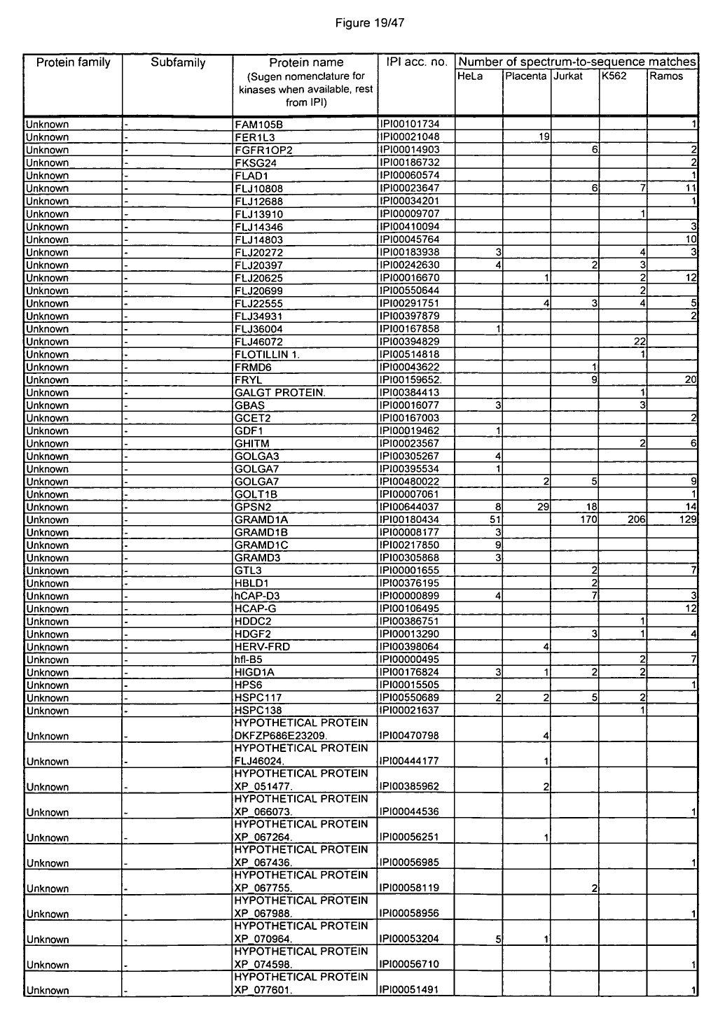

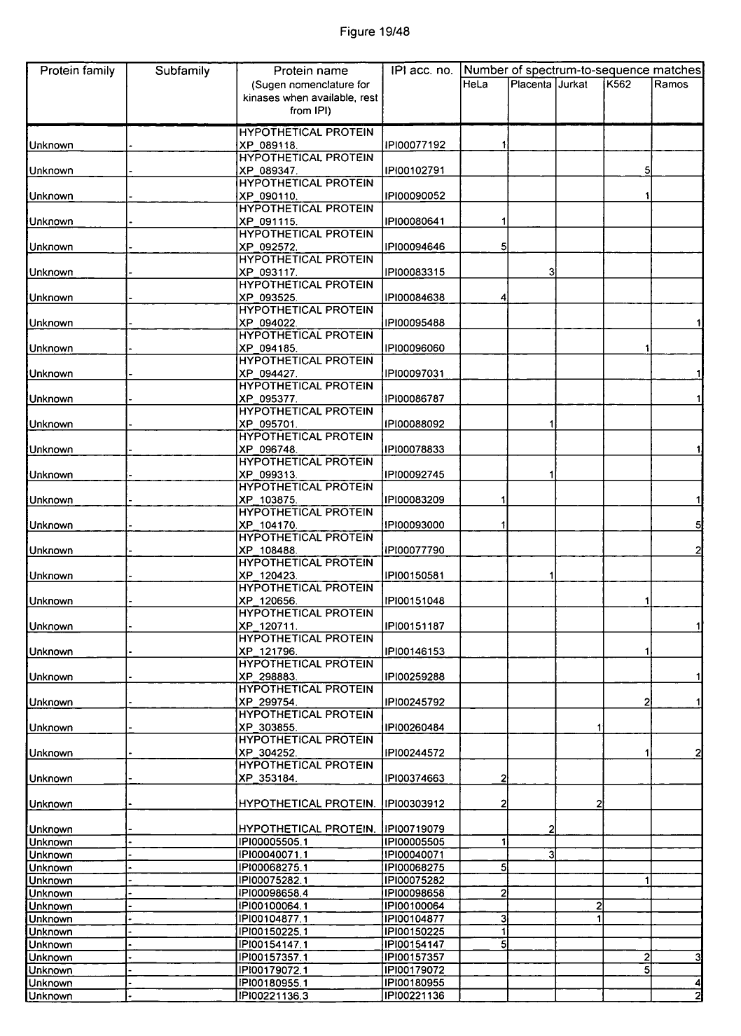

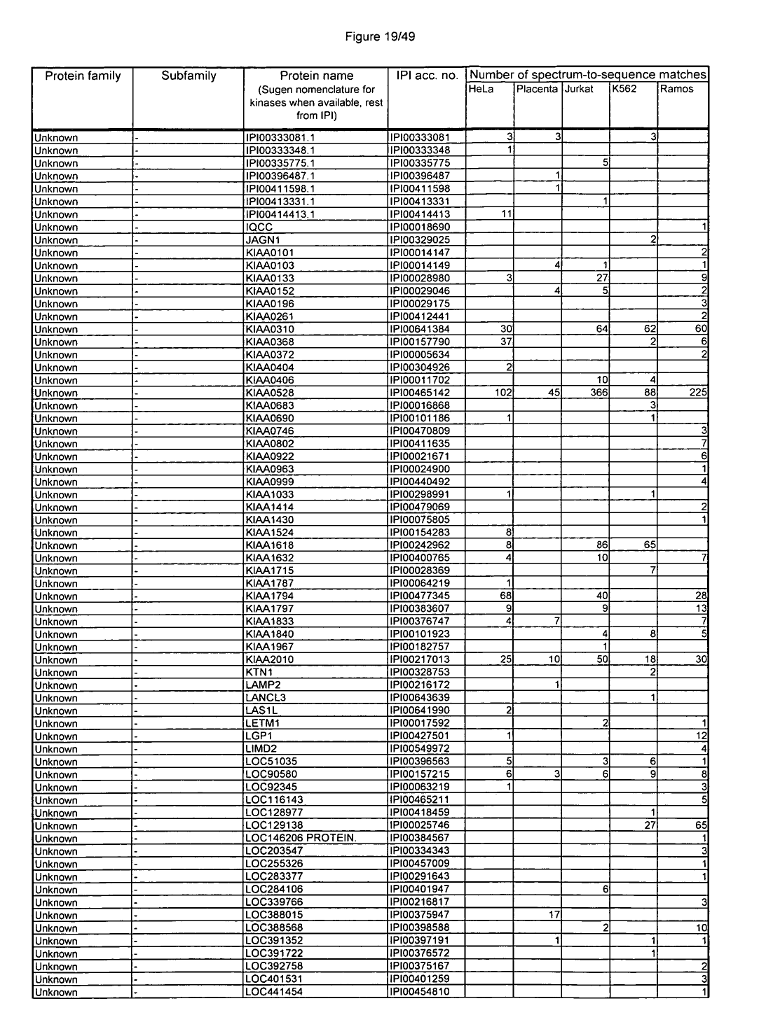

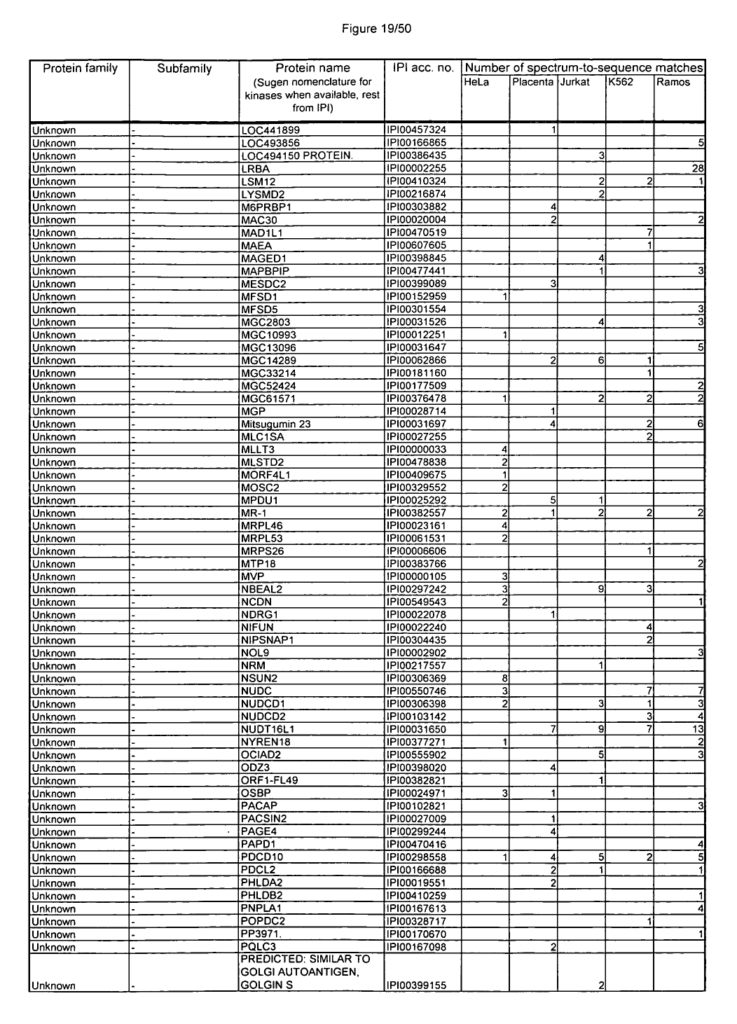

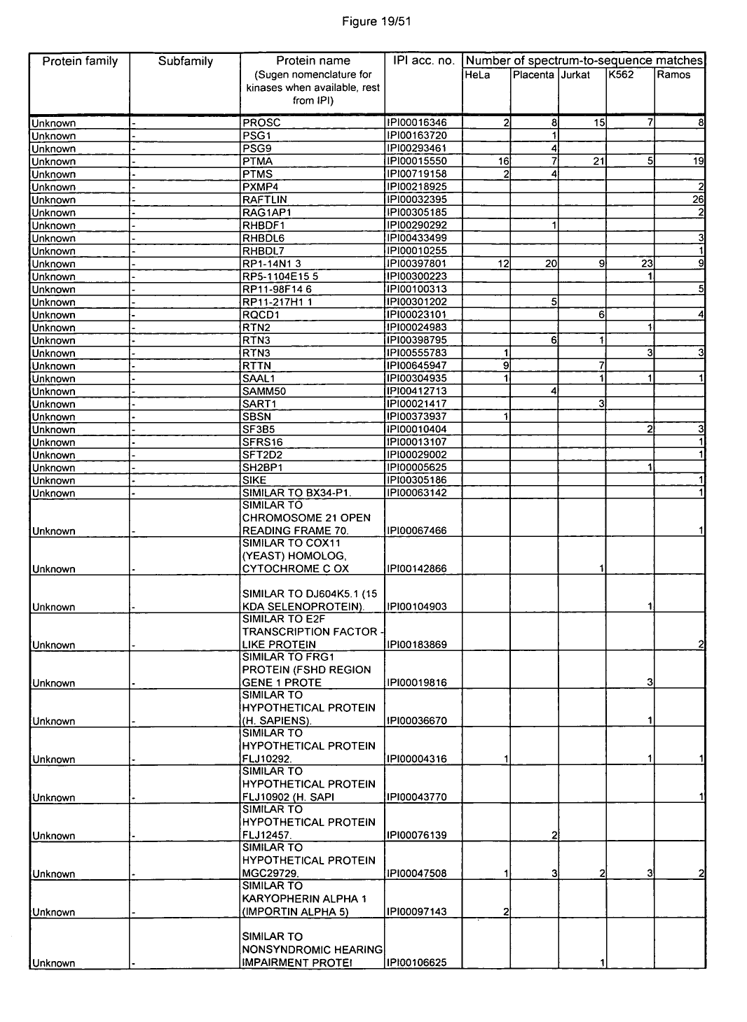

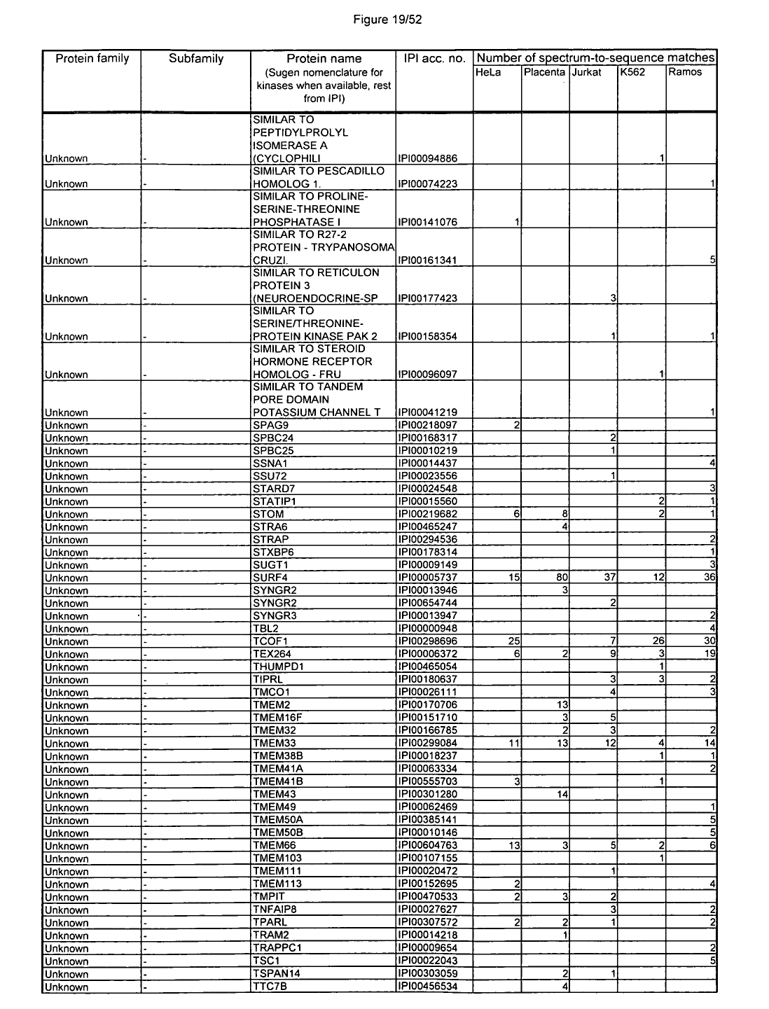

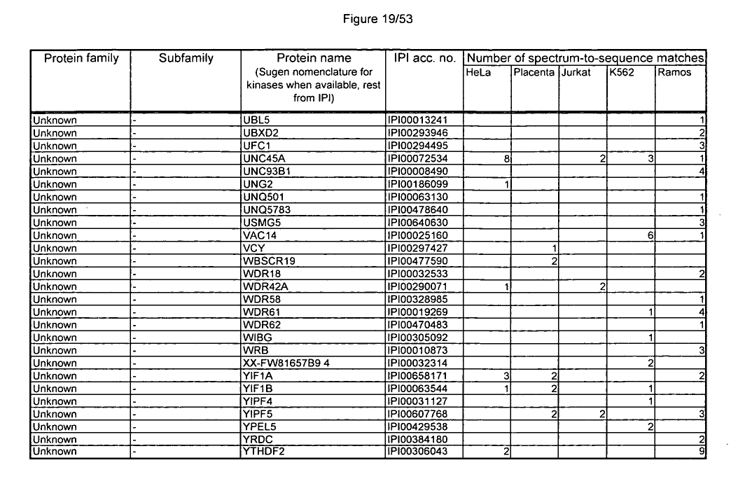

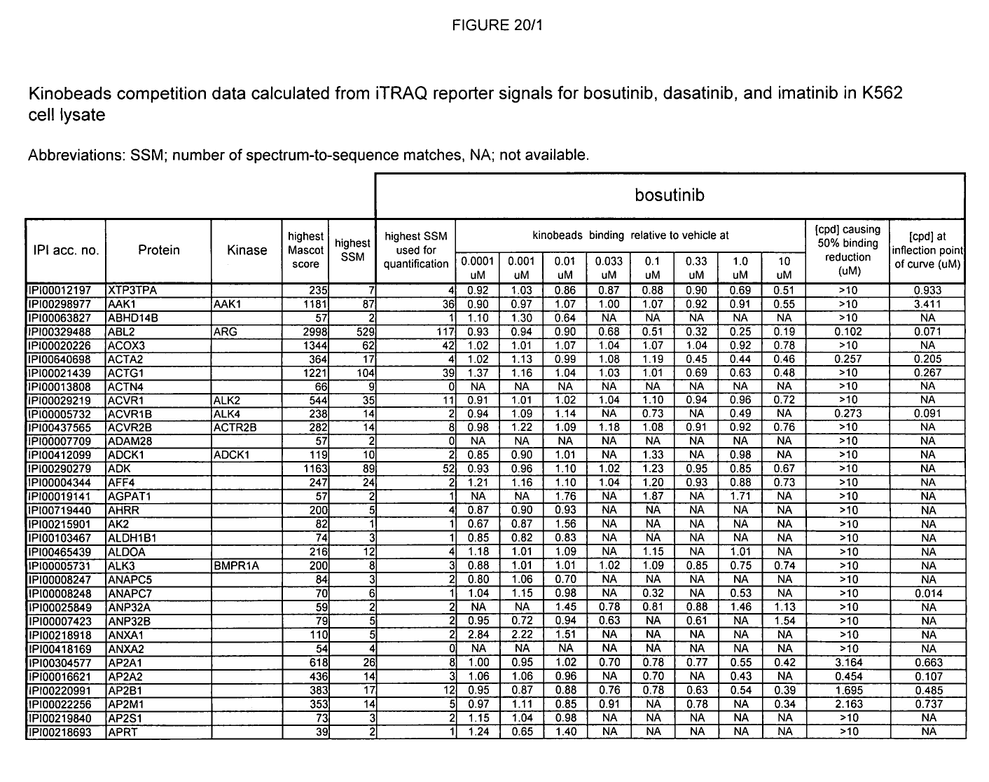

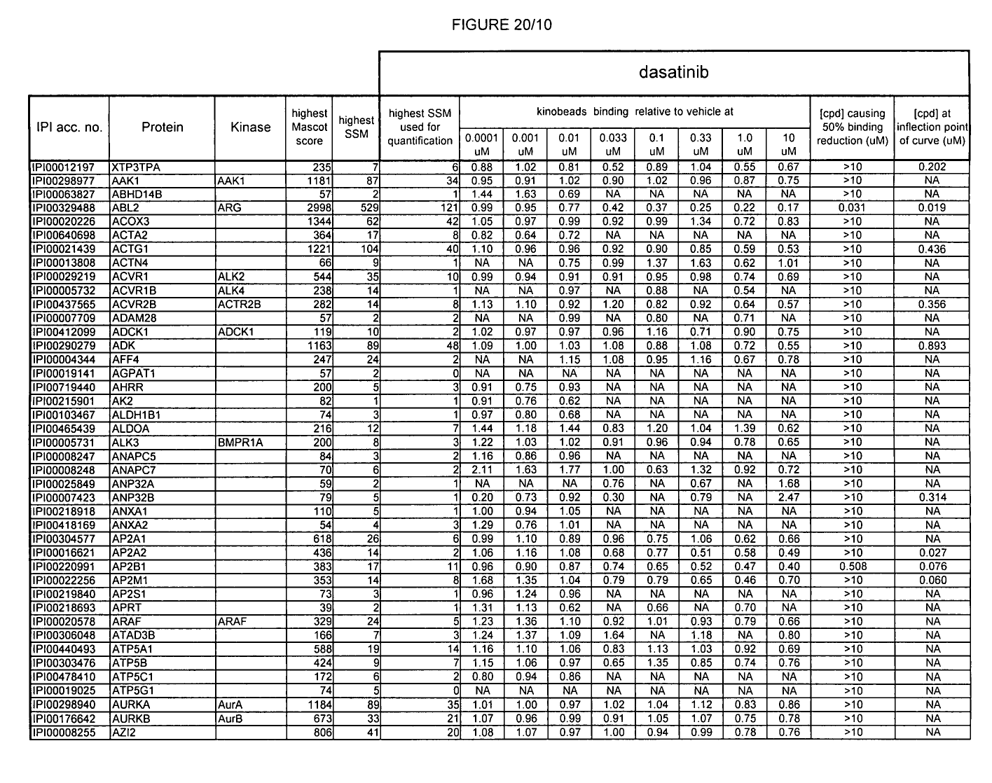

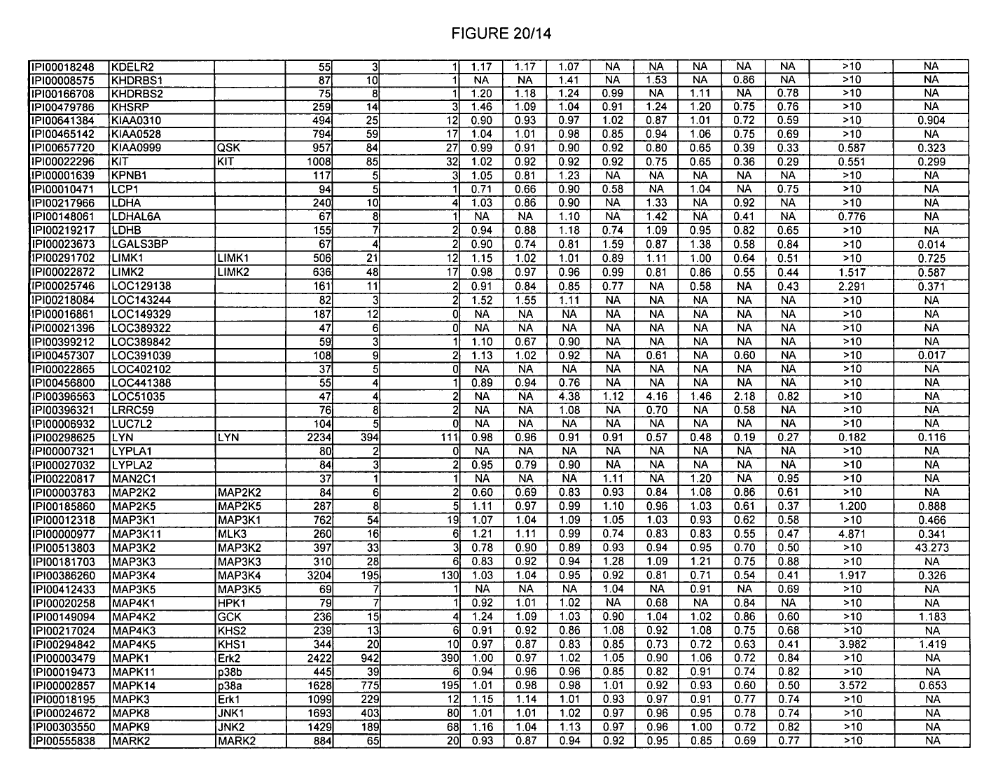

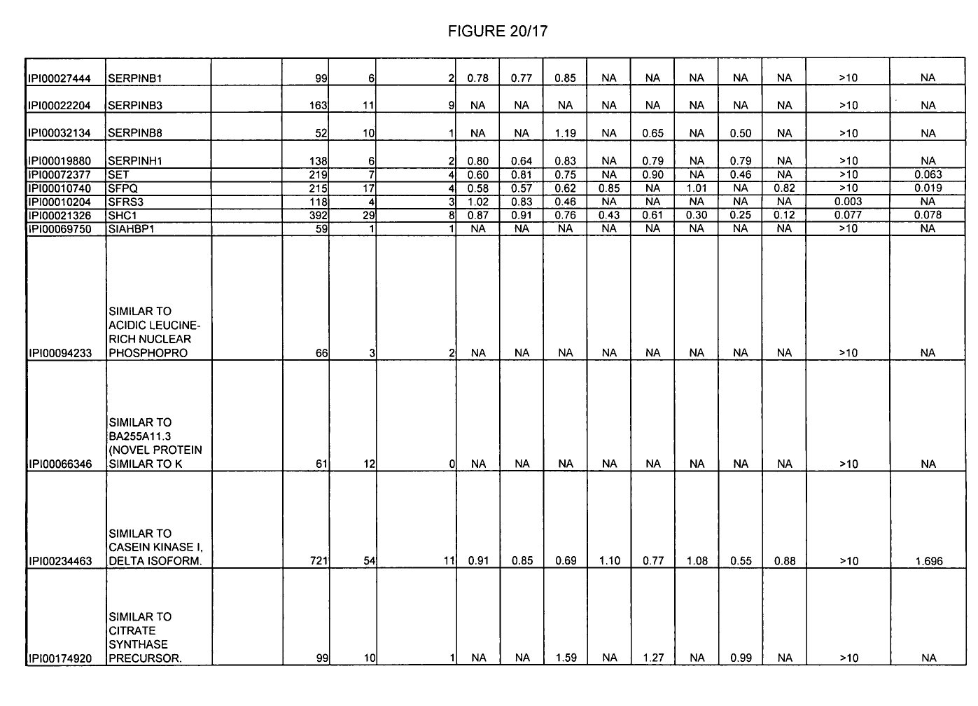

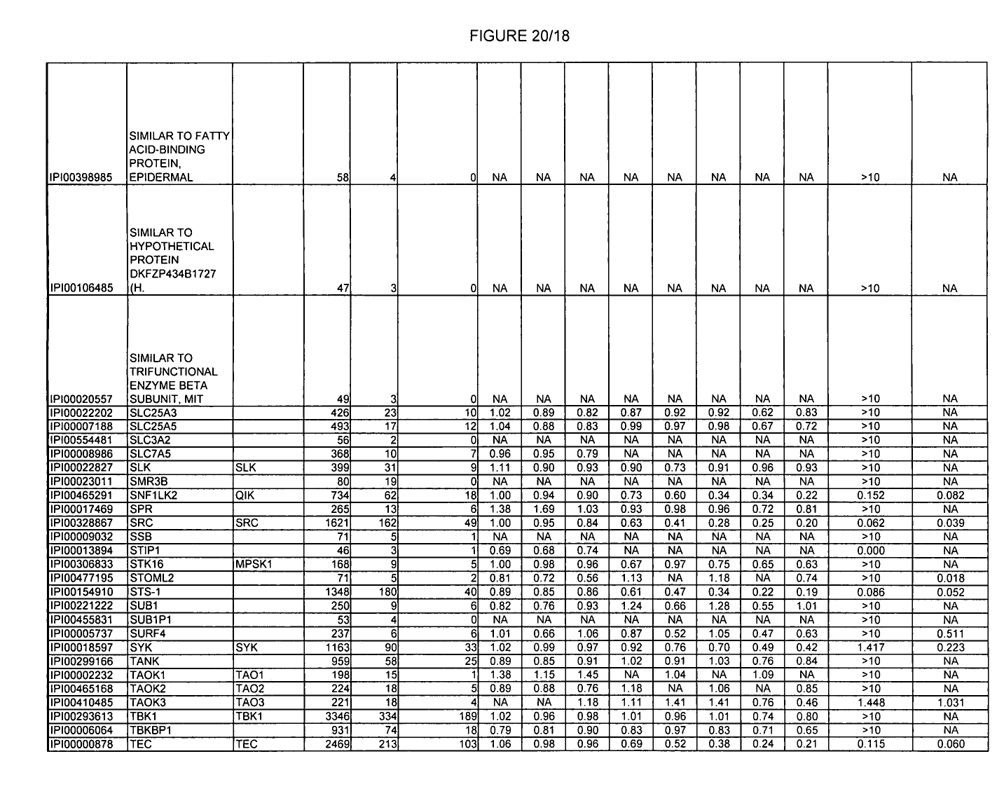

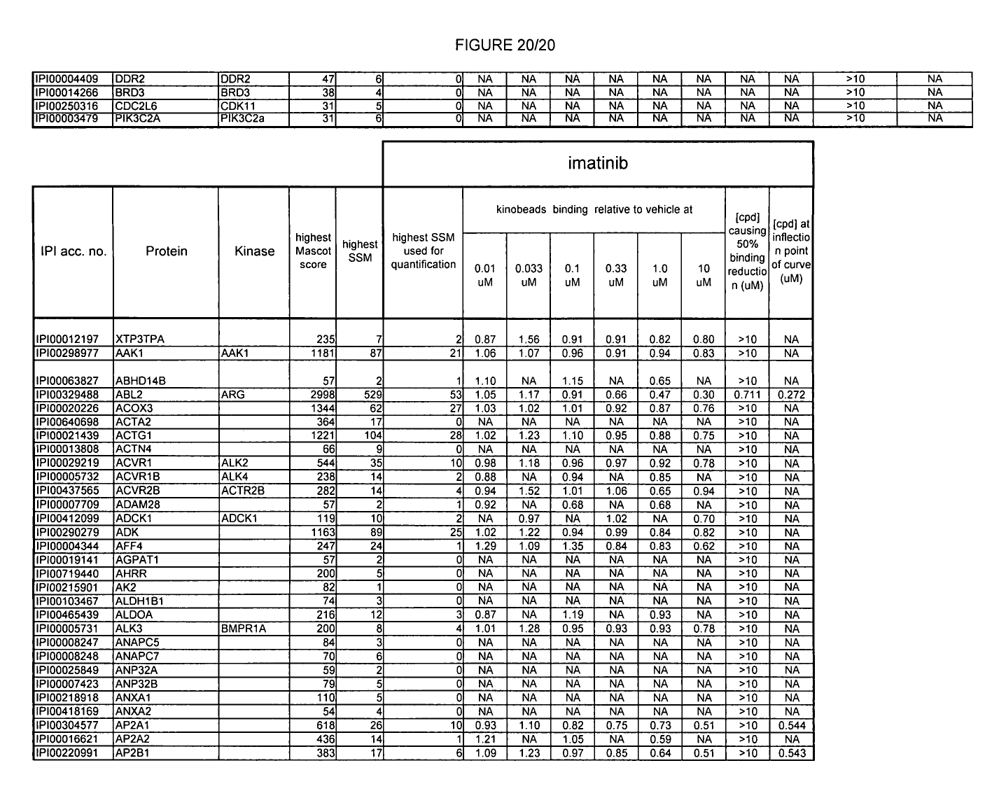

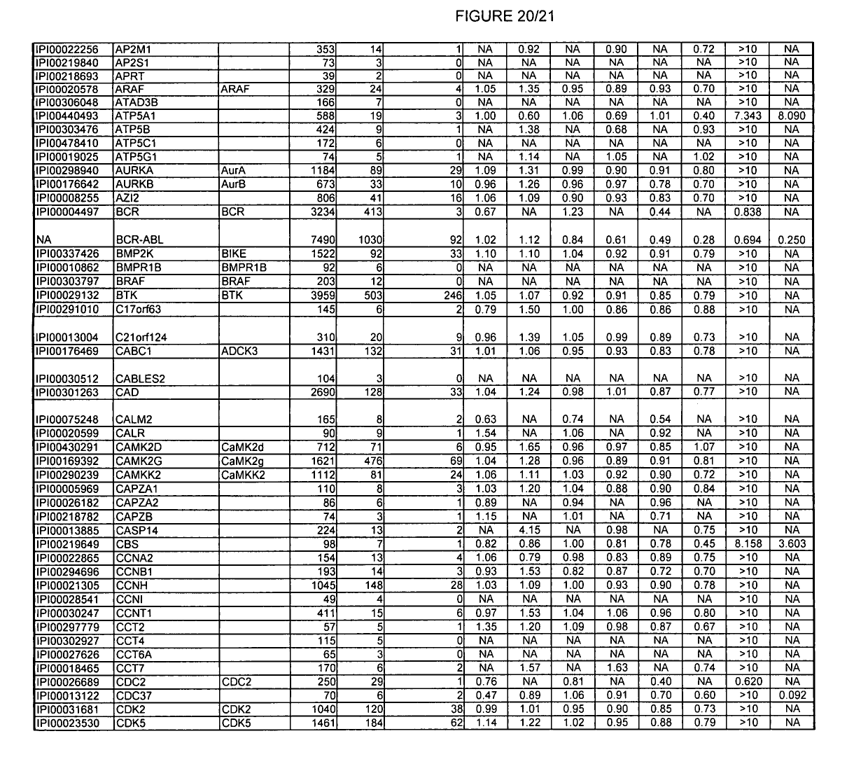

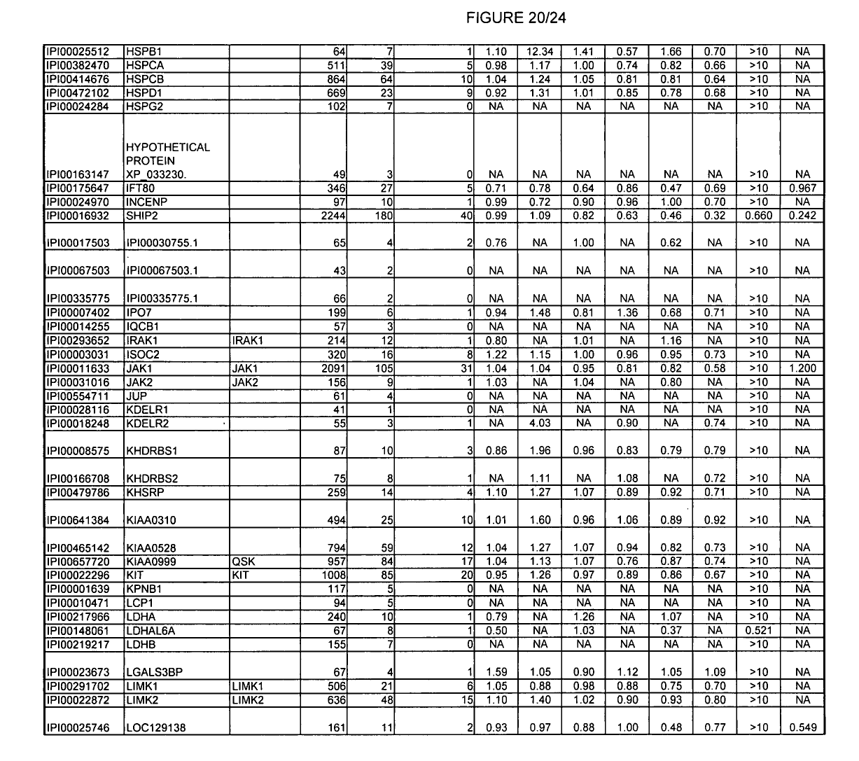

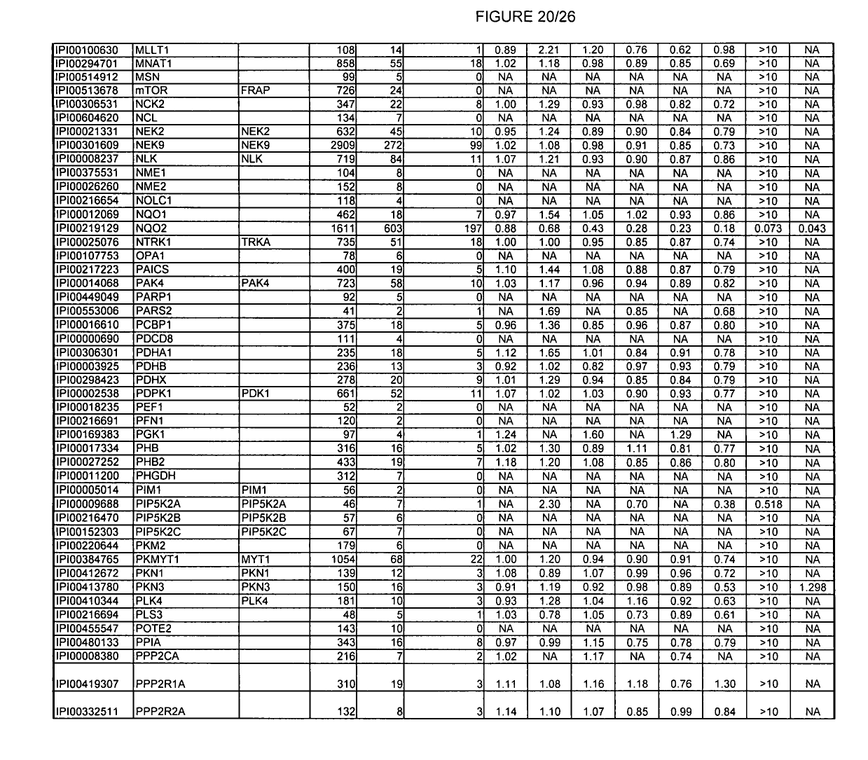

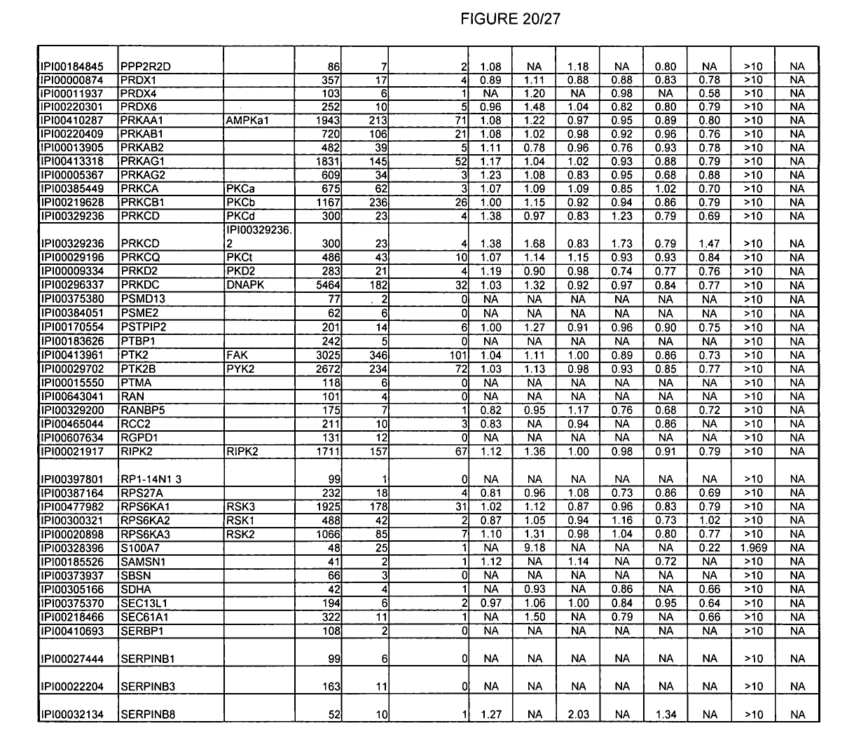

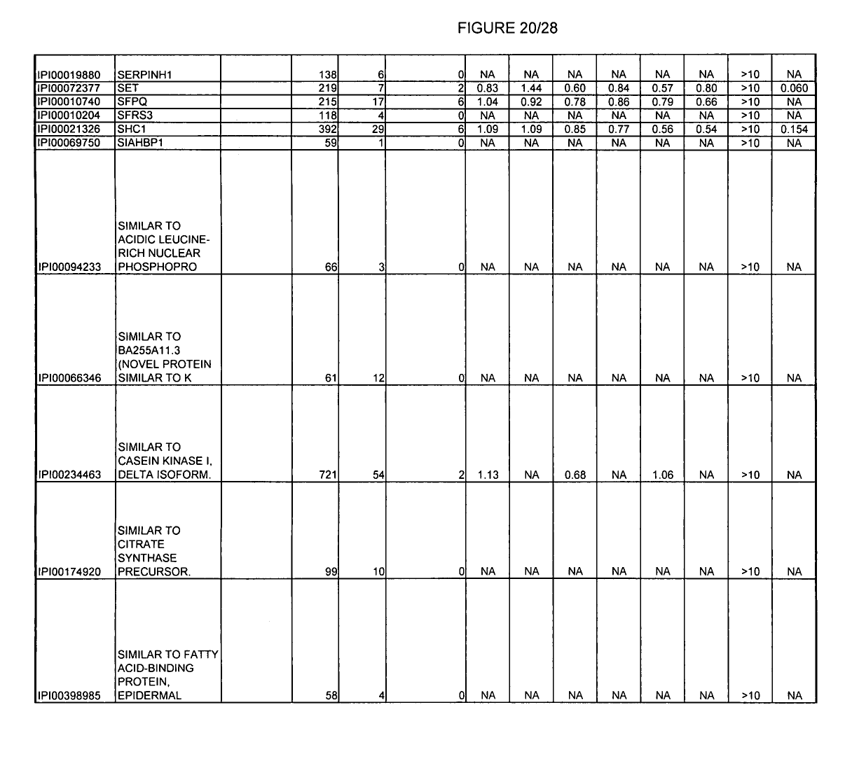

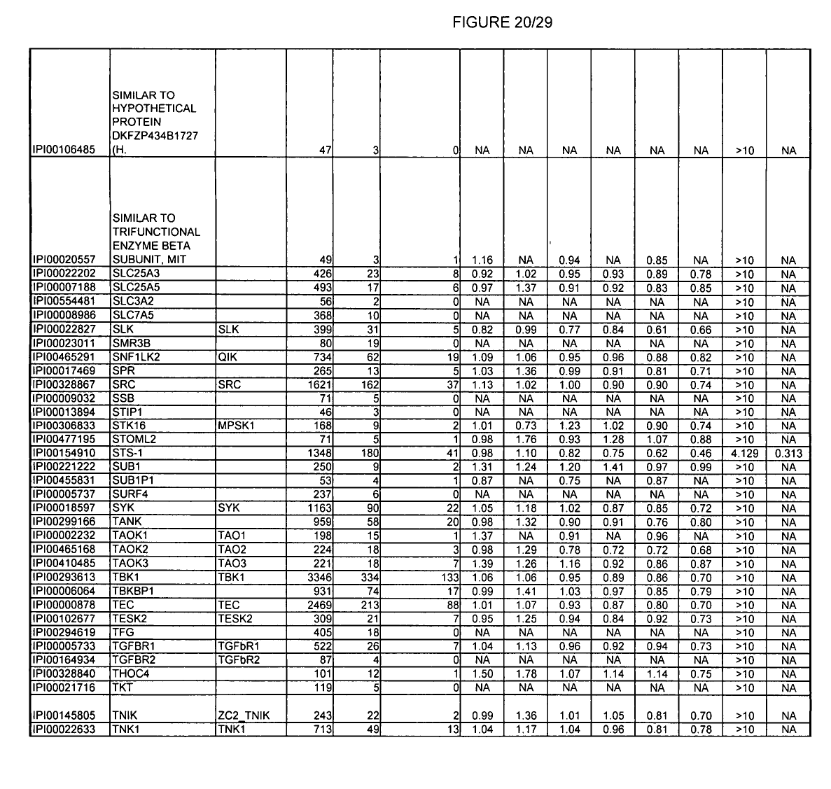

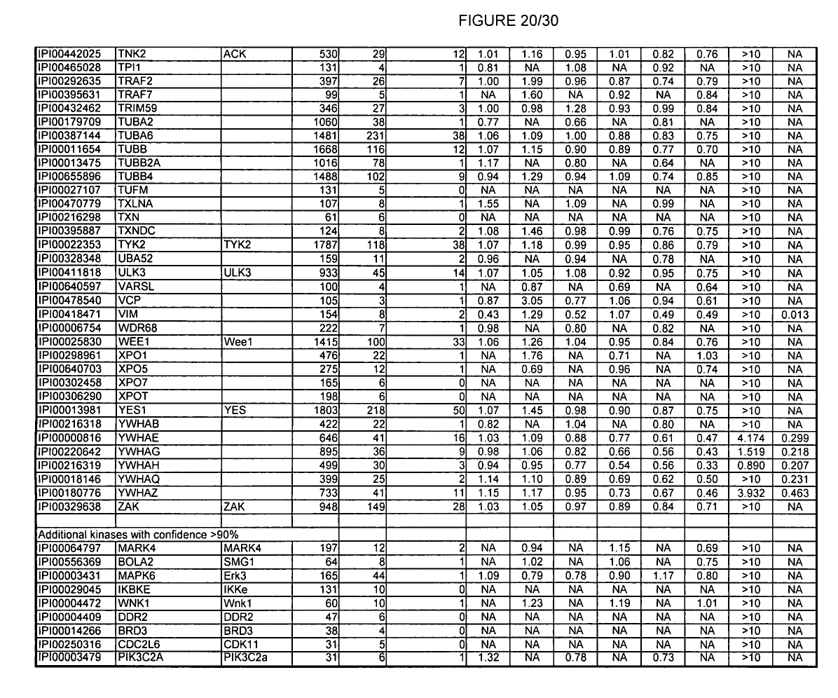

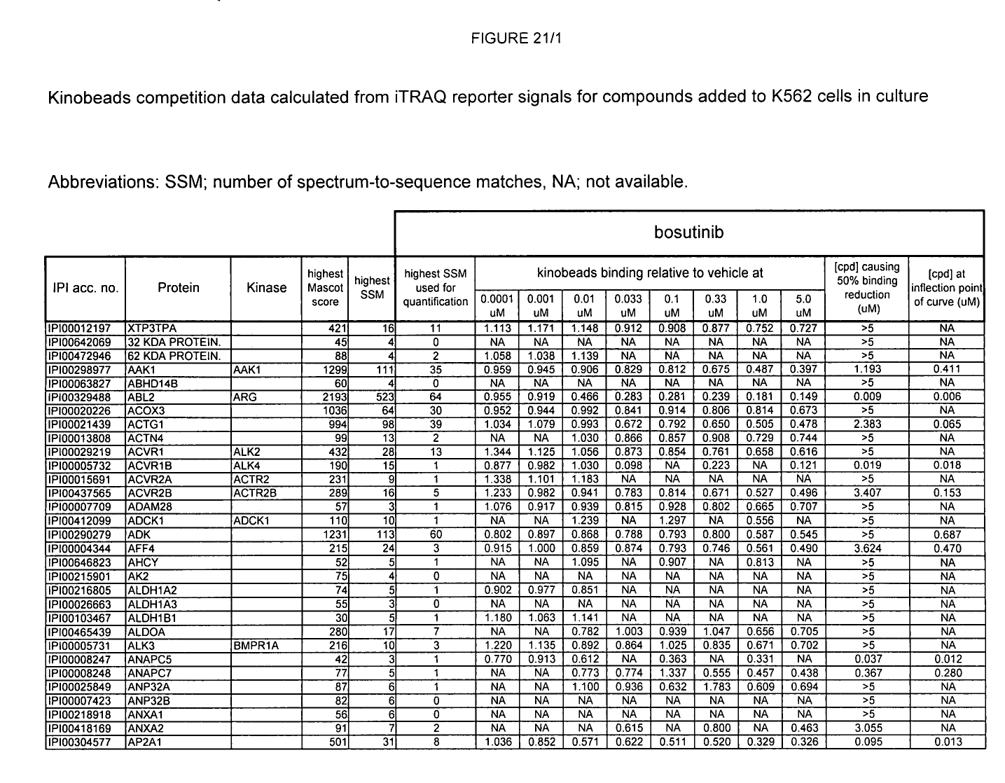

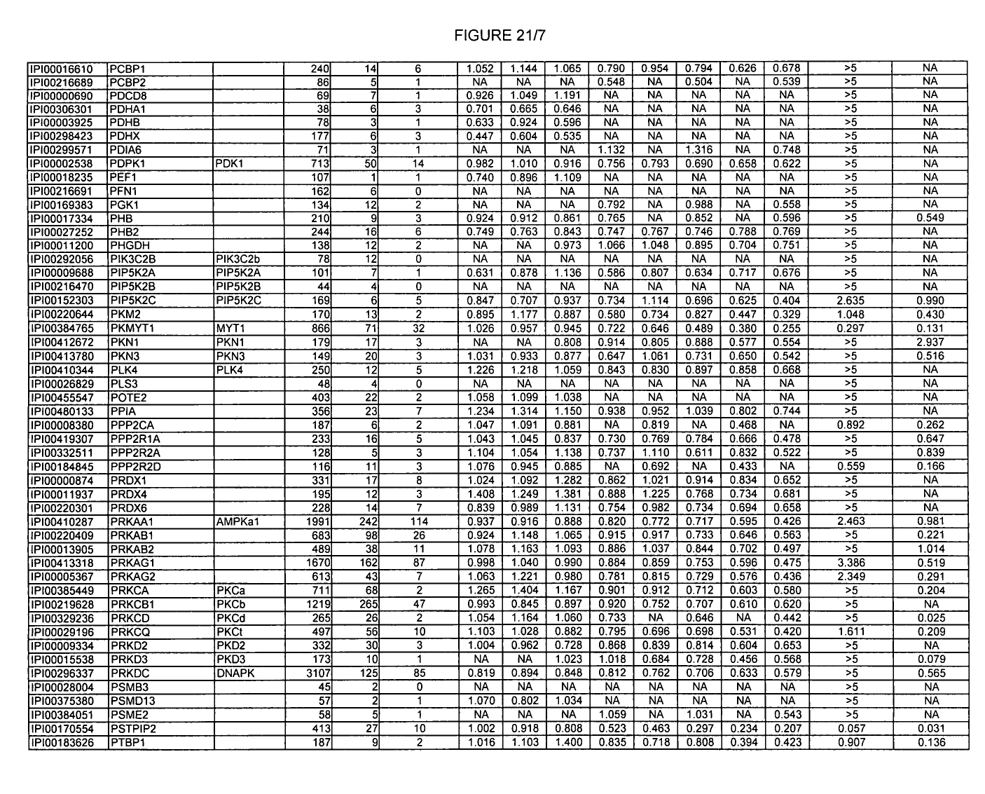

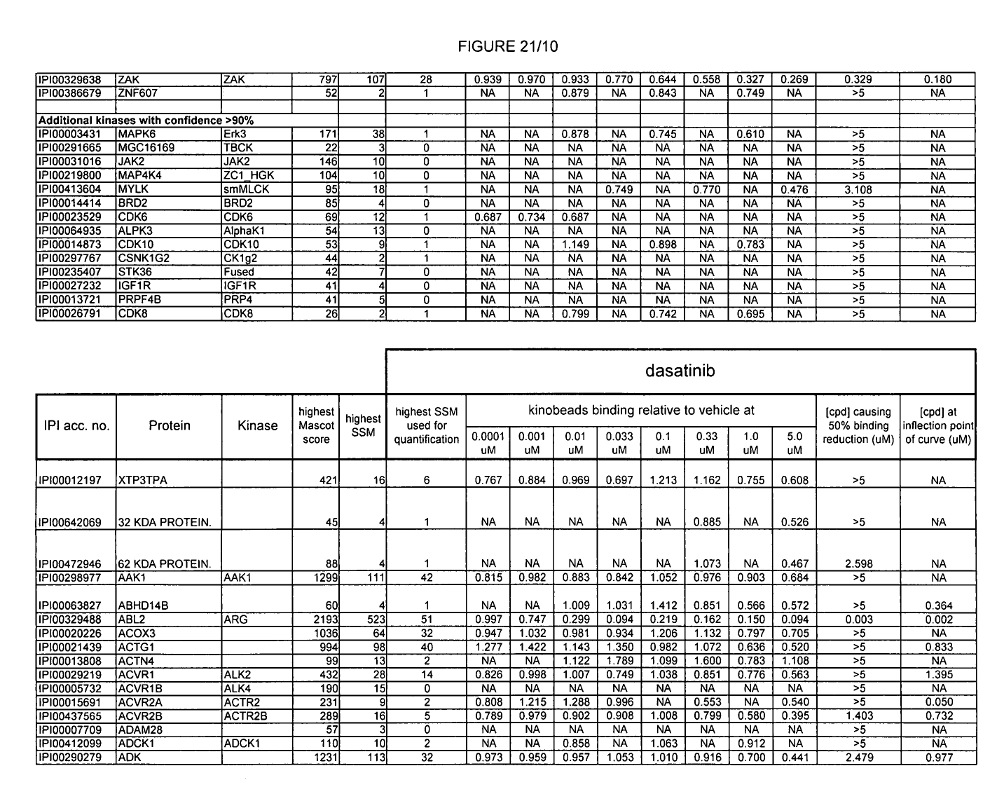

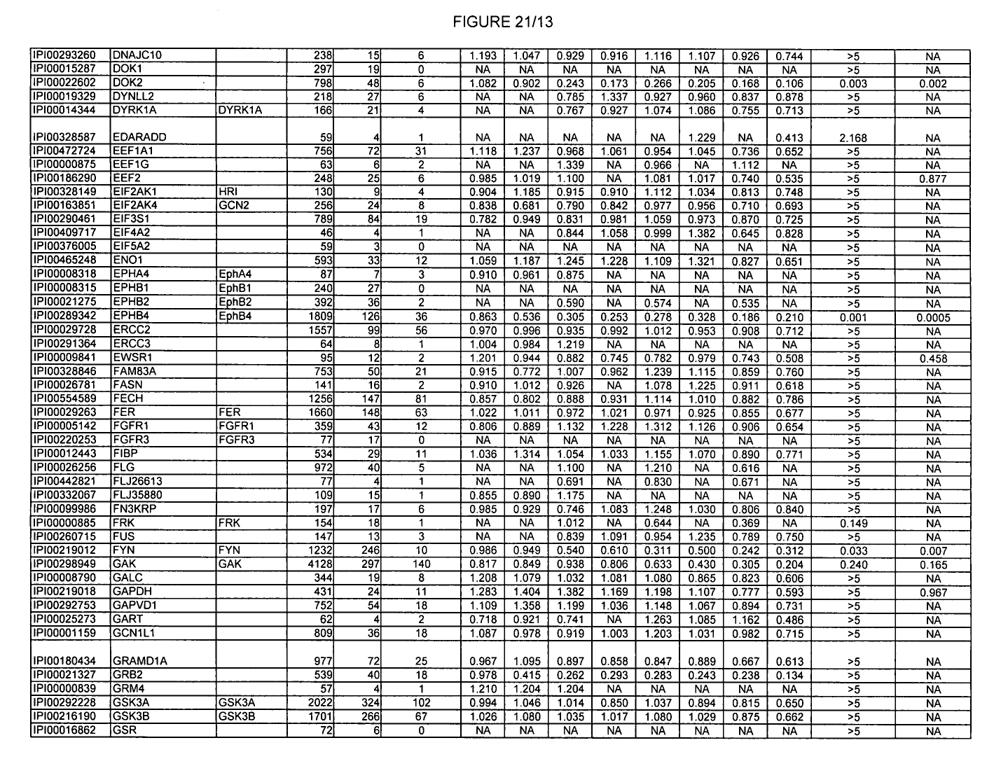

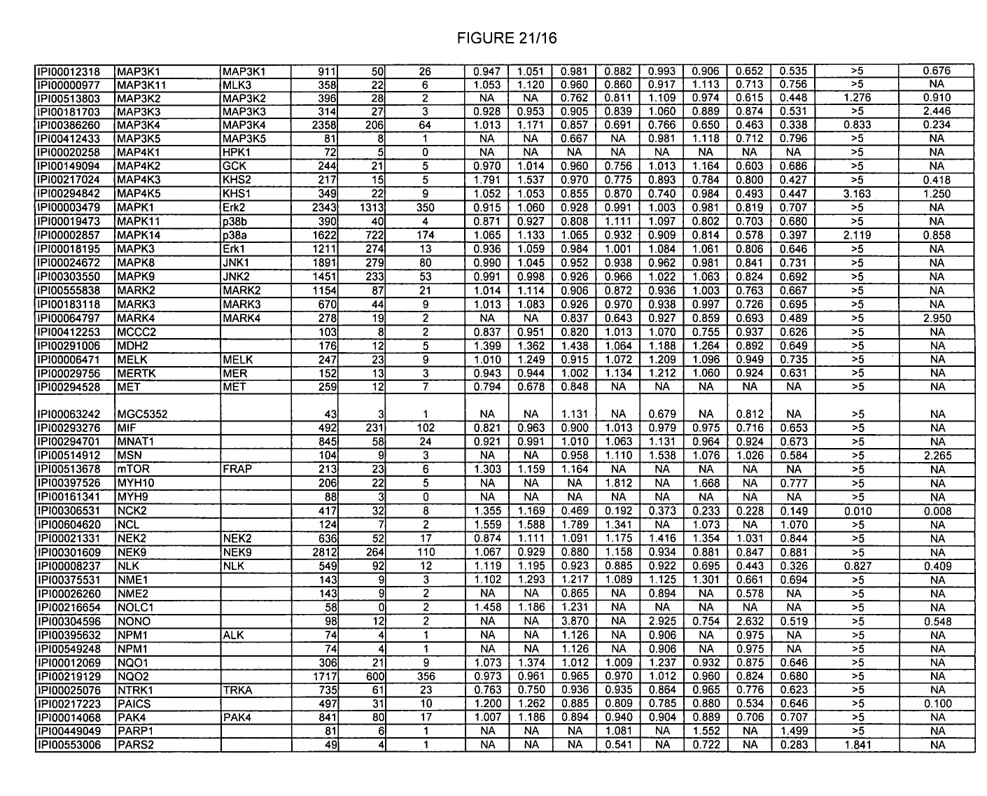

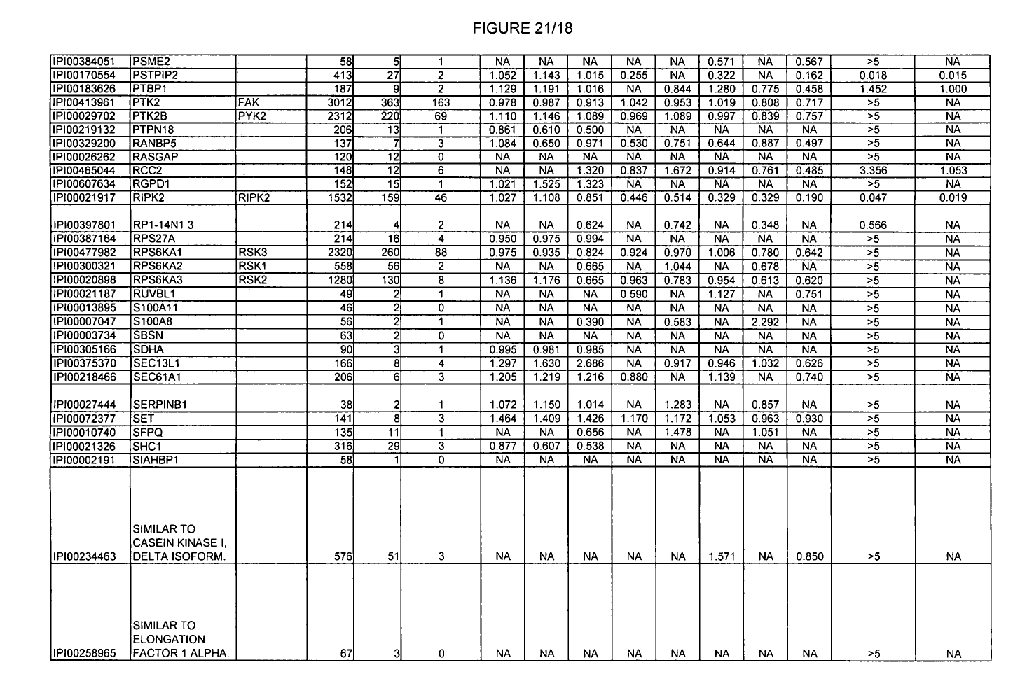

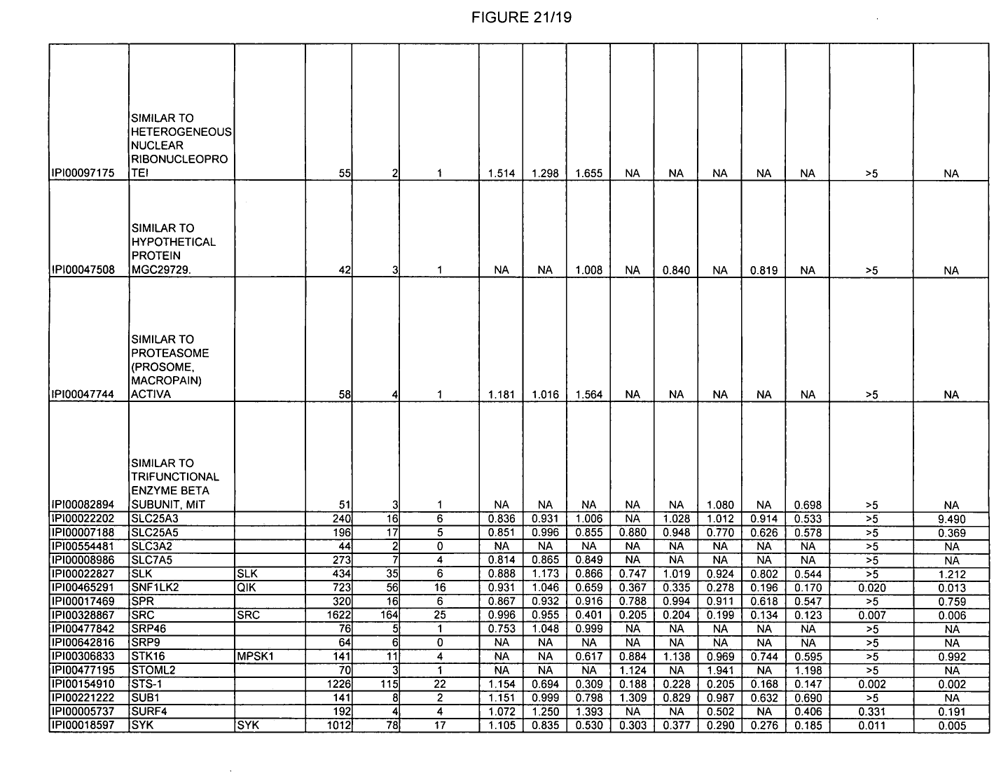

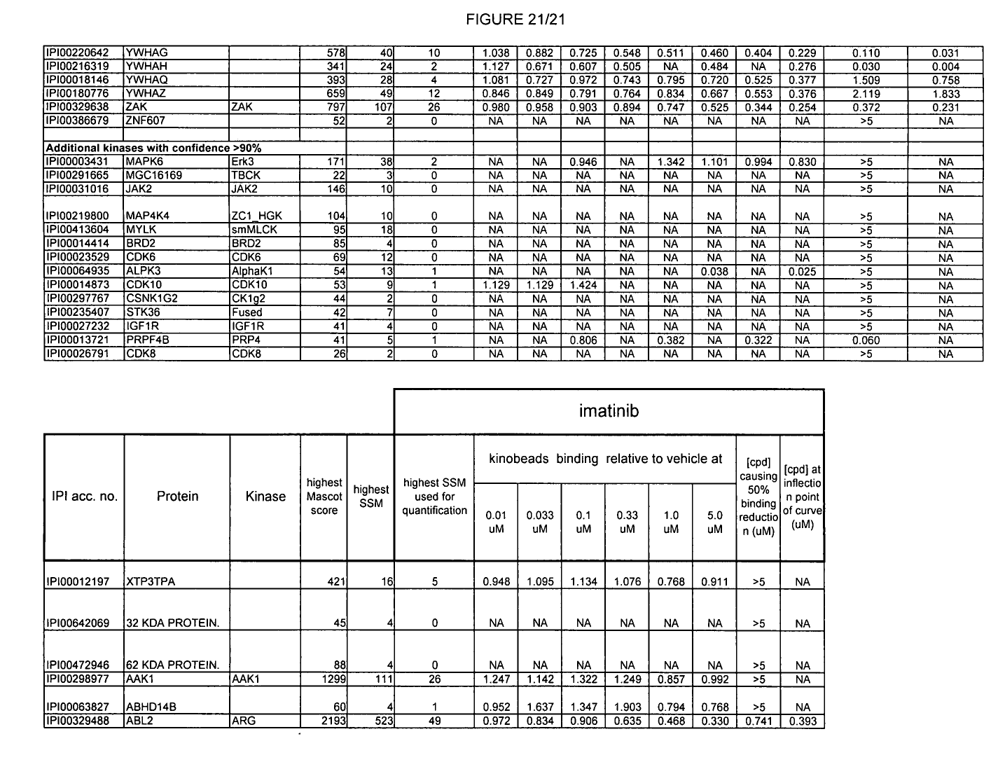

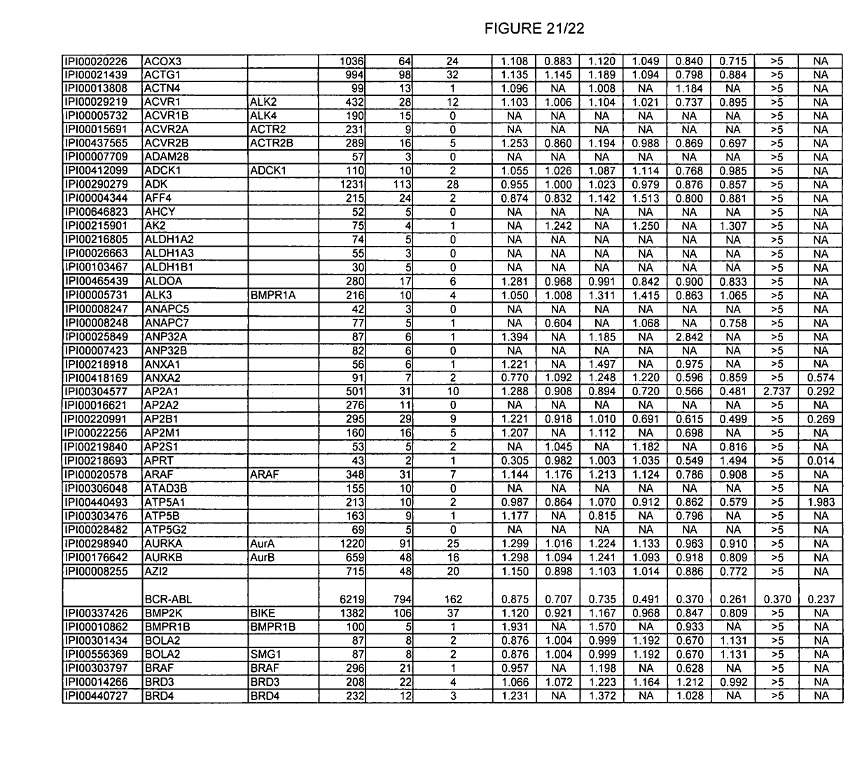

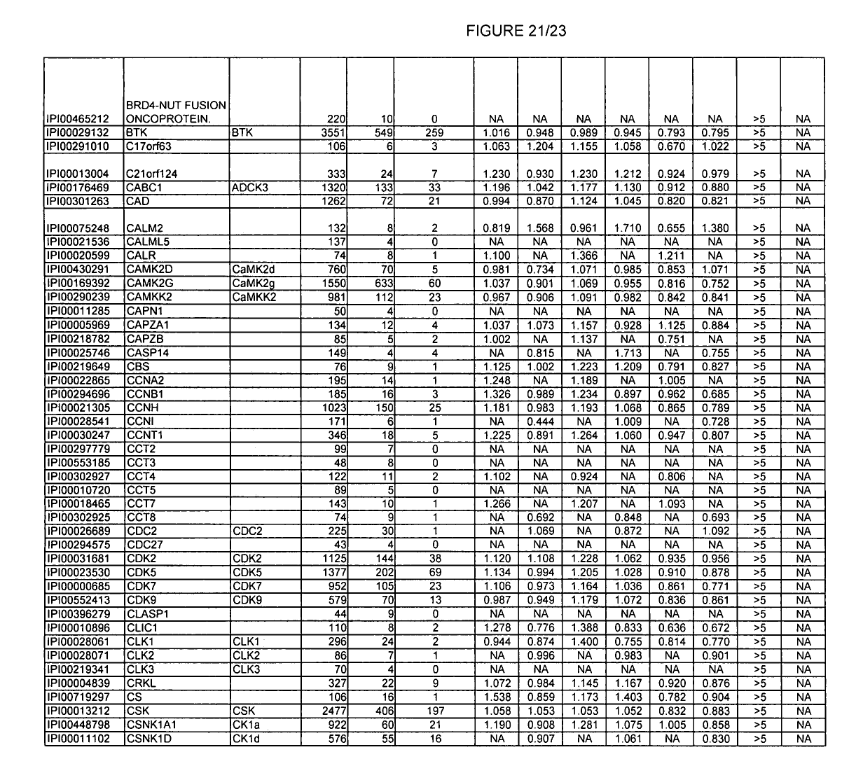

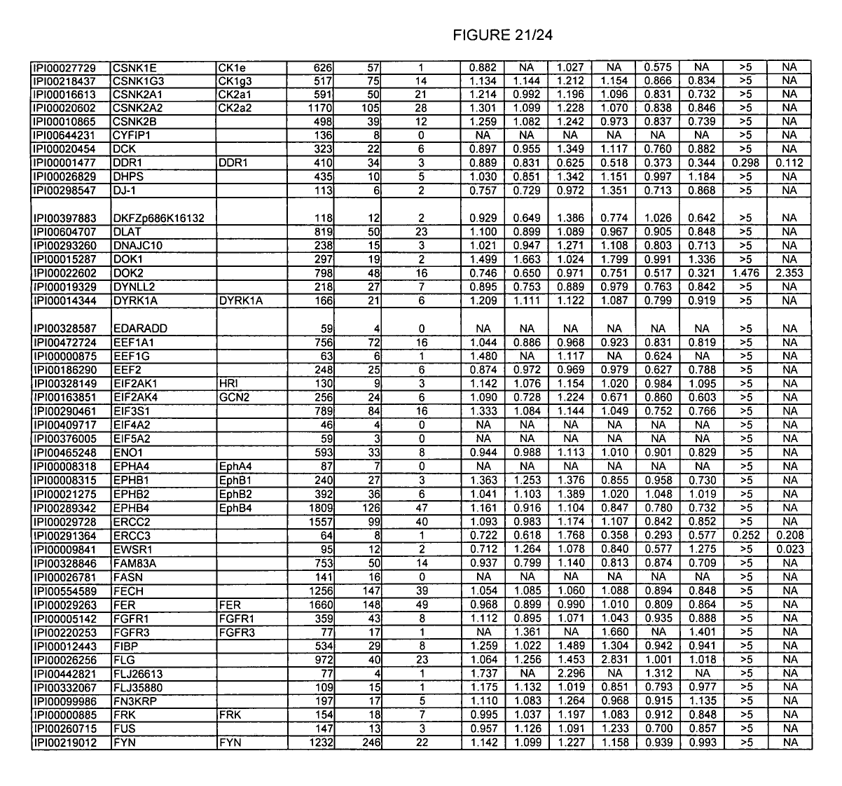

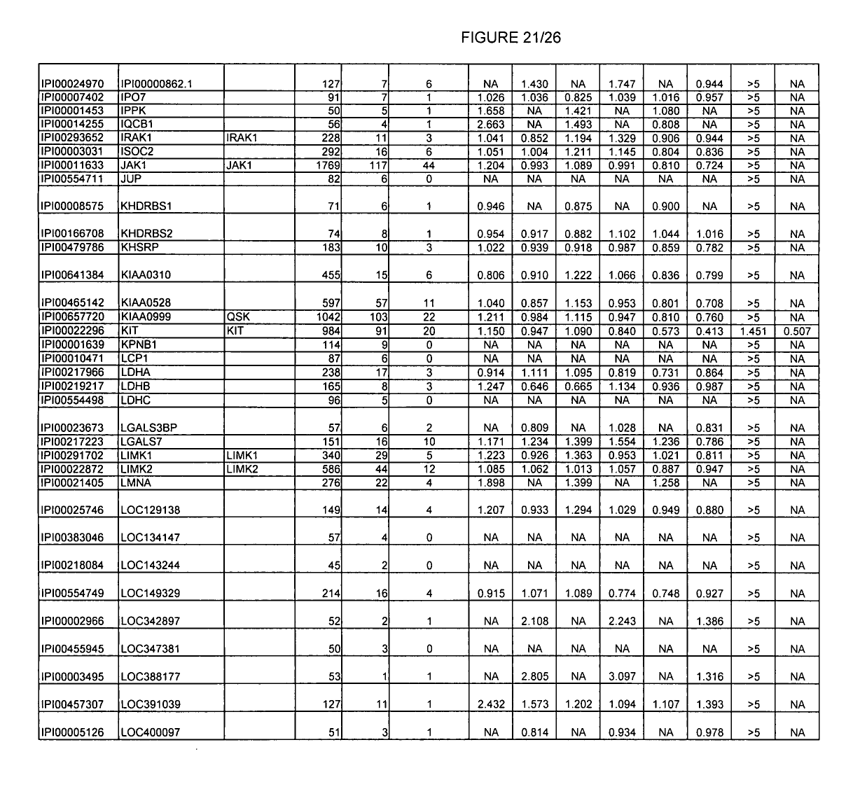

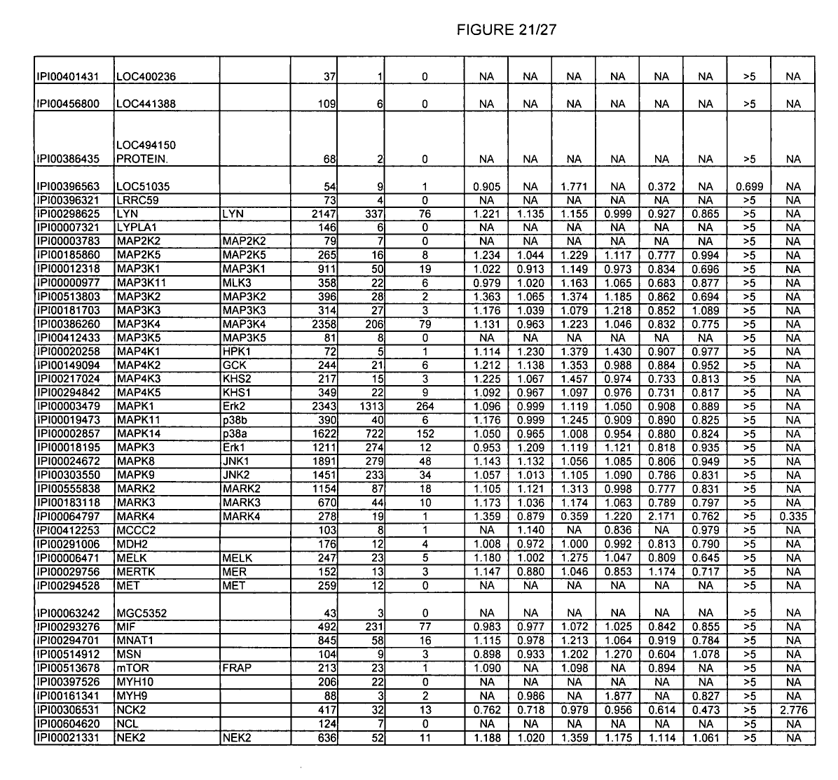

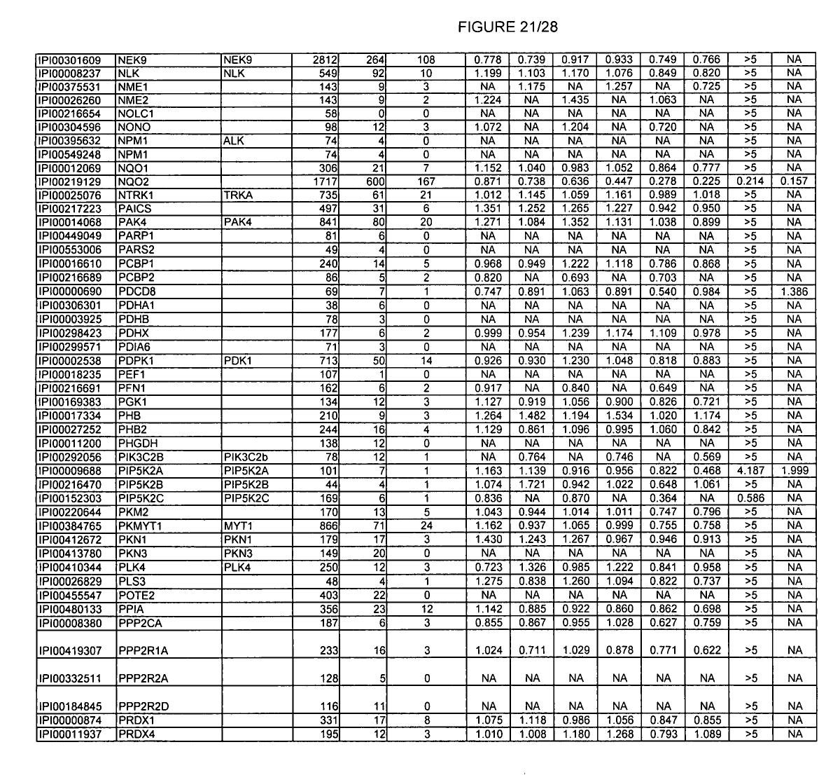

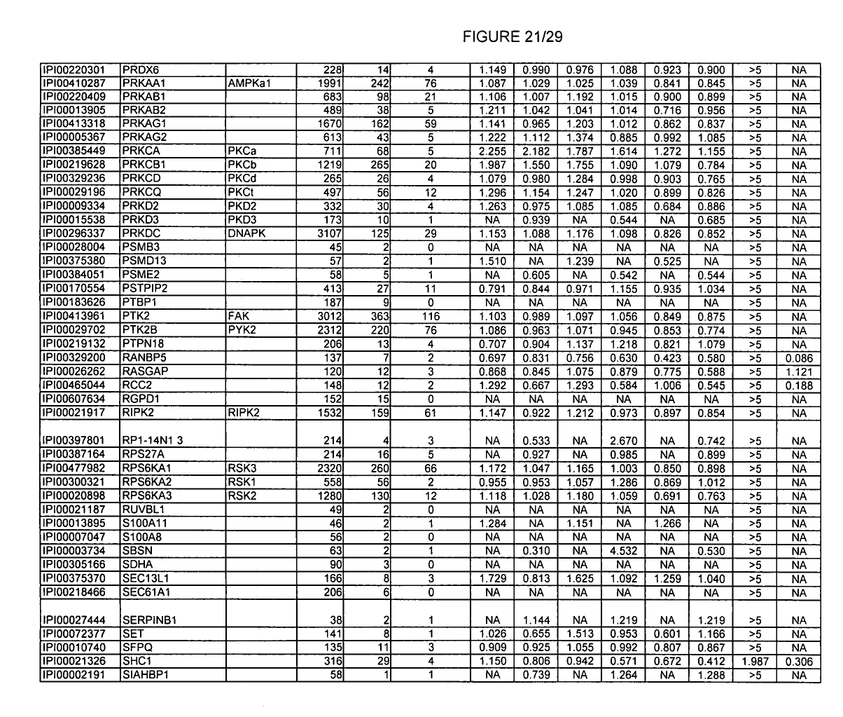

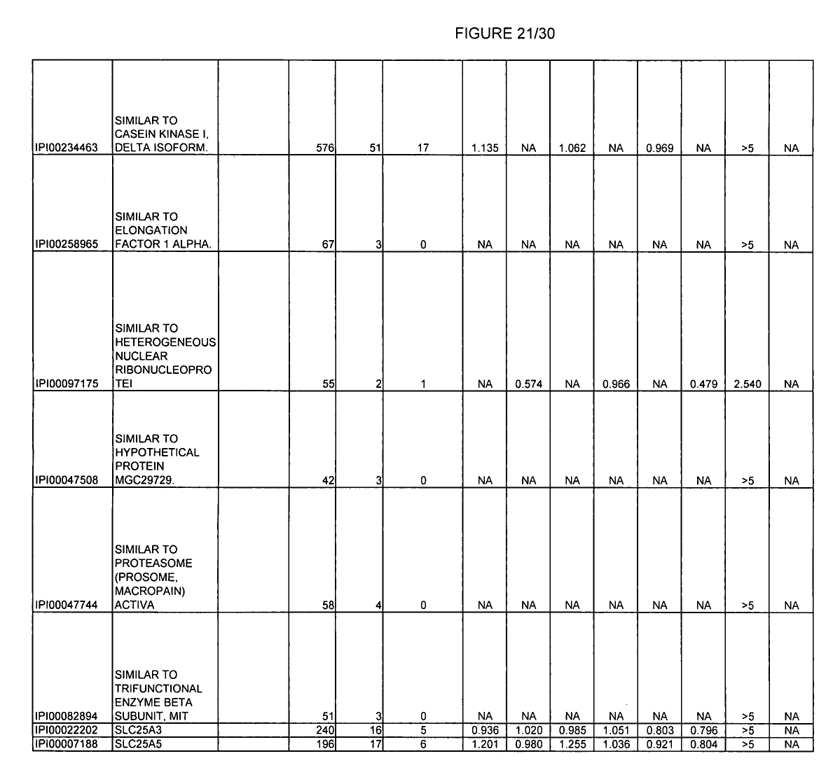

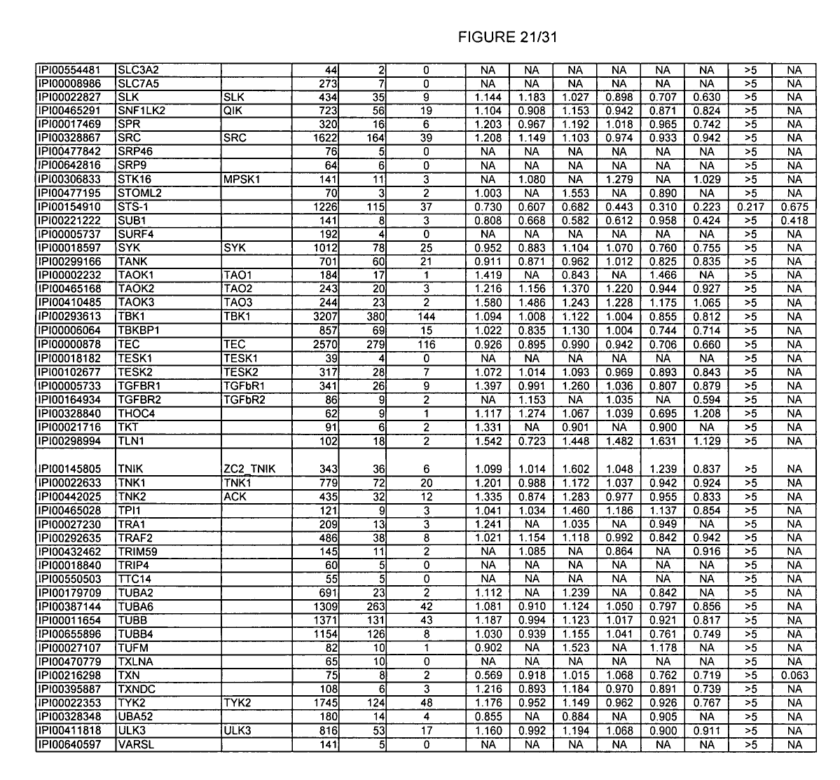

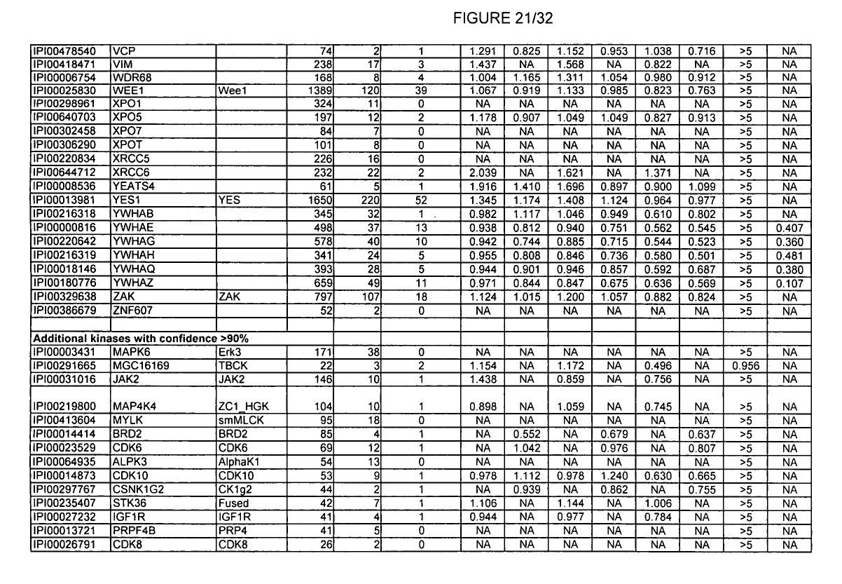

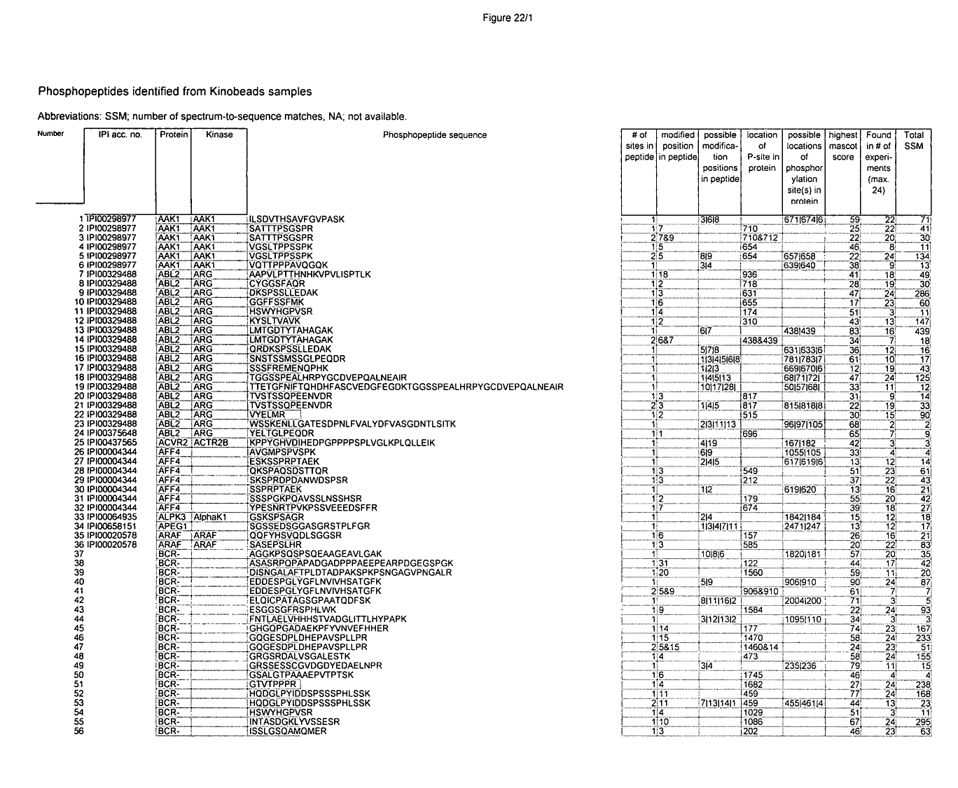

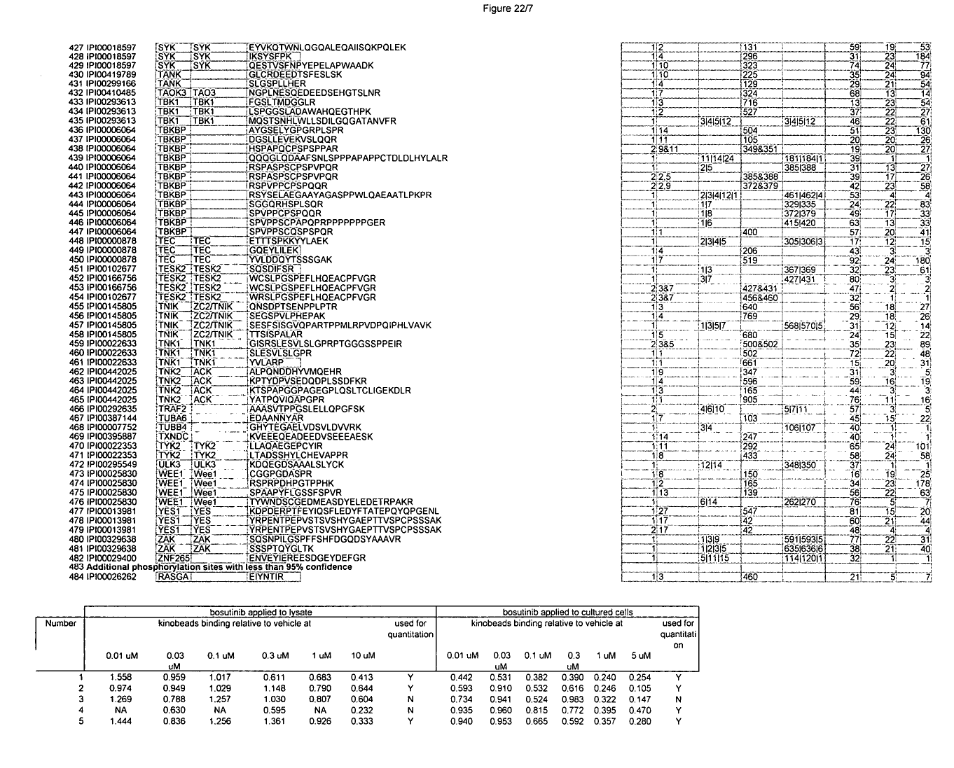

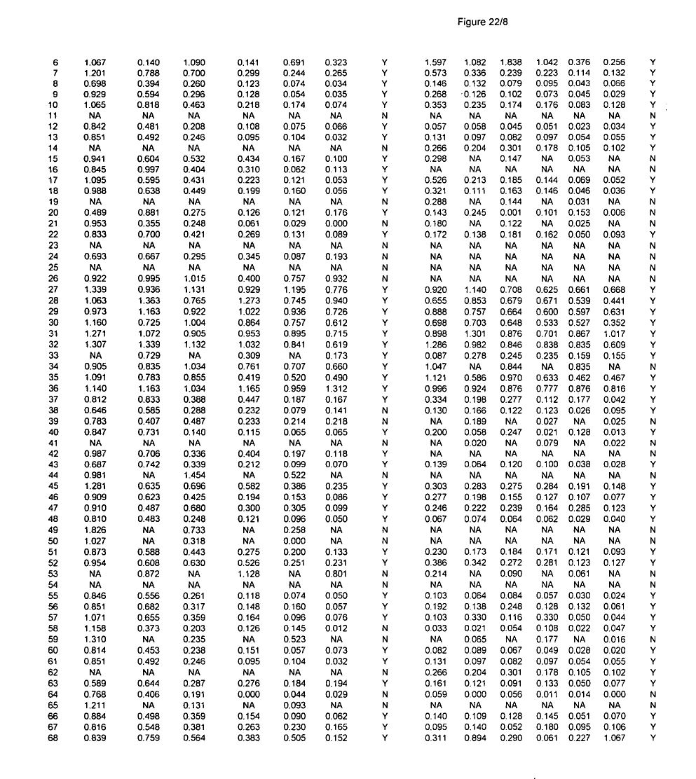

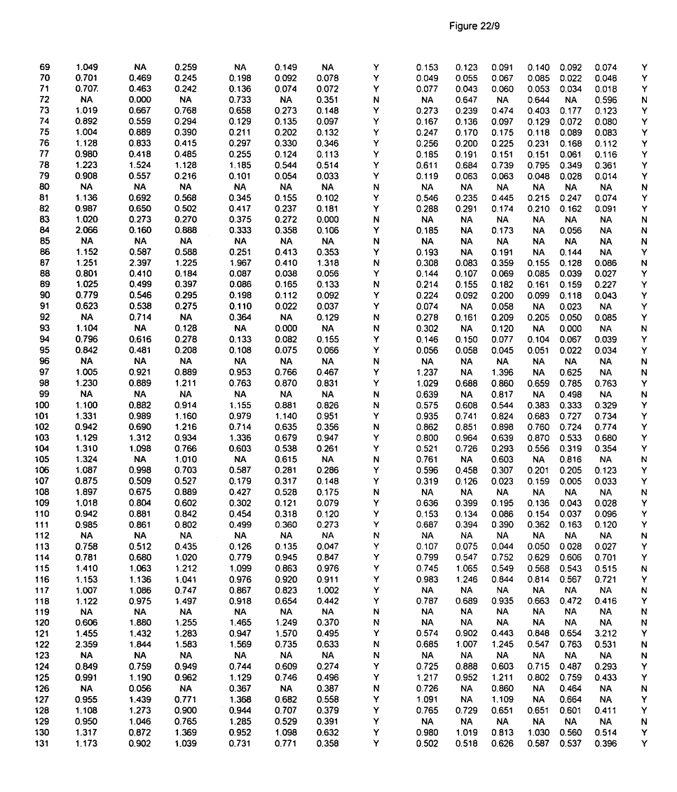

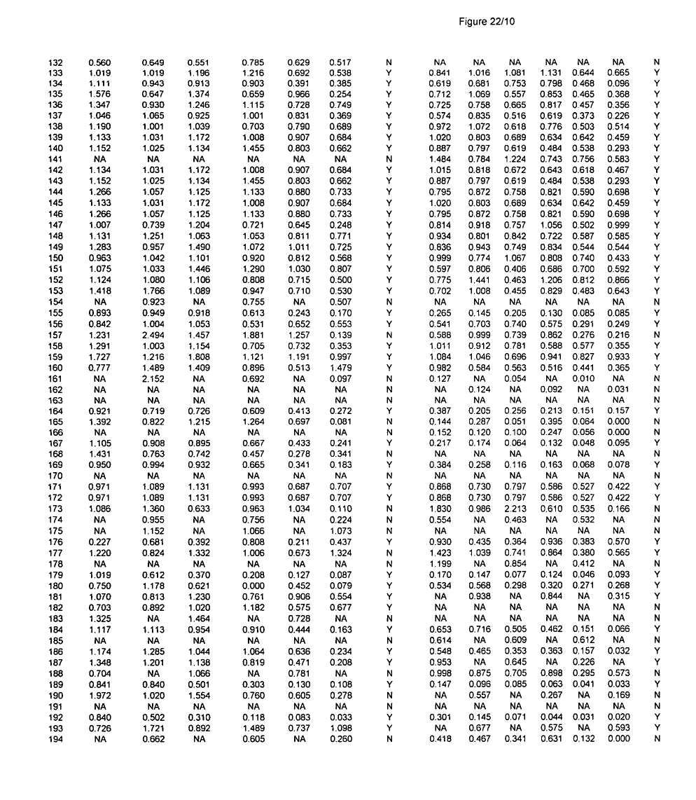

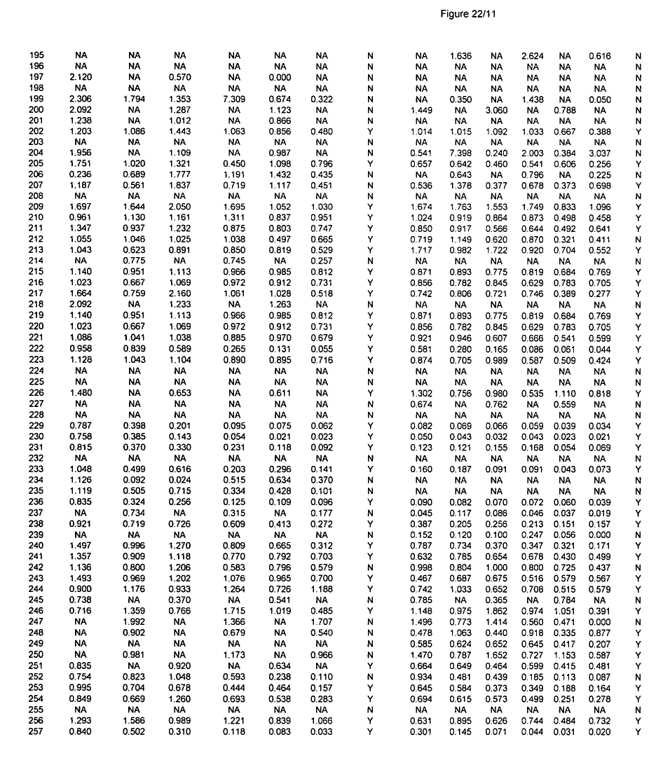

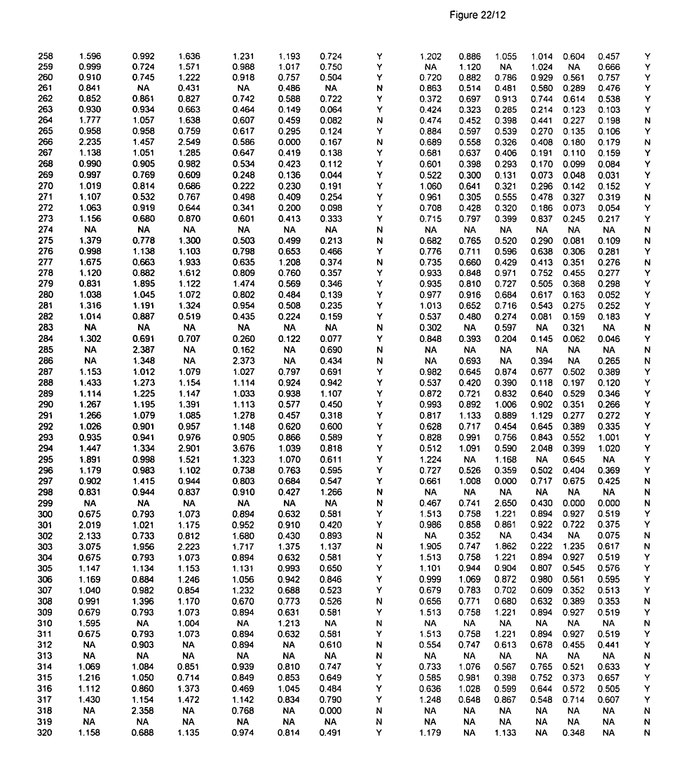

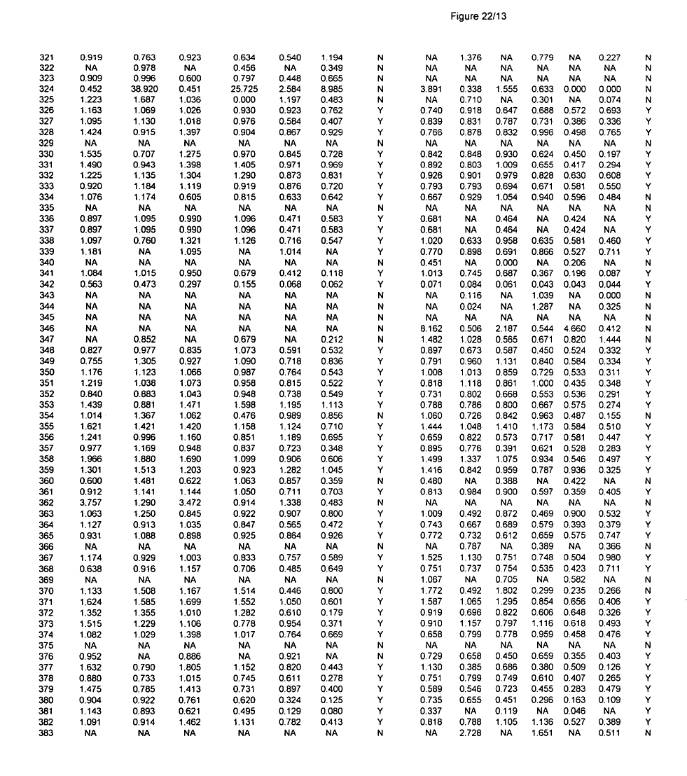

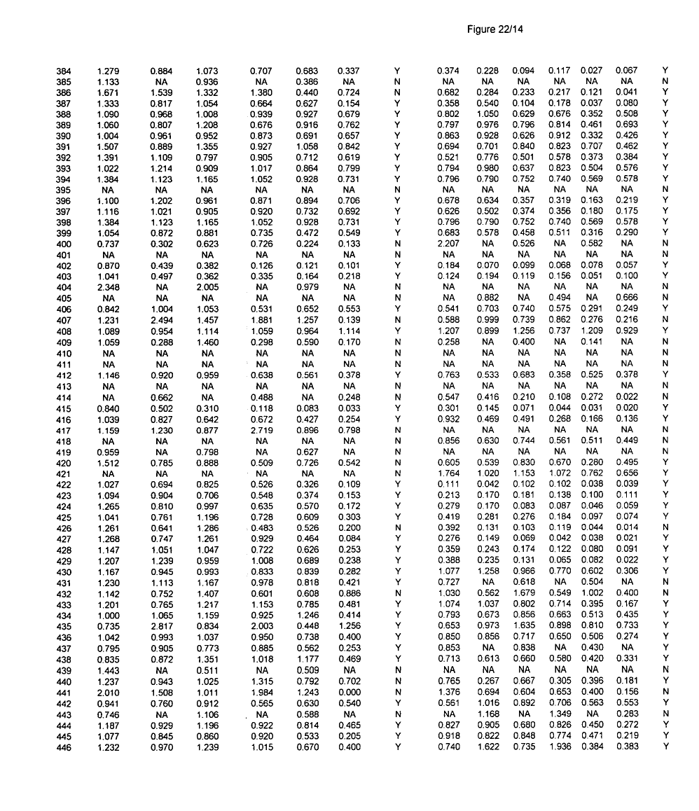

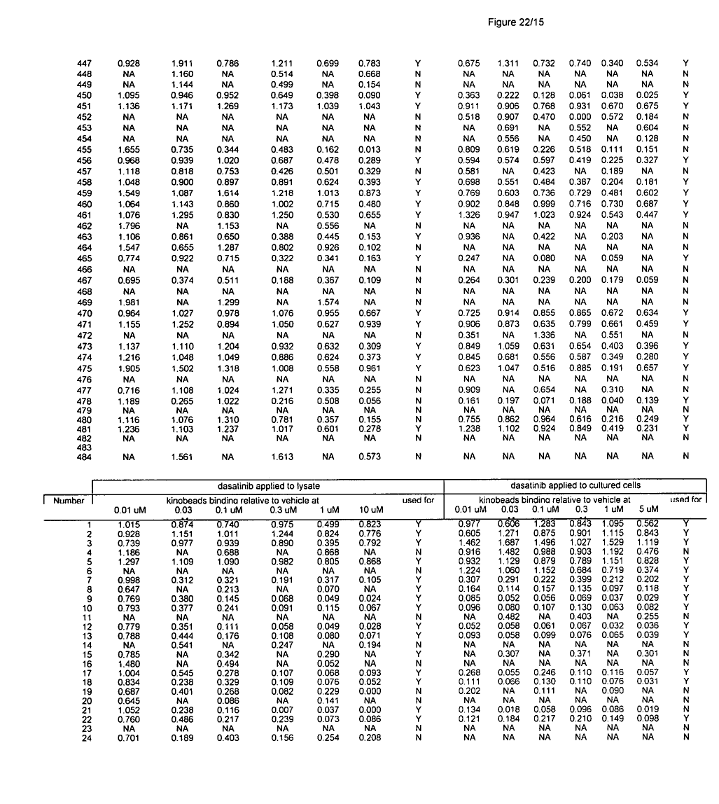

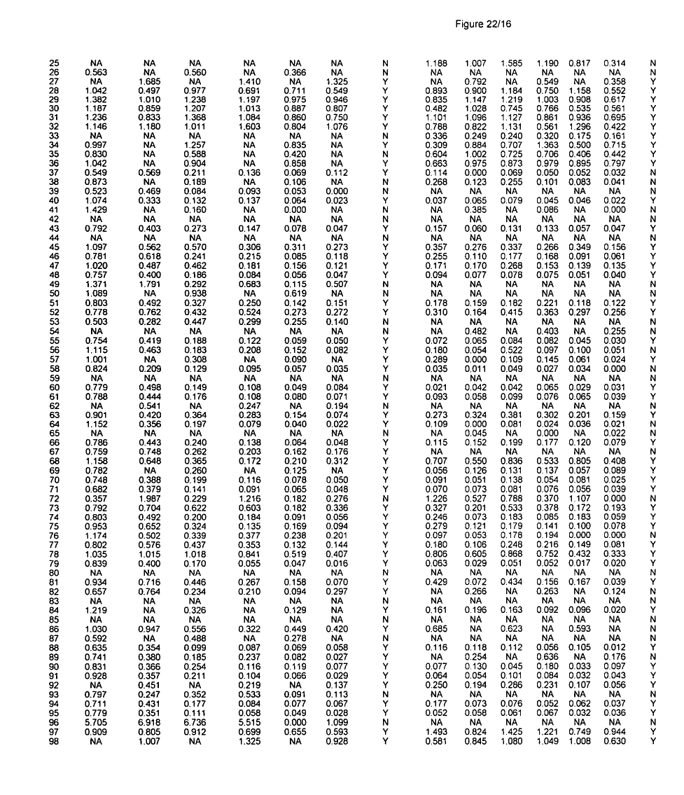

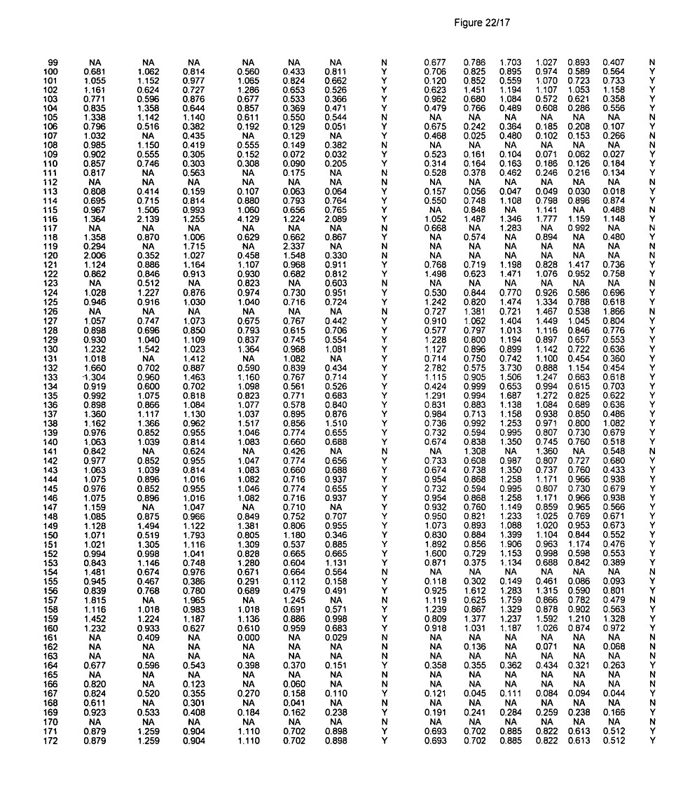

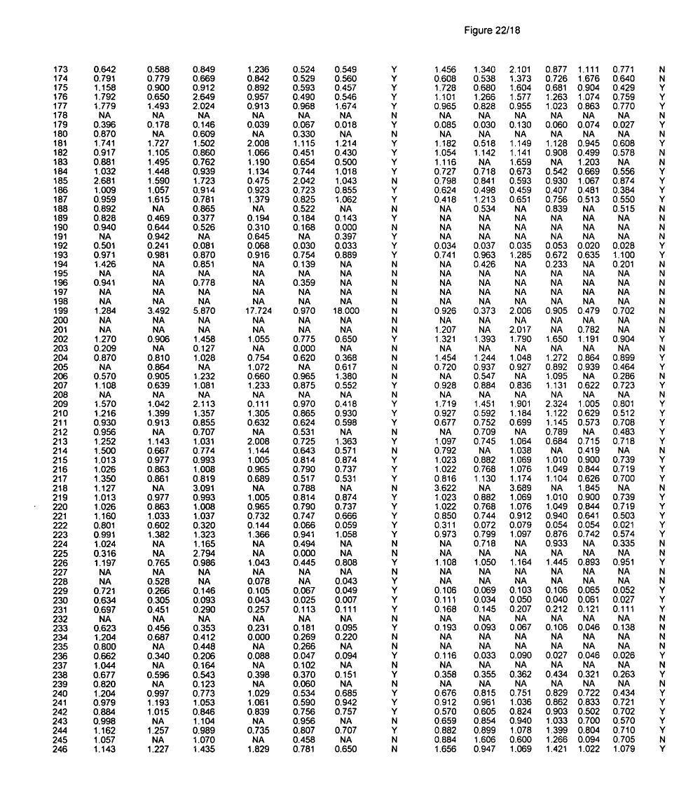

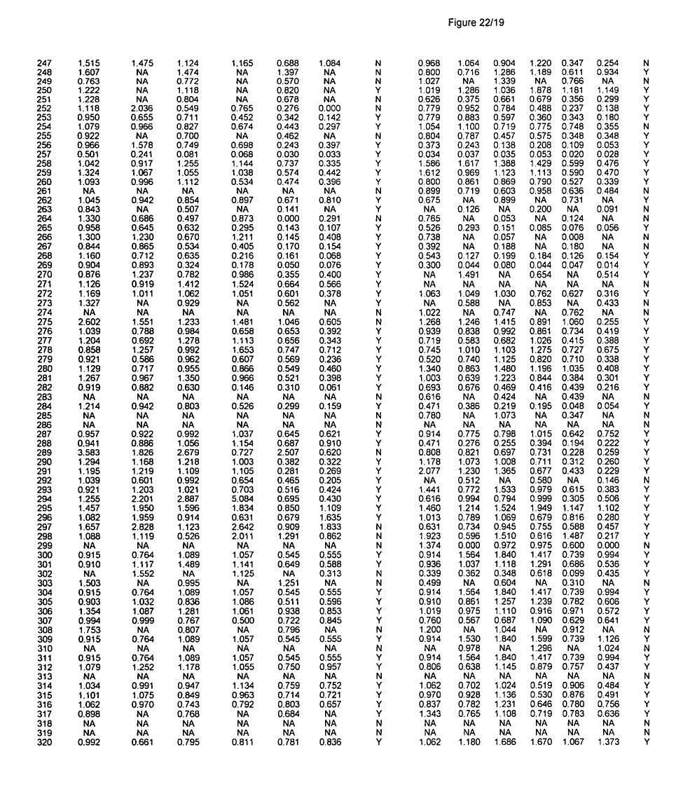

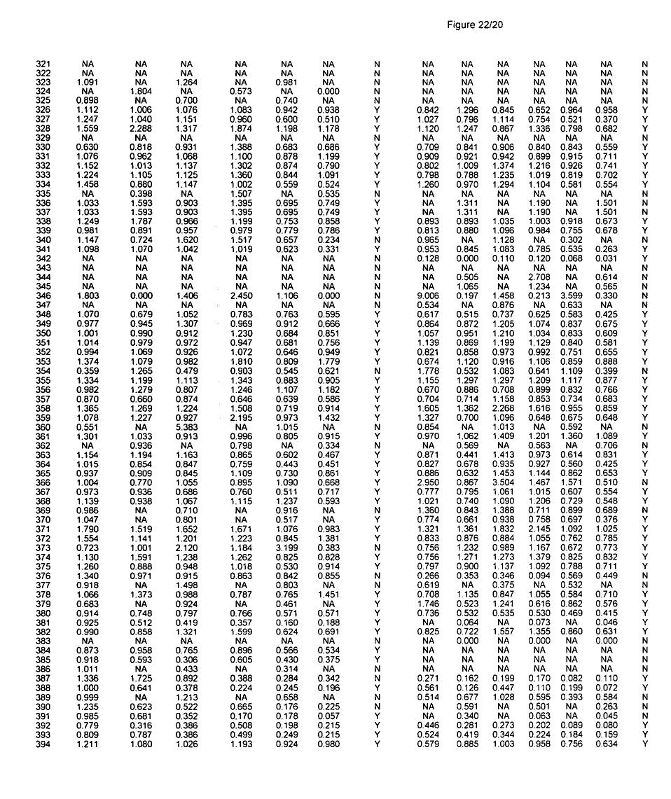

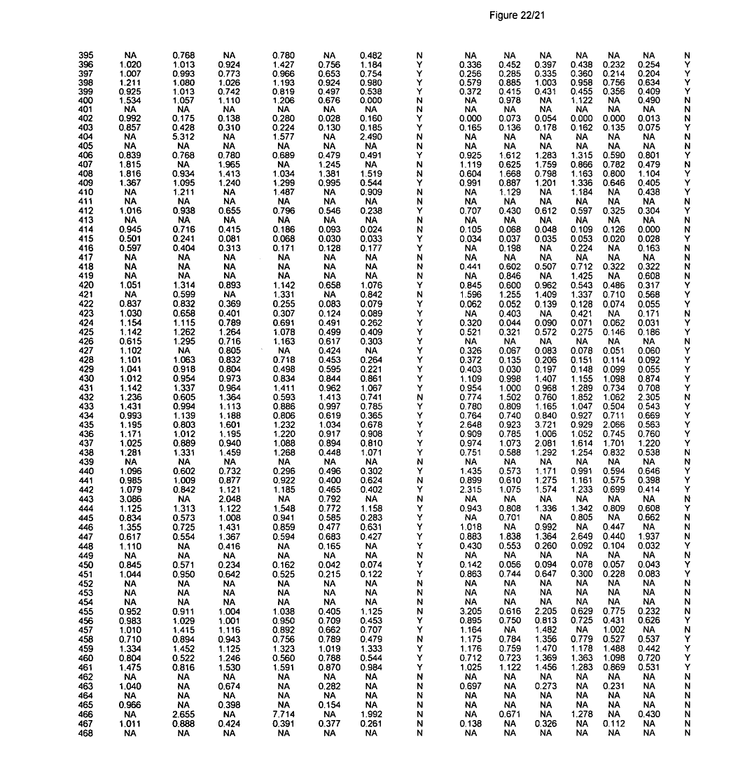

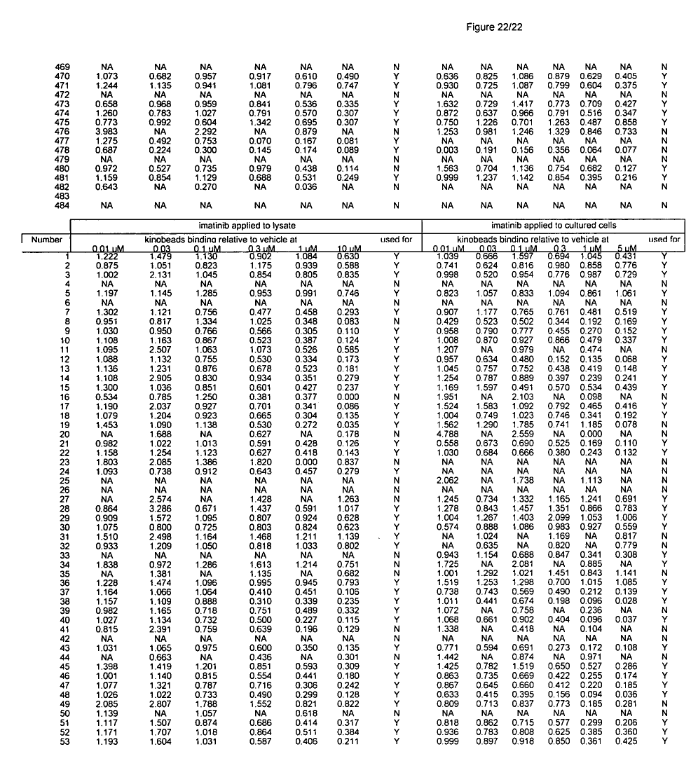

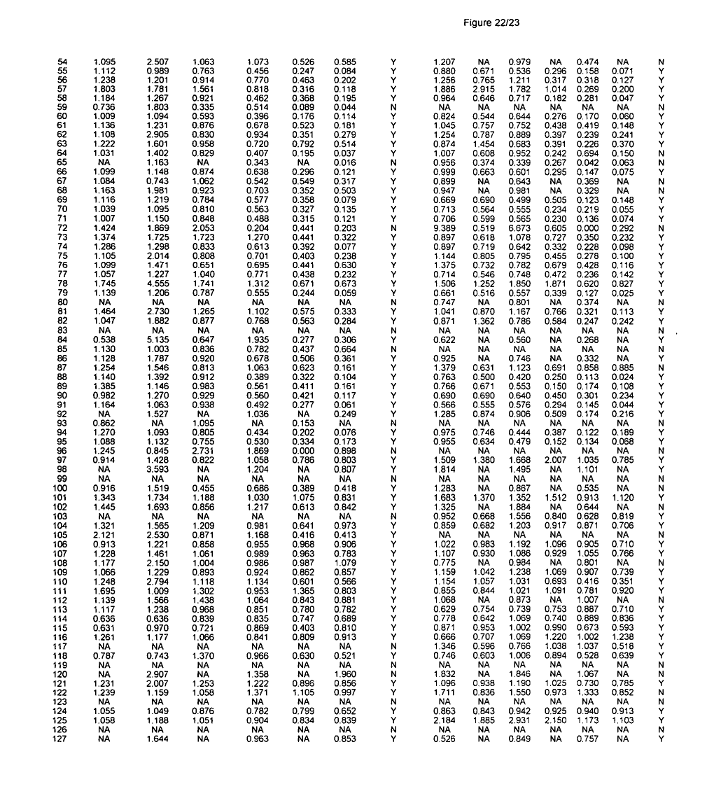

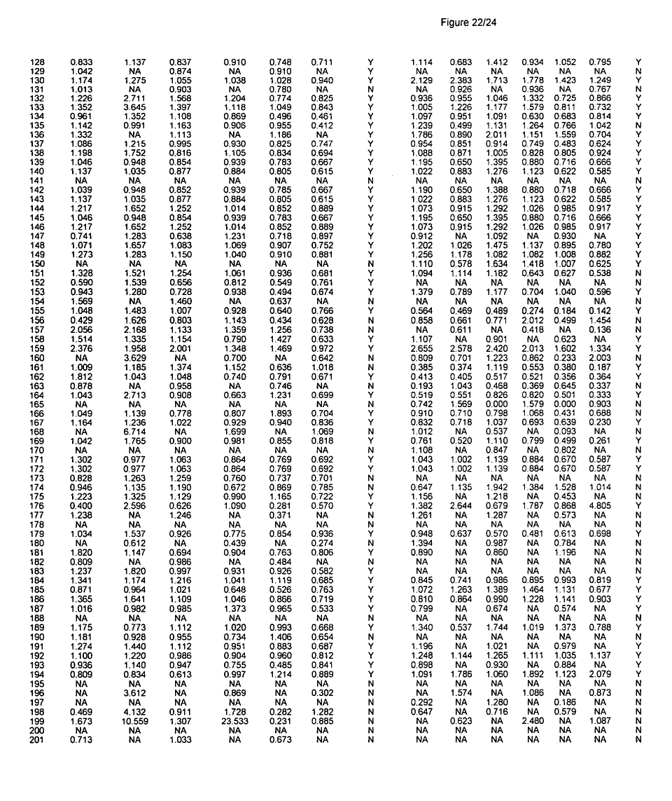

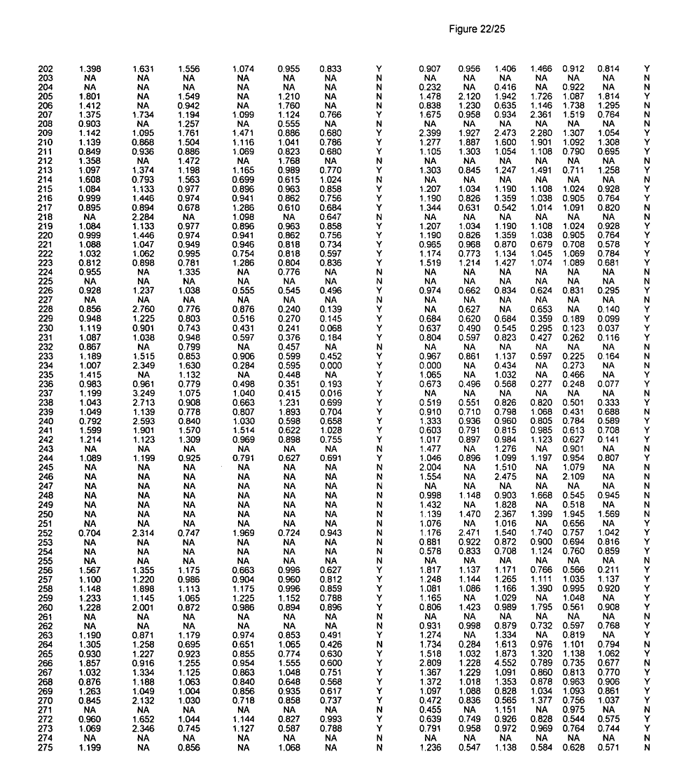

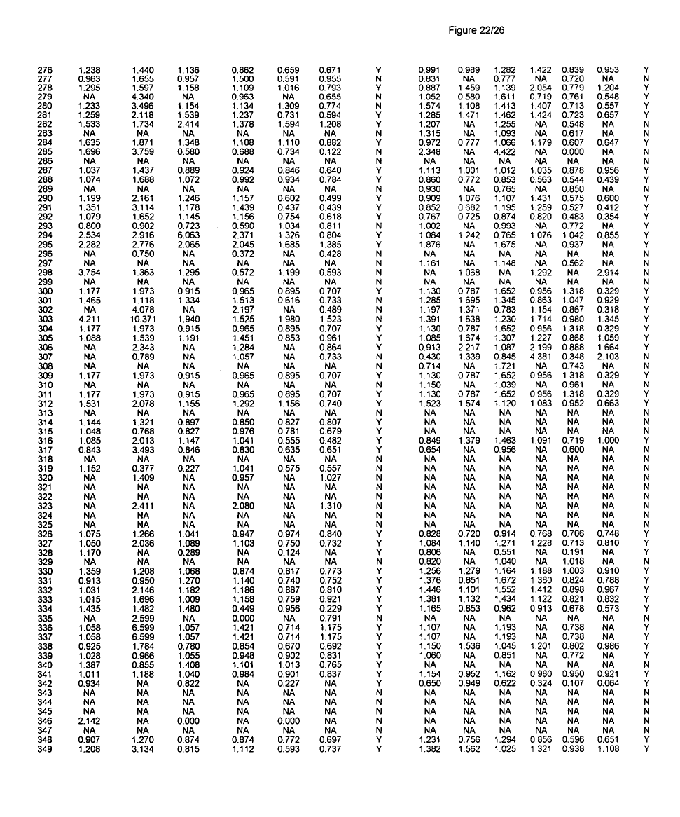

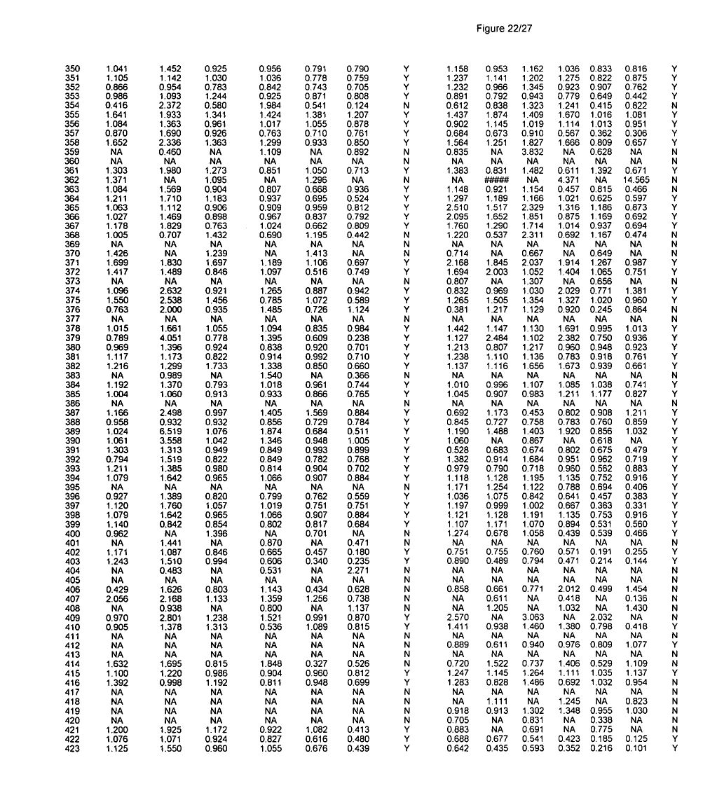

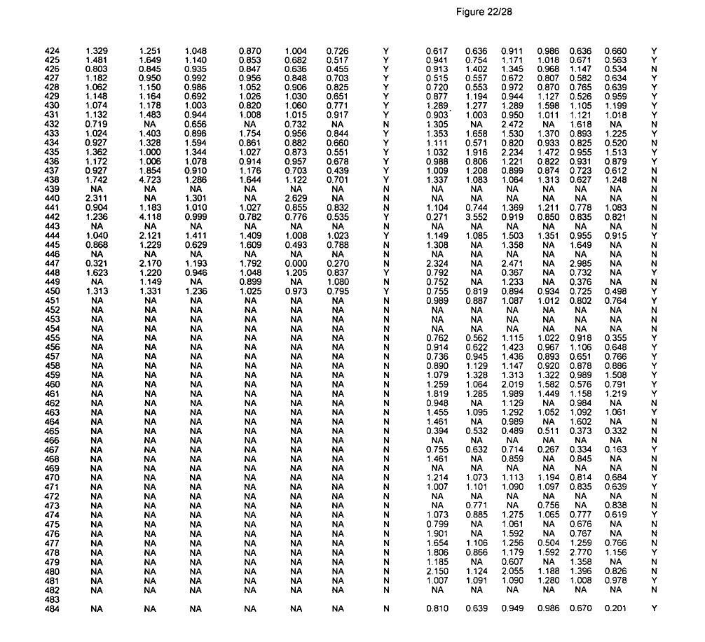

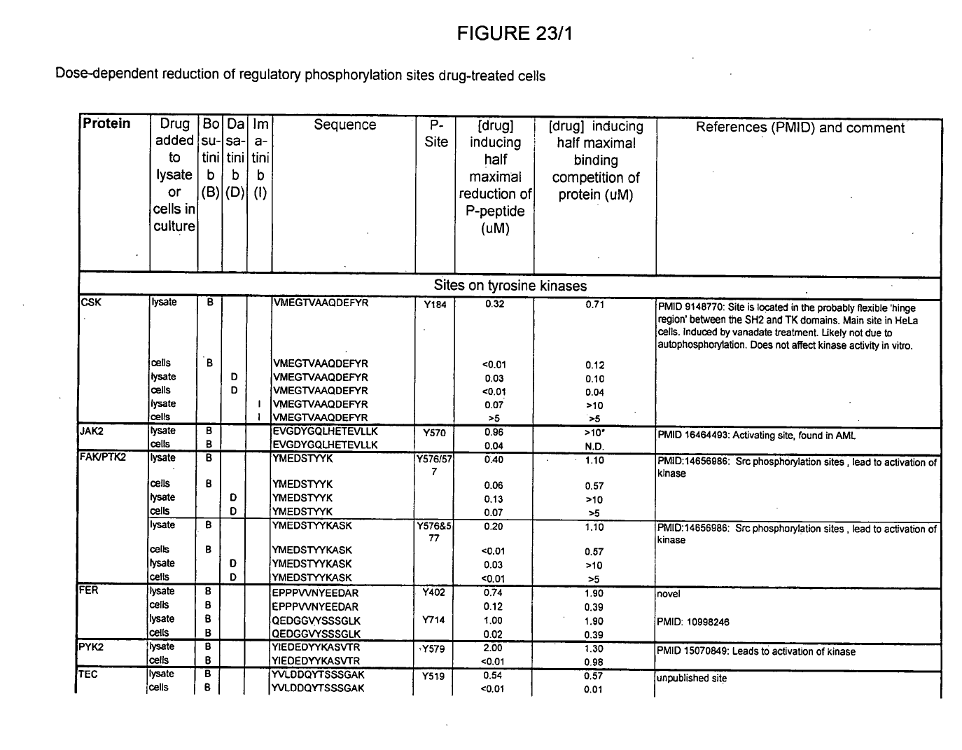

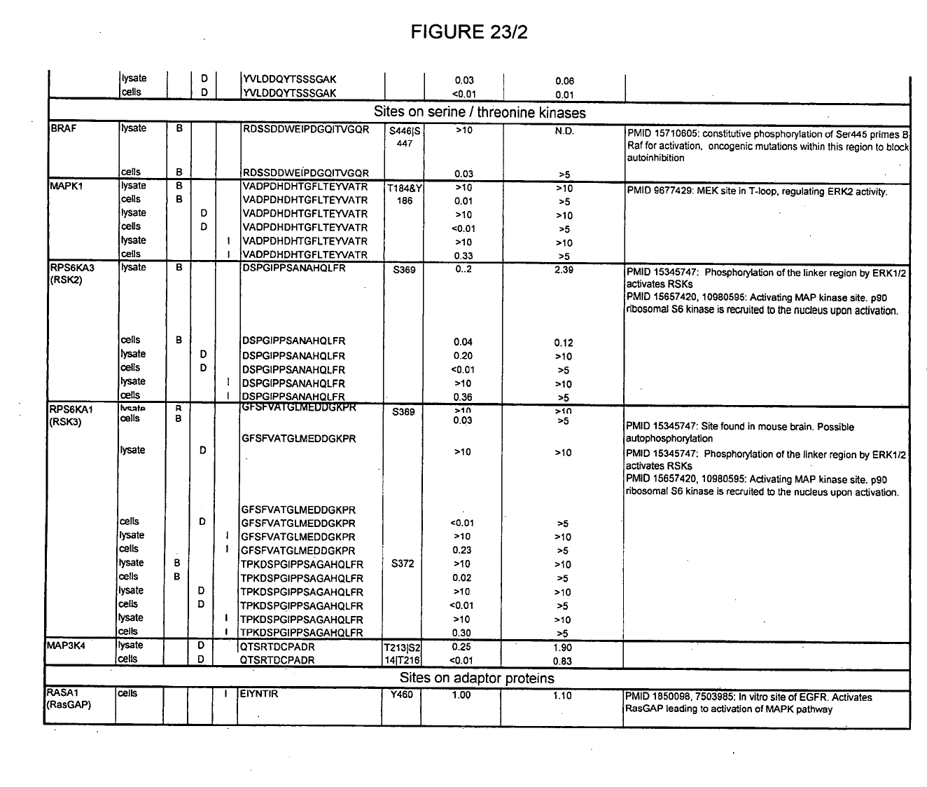

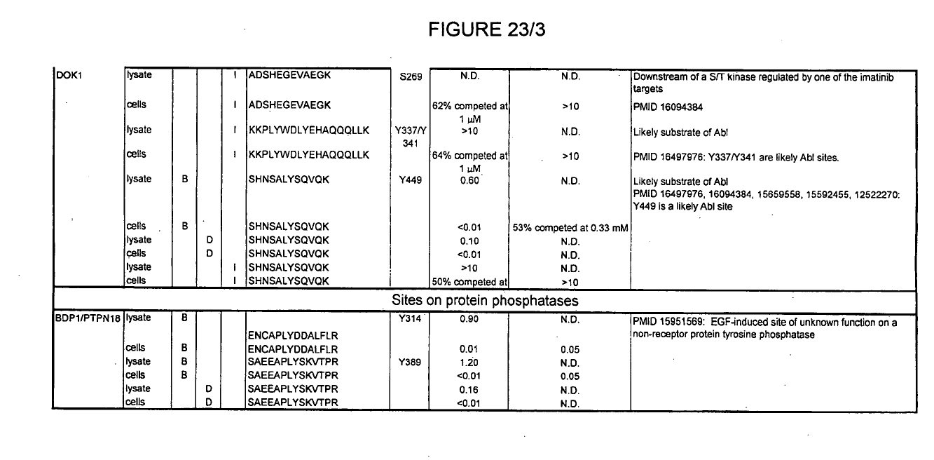

The following four broad specificity ligands (Kinobead ligands 1 to 4; FIG. 1) were covalently coupled to beads in separate reactions as described below and then the four types of beads were mixed and used for the drug pulldown experiments.

Kinobead ligand 1: Bisindolylmaleimide VIII-Acetate (Chemical Formula: C₂₄H₂₂N₄O₂. CH₃COOH; MW 398.5; CAS number 138516-31-1; Alexis Biochemicals, AXXORA Deutschland GmbH, Grunberg; Cat-ALX-270-056).

Kinobead ligand 2: Purvalanol B (Chemical composition: C₂₀H₂₅ClN₆O₃; MW 441.92; CAS number 212844-54-7; Tocris Biochemicals Cat-1581, BIOTREND Chemikalien GmbH Koln, Germany).

Kinobead ligand 3: CZC00007324; (7-(4-Aminomethyl-phenylamino)-3-(2,6-dichloro-phenyl)-1-methyl-1H-[1,6]naphthyridin-2-one).

### Synthesis of kinobead ligand 1: 7-(4-Aminomethyl-phenylamino)-3-(2,6-dichloro-phenyl)-1-methyl-1H-[1,6]naphthyridin-2-one

The first seven steps of the synthesis of CZC00007324 were performed as described in Klutchko, S. R. et al., 1998, Journal of Medicinal Chemistry 41, 3276-3292. The remaining steps were performed as described below.

Steps 1-7: 6-(2,6-Dichlorophenyl)-2-methanesulfonyl-8-methyl-8H-pyrido[2,3-d]pyrimidin-7-one was synthesized from 4-chloro-2-methylsulfanyl-5-pyrimidinecarboxylate ethyl ester following the procedure in J. Med. Chem. 1998, 41, 3276-3292.

### Step 8: {4-[3-(2,6-Dichloro-phenyl)-1-methyl-2-oxo-1,2-dihydro-[1,6]naphthyridin-7 ylamino]benzyl}-carbamic acid tert-butyl ester

6-(2,6-Dichlorophenyl)-2-methanesulfonyl-8-methyl-8H-pyrido[2,3-d]pyrimidin-7-one

(0.100 g, 0.2 mmol) and 3-(N-Boc-methylamino)aniline (0.421 g, 2.0 mmol) were mixed as solids and heated to 140° C. for 30 mins. The crude reaction mixture was dissolved in dichloromethane and washed with 2N HCl (aq)×2. The organic layer was dried with anhydrous magnesium sulfate, filtered and concentrated. The crude product was recrystallised from hot ethyl acetate to afford {4-[3-(2,6-Dichloro-phenyl)-1-methyl-2-oxo-1,2-dihydro-[1,6]naphthyridin-7-ylamino]benzyl}-carbamic acid tert-butyl ester as a yellow solid (0.031 g-25%). ¹H NMR (DMSO-d₆) δ 10.18 (s, 11H); 8.83 (s, 11H); 7.76 (d, 2H); 7.58 (d, 2H); 7.46 (dd, 1H); 7.32 (brt, 1H); 7.23 (d, 21H); 4.10 (d, 2H); 3.66 (s, 3H); 1.40 (s, 9H). LCMS: method A, RT=5.60 min.

### Step 9: 7-(4-Aminomethyl-phenylamino)-3-(2,6-dichloro-phenyl)-1-methyl-1H-[1,6]naphthyridin-2-one

{4-[3-(2,6-Dichloro-phenyl)-1-methyl-2-oxo-1,2-dihydro-[1,6]naphthyridin-7-ylamino]benzyl}-carbamic acid tert-butyl ester (0.026 g, 0.05 mmol) was dissolved in methanol (3 ml) and hydrochloric acid (4N in dioxane, 1.2 ml) was added. The reaction was stirred at room temperature for 1.5 hours when HPLC showed no remaining starting material. The solvent was removed in vacuo. The residue was dissolved in water and the solution basified with sodium carbonate (sat., aq.). The resulting precipitate was collected and dried to afford 7-(4-Aminomethyl-phenylamino)-3-(2,6-dichloro-phenyl)-1-methyl-1H-[1,6]naphthyridin-2-one (0.021 g-100%) as a yellow solid. ¹H NMR (DMSO-d₆) [symbol] 10.20 (brd, 1H); 8.83 (d, 1H); 7.90 (d, 1H); 7.76 (d, 1H); 7.72 (d, 1H); 7.60 (dd, 2H); 7.47 (ddd, 1H); 7.33 (d, 1H); 7.24 (d, 1H); 4.07 (d, 2H); 3.66 (s, 3H). LCMS: method A, RT=4.44 min, [MH⁺=426].

Kinobead ligand 4: CZCQ00008004. 2-(4′-aminomethyl phenylamine)-5-fluoro-pyrimidin-4-yl)-phenyl-amine. Chemical formula C₁₇H₁₆N₅F. MW 309.34. This is an analog of CZC00004919.

### Synthesis of CZC00008004-(2-(4′-aminomethyl phenylamine)-5-fluoro-pyrimidin-4-yl)-phenyl-amine

### Step 1: 2,4-Dichloro-5-fluoro-pyrimidine

Phosphorus oxychloride (2 ml) was added to 5-fluorouracil (1 g, 7.688 mmol) followed by phosphorus pentachloride (3.28 g, 15.76 mmol), the mixture was heated and stirred at 110° C. for 5 hours and then allowed to cool. The excess phosphorus oxychloride was slowly hydrolised in a bath of ice/water mixture (10 ml). The aqueous mixture was extracted with diethyl ether (3×10 ml). The organic layers were combined, washed with saturated sodium bicarbonate (10 ml) followed by saturated sodium chloride (10 ml), dried with anhydrous magnesium sulfate and then filtered. The solvent was removed by evaporation at 350 mmHg to leave a viscous oil which slowly crystallised to afford the title compound (1.22 g-95%). The compound was used without further analysis on the next step.

### Step 2: (2-Chloro-5-fluoro-pyrimidin-4-yl)-phenylamine

To a solution of 2,4-Dichloro-5-fluoro-pyrimidine (0.550 g, 3.30 mmol) in dimethylformamide (13 ml) was added aniline (0.301 ml, 3.30 mmol) and N-ethyldiisopropylamine (0.602 ml, 3.63 mmol) and the mixture stirred at room temperature for 18 hours. The mixture was quenched with ethyl acetate (20 ml) and washed with saturated ammonium chloride (20 ml) and the organic layer removed. The aqueous layer was washed with ethyl acetate (20 ml) and the combined organic layers washed with water (20 ml), saturated sodium chloride (10 ml) and dried with anhydrous magnesium sulfate. The solvent was removed by evaporation and the resulting solid subjected to column chromatography (silica, ethyl acetate (0 to 20%)/petrol ether) to afford the title compound (0.532 g-72%). LCMS: method A, RT=4.67 min, [MH⁺=224].

### Step 3: [4-(5-Fluoro-4-phenylamino-pyrimidin-2-ylamino)-benzyl]-carbamicacid tert-butyl ester

(2-Chloro-5-fluoro-pyrimidin-4-yl)-phenylamine (0.087 g, 0.39 mmol) and 4-[N-Boc aminomethyl]aniline (0.087 g, 0.39 mmol) were mixed together. A stirrer bar was added and the flask placed in a oil bath at 110° C. for 20 mins. The mixture was cooled, the residue dissolved in 3 ml of Dichloromethane/Methanol (99:5) and loaded up on a Flash Chromatography cartridge and purified using ethyl acetate (20 to 60%) in petrol ether to give the desired compound as a yellow solid (0.051 g-32%). ¹H NMR (400 MHz, CDCl₃-d₆) δ 7.85 (d, 1H); 7.50 (dd, 2H); 7.39 (d, 2H); 7.27 (t, 2H); 7.12-7.00 (m, 3H); 6.81 (s, 1H); 6.66 (s, 1H); 4.67 (s, 1H), 4.17 (d, 2H), 1.36 (s, 9H). LCMS: method B, RT=9.20 min, [MH⁺=410].

### Step 4: (2-(4′-aminomethyl phenylamine)-5-fluoro-pyrimidin-4-yl)-phenyl-amine

To a solution of [4-(5-Fluoro-4-phenylamino-pyrimidin-2-ylamino)-benzyl]-carbamicacid tert-butyl ester (0.055 g, 0.134 mmol) in methanol (5 ml) HCl (4N in dioxane) (2 ml) and the reaction was stirred at room temperature for 1 hour. The solvent was removed by evaporation. Water (5 ml) was added and the pH of the solution was raised to 8 by addition of sodium bicarbonate. The resulting precipitate was filtered and dried to afford the title compound (0.035 g-84%). ¹H NMR (400 MHz, DMSO-d₆) δ 7.90 (d, 1H); 7.70 (dd, 2H); 7.55 (dd, 2H); 7.35 (t, 2H); 7.20 (d, 2H); 7.10 (t, 1H); 3.80 (s, 2H). LCMS: method B, RT=5.022 min, [MH⁺=310].

All reactions were carried out under inert atmosphere. NMR spectra were obtained on a Bruker dpx400. LCMS was carried out on an Agilent 1100 using a zorbax SBC-18, 4.6 mm×150 mm-5μ column. Column flow was 1 mL/min and solvents used were water and acetonitrile (0.1% TFA) with an injection volume of 10 ul. Wavelengths were 254 and 210 nm. Methods are described below.

TABLE 1
Analytical methods

| | Easy Access | ChemStation | Flow | | Run |
|---|---|---|---|---|---|
| Method | Method Name | Method Name | Rate | Solvent | Time |
| A | Analytical positive | ANL_POS7.M | 1 ml/min | 0-2.5 min | 7 min |
| | 7 mn | | | 5-95% MeCN | |
| | | | | 2.5-6 min | |
| | | | | 95% MeCN | |
| B | Analytical positive | ANAL_POS.M | 1 ml/min | 0-11 min | 15 min |
| | Ion | | | 5-95% MeCN | |
| | | | | 11-13 min | |
| | | | | 95% MeCN | |

TABLE 2
Abbreviations used in chemistry protocols

| | |
|---|---|
| aq | aqueous |
| d | doublet |
| DMSO | dimethyl sulfoxide |
| g | gram |
| HCl | Hydrochloric acid |
| HPLC | high pressure liquid chromatography |
| LCMS | liquid chromatography-mass spectrometry |
| m | multiplet |
| mins | minute |
| mmol | millimole |
| N | Normal |
| NMR | nuclear magnetic resonance |
| q | quartet |
| RT | retention time |
| s | singlet |
| sat | saturated |
| t | triplet |

### 2. Immobilization of Ligands Containing Amine Groups

NHS-activated Sepharose 4 Fast Flow (Amersham Biosciences, 17-0906-01) was equilibrated with anhydrous DMSO (Dimethylsulfoxid, Fluka, 41648, H20<=0.005%). 1 ml of settled beads was placed in a 15 ml Falcon tube, compound stock solution (usually 100 mM in DMF or DMSO) was added (final concentration 0.2-2 lμmol/ml beads) as well as 15 μl of triethylamine (SIGMA, T-0886, 99% pure). Beads were incubated at room temperature in darkness on an end-over-end shaker (Roto Shake Genie, Scientific Industries Inc.) for 16-20 hours. Coupling efficiency is determined by HPLC. Non-reacted NHS-groups were blocked by incubation with aminoethanol at roomtemperature on the end-over-end shaker over night. Washed beads were stored in isopropanol or immediately used for binding reactions.

### 3. Immobilization of Ligands Containing Carboxyl Groups

The compounds were coupled under basic condition to reversed NHS-Sepharose beads (PyBroP chemistry) as outlined below.

### Washing of Beads

Step 1: Use 1 ml (settled volume) NHS-sepharose/beads for a standard coupling reaction (NHS-activated Sepharose 4 Fast Flow provided in isopropanol, Amersham Biosciences, 17-0906-01).

Step 2: Wash the beads 3 times with 10 ml DMSO and once with 10 ml anhydrous DMSO (Dimethylsulfoxid, Fluka, 41648, H₂0<=0.005%); centrifugation steps: 1 min, 1.200 rpm, room temperature; discard supernatant into non-halogenous solvent waste.

Step 3: After last washing step resuspend beads in one volume of an hydrous DMSO.

### Reversing the NHS Beads

This step is designed for 1 mL beads, adjust accordingly for any other bead volumes. NHS beads have a capacity of 20 μmol/mL. Therefore, 20% reversed beads should have a capacity of 4 μmol/mL.
Make a 4:1 ratio mixture of Aminoethanol and Ethylenediamine, then add Triethylamine to the mixture.
(200 Fmol total) (10× capacity of 1 mL NHS beads)
   1: 9.66 μL 16.56 M Aminoethanol (2-Aminoethanol, Aldrich, 11.016-7) (total 160 μmol)
   2: 2.68 μL 14.92M Ethylenediamine (Fluka, 03350) (total 40 μmol).
   3: 15 μL 7.2 M Triethylamine (TEA) (SIGMA, T-0886, 99% pure) Mixture will be split in two phases, but that is OK.

Add mixture to washed/resuspended NHS beads and incubate 16 hours (over night) at room temperature on the end-over-end shaker.

PyProB coupling. This procedure does not require a preactivation step, activation and coupling occurs in-situ.
1. Dissolve PyBroP (Bromo-tris-pyrrolidino-phosphonium hexafluorophosphate; supplier: NOVABIOCHEM) in waterfree Dimethylformamide (DMF) to a final concentration of 100 mM (46.62 mg/mL), the solution can be used for up to one day.
2. Dissolve 35 μL of Diisopropylethylamine (DEEA) in 1 mL waterfree DMF, final concentration 200 mM.
3. Wash 1 mL of reversed beads 3 times with 15 mL DMSO. Centrifugation step: 1 minute at 1,200 rpm room temperature. Discard supernatant.
4. Wash reversed beads 3 times with 15 mL water-free DMF. Centrifugation step 1 minute, 1,200 rpm room temperature. Discard supernatant.
5. Suspend reversed sepharose beads in 1 mL of waterfree DMF.
6. Add 100 μL of Diisopropylethylamine (DIEA) solution.
7. Add compound (1 μmol/mL reversed Sepharose beads; corresponds to 10 μL 100 mM compound/mL reversed sepharose beads) from DMF-solution in the required amount to the beads.
8. Mix until a homogenous suspension is obtained.
9. Centrifuge 1 minute at 1200 rpm at room temperature, remove 20 μL supernatant and dilute in 30 μL methanol (MeOH) for the starting value for HPLC analysis.
10. Add 100 μL of PyBroP solution to the suspension and mix again.
11. Incubate over night on the end-over-end mixer at room temperature.
12. Centrifuge 1 minute at 1200 rpm at room temperature, remove 20 μL supernatant and dilute in 30 μL methanol (MeOH) for the coupled value for HPLC analysis.

### Blocking of the Beads

1. Block the beads by adding 100 μL 100 mM NHS-Acetate (see below how to make blocking reagent).
2. Incubate over-night at room temperature on the end-over-end shaker.

### Blocking Reagent (NHS Activated Acetic Acid):

1. Prepare 200 mM solutions of DCCD and NHS in acetonitrile (˜5 mL of each).
2. Mix equal volumes of the 200 mM NHS and DCCD in a 20 mL clear glass vial.
3. Per 1 mL total volume NHS/DCCD mix, add 11.4 μL 17.49 M acetic acid (2× molar excess) (Merck, 1.00063.1000).
4. Mix thoroughly. A precipitate will form after about 2 minutes (crystals of the urea derivative).
5. Allow the reaction to sit at room temperature at least overnight before further use.

### Washing of Beads

1. Wash the beads 2 times with 14 ml DMSO (e.g. FLUKA, 34869 or equivalent), then with 2×14 mL isopropanol (Merck, 1.00983.1000, pro analysis). Centrifuge steps: 1 minute at 1200 rpm at room temperature. Remove supernatant between washes.
2. Resuspend the beads with 1 mL isopropanol to make a 50% slurry for storage at −20° C. or use immediately for binding reactions with cell lysates.

TABLE 3
Abbreviations used in coupling protocols

| | |
|---|---|
| DCCD | Dicyclohexylcarbodiimide |
| DIEA | Disopropylethylamine |
| DMSO | Dimethyl sulfoxide |
| DMF | Dimethylformamide |
| NHS | N-hydroxysuccinimide |
| PyBroP | Bromo-tris-pyrrolidino-phosphonium |
| | hexafluorophosphate |
| TEA | Triethylamine |

### Example 2

### Signalokinome

This example illustrates the treatment of cells with compounds (see particularly the second aspect of the invention). HeLa cells were treated with epidermal growth factor (EGF), a cell lysate was prepared and analysed using Kinobeads (experimental protocol in section 2.1) and mass spectrometry. The preparation of Kinobeads is described in Example 1.

In parallel the cell lysate was subjected to immunoprecipitation with an anti-phosphosphotyrosine antibody (experimental protocol in section 2.2) and analysed by mass spectrometry.

The result (FIG. 2) shows that kinobeads identify significantly more kinases (Table 8) compared to the immunoprecipitation procedure (Table 6).

### 1. Preparation of the Biological Sample (Cell Lysate)

### 1.1 Cell Culture and Treatment of Cells

Cell culture. HeLa cells (American Type Culture Collection-No CCL-2) were grown in MEM medium (without L-Arginine and without L-Glutamine; Promocell C-75280), 10% dialyzed Fetal Bovine Serum (Gibco, 26400-044), 1% 100× non-essential amino acids (Cambrex, BE13-114E), 1 mM Sodium Pyruvate (Gibco, 11360-039), 2 mM L-glutamine (Gibco, 25030-032), 40 mg/L 12C or 13C L-Arginine (12C Arginine-Sigma, A6969) (13C Arginine-Cambridge Isotope Laboratories Inc., CLM-2265) at 37° C., 5% CO₂.

Cell propagation. After cells had reached confluency in a 15 cm dish, cells were split 1 to 10 for further growth. Cells were split by first removing the supernatant media, then briefly washing the cells with 15 mL PBS buffer (Gibco, 14190-094). After removal of the PBS, the cells were detached from the plate by adding 2 mL trypsin-EDTA solution (Gibco, 25300-054) per 15 cm plate and incubating the plate for 10 minutes at 37° C. After detachment of the cells, 8 mL MEM growth medium (see above) was added per 15 cm plate. 1 mL of this solution was put on fresh 15 cm plates and 24 mL MEM media (see above) was added. Plates were again incubated at 37C 5% CO₂ until the cells were confluent (˜3-4 days).

EGF treatment of cells. One day prior to treatment of the cells with Epidermal Growth Factor (EGF), the cell growth medium was removed by aspiration and 20 mL fresh MEM medium (see above) was added except that the medium was supplemented with 0.1% Fetal Bovine Serum (FBS) instead of 10% FBS. The cells were incubated in this starvation medium overnight at 37° C., 5% CO2. After cell starvation, 3 μL 1 mg/mL recombinant human EGF (Biomol, 50349-1) was added to each 15 cm plate (final EGF concentration=150 ng/mL medium). The plates were incubated at 37° C., 5% CO2 for 10 minutes prior to harvesting.

Cell harvesting. Cells were harvested by pouring off of the EGF-containing medium, washing of each 15 cm plate once with 10 mL ice-cold PBS buffer, and scraping the plate with a rubber policeman in order to detach the cells. The cells were transferred into a 50 mL Falcon tubes (Becton Dickinson, 352070) and centrifuged for 10 minutes at 1500 rpm in a Heraeus Multifuge 3SR. The supernatant was aspirated and the cell pellet was resuspended in 50 mL ice-cold PBS buffer. After centrifugation and aspiration of the supernatant cell pellets were quickly frozen in liquid nitrogen and then stored at −80° C.

### 1.2 Preparation of Cell Lysates

The HeLa cell lysate was prepared by mechanical disruption in lysis buffer solution under gentle conditions that maintain the structure and function of proteins.

The following steps were carried out:
Thaw the tissue quickly at room temperature or 37° C., then transfer tissue to a glass bottle containing the 1× lysis buffer (use a vial big enough to be used with Polytron PT 3100 homogenizer)
Lyse the organ/tissue with 4×10 sec pulses at 5000-7000 rpm at 4° C. in the cold room
Transfer the homogenate into precooled 50 ml falcon tubes
Incubate homogenate on ice for 30 min
Spin cells for 10 min at 6000 g at 4° C. (6.000 rpm in Sorvall SLA600, precooled)
Transfer supernatant to a UZ-polycarbonate tube (Beckmann, 355654)
Spin supernatant for 1 h at 145.000 g at 4° C. (40.000 rpm in Ti50.2, precooled)
Save supernatant (remove and discard most of the lipid layer if possible), transfer supernatant into a glass bottle and store on ice
Determine protein concentration by Bradford assay (BioRad). Typical protein concentrations are in the range of 5-10 mg/ml.
Prepare aliquots in 15 to 50 ml Falcon tubes
Freeze aliquots in liquid nitrogen and store them at −80° C.
   Preparation of 100 ml 1× Lysis Buffer with 0.8% NP40:

Combine the following solutions or reagents and add destined water to a final volume of 100 ml: 20 ml 5× lysis buffer (see below), 100 μl 1 M DTT, 5 ml 0.5 M NaF, 4 ml 20% NP40, 4 complete EDTA-free tablets (protease inhibitor cocktail, Roche Diagnostics, I 873 580), add distilled water to 100 ml.

TABLE 4
Preparation of 5x-lysis buffer

| | | Final conc. in | Add for |
|---|---|---|---|
| | Stock | 1 x lysis | 11 5 x lysis |
| Substance: | solution | buffer | buffer |
| Tris/HCl pH 7.5 | 1 M | 50 mM | 250 ml |
| Glycerol | 87% | 5% | 288 ml |
| MgCl₂ | 1 M | 1.5 mM | 7.5 ml |
| NaCl | 5 M | 150 mM | 150 ml |
| Na₃VO₄ | 100 mM | 1 mM | 50 ml |

These solutions were obtained from the following suppliers:

1M Tris/HCl pH 7.5: Sigma, T-2663; 87% Glycerol: Merck, cat. no. 04091.2500; 1 M MgCl₂: Sigma, M-1028; 5 M NaCl: Sigma, S-5150.

The 5× concentrated lysis buffer was filtered through a 0.22 μM filter and stored in 40 ml aliquots at −80° C.

Preparation of Stock Solutions Used in this Protocol:
Preparation of 100 mM Na₃VO₄ stock solution:

Dissolve 9.2 g Na₃VO₄ in 400 ml distilled water.

1) Adjust the pH to 10.0 using either 1 N NaOH or 1 N HCl. The starting pH of the sodium orthovanadate solution may vary with batch. At pH 10.0 the solution will be yellow.

2) Boil the solution until it turns colorless (approximately 10 min).

3) Cool to room temperature.

4) Readjust the pH to 10.0 and repeat steps 2 and 3 until solution remains colorless and the pH stabilizes at 10.0.

5) Adjust the volume to 500 ml with distilled water.

6) Freeze aliquots at −20° C. Aliquots can be stored for several months.

### Preparation of 500 mM NaF Stock Solution:

Dissolve 21.0 g NaF (Sigma, S7920) in 500 ml distilled water. Filter solution through a 0.22 μm filter and store at 4° C.

### Preparation of 20% NP40-Solution:

Weigh 40.0 g NP40 (Sigma, Igepal-CA630, catatogue No. 13021). Add distilled water up to 200 g. Mix completely and store solution at room temperature.

Preparation of 1 M DTT solution:

Dissolve 7.7 g DTT (Biomol, catalogue No. 04010) in 50 nil distilled water. Filter solution through 0.22 μm filter and freeze 400 μl aliquots at −20° C. Aliquots can be stored for several months.

### 2. Binding Reactions

2.1 Contacting of the “Kinobeads” (Immobilized Capturing Ligands) with the Cell Lysate

The kinobeads (immobilized capturing ligands) were contacted with cell lysate prepared from HeLa cells under conditions that allow the binding of the proteins in the lysate to the ligands. The binding conditions were close to physiological by choosing suitable buffer conditions preserving the function of the proteins. After removing non-captured proteins through a gentle washing procedure the bound proteins were contacted with a test compound which led to the elution of proteins.

### 2.1.1 Preparation of DP-buffers

TABLE 5
Preparation of 5x-DP buffer

| | | Final conc. in | Add for |
|---|---|---|---|
| | Stock | 1 x lysis | 11 5 x lysis |
| Substance: | solution | buffer | buffer |
| Tris/HCl pH 7.5 | 1 M | 50 mM | 250 ml |
| Glycerol | 87% | 5% | 288 ml |
| MgCl₂ | 1 M | 1.5 mM | 7.5 ml |
| NaCl | 5 M | 150 mM | 150 ml |
| Na₃VO₄ | 100 mM | 1 mM | 50 ml |

The 5×-DP buffer was filtered through 0.22 pun filter and stored in 40 ml-aliquots at −80° C. These solutions were obtained from the following suppliers: 1.0 M Tris/HCl pH 7.5 (Sigma, T-2663), 87% Glycerol (Merck, catalogue number 04091.2500); 1.0 M MgCl₂ (Sigma, M-1028); 5.0 MNaCl (Sigma, S-5150).

The following 1×DP Buffers were Prepared:
1×DP buffer (for bead equilibration)
1×DP buffer/0.4% NP40 (for bead equilibration and first wash step of beads)
1×DP buffer/0.2% NP40 (for second wash step of beads and for compound elution)
1×DP buffer/protease inhibitors (for first lysate dilution step); add one protease inhibitor tablet per 25 ml lysis buffer (EDTA-free tablet, protease inhibitor cocktail, Roche Diagnostics, 1 873 580)
1×DP buffer/0.4% NP40/protease inhibitors (for second lysate dilution step)

### Example for Preparation of 1×DP-Buffer/0.4% NP40 (100 ml):

Combine the following solutions and reagents and add distilled water up to a final volume of 100 ml: 20 ml 5×DP buffer, 5 ml 0.5 M NaF, 2 ml 20% NP40, 100 μl 1 M DTT, and add distilled water up to 100 ml. All buffers contain 1 mM DTT final concentration.

2.1.2 Washing and Equilibration of Beads The kinobeads (Example 1) were prepared for the binding reaction by washing with a suitable buffer and equilibration in the buffer.

The following steps were carried out:

1. Use 15 ml Falcon tubes for all washing steps.
2. Use 100 μl KinoBeads per experiment (settled bead volume): mix equal amounts (25 μl) of each bead type coupled with the following 4 ligands (coupling density of 1 μmol/ml): Bis VIII (CZC00001056), Purvalanol B (CZC00007097), PD173955 derivative (CZC00007324), and CZC00008004.
3. Wash beads two times with 3 ml 1×DP buffer and once with 3 ml 1×DP buffer/0.4% NP40. During each wash step invert tubes 3-5 times, centrifuge 2 minutes at 1200 rpm at 4° C. in a Heraeus centrifuge. Supernatants are aspirated supernatants and discarded. After the last washing step prepare a 1: I slurry (volume/volume) with 1×DP buffer/0.4% NP40.

### 2.1.3 Preparation of Diluted Cell Lysate

The cell lysate as described under section (1.2) was prepared for the binding reaction by dilution in a suitable buffer and clearing through a centrifugation step. The following steps were carried out:

1. Use a volume of cell lysate corresponding to 50 mg protein per experiment.
2. Thaw the lysate quickly in a 37° C. water bath, then keep the sample on ice.
3. Dilute the lysate in the following way:
1) dilute lysate with 1×DP buffer/protease inhibitors to reduce detergent concentration from 0.8% to 0.4% NP-40.
2) dilute lysate further with 1×DP buffer/0.4% NP40/protease inhibitors to reach a final protein concentration of 5 mg/ml.
   4. Transfer diluted lysate into UZ-polycarbonate tube (Beckmann, 355654).
   5. Clear diluted lysate through ultracentrifugation (20 min, 4° C., 100.000 g, T150.2 rotor, precooled ultracentrifuge).
   6. Save supernatant and keep it on ice.

### 2.1.4 Binding Reaction and Washing

The washed and equilibrated beads from section (2.1.2) were contaced with the diluted cell lysate from step (2.1.3) in order to allow binding of proteins to the ligands. Non-specifically bound proteins were removed by gentle washing in order to reduce background binding.

1. Combine diluted cleared lysate with 100 μl of washed KinoBeads in 15 ml or 50 ml Falcon tube.
2. Incubate for 2 hours at 4° C., rotate on ROTO SHAKE GENIE (Scientific Industries, Inc.) in cold room.
3. After incubation centrifuge for 3 minutes at 1200 rpm in a Heraeus centrifuge or equivalent at 4° C.
4. Remove supernatant carefully without loosing the beads.
5. Transfer the beads to a Mobicol-columns with 90 μm filter (MoBiTec, Goettingen, Cat. no: M1002-90).
6. Wash beads with 10 ml 1×DP buffer/0.4% NP-40 and the 5 ml 1×DP buffer/0.2% NP-40.
7. Let washing buffer run through the column completely before proceeding with next step
8. Place column in Eppendorf tube and centrifuge them for 1 minute at 800 rpm at 4° C. Close columns with lower lid.

### 2.1.5 Elution of Proteins

1. Add 60 μl 2× NuPAGE SDS Sample Buffer (Invitrogen, NP0007; dilute 4× buffer 1; 1 with distilled water before use).
2. Incubate samples for 30 minutes at 50° C.
3. Open lower lid of column and centrifuge MobiTec columns 1 minute at 2000 rpm to separate eluate from beads.
4. Add 1/10 volume of 200 mg/ml iodoacetamide, incubate for 30 minutes at room temperature, protect from light. This reaction leads to the alkylation of cysteines for mass spectrometry analysis.
5. Before loading samples onto the gel, centrifuge samples for 5 minutes at 15.000 rpm in order to remove precipitates.
6. For protein separation apply 60 μl sample to NuPAGE 4-12% Bis-Tris gel (Invitrogen, NP0335).
2.1.6 Preparation of Stock Solutions used in this Protocol
Preparation of a 100 mM Na_VO₄ stock solution:
1) Dissolve 9.2 g Na₃VO₄ in 400 ml distilled water.
2) Adjust the pH to 10.0 using either 1 N NaOH or 1 N HCl. The starting pH of the sodium orthovanadate solution may vary with batch. At pH 10.0 the solution will be yellow.
3) Boil the solution until it turns colorless (approximately 10 min).
4) Cool to room temperature.
5) Readjust the pH to 10.0 and repeat steps 2 and 3 until solution remains colorless and the pH stabilizes at 10.0.
6) Adjust the volume to 500 ml with distilled water.
7) Freeze aliquots at −20° C. Aliquots can be stored for several months.

### Preparation of a 500 mM NaF Stock Solution:

Dissolve 21.0 g NaF (Sigma, S7920) in 500 ml distilled water. Filter solution through a 0.22 μm filter and store at 4° C.

### Preparation of a 20% NP40-Solution:

Weigh 40.0 g NP40 (Sigma, Igepal-CA630, cat. no. 13021). Add distilled water up to 200

g. Mix completely and store solution at room temperature.

### Preparation of a 1 M DTT Solution:

Dissolve 7.7 g DTT (Biomol, catalogue number 04010) in 50 ml distilled water. Filter solution through 0.22 μm filter and freeze 400 μl aliquots at −20° C. Aliquots can be stored for several months.

Preparation of a Iodoacetamide Stock Solution (200 mg/ml):

Dissolve 2.0 g Iodoacetamide (Sigma, 1-6125) in 10 ml distilled water. Filter solution through 0.22 μm filter and freeze 400 μl aliquots at −20° C. Aliquots can be stored for several months.

2.2 Immunoprecipitation using Anti-Phosphotyrosine Antibody Beads

### 2.2.1 Buffers and Antiphosphotyrosine Antibody Beads

All buffers used in the immunoprecipitation experiment with immobilized anti-phosphotyrosine antibodies were identical to those used for the kinobead experiment (see above) except that no DTT was added and 50 μL 1 mM okadaic acid (Biomol, El-181; phosphatase inhibitor) was added so that a final concentration of 500 nM okadaic acid was reached. Antiphosphotyrosine antibody beads (Agarose beads with covalently coupled recombinant 4G10 anti-phosphotyrosine antibody) were obtained from Biomol (Catalogue number 16-199).

### 2.2.2 Washing and Equilibration of Anti-Phosphotyrosine Beads

The anti-phosphotyrosine beads were prepared for the binding reaction by washing with a suitable buffer and equilibration in the buffer.

1. Use 15 ml Falcon tubes for all washing steps.
2. Use 100 μl anti-phosphotyrosine beads per experiment.
3. Wash beads two times with 3 ml 1×DP buffer (-DTT) and once with 3 ml 1×DP buffer/0.4% NP40 (-DTT). During each wash step invert tubes 3-5 times, centrifuge 2 minutes at 1200 rpm at 4° C. in a Heraeus centrifuge. Supernatants are aspirated and discarded.

After the last washing step prepare a 1:1 slurry (volume/volume) with 1×DP buffer/0.4% NP40 (-DTT).

### 2.2.3 Preparation of Diluted Cell Lysate

The cell lysate was prepared for the binding reaction by dilution in a suitable buffer and clearing through a centrifugation step.

1. Use a volume of cell lysate corresponding to 50 mg protein per experiment.
2. Thaw the lysate quickly in a 37° C. water bath, then keep the sample on ice.
3. Dilute the lysate in the following way:

First dilution step: dilute lysate with 1×DP buffer/protease inhibitors/okadaic acid/without DTT to reduce detergent concentration from 0.8% to 0.4% NP-40.

Second dilution step: dilute lysate further with 1×DP buffer/0.4% NP40/protease inhibitors/okadaic acid/without DTT to reach a final protein concentration of 5 mg/ml.

(Note: The second dilution step is only required if the protein concentration of the lysate after the first dilution step is higher than 5 mg/ml).

4. Transfer diluted lysate into UZ-polycarbonate tube (Beckmann, 355654).
5. Clear diluted lysate through ultracentrifugation (20 min, 4° C., 100.000 g, T150.2 rotor, precooled ultracentrifuge).
6. Save supernatant and keep it on ice.

### 2.2.4 Binding Reaction and Washing Steps

The washed and equilibrated anti-phosphotyrosine beads were contaced with the diluted cell lysate from section 2.2.3 in order to allow binding of proteins to the anti-phosphotyrosine beads. Non-specifically bound proteins were removed by gentle washing in order to reduce background binding.

1. Combine diluted cleared lysate with 100 μl of washed anti-phosphotyrosine beads in a 15 ml or 50 ml Falcon tube.
2. Incubate for 4 hours at 4° C., rotate on ROTO SHAKE GENIE (Scientific Industries, Inc.) in the cold room.
3. After incubation centrifuge for 3 minutes at 1200 rpm in a Heraeus centrifuge or equivalent at 4° C.
4. Remove supernatant carefully without loosing the beads.
5. Transfer the beads to a Mobicol-column with 90 μm filter (MoBiTec, Goettingen, Cat. no: M1002-90).
6. Wash beads with 10 ml 1×DP buffer/0.4% NP-40/without DTT and 5 ml 1×DP buffer/0.2% NP-40/without DTT.
7. Let washing buffer run through the column completely before proceeding with next step.
8. Place column in Eppendorf tube and centrifuge them for 1 minute at 800 rpm at 4° C. Close columns with lower lid.

### 2.2.5 Elution of Bound Proteins

1. Add 60 μl 2× NuPAGE SDS Sample Buffer (Invitrogen, NP0007; dilute 4× buffer 1; 1 with distilelled water before use).
2. Incubate samples for 30 minutes at 50° C.
3. Open lower lid of column and centrifuge MobiTec columns 1 minute at 2000 rpm to separate eluate from beads.
4. Add 1/10 volume of 200 mg/ml iodoacetamide, incubate for 30 minutes at room temperature, protect from light. This reaction leads to the alkylation of cysteines for mass spectrometry analysis.
5. Before loading samples onto the gel, centrifuge samples for 5 minutes at 15.000 rpm in order to remove precipitates.
6. For protein separation apply 60 II sample to NuPAGE 4-12% Bis-Tris gel (Invitrogen, NP0335).
3. Mass Spectrometric Analysis of Eluted Enzymes (e.g. Kinases)

### Description of Proteotypic Peptides

Tryptic digestion of a SDS-PAGE-separated protein mixture generates for each protein numerous distinct peptide fragments with different physico-chemical properties. These peptides differ in compatibility with the mass spectrometry-based analytical platform used for protein identification (ID), here nanocapillary reversed phase-liquid chromatography electrospray ionization tandem mass spectrometry (RP-LC-MS/MS). As a result, peptide frequencies for peptides from the same source protein differ by a great degree, the most frequently observed peptides that “typically” contribute to the identification of this protein being termed “proteotypic” peptides. Thus, a “proteotypic peptide” is an experimentally well observable peptide that uniquely identifies a specific protein or protein isoform.

### Advantages of Proteotypic Peptides

The use of proteotypic peptides for protein identification allows rapid and focussed identification and quantitation of multiple known target proteins by focusing the protein identification process on a screening for the presence of information-rich signature peptides.

### Experimental Identification of Proteotypic Peptides

One strategy to generate a list of proteotypic peptides is to collect peptide-identification data empirically and to search the dataset for commonly observed peptides that uniquely identify a protein.

For each IPI database protein entry with at least 10 unequivocal identifications in the CZ dataset (multipeptide IDs or manually verified single peptide IDs), peptide frequencies for contributing peptides are calculated. Only specific, best peptide-to-spectrum matches according to the database search engine Mascot™ (Matrix Science) are considered.

For definition of proteotypic peptides for a specific protein, peptides are ordered by descending peptide frequency and a cumulative peptide presence is calculated: This value gives for each peptide the ratio of identifications where this peptide or any of the peptides with a higher peptide frequency was present. Proteotypic peptides are defined using a cut-off for cumulative presence of 95%, i.e. at least 95% of identification events for this protein were based on at least one proteotypic peptide.

### 3.1 Protein Digestion and Sample Preparation Prior to Mass Spectrometric Analysis

Proteins were concentrated, separated on 4-12% NuPAGE® Novex gels (Invitrogen, Carlsbad, Calif.), and stained with colloidal Coomassie blue. Gel lanes were systematically cut across the entire separation range into ≦48 slices (bands and interband regions) and subjected to in-gel tryptic digestion essentially as described by Shevchenko et al., 1996, Analytical Chemistry 68: 850-858. Briefly, gel plugs were destained overnight in 5 mM NH₄HCO₃ in 50% EtOH, digested with for 4 hours with trypsin at 12.5 ng/μl in 5 mM NH₄HCO₃. Peptides were extracted with 1% formic acid, transferred into a second 96 well plate and dried under vacuum. Dry peptides were resuspended 10 μl 0.1% formic acid in water and 5 μl were injected into the LC-MS/MS system for protein identification.

### 3.2 Mass Spectrometric Data Acquisition

Peptide samples were injected into a nano LC system (CapLC, Waters or Ultimate, Dionex) which was directly coupled either to a quadrupole time-of-fligth (QTOF2, QTOF Ultima, QTOF Micro, Micromass or QSTAR Pulsar, Sciex) or ion trap (LCQ Deca XP, LTQ, Thermo-Finnigan) mass spectrometer. Peptides were separated on the LC system using a gradient of aqueous and organic solvents with a typical gradient time of between 15 and 45 min. Solvent A was 5% acetonitrile in 0.1% formic acid and solvent B was 70% acetonitrile in 0.1% formic acid.

### 3.3 Protein Identification

The peptide mass and fragmentation data generated in the LC-MS/MS experiments were used to query an in-house curated version of the International Protein Index (IPI) protein sequence database (EBI) Proteins were identified by correlating the measured peptide mass and fragmentation data with the same data computed from the entries in the database using the software tool Mascot (Matrix Science; Perkins et al., 1999, Electrophoresis 20: 3551-3567). Search criteria varied depending on which mass spectrometer was used for the analysis.

### 4. Results

The results of the Signalokinome experiment are shown in FIG. 2. This figure shows a comparison of Drug pulldowns using kinobeads (left circle) and conventional immunoprecipitations with anti-phosphotyrosine antibody beads (right circle). In the Kinobeads experiment a total of 626 proteins were identified, of these were 100 kinases. In the immunoprecipitation (IP) experiment a total of 503 proteins were identified, 12 of these were kinases. Kinases identified with both experiments (common kinases) are listed in the overlap area. The result shows that with the kinobeads significantly more kinases were identified (100 kinases) compared to the anti-phosphotyrosine antibody beads (12 kinases).

The identified kinases for both experimental approaches are listed in the following tables. In addition, the sequences of proteotypic peptides for the kinases are listed in separate tables.

TABLE 6
Kinases identified after immunoprecipitation (IP). Kinase names are in
accordance with the human kinase nomenclature (http://kinase.com)
and Manning et al., 2002, Science 298, 1912-1934 as specified in
supplementary material).

| | | | | Experimental |
|---|---|---|---|---|
| | | | | proteotypic |
| | | | Number of | peptide |
| Kinase | Kinase | | identified | available and |
| identification | Name | Score | peptides | identified |
| 1 | DNAPK | 606 | 20 | X |
| 2 | TIF1b | 202 | 6 | X |
| 3 | p38a | 343 | 11 | X |
| 4 | CDK2 | 196 | 4 | X |
| 5 | TYK2 | 77 | 3 | X |
| 6 | LYN | 67 | 2 | X |
| 7 | EGFR | 2463 | 162 | X |
| 8 | FAK | 1203 | 51 | X |
| 9 | ACK | 160 | 6 | |
| 10 | KHS2 | 65 | 2 | |

TABLE 7
Sequences of proteotypic peptides for kinases identified after
immunoprecipitation (IP, see Table 6). Kinase names are in
accordance with the human kinase nomenclature (http://kinase.com)
and Manning et al., 2002, Science 298, 1912-1934 as specified in
supplementary material).

| | KINASE | IDENTIFIED PROTEOTYPIC |
|---|---|---|
| EXPERIMENT | NAME | PEPTIDE |
| IP | CDK2 | ALFPGDSEIDQLFR |
| IP | CDK2 | IGEGTYGVVYK |
| IP | CDK2 | FMDASALTGIPLPLIK |
| IP | DNAPK | HGDLPDIQIK |
| IP | DNAPK | LACDVDQVTR |
| IP | DNAPK | AALSALESFLK |
| IP | DNAPK | DQNILLGTTYR |
| IP | DNAPK | FMNAVFFLLPK |
| IP | DNAPK | VTELALTASDR |
| IP | DNAPK | LGASLAFNNIYR |
| IP | DNAPK | LNESTFDTQITK |
| IP | DNAPK | QLFSSLFSGILK |
| IP | DNAPK | NILEESLCELVAK |
| IP | DNAPK | TVSLLDENNVSSYLSK |
| IP | DNAPK | TVGALQVLGTEAQSSLLK |
| IP | EGFR | VLGSGAFGTVYK |
| IP | EGFR | IPLENLQIIR |
| IP | EGFR | GDSFTHTPPLDPQELDILK |
| IP | FAK | LGDFGLSR |
| IP | FAK | FLQEALTMR |
| IP | FAK | FFEILSPVYR |
| IP | FAK | LLNSDLGELINK |
| IP | FAK | TLLATVDETIPLLPASTHR |
| IP | HER2/ErbB2 | VLGSGAFGTVYK |
| IP | LYN | EEPIYIITEYMAK |
| IP | p38a | LTDDHVQFLIYQILR |
| IP | p38a | NYIQSLTQMPK |
| IP | p38a | LTGTPPAYLINR |
| IP | TIF1b | IVAERPGTNSTGPAPMAPPR |
| IP | TYK2 | HGIPLEEVAK |
| IP | TYK2 | LSDPGVGLGALSR |

TABLE 8
Kinases identified after drug pulldown with kinobeads. Kinase names
are in accordance with the human kinase nomenclature
(http://kinase.com) and Manning et al., 2002, Science 298, 1912-1934
as specified in supplementary material).

| | | | | Experimental | |
|---|---|---|---|---|---|
| | | | | proteotypic | Identified |
| | | | Number of | peptide | also in IP |
| Kinase | Kinase | | peptides | available and | (see table |
| identification | Name | Score | identified | identified | 1) |
| 1 | DNAPK | 6592 | 263 | X | IP |
| 2 | FRAP | 356 | 9 | X | |
| 3 | ATM | 113 | 3 | X | |
| 4 | ATR | 91 | 2 | X | |
| 5 | CDC2 | 309 | 11 | X | |
| 6 | YES | 1747 | 100 | X | |
| 7 | CaMK2d | 1003 | 41 | X | |
| 8 | JNK1 | 1011 | 45 | X | |
| 9 | JNK2 | 317 | 11 | X | |
| 10 | CaMK2g | 811 | 21 | X | |
| 11 | p38a | 1145 | 90 | X | IP |
| 12 | TNK1 | 81 | 4 | X | |
| 13 | GSK3B | 1408 | 81 | X | |
| 14 | FYN | 1845 | 115 | X | |
| 15 | SRC | 1780 | 91 | X | |
| 16 | Erk2 | 1677 | 200 | X | |
| 17 | CaMK2b | 122 | 3 | X | |
| 18 | JNK3 | 590 | 26 | X | |
| 19 | CK2a1 | 316 | 11 | X | |
| 20 | GSK3A | 1390 | 117 | X | |
| 21 | AurA | 198 | 8 | X | |
| 22 | RIPK2 | 634 | 19 | X | |
| 23 | CDK2 | 1172 | 50 | X | IP |
| 24 | ADCK1 | 88 | 4 | | |
| 25 | CK1a | 566 | 21 | X | |
| 26 | GAK | 1995 | 61 | X | |
| 27 | AAK1 | 123 | 3 | X | |
| 28 | CDK9 | 379 | 13 | X | |
| 29 | CK1d | 206 | 7 | X | |
| 30 | Erk1 | 566 | 26 | X | |
| 31 | NEK9 | 1947 | 102 | X | |
| 32 | TYK2 | 222 | 6 | X | IP |
| 33 | AMPKa1 | 93 | 3 | X | |
| 34 | LYN | 1783 | 67 | X | IP |
| 35 | AurB | 287 | 10 | X | |
| 36 | CK1e | 314 | 10 | X | |
| 37 | EGFR | 165 | 3 | X | IP |
| 38 | EphB2 | 1165 | 43 | X | |
| 39 | ALK2 | 299 | 11 | X | |
| 40 | DDR1 | 759 | 28 | X | |
| 41 | TBK1 | 2042 | 72 | X | |
| 42 | CSK | 1584 | 102 | X | |
| 43 | TEC | 50 | 2 | | |
| 44 | ZAK | 499 | 16 | X | |
| 45 | ALK4 | 472 | 9 | X | |
| 46 | ADCK3 | 309 | 8 | X | |
| 47 | MAP2K5 | 795 | 32 | | |
| 48 | EphB4 | 2237 | 165 | X | |
| 49 | ARG | 2128 | 69 | X | |
| 50 | FER | 2762 | 130 | X | |
| 51 | RSK2 | 2100 | 99 | X | |
| 52 | CK2a2 | 930 | 32 | X | |
| 53 | BMPR1A | 676 | 19 | X | |
| 54 | TGFbR1 | 478 | 15 | X | |
| 55 | EphA5 | 114 | 4 | X | |
| 56 | BRK | 107 | 3 | | |
| 57 | FAK | 1933 | 95 | X | IP |
| 58 | ILK | 209 | 6 | X | |
| 59 | CDK5 | 699 | 27 | X | |
| 60 | DDR2 | 1073 | 44 | X | |
| 61 | JAK1 | 1083 | 31 | | |
| 62 | MYT1 | 152 | 5 | | |
| 63 | PKCd | 53 | 4 | | |
| 64 | RSK3 | 2227 | 117 | X | |
| 65 | Wee1 | 550 | 18 | X | |
| 66 | ACTR2 | 193 | 5 | | |
| 67 | MAP3K2 | 383 | 15 | | |
| 68 | ACTR2B | 82 | 2 | | |
| 69 | Wee1B | 550 | 18 | X | |
| 70 | ABL | 1133 | 35 | X | |
| 71 | MET | 503 | 12 | X | |
| 72 | BRAF | 361 | 7 | | |
| 73 | INSR | 1807 | 64 | X | |
| 74 | KHS1 | 393 | 12 | | |
| 75 | PAK4 | 448 | 15 | X | |
| 76 | PKD1 | 58 | 2 | | |
| 77 | PKD2 | 58 | 2 | X | |
| 78 | IGF1R | 935 | 28 | X | |
| 79 | PHKg2 | 519 | 16 | X | |
| 80 | CaMKK2 | 199 | 5 | X | |
| 81 | MAP2K2 | 294 | 6 | X | |
| 82 | TGFbR2 | 49 | 2 | | |
| 83 | Fused | 42 | 2 | | |
| 84 | EphA2 | 2099 | 90 | X | |
| 85 | ACK | 169 | 4 | | IP |
| 86 | NLK | 46 | 2 | | |
| 87 | CDK7 | 623 | 20 | X | |
| 88 | CHK2 | 58 | 2 | X | |
| 89 | KHS2 | 60 | 4 | | IP |
| 90 | MLK3 | 59 | 1 | X | |
| 91 | PKCe | 53 | 4 | X | |
| 92 | PKCh | 53 | 4 | X | |
| 93 | PYK2 | 106 | 3 | X | |
| 94 | TAO2 | 120 | 3 | | |
| 95 | CK1g1 | 45 | 2 | X | |
| 96 | LIMK1 | 271 | 6 | | |
| 97 | MAP2K1 | 480 | 14 | | |
| 98 | HRI | 52 | 1 | | |
| 99 | SIK | 36 | 1 | | |
| 100 | Erk7 | 67 | 1 | | |
| 101 | NEK2 | 145 | 4 | | |
| 102 | NEK7 | 175 | 5 | | |
| 103 | BMPR1B | 179 | 5 | X | |
| 104 | MAP2K3 | 133 | 5 | | |
| 105 | FGFR2 | 514 | 17 | | |

TABLE 9
Sequences of proteotypic peptides for kinases identified after drug
pulldowns with kinobeads (see Table 8)

| | KINASE | IDENTIFIED PROTEOTYPIC |
|---|---|---|
| EXPERIMENT | NAME | PEPTIDE |
| KinobeadDP | AAK1 | ADIWALGCLLYK |
| KinobeadDP | ABL | GQGESDPLDHEPAVSPLLPR |
| KinobeadDP | ABL | WTAPESLAYNK |
| KinobeadDP | ABL | VADFGLSR |
| KinobeadDP | ABL | NGQGWVPSNYITPVNSLEK |
| KinobeadDP | ADCK3 | DKLEYFEERPFAAASIGQVHLAR |
| KinobeadDP | ADCK3 | SFTDLYIQIIR |
| KinobeadDP | ADCK3 | VALLDFGATR |
| KinobeadDP | ADCK3 | AVLGSSPFLSEANAER |
| KinobeadDP | ALK2 | WFSDPTLTSLAK |
| KinobeadDP | ALK4 | TIVLQEIIGK |
| KinobeadDP | ALK4 | EAEIYQTVMLR |
| KinobeadDP | AMPKa1 | IGHYILGDTLGVGTFGK |
| KinobeadDP | ARG | AASSSSVVPYLPR |
| KinobeadDP | ARG | ESESSPGQLSISLR |
| KinobeadDP | ARG | GAQASSGSPALPR |
| KinobeadDP | ARG | VLGYNQNGEWSEVR |
| KinobeadDP | ARG | VPVLISPTLK |
| KinobeadDP | ARG | WTAPESLAYNTFSIK |
| KinobeadDP | ARG | VADFGLSR |
| KinobeadDP | ARG | NGQGWVPSNYITPVNSLEK |
| KinobeadDP | ATM | LVVNLLQLSK |
| KinobeadDP | ATR | APLNETGEVVNEK |
| KinobeadDP | AurA | SKQPLPSAPENNPEEELASK |
| KinobeadDP | AurA | VEFTFPDFVTEGAR |
| KinobeadDP | AurB | IYLILEYAPR |
| KinobeadDP | AurB | SNVQPTAAPGQK |
| KinobeadDP | BMPR1A | FNSDTNEVDVPLNTR |
| KinobeadDP | BMPR1A | WNSDECLR |
| KinobeadDP | BMPR1A | YEGSDFQCK |
| KinobeadDP | BMPR1A | YMAPEVLDESLNK |
| KinobeadDP | BMPR1A | ETEIYQTVLMR |
| KinobeadDP | BMPR1A | HENILGFIAADIK |
| KinobeadDP | BMPR1A | VFFTTEEASWFR |
| KinobeadDP | BMPR1A | YGEVWMGK |
| KinobeadDP | BMPR1B | ETEIYQTVLMR |
| KinobeadDP | BMPR1B | HENILGFIAADIK |
| KinobeadDP | BMPR1B | VFFTTEEASWFR |
| KinobeadDP | BMPR1B | YGEVWMGK |
| KinobeadDP | CaMK2b | LTQYIDGQGRPR |
| KinobeadDP | CaMK2b | NLINQMLTINPAK |
| KinobeadDP | CaMK2b | AGAYDFPSPEWDTVTPEAK |
| KinobeadDP | CaMK2b | DLKPENLLLASK |
| KinobeadDP | CaMK2d | FYFENALSK |
| KinobeadDP | CaMK2d | AGAYDFPSPEWDTVTPEAK |
| KinobeadDP | CaMK2d | DLKPENLLLASK |
| KinobeadDP | CaMK2g | FYFENLLSK |
| KinobeadDP | CaMK2g | LTQYIDGQGRPR |
| KinobeadDP | CaMK2g | NLINQMLTINPAK |
| KinobeadDP | CaMK2g | AGAYDFPSPEWDTVTPEAK |
| KinobeadDP | CaMK2g | DLKPENLLLASK |
| KinobeadDP | CaMKK2 | GPIEQVYQEIAILK |
| KinobeadDP | CaMKK2 | LAYNENDNTYYAMK |
| KinobeadDP | CDC2 | DLKPQNLLIDDKGTIK |
| KinobeadDP | CDC2 | NLDENGLDLLSK |
| KinobeadDP | CDC2 | SPEVLLGSAR |
| KinobeadDP | CDC2 | IGEGTYGVVYK |
| KinobeadDP | CDK2 | APEILLGCK |
| KinobeadDP | CDK2 | FMDASALTGIPLPLIK |
| KinobeadDP | CDK2 | ALFPGDSEIDQLFR |
| KinobeadDP | CDK2 | IGEGTYGVVYK |
| KinobeadDP | CDK5 | IGEGTYGTVFK |
| KinobeadDP | CDK5 | SFLFQLLK |
| KinobeadDP | CDK7 | APELLFGAR |
| KinobeadDP | CDK7 | VPFLPGDSDLDQLTR |
| KinobeadDP | CDK9 | GSQITQQSTNQSR |
| KinobeadDP | CDK9 | IGQGTFGEVFK |
| KinobeadDP | CK1a | HPQLLYESK |
| KinobeadDP | CK1a | LFLIDFGLAK |
| KinobeadDP | CK1d | FDDKPDYSYLR |
| KinobeadDP | CK1d | GNLVYIIDFGLAK |
| KinobeadDP | CK1d | TVLLLADQMISR |
| KinobeadDP | CK1e | FDDKPDYSYLR |
| KinobeadDP | CK1e | GNLVYIIDFGLAK |
| KinobeadDP | CK1e | TVLLLADQMISR |
| KinobeadDP | CK1g1 | TLFTDLFEK |
| KinobeadDP | CK2a1 | GGPNIITLADIVKDPVSR |
| KinobeadDP | CK2a1 | QLYQTLTDYDIR |
| KinobeadDP | CK2a1 | TPALVFEHVNNTDFK |
| KinobeadDP | CK2a2 | HLVSPEALDLLDKLLR |
| KinobeadDP | CK2a2 | LIDWGLAEFYHPAQEYNVR |
| KinobeadDP | CK2a2 | QLYQILTDFDIR |
| KinobeadDP | CK2a2 | TPALVFEYINNTDFK |
| KinobeadDP | CK2a2 | VLGTEELYGYLK |
| KinobeadDP | CSK | HSNLVQLLGVIVEEK |
| KinobeadDP | CSK | LLYPPETGLFLVR |
| KinobeadDP | CSK | VMEGTVAAQDEFYR |
| KinobeadDP | DDR1 | AQPFGQLTDEQVIENAGEFFR |
| KinobeadDP | DDR1 | NCLVGENFTIK |
| KinobeadDP | DDR1 | NLYAGDYYR |
| KinobeadDP | DDR1 | QVYLSRPPACPQGLYELMLR |
| KinobeadDP | DDR1 | WMAWECILMGK |
| KinobeadDP | DDR1 | YLATLNFVHR |
| KinobeadDP | DDR2 | DVAVEEFPR |
| KinobeadDP | DDR2 | FMATQIASGMK |
| KinobeadDP | DDR2 | HEPPNSSSSDVR |
| KinobeadDP | DDR2 | IFPLRPDYQEPSR |
| KinobeadDP | DDR2 | NLYSGDYYR |
| KinobeadDP | DDR2 | QTYLPQPAICPDSVYK |
| KinobeadDP | DDR2 | WMSWESILLGK |
| KinobeadDP | DDR2 | YLSSLNFVHR |
| KinobeadDP | DNAPK | AALSALESFLK |
| KinobeadDP | DNAPK | DQNILLGTTYR |
| KinobeadDP | DNAPK | FMNAVFFLLPK |
| KinobeadDP | DNAPK | FVPLLPGNR |
| KinobeadDP | DNAPK | GLSSLLCNFTK |
| KinobeadDP | DNAPK | HGDLPDIQIK |
| KinobeadDP | DNAPK | INQVFHGSCITEGNELTK |
| KinobeadDP | DNAPK | IPALDLLIK |
| KinobeadDP | DNAPK | LACDVDQVTR |
| KinobeadDP | DNAPK | LAGANPAVITCDELLLGHEK |
| KinobeadDP | DNAPK | LGASLAFNNIYR |
| KinobeadDP | DNAPK | LGNPIVPLNIR |
| KinobeadDP | DNAPK | LLEEALLR |
| KinobeadDP | DNAPK | LNESTFDTQITK |
| KinobeadDP | DNAPK | LPLISGFYK |
| KinobeadDP | DNAPK | LPSNTLDR |
| KinobeadDP | DNAPK | LQETLSAADR |
| KinobeadDP | DNAPK | MSTSPEAFLALR |
| KinobeadDP | DNAPK | NILEESLCELVAK |
| KinobeadDP | DNAPK | NLLIFENLIDLK |
| KinobeadDP | DNAPK | NLSSNEAISLEEIR |
| KinobeadDP | DNAPK | QITQSALLAEAR |
| KinobeadDP | DNAPK | QLFSSLFSGILK |
| KinobeadDP | DNAPK | SLGPPQGEEDSVPR |
| KinobeadDP | DNAPK | SQGCSEQVLTVLK |
| KinobeadDP | DNAPK | TVGALQVLGTEAQSSLLK |
| KinobeadDP | DNAPK | TVSLLDENNVSSYLSK |
| KinobeadDP | DNAPK | VCLDIIYK |
| KinobeadDP | DNAPK | VTELALTASDR |
| KinobeadDP | DNAPK | VVQMLGSLGGQINK |
| KinobeadDP | EGFR | GDSFTHTPPLDPQELDILK |
| KinobeadDP | EphA2 | DGEFSVLQLVGMLR |
| KinobeadDP | EphA2 | FADIVSILDK |
| KinobeadDP | EphA2 | LPSTSGSEGVPFR |
| KinobeadDP | EphA2 | NILVNSNLVCK |
| KinobeadDP | EphA2 | VSDFGLSR |
| KinobeadDP | EphA5 | WTAPEAIAFR |
| KinobeadDP | EphA5 | VSDFGLSR |
| KinobeadDP | EphB2 | AGAIYVFQVR |
| KinobeadDP | EphB2 | FGQIVNTLDK |
| KinobeadDP | EphB2 | TFPNWMENPWVK |
| KinobeadDP | EphB2 | VDTIAADESFSQVDLGGR |
| KinobeadDP | EphB2 | NILVNSNLVCK |
| KinobeadDP | EphB2 | VSDFGLSR |
| KinobeadDP | EphB4 | APSGAVLDYEVK |
| KinobeadDP | EphB4 | FPQVVSALDK |
| KinobeadDP | EphB4 | LNDGQFTVIQLVGMLR |
| KinobeadDP | EphB4 | WTAPEAIAFR |
| KinobeadDP | EphB4 | NILVNSNLVCK |
| KinobeadDP | EphB4 | VSDFGLSR |
| KinobeadDP | Erk1 | NYLQSLPSK |
| KinobeadDP | Erk1 | APEIMLNSK |
| KinobeadDP | Erk1 | YIHSANVLHR |
| KinobeadDP | Erk1 | ICDFGLAR |
| KinobeadDP | Erk2 | FRHENIIGINDIIR |
| KinobeadDP | Erk2 | GQVFDVGPR |
| KinobeadDP | Erk2 | LKELIFEETAR |
| KinobeadDP | Erk2 | NYLLSLPHK |
| KinobeadDP | Erk2 | VADPDHDHTGFLTEYVATR |
| KinobeadDP | Erk2 | APEIMLNSK |
| KinobeadDP | Erk2 | YIHSANVLHR |
| KinobeadDP | Erk2 | ICDFGLAR |
| KinobeadDP | FAK | FFEILSPVYR |
| KinobeadDP | FAK | FLQEALTMR |
| KinobeadDP | FAK | LLNSDLGELINK |
| KinobeadDP | FAK | TLLATVDETIPLLPASTHR |
| KinobeadDP | FAK | LGDFGLSR |
| KinobeadDP | FER | LQDWELR |
| KinobeadDP | FER | QEDGGVYSSSGLK |
| KinobeadDP | FER | SGVVLLNPIPK |
| KinobeadDP | FER | WTAPEALNYGR |
| KinobeadDP | FRAP | GPTPAILESLISINNK |
| KinobeadDP | FRAP | VLGLLGALDPYK |
| KinobeadDP | FYN | EVLEQVER |
| KinobeadDP | FYN | WTAPEAALYGR |
| KinobeadDP | FYN | LIEDNEYTAR |
| KinobeadDP | FYN | GSLLDFLK |
| KinobeadDP | FYN | IADFGLAR |
| KinobeadDP | GAK | ALVEEEITR |
| KinobeadDP | GAK | AMLQVNPEER |
| KinobeadDP | GAK | AVVMTPVPLFSK |
| KinobeadDP | GAK | IAVMSFPAEGVESALK |
| KinobeadDP | GAK | TPEIIDLYSNFPIGEK |
| KinobeadDP | GSK3A | VIGNGSFGVVYQAR |
| KinobeadDP | GSK3A | VTTVVATLGQGPER |
| KinobeadDP | GSK3A | YFFYSSGEK |
| KinobeadDP | GSK3B | LLEYTPTAR |
| KinobeadDP | GSK3B | LYMYQLFR |
| KinobeadDP | GSK3B | VIGNGSFGVVYQAK |
| KinobeadDP | GSK3B | YFFYSSGEK |
| KinobeadDP | IGF1R | AENGPGPGVLVLR |
| KinobeadDP | IGF1R | HSHALVSLSFLK |
| KinobeadDP | IGF1R | MYFAFNPK |
| KinobeadDP | IGF1R | TTINNEYNYR |
| KinobeadDP | IGF1R | VAGLESLGDLFPNLTVIR |
| KinobeadDP | IGF1R | DIYETDYYR |
| KinobeadDP | IGF1R | IEFLNEASVMK |
| KinobeadDP | IGF1R | IGDFGMTR |
| KinobeadDP | ILK | MYAPAWVAPEALQK |
| KinobeadDP | INSR | DLLGFMLFYK |
| KinobeadDP | INSR | ELGQGSFGMVYEGNAR |
| KinobeadDP | INSR | ESLVISGLR |
| KinobeadDP | INSR | TIDSVTSAQELR |
| KinobeadDP | INSR | TVNESASLR |
| KinobeadDP | INSR | WEPYWPPDFR |
| KinobeadDP | INSR | DIYETDYYR |
| KinobeadDP | INSR | IEFLNEASVMK |
| KinobeadDP | INSR | WMAPESLK |
| KinobeadDP | INSR | IGDFGMTR |
| KinobeadDP | JNK1 | NIIGLLNVFTPQK |
| KinobeadDP | JNK1 | APEVILGMGYK |
| KinobeadDP | JNK1 | ILDFGLAR |
| KinobeadDP | JNK2 | NIISLLNVFTPQK |
| KinobeadDP | JNK2 | APEVILGMGYK |
| KinobeadDP | JNK2 | ILDFGLAR |
| KinobeadDP | JNK3 | NIISLLNVFTPQK |
| KinobeadDP | JNK3 | APEVILGMGYK |
| KinobeadDP | JNK3 | ILDFGLAR |
| KinobeadDP | LYN | EEPIYIITEYMAK |
| KinobeadDP | LYN | GSLLDFLK |
| KinobeadDP | LYN | IADFGLAR |
| KinobeadDP | MAP2K2 | ISELGAGNGGVVTK |
| KinobeadDP | MAP2K2 | YPIPPPDAK |
| KinobeadDP | MET | AFFMLDGILSK |
| KinobeadDP | MET | DLIGFGLQVAK |
| KinobeadDP | MLK3 | ITVQASPGLDR |
| KinobeadDP | NEK9 | AGGGAAEQEELHYIPIR |
| KinobeadDP | NEK9 | LGINLLGGPLGGK |
| KinobeadDP | NEK9 | LQQENLQIFTQLQK |
| KinobeadDP | NEK9 | SSTVTEAPIAVVTSR |
| KinobeadDP | NEK9 | TSEVYVWGGGK |
| KinobeadDP | p38a | LTGTPPAYLINR |
| KinobeadDP | p38a | NYIQSLTQMPK |
| KinobeadDP | p38a | SLEEFNDVYLVTHLMGADLNNIVK |
| KinobeadDP | p38a | LTDDHVQFLIYQILR |
| KinobeadDP | p38a | ILDFGLAR |
| KinobeadDP | PAK4 | AALQLVVDPGDPR |
| KinobeadDP | PHKg2 | LTAEQALQHPFFER |
| KinobeadDP | PHKg2 | SLLEAVSFLHANNIVHR |
| KinobeadDP | PHKg2 | VAVWTVLAAGR |
| KinobeadDP | PHKg2 | LSPEQLEEVR |
| KinobeadDP | PYK2 | EIITSILLSGR |
| KinobeadDP | PYK2 | EVGLDLFFPK |
| KinobeadDP | PYK2 | VLANLAHPPAE |
| KinobeadDP | PYK2 | LGDFGLSR |
| KinobeadDP | RIPK2 | CLIELEPVLR |
| KinobeadDP | RIPK2 | SLPAPQDNDFLSR |
| KinobeadDP | RIPK2 | SPSLNLLQNK |
| KinobeadDP | RIPK2 | TQNILLDNEFHVK |
| KinobeadDP | RSK2 | EASAVLFTITK |
| KinobeadDP | RSK2 | LGMPQFLSPEAQSLLR |
| KinobeadDP | RSK2 | NQSPVLEPVGR |
| KinobeadDP | RSK2 | LYLILDFLR |
| KinobeadDP | RSK3 | APQAPLHSVVQQLHGK |
| KinobeadDP | RSK3 | EASFVLHTIGK |
| KinobeadDP | RSK3 | LGMPQFLSTEAQSLLR |
| KinobeadDP | RSK3 | LYLILDFLR |
| KinobeadDP | SRC | AANILVGENLVCK |
| KinobeadDP | SRC | WTAPEAALYGR |
| KinobeadDP | SRC | LIEDNEYTAR |
| KinobeadDP | SRC | GSLLDFLK |
| KinobeadDP | TBK1 | EPLNTIGLIYEK |
| KinobeadDP | TBK1 | FGSLTMDGGLR |
| KinobeadDP | TBK1 | IISSNQELIYEGR |
| KinobeadDP | TBK1 | LFAIEEETTTR |
| KinobeadDP | TBK1 | TTEENPIFVVSR |
| KinobeadDP | TBK1 | VIGEDGQSVYK |
| KinobeadDP | TGFbR1 | HDSATDTIDIAPNHR |
| KinobeadDP | TGFbR1 | TIVLQESIGK |
| KinobeadDP | TGFbR1 | TLSQLSQQEGIK |
| KinobeadDP | TGFbR1 | VPNEEDPSLDRPFISEGTTLK |
| KinobeadDP | TGFbR1 | EAEIYQTVMLR |
| KinobeadDP | TNK1 | MLPEAGSLWLLK |
| KinobeadDP | TYK2 | LGLAEGTSPFIK |
| KinobeadDP | Wee1B | IPQVLSQEFTELLK |
| KinobeadDP | Wee1B | ISSPQVEEGDSR |
| KinobeadDP | YES | LLLNPGNQR |
| KinobeadDP | YES | LPQLVDMAAQIADGMAYIER |
| KinobeadDP | YES | SDVWSFGILQTELVTK |
| KinobeadDP | YES | AANILVGENLVCK |
| KinobeadDP | YES | EVLEQVER |
| KinobeadDP | YES | FQIINNTEGDWWEAR |
| KinobeadDP | YES | WTAPEAALYGR |
| KinobeadDP | YES | LIEDNEYTAR |
| KinobeadDP | YES | GSLLDFLK |
| KinobeadDP | YES | IADFGLAR |
| KinobeadDP | ZAK | CEIEATLER |
| KinobeadDP | ZAK | GLEGLQVAWLVVEK |
| KinobeadDP | ZAK | LTIPSSCPR |
| KinobeadDP | ZAK | NVVIAADGVLK |

### Example 3

### Screening Assay using Test Compounds for Protein Elution

This example illustrates competitive elution of proteins bound to kinobeads with non-modified test compounds (see particularly the fourth aspect of the invention). The kinobeads (as described in example 1) were contacted with mouse brains lysate, bound proteins were eluted with various test compounds and the released proteins were analysed by mass spectrometry.

### 1. Preparation of the Biological Sample (Tissue Lysate)

### 1.1 Preparation of Lysates

A mouse brain lysate was prepared by mechanical disruption in lysis buffer (5 ml buffer per mouse brain) under gentle conditions that maintain the structure and function of proteins. The following steps were performed:
Thaw the tissue quickly at room temperature or 37° C., then transfer tissue to a glass bottle containing the 1× lysis buffer (use a vial big enough to be used with Polytron PT 3100 homogenizer)
Lyse the organ/tissue with 4×10 sec pulses at 5000-7000 rpm at 4° C. in the cold room
Transfer the homogenate into precooled 50 ml falcon tubes
Incubate homogenate on ice for 30 min
Spin cells for 10 min at 6000 g at 4° C. (6.000 rpm in Sorvall SLA600, precooled)
Transfer supernatant to a UZ-polycarbonate tube (Beckmann, 355654)
Spin supernatant for 1 h at 145.000 g at 4° C. (40.000 rpm in Ti50.2, precooled)
Save supernatant (remove and discard most of the lipid layer if possible), transfer supernatant into a glass bottle and store on ice
Determine protein concentration by Bradford assay (BioRad). Typical protein concentrations are in the range of 5-10 mg/ml.
Prepare aliquots in 15 to 50 ml Falcon tubes
Freeze aliquots in liquid nitrogen and store them at −80° C.

### 1.2 Preparation of Lysis Buffer and Stock Solutions

Preparation of 100 ml 1× Lysis Buffer with 0.8% NP40

Combine the following solutions or reagents and add destilled water to a final volume of 100 ml: 20 ml 5× lysis buffer (see below), 100 μl 1 M DTT, 5 ml 0.5 M NaF, 4 ml 20% NP40, 4 complete EDTA-free tablets (protease inhibitor cocktail, Roche Diagnostics, 1 873 580), add distilled water to 100 ml.

TABLE 10
Preparation of 5x-lysis buffer

| | Stock | | Final conc. in 1 x | | Add for 1l 5 x lysis | |
|---|---|---|---|---|---|---|
| Substance: | solution | | lysis buffer | | buffer | |
| Tris/HCl pH 7.5 | 1 | M | 50 | mM | 250 | ml |
| Glycerol | 87% | | 5% | | 288 | ml |
| MgCl₂ | 1 | M | 1.5 | mM | 7.5 | ml |
| NaCl | 5 | M | 150 | mM | 150 | ml |
| Na₃VO₄ | 100 | mM | 1 | mM | 50 | ml |

These solutions were obtained from the following suppliers:

1M Tris/HCl pH 7.5: Sigma, T-2663; 87% Glycerol: Merck, cat. no. 04091.2500; 1 M MgCl₂: Sigma, M-1028; 5 MNaCl: Sigma, S-5150.

The 5× concentrated lysis buffer was filtered through a 0.22 μm filter and stored in 40 ml aliquots at −80° C.

### Preparation of Stock Solutions

Preparation of 100 mM Na₃VO₄ stock solution:

Dissolve 9.2 g Na₃VO₄ in 400 ml distilled water.

1) Adjust the pH to 10.0 using either 1 N NaOH or 1 N HCl. The starting pH of the sodium orthovanadate solution may vary with batch. At pH 10.0 the solution will be yellow.
2) Boil the solution until it turns colorless (approximately 10 min).
3) Cool to room temperature.
4) Readjust the pH to 10.0 and repeat steps 2 and 3 until solution remains colorless and the pH stabilizes at 10.0.
5) Adjust the volume to 500 ml with distilled water.
6) Freeze aliquots at −20° C. Aliquots can be stored for several months.

### Preparation of 500 mM NaF Stock Solution:

Dissolve 21.0 g NaF (Sigma, S7920) in 500 ml distilled water. Filter solution through 0.22 μm filter and store at 4° C.

### Preparation of 20% NP40-Solution:

Weigh 40.0 g NP40 (Sigma, Igepal-CA630, catalogue No. 13021). Add distilled water up to 200 g. Mix completely and store solution at room temperature.

### Preparation of 1 M DTT Solution:

Dissolve 7.7 g DTT (Biomol, catalogue No. 04010) in 50 ml distilled water. Filter solution through 0.22 μm filter and freeze 400 μl aliquots at −20° C. Aliquots can be stored for several months.

2. Contacting of the Kinobeads with the Cell Lysate and Elution of Bound Proteins by Test Compounds

The kinobeads were contacted with mouse brain lysate under conditions that allow the binding of the proteins in the lysate to the ligands. The binding conditions were close to physiological by choosing suitable buffer conditions preserving the function of the proteins. After removing non-captured proteins through a gentle washing procedure the bound proteins were contacted with a test compound for protein elution.

### 2.1 Preparation of DP-buffers

TABLE 11
Preparation of 5x-DP buffer

| | Stock | | Final conc. in 1 x | | Add for 1l 5 x lysis | |
|---|---|---|---|---|---|---|
| Substance: | solution | | lysis buffer | | buffer | |
| Tris/HCl pH 7.5 | 1 | M | 50 | mM | 250 | ml |
| Glycerol | 87% | | 5% | | 288 | ml |
| MgCl₂ | 1 | M | 1.5 | mM | 7.5 | ml |
| NaCl | 5 | M | 150 | mM | 150 | ml |
| Na₃VO₄ | 100 | mM | 1 | mM | 50 | ml |

The 5×-DP buffer is filtered through 0.22 μm filter and stored in 40 ml-aliquots at −80° C. (Note: the Same Buffer is Also Used for the Preparation of Total Cell Lysates.)

These solutions were obtained from the following suppliers: 1.0 M Tris/HCl pH 7.5 (Sigma, T-2663), 87% Glycerol (Merck, catalogue number 04091.2500); 1.0 M MgCl₂ (Sigma, M-1028); 5.0 M NaCl (Sigma, S-5150).

The following 1× DP Buffers were prepared
1×DP buffer (for bead equilibration)
1×DP buffer/0.4% NP40 (for bead equilibration and first wash step of beads)
1×DP buffer/0.2% NP40 (for second wash step of beads and for compound elution)
1×DP buffer/protease inhibitors (for first lysate dilution step); add one protease inhibitor tablet per 25 ml lysis buffer (EDTA-free tablet, protease inhibitor cocktail, Roche Diagnostics, 1 873 580)
1×DP buffer/0.4% NP40/protease inhibitors (for second lysate dilution step)

### Example for Preparation of 1× DP-Buffer/0.4% NP40 (100 ml)

Combine the following solutions and reagents and add distilled water up to a final volume of 100 ml: 20 ml 5×DP buffer, 5 ml 0.5 M NaF, 2 ml 20% NP40, 100 μl 1 M DTF, and add distilled water up to 100 ml. All buffers contain 1 mM DTT final concentration.

### 2.2 Washing and Equilibration of Beads

The kinobeads (Example 1) were prepared for the binding reaction by washing with a suitable buffer and equilibration in the buffer.

1. Use 15 ml Falcon tubes for all washing steps.
2. Use 100 μl KinoBeads per experiment (settled bead volume): mix equal amounts (25 μl) of each bead type coupled with the following 4 ligands (coupling density of 1 μmol/ml): Bis VIII (CZC00001056), Purvalanol B (CZC00007097), PD173955 derivative (CZC00007324), and CZC00008004.
3. Wash beads two times with 3 ml 1×DP buffer and once with 3 ml 1×DP buffer/0.4% NP40. During each wash step invert tubes 3-5 times, centrifuge 2 minutes at 1200 rpm at 4° C. in a Heraeus centrifuge. Supernatants are aspirated supernatants and discarded. After the last washing step prepare a 1:1 slurry (volume/volume) with 1×DP buffer/0.4% NP40.

2.3 Preparation of Diluted Cell Lysate

The mouse brain lysate was prepared for the binding reaction by dilution in a suitable buffer and clearing through a centrifugation step.

1. Use a volume of cell lysate corresponding to 50 mg protein per experiment.
2. Thaw the lysate quickly in 37° C. water bath, then keep the sample on ice.
3. Dilute the lysate in the following way:
3) dilute lysate with 1×DP buffer/protease inhibitors to reduce detergent concentration from 0.8% to 0.4% NP-40.
4) dilute lysate further with 1×DP buffer/0.4% NP40/protease inhibitors to reach a final protein concentration of 5 mg/ml.

(Note: The second dilution step is only required if the protein concentration of the lysate after the first dilution step is higher than 5 mg/ml).

4. Transfer diluted lysate into UZ-polycarbonate tube (Beckmann, 355654).
5. Clear diluted lysate through ultracentrifugation (20 min, 4° C., 100.000 g, T150.2 rotor, precooled ultracentrifuge).
6. Save supernatant and keep it on ice.

### 2.4 Binding Reaction and Washing

The washed and equilibrated beads (section 2.2) were contaced with the diluted cell lysate (section 2.3) in order to allow binding of proteins to the ligands. Non-specifically bound proteins were removed by gentle washing in order to reduce background binding.

1. Combine diluted cleared lysate with 100 μl of washed KinoBeads in 15 ml or 50 ml Falcon tube.
2. Incubate for 2 hours at 4° C., rotate on ROTO SHAKE GENIE (Scientific Industries, Inc.) in cold room.
3. After incubation centrifuge for 3 minutes at 1200 rpm in a Heraeus centrifuge or equivalent at 4° C.
4. Remove supernatant carefully without loosing the beads.
5. Transfer the beads to a Mobicol-columns with 90 μm filter (MoBiTec, Goettingen, Cat. no: M1002-90).
6. Wash beads with 10 ml 1×DP buffer/0.4% NP-40 and 5 ml 1×DP buffer/0.2% NP-40.
7. Let washing buffer run through the column completely before proceeding with next step
8. Place column in Eppendorf tube and centrifuge them for 1 minute at 800 rpm at 4° C. Close columns with lower lid.
2.5 Elution of Bound Proteins with Test Compounds

Various test compounds (see section 2.7) were used to release bound proteins following these steps:

1. Resuspend beads with bound proteins (section 2.4) in 1×DP buffer/0.2% NP-40 as 1:3 slurry (volume/volume).
2. Transfer 20 μl the 1:3 slurry into MoBiTech columns (equivalent to 5 μl of beads).
3. Place the column into an Eppendorf tube and centrifuge it for 15 seconds at 800 rpm at 4° C. Close columns with lower lid.
4. Add 10 μl elution buffer containing 1.0 mM of the test compound (concentration ranges of 0.1 to 1.0 mM are suitable). As a control use elution buffer containing 2% DMSO or 2% DMF dependent on the solvent used for dissolving the test compound.
Close column with upper lid.
5. Incubate for 30 minutes at 4° C. in an Eppendorf incubator at 700 rpm.
6. Open column (first top, then bottom), put column back into siliconized tube (SafeSeal Microcentrifuge Tubes, cat. no 11270, Sorenson BioScience, Inc.). To harvest the eluate centrifuge 2 minutes at 2.000 rpm in table top centrifuge at room temperature. The typical volume of the eluate is approximately 15 μl.
7. Add 5 μl 4× NuPAGE SDS Sample Buffer (Invitrogen, NP0007) containing 100 mM DTT (DTT has to be added just prior to use).
8. Incubate for 30 minutes at 50° C.
9. Add 1/10 volume of 200 mg/ml iodoacetamide, incubate for 30 min at room temperature, protect from light. This reaction leads to the alkylation of cysteines for mass spectrometry.
10. Before loading samples onto the gel, centrifuge samples for 5 minutes at 15.000 rpm in order to remove precipitates.
11. For protein separation apply 10 μl sample to NuPAGE 4-12% Bis-Tris gel (Invitrogen, NPO₃₃₅).
2.6 Preparation of Stock Solutions used in this Protocol

### Preparation of a 100 mM Na₃VO₄ Stock Solution

1) Dissolve 9.2 g Na₃VO₄ in 400 ml distilled water.
2) Adjust the pH to 10.0 using either 1 N NaOH or 1 N HCl. The starting pH of the sodium orthovanadate solution may vary with batch. At pH 10.0 the solution will be yellow.
3) Boil the solution until it turns colorless (approximately 10 min).
4) Cool to room temperature.
5) Readjust the pH to 10.0 and repeat steps 2 and 3 until solution remains colorless and the pH stabilizes at 10.0.
6) Adjust the volume to 500 ml with distilled water.
7) Freeze aliquots at −20° C. Aliquots can be stored for several months.

### Preparation of a 500 mM NaF Stock Solution

Dissolve 21.0 g NaF (Sigma, S7920) in 500 ml distilled water. Filter solution through a 0.22 μm filter and store at 4° C.

### Preparation of a 20% NP40-Solution

Weigh 40.0 g NP40 (Sigma, Igepal-CA630, cat. no. 13021). Add distilled water up to 200 g. Mix completely and store solution at room temperature.

### Preparation of a 1 M DTT Solution

Dissolve 7.7 g DTT (Biomol, catalogue number 04010) in 50 ml distilled water. Filter solution through 0.22 μm filter and freeze 400 μl aliquots at −20° C. Aliquots can be stored for several months.

Preparation of a Iodoacetamide Stock Solution (200 mg/ml)

Dissolve 2.0 g Iodoacetamide (Sigma, 1-6125) in 10 ml distilled water. Filter solution through 0.22 μm filter and freeze 400 μl aliquots at −20° C. Aliquots can be stored for several months.

### 2.7 Test Compounds for Elution

The test compounds listed below were used for elution experiments after dilution as described below.

### Preparation of Test Compound Stocks:

Typically all compounds are dissolved in 100% DMSO (Fluka, cat. no 41647) at a concentration of 100 mM. Alternatively, 100% DMF (Fluka, cat. no 40228) can be used for those compounds which cannot be dissolved in DMSO. Compounds are stored at −20° C.

### Dilution of Test Compound for Elution Experiments:

Prepare 50 mM stock by diluting the 100 mM stock 1:1 with 100% DMSO. For elution experiments further dilute the compound 1:50 with elution buffer (=1×DP-buffer/0,2% NP40).

CZC1038: Bisindolylmaleimide III (Supplier: Alexis Biochemicals; catalogue number 270-051-MO05; Chemical Formula-C₂₃H₂₀N₄O₂; MW 384.4).

CZC7097: Purvalanol B (Supplier: Tocris Biochemicals, catalogue number 1581; chemical composition C₂₀H₂₅ClN₆O₃; MW 441.92; CAS number 212844-54-7).

CZC00007324 (PD173955 derivative; the syntheis is described in Example 1.

CZC00008004: This is an analog of CZC00004919 (synthesis is described in Example 1).

CZC00007098: SB202190 (Supplier: TOCRIS 1264 1A/46297; chemical formula C₂₀H₁₄N₃OF; MW 331.34).

CZC00009280: Staurosporine (Supplier: SIGMA-ALDRICH: S4400; broad range kinase inhibitor; chemical formula C₂₈H₂₆N₄O₃; MW 466.53).

CZC00007449: SP600125 (Supplier: Merck Biosciences #420119, JNKl/2 inhibitor; chemical formula C₁₄H₈N₂O; MW 220.2).

3. Mass Spectrometric Analysis of Eluted Enzymes (e.g. Kinases)

### Description of Proteotypic Peptides

Tryptic digestion of a SDS-PAGE-separated protein mixture generates for each protein numerous distinct peptide fragments with different physico-chemical properties. These peptides differ in compatibility with the mass spectrometry-based analytical platform used for protein identification (ID), here nanocapillary reversed phase-liquid chromatography electrospray ionization tandem mass spectrometry (RP-LC-MS/MS). As a result, peptide frequencies for peptides from the same source protein differ by a great degree, the most frequently observed peptides that “typically” contribute to the identification of this protein being termed “proteotypic” peptides. Thus, a “proteotypic peptide” is an experimentally well observable peptide that uniquely identifies a specific protein or protein isoform.

### Advantages of Proteotypic Peptides

The use of proteotypic peptides for protein identification allows rapid and focussed identification and quantitation of multiple known target proteins by focusing the protein identification process on a screening for the presence of information-rich signature peptides.

Experimental Identification of Proteotypic Peptides

One strategy to generate a list of proteotypic peptides is to collect peptide-identification data empirically and to search the dataset for commonly observed peptides that uniquely identify a protein.

For each IPI database protein entry with at least 10 unequivocal identifications in the CZ dataset (multipeptide IDs or manually verified single peptide IDs), peptide frequencies for contributing peptides are calculated. Only specific, best peptide-to-spectrum matches according to the database search engine Mascot™ (Matrix Science) are considered.

For definition of proteotypic peptides for a specific protein, peptides are ordered by descending peptide frequency and a cumulative peptide presence is calculated: This value gives for each peptide the ratio of identifications where this peptide or any of the peptides with a higher peptide frequency was present. Proteotypic peptides are defined using a cut-off for cumulative presence of 95%, i.e. at least 95% of identification events for this protein were based on at least one proteotypic peptide.

### 3.1 Protein Digestion and Sample Preparation Prior to Mass Spectrometric Analysis

Proteins were concentrated, separated on 4-12% NuPAGE® Novex gels (Invitrogen, Carlsbad, Calif.), and stained with colloidal Coomassie blue. Gel lanes were systematically cut across the entire separation range into ≦48 slices (bands and interband regions) and subjected to in-gel tryptic digestion essentially as described by Shevchenko et al., 1996, Analytical Chemistry 68: 850-858. Briefly, gel plugs were destained overnight in 5 mM NH₄HCO₃ in 50% EtOH, digested with for 4 hours with trypsin at 12.5 ng/μl in 5 mM NH₄HCO₃. Peptides were extracted with 1% formic acid, transferred into a second 96 well plate and dried under vacuum. Dry peptides were resuspended 10 μl 0.1% formic acid in water and 5 μl were injected into the LC-MS/MS system for protein identification.

### 3.2 Mass Spectrometric Data Acquisition

Peptide samples were injected into a nano LC system (CapLC, Waters or Ultimate, Dionex) which was directly coupled either to a quadrupole time-of-flight (QTOF2, QTOF Ultima, QTOF Micro, Micromass or QSTAR Pulsar, Sciex) or ion trap (LCQ Deca XP, LTQ, Thermo-Finnigan) mass spectrometer. Peptides were separated on the LC system using a gradient of aqueous and organic solvents with a typical gradient time of between 15 and 45 min. Solvent A was 5% acetonitrile in 0.1% formic acid and solvent B was 70% acetonitrile in 0.1% formic acid.

### 3.3 Protein Identification

The peptide mass and fragmentation data generated in the LC-MS/MS experiments were used to query an in-house curated version of the International Protein Index (IPI) protein sequence database (EBI) Proteins were identified by correlating the measured peptide mass and fragmentation data with the same data computed from the entries in the database using the software tool Mascot (Matrix Science; Perkins et al., 1999, Electrophoresis 20: 3551-3567). Search criteria varied depending on which mass spectrometer was used for the analysis.

### 4. Results

The method allows to establish a profile of the eluted kinases for any given test compound thereby allowing to assess the selectivity of the test compound. One limitation is that only the kinases captured on the kinobeads during the first step can be assessed, which might represent a subfraction of all kinases contained within the cell lysate.

TABLE 12
Released kinases identified by mass spectrometry analysis after specific elution
with test compounds (numbers represent mass spectrometry score/number of identified
peptides). Kinase names are in accordance with the human kinase nomenclature
(http://kinase.com) and Manning et al., 2002, Science 298, 1912-1934 as specified in
supplementary material). The first four compounds in table 12 are the non-immobilized
versions of the kinobead ligands 1-4.

| Kinase | 2% DMSO | CZC1038 | CZC7097 | CZC7324 | CZC8004 | CZC1355 | CZC7098 | CZC7479 | CZC9280 | SDS |
|---|---|---|---|---|---|---|---|---|---|---|
| AAK1 | 130/5 | 562/38 | 1036/51 | 582/35 | 718/35 | 638/27 | 198/4 | 593/32 | 777/44 | 240/32 |
| ALK2 | | | | | | | | | | 65/2 |
| AMPKa1 | | | | | 64/2 | | | | | |
| ARG | | | | 59/2 | | | | | | 452/28 |
| BRAFps | | | | 70/4 | | | | | | |
| CaMK2a | | 911/53 | 978/27 | 513/11 | 966/43 | 288/9 | | 509/11 | 1027/69 | 786/74 |
| CaMK2b | | 834/52 | 679/16 | 499/14 | 685/30 | 572/19 | | 143/5 | 1182/72 | 615/75 |
| CaMK2d | | 702/17 | 473/12 | 328/7 | 444/11 | 260/8 | | | 822/40 | 434/36 |
| CaMK2g | | 550/18 | | | 527/27 | | | | 809/25 | 379/42 |
| CaMKK2 | | 314/6 | 465/9 | 101/3 | 522/12 | 93/2 | | | 282/10 | 338/10 |
| CDK5 | 303/7 | 831/19 | 1086/41 | 462/11 | 794/27 | 856/28 | 166/4 | 504/11 | 861/43 | 604/21 |
| CDKL5 | | | 48/1 | | | | | | | |
| CK1e | | | | | | | | | | |
| CR1K | | 90/5 | | | | | | | | |
| CSK | | | 492/12 | 809/19 | | | | | 307/8 | 365/15 |
| EphA4 | | | 1702/37 | 1233/32 | 138/3 | | | | 332/11 | 727/33 |
| EphA5 | | | 330/14 | 144/3 | | | | | | 246/9 |
| EphA7 | | | | | | | | | | |
| EphB1 | | | 366/10 | | | | | | | 180/10 |
| EphB2 | | | 1334/30 | 1004/22 | 149/5 | | | | 114/4 | 437/19 |
| Erk1ps5 | | 418/14 | 749/48 | 557/77 | 386/26 | 270/13 | | | 71/2 | 239/8 |
| Erk2 | 751/25 | 754/50 | 1437/115 | 1179/235 | 810/46 | 1036/82 | 506/20 | 641/24 | 955/50 | 1023/102 |
| FAK | | | 269/6 | 322/10 | 257/8 | | | | 379/19 | 160/6 |
| FER | | 237/6 | 322/8 | 388/11 | 100/2 | | | | 818/21 | 275/11 |
| FYN | | | 268/6 | 279/6 | | | | | 314/6 | 407/17 |
| GAK | | | | 278/7 | 733/16 | | | 90/3 | 255/6 | 166/8 |
| GSK3Aps | 166/6 | 1232/60 | 357/6 | 154/3 | 635/27 | 393/21 | 406/8 | 132/4 | 868/47 | 585/77 |
| GSK3B | | 1176/54 | 1037/30 | 789/19 | 881/26 | 536/14 | 743/34 | 345/8 | 824/59 | 763/72 |
| JNK1 | | | 311/10 | 448/17 | 590/48 | 559/18 | 540/19 | 678/28 | 457/16 | 389/47 |
| JNK2 | | 287/11 | 277/7 | 702/21 | 662/18 | 405/12 | 170/5 | 429/30 | | 369/20 |
| JNK3 | | | 284/7 | 782/49 | 642/23 | 620/37 | 542/30 | 578/39 | 363/10 | 447/27 |
| LIMK1ps | | | | | 186/4 | | | | | |
| LYN | | | | | | | | | | 134/6 |
| MAP2K1 | | 208/6 | 78/2 | | | | | 74/2 | 49/2 | 90/4 |
| MARK2 | | 49/1 | | | | | | | | |
| NEK9 | | | | | 61/2 | | | | | |
| p38a | | | | 127/6 | | | 295/10 | | | 104/7 |
| p38b | | | | 60/1 | | | 80/2 | | | 95/3 |
| PAK5 | | | | | | | | | 64/1 | |
| PKCa | | | | | | | | | 64/1 | |
| PKCg | | 107/2 | | | | | | | 53/1 | |
| PYK2 | 159/4 | 291/7 | 1909/48 | 1401/42 | 1228/58 | 88/2 | | 422/10 | 1978/82 | 809/39 |
| QSK | | | | | | | | | | 153/7 |
| RSK1 | | 292/7 | 1177/32 | 626/19 | 312/9 | 435/11 | | | 1634/47 | 825/45 |
| RSK3 | | | 401/11 | 312/12 | | | | | 721/21 | 107/6 |
| SRC | | | 544/13 | 351/9 | | | | | 546/13 | 419/25 |
| TBK1 | | | | | | | | | 48/1 | |
| TRKB | | | 207/4 | | | | | | 165/4 | |
| TRKC | | | | | | | | | | |
| YESps | | | 304/7 | | 110/3 | | | | 480/12 | 236/13 |

TABLE 13
Released kinases identified by mass spectrometry analysis after specific elution
with test compounds (mass spectrometry score/number of identified peptides). The test
compounds are selected from a library of organic small molecules (kinase scaffold
compounds). Kinase names are in accordance with the human kinase nomenclature
(http://kinase.com) and Manning et al., 2002, Science 298, 1912-1934 as specified in
supplementary material).

| Kinase | CZC9413 | CZC9414 | CZC9459 | CZC9482 | CZC9493 | CZC9494 | CZC9489 | CZC9488 | CZC9484 | CZC9490 |
|---|---|---|---|---|---|---|---|---|---|---|
| AAK1 | 1110/54 | 1138/69 | 1288/69 | 1459/75 | 823/35 | 777/44 | 989/48 | 1032/57 | 663/36 | 352/16 |
| ALK2 | 85/2 | | 80/2 | 100/2 | | | | 49/1 | 106/3 | |
| AMPKa1 | | | | | | | | | | |
| ARG | | | | | | | | | | |
| BRAFps | | 96/4 | | | | | | 78/2 | | |
| CaMK2a | 909/33 | 357/8 | 1294/57 | 417/25 | 1023/49 | 1002/32 | 1430/69 | 1307/78 | 1187/83 | 71/2 |
| CaMK2b | 1128/71 | 151/5 | 1315/99 | 626/18 | 1077/77 | 806/21 | 939/67 | 1251/72 | 969/65 | |
| CaMK2d | 736/21 | | 760/42 | 414/10 | 752/24 | 376/9 | 712/39 | 720/35 | 716/24 | |
| CaMK2g | 970/25 | | 812/32 | | 709/27 | | 741/17 | 929/26 | 620/26 | |
| CaMKK2 | 242/8 | | 178/4 | 287/10 | 225/7 | | 140/4 | 187/6 | 237/6 | |
| CDK5 | 677/33 | 513/12 | 525/14 | 773/20 | 906/44 | 580/13 | 838/38 | 907/24 | 951/30 | 204/5 |
| CDKL5 | | | | | | | | | | |
| CK1e | | | | | | | | 80/2 | | |
| CRIK | | | 50/1 | | | | | | | |
| CSK | | | | | 73/2 | 66/2 | 121/2 | | 73/2 | |
| EphA4 | | | | 123/4 | | | | | | |
| EphA5 | | | | | | | | | | |
| EphA7 | | 88/2 | | | | | | | | |
| EphB1 | | | | | | | | | | |
| EphB2 | | | 74/2 | | | | | | 247/7 | |
| Erk1ps5 | 121/4 | 144/5 | 282/8 | 469/15 | 176/5 | 333/10 | 89/3 | 597/19 | 513/14 | |
| Erk2 | 913/36 | 852/37 | | 837/51 | 676/40 | 837/44 | 822/24 | 896/30 | 994/51 | 759/28 |
| FAK | | | | 302/8 | | 81/2 | 193/6 | 153/6 | | |
| FER | | | | 118/3 | | | 154/6 | 344/9 | 149/4 | |
| FYN | | | | | | | | 53/1 | | |
| GAK | 234/6 | 663/20 | 388/10 | 63/1 | 187/5 | 732/21 | 161/4 | 530/14 | 201/6 | |
| GSK3Aps | 586/18 | 891/23 | 335/7 | 230/5 | | 201/5 | 630/14 | 745/15 | 1030/55 | 78/2 |
| GSK3B | 663/32 | 471/23 | 294/14 | 143/5 | 282/8 | 257/7 | 343/8 | 567/29 | 908/60 | |
| JNK1 | | | | 595/36 | | | 553/17 | 964/34 | 703/23 | |
| JNK2 | | 394/23 | | 469/15 | 84/3 | | 523/17 | 562/30 | 494/33 | |
| JNK3 | 371/10 | 555/33 | 610/30 | 662/44 | 346/10 | 164/5 | 635/37 | 968/45 | 789/47 | |
| LIMK1ps | | | | | | | 46/1 | | | |
| LYN | | | | | | | | | | |
| MAP2K1 | 95/4 | | 253/8 | 93/3 | 234/8 | 152/5 | 105/4 | 481/12 | 338/10 | 48/1 |
| MARK2 | | | | | | | | | | |
| NEK9 | 122/3 | | | | | | | 46/1 | | |
| p38a | | | | | | | | | | |
| p38b | | | | | | | | | | |
| PAK5 | | | | | | | | | | |
| PKCa | | | | | | | | | | |
| PKCg | | | | | | 210/6 | | 145/4 | 167/5 | |
| PYK2 | 133/3 | 292/7 | 430/12 | 833/21 | 219/6 | 47/1 | 1712/42 | 1769/51 | 703/17 | 51/1 |
| QSK | | | | | | | | | | |
| RSK1 | 445/11 | 100/3 | | 193/4 | 207/4 | 511/12 | 466/14 | 1471/38 | 634/17 | |
| RSK3 | 272/8 | | 94/2 | 211/5 | | 354/11 | | 437/14 | 301/10 | |
| SRC | | | | | | | | | 135/3 | |
| TBK1 | | | | | | | | | | |
| TRKB | | | | | 139/4 | | | 111/3 | | |
| TRKC | | | | 97/3 | | | | | | |
| YESps | | | | | | | | | | |

### Example 4

### Comparison of co-Immobilized Ligands Versus Separately Coupled Ligands

The purpose of this experiment was to assess whether a mixture of beads containing one type of ligand (separately coupled ligands) yields similar results in terms of identified kinases compared to co-immobilized ligands (simultaneous coupling).

The result shows that there is a wide overlap of identified kinases in both experiments demonstrating that both approaches are feasible.

Two ligands were coupled either individually or simultaneously to Sepharose beads and then used for puildown experiments using HeLa cell lysates (ligand 1: BisVIII; ligand 2: CZC00008004; details as in Example 1: Preparation of kinobeads). Coupling of the compounds individually was performed as detailed in example 1. Simultaneous coupling of the two ligands was also performed as in Example 1 except that the compounds were coupled onto the same beads at a concentration of 0.5 μmol/mL instead of the standard 1 μmol/mL beads. Coupling success of the individually or simultaneously immobilized compounds was controlled via HPLC analysis. The beads were washed and stored as described in example 1.

The preparation of HeLa cell lysates, bead washing, contacting of the beads with lysate, washing and analysis of released proteins by mass spectrometry drug were preformed as described in Example 2 (Signalokinome experiment).

TABLE 14
Kinases identified by mass spectrometry analysis. Comparison of the
experiment with two simultaneously coupled ligands (left part) versus individually coupled
compounds (mixed beads; right part). Kinase names are in accordance with the human
kinase nomenclature (http://kinase.com) and Manning et al., 2002, Science 298, 1912-1934
as specified in supplementary material).

| Simultaneous coupling | | | | | Beads mixed post-coupling | | | | |
|---|---|---|---|---|---|---|---|---|---|
| (Bis VIII and CZC8004 pre-mixed) | | | | | (Bis VIII and CZC8004) | | | | |
| | | | | also | | | | | also |
| | | | Number | in | | | | Number | in |
| | | MS | of | post | | | MS | of | pre |
| Identification | Kinase | Score | peptides | mix | Identification | Kinase | Score | peptides | mix |
| 1 | TBK1 | 1531 | 63 | X | 1 | TBK1 | 1365 | 57 | X |
| 2 | GSK3B | 932 | 52 | X | 2 | NEK9 | 1113 | 36 | X |
| 3 | AurA | 895 | 40 | X | 3 | TNK1 | 921 | 44 | X |
| 4 | TYK2 | 841 | 29 | X | 4 | TYK2 | 917 | 34 | X |
| 5 | TNK1 | 771 | 46 | X | 5 | AurA | 910 | 46 | X |
| 6 | CDK2 | 741 | 34 | X | 6 | GSK3A | 814 | 47 | X |
| 7 | NEK9 | 736 | 22 | X | 7 | CaMK2g | 781 | 38 | X |
| 8 | GSK3A | 707 | 43 | X | 8 | GSK3B | 745 | 33 | X |
| 9 | JNK2 | 654 | 24 | X | 9 | JAK1 | 731 | 27 | X |
| 10 | JNK1 | 548 | 23 | X | 10 | CaMK2d | 641 | 26 | X |
| 11 | CaMK2g | 494 | 24 | X | 11 | AurB | 631 | 28 | X |
| 12 | CaMK2d | 456 | 21 | X | 12 | DNAPK | 610 | 24 | X |
| 13 | AAK1 | 450 | 17 | X | 13 | JNK2 | 578 | 23 | X |
| 14 | AurB | 424 | 15 | X | 14 | JNK1 | 499 | 21 | X |
| 15 | JAK1 | 187 | 6 | X | 15 | CDK2 | 485 | 19 | X |
| 16 | CDK9 | 160 | 6 | 0 | 16 | FER | 423 | 17 | X |
| 17 | YES | 108 | 5 | X | 17 | AAK1 | 368 | 17 | X |
| 18 | FER | 106 | 3 | X | 18 | TEC | 159 | 7 | 0 |
| 19 | MPSK1 | 80 | 3 | 0 | 19 | BIKE | 124 | 4 | 0 |
| 20 | DNAPK | 72 | 4 | X | 20 | YES | 108 | 6 | X |
| 21 | ULK3 | 64 | 1 | 0 | 21 | FRAP | 59 | 3 | 0 |

TABLE 15
Kinases and sequences of proteotypic peptides identified in the
simultaneous coupling experiment

| KINASE NAME | PROTEOTYPIC PEPTIDE |
|---|---|
| AAK1 | APEMVNLYSGK |
| AURKB | IYLILEYAPR |
| AURKB | LPL |
| AURKB | SNVQPTAAPGQK |
| CAMK2D | DLKPENLLLASK |
| CAMK2D | FTDEYQLFEELGK |
| CAMK2D | GAILTTMLATR |
| CAMK2D | IPTGQEYAAK |
| CAMK2G | LTQYIDGQGRPR |
| CDK2 | ALFPGDSEIDQLFR |
| CDK2 | FMDASALTGIPLPLIK |
| CDK9 | IGQGTFGEVFK |
| CDK9 | LLVLDPAQR |
| CDK9 | NPATTNQTEFER |
| GSK3A | SQEVAYTDIK |
| GSK3A | VIGNGSFGVVYQAR |
| GSK3A | VTTVVATLGQGPER |
| GSK3A | YFFYSSGEK |
| GSK3B | DIKPQNLLLDPDTAVLK |
| GSK3B | LYMYQLFR |
| GSK3B | VIGNGSFGVVYQAK |
| JAK1 | LIMEFLPSGSLK |
| JAK1 | LSDPGIPITVLSR |
| MAPK8 | APEVILGMGYK |
| MAPK9 | ILDFGLAR |
| MAPK9 | NIISLLNVFTPQK |
| MAPK9 | VIEQLGTPSAEFMK |
| NEK9 | LGINLLGGPLGGK |
| NEK9 | LNPAVTCAGK |
| NEK9 | LQQENLQIFTQLQK |
| NEK9 | SSTVTEAPIAVVTSR |
| NEK9 | VLACGLNEFNK |
| PRKDC | INQVFHGSCITEGNELTK |
| PRKDC | NELEIPGQYDGR |
| PRKDC | QLFSSLFSGILK |
| PRKDC | YPEETLSLMTK |
| STK16 | APELFSVQSHCVIDER |
| STK16 | GTLWNEIER |
| STK6 | SKQPLPSAPENNPEEELASK |
| STK6 | VEFTFPDFVTEGAR |
| TBK1 | IASTLLLYQELMR |
| TBK1 | LFAIEEETTTR |
| TBK1 | TTEENPIFVVSR |
| TBK1 | VIGEDGQSVYK |
| TNK1 | AAALSGGLLSDPELQR |
| TNK1 | VADFGLVRPLGGAR |
| TYK2 | LGLAEGTSPFIK |
| TYK2 | LSDPGVGLGALSR |
| TYK2 | MVVAQQLASALSYLENK |
| TYK2 | SLQLVMEYVPLGSLR |
| TYK2 | SQAPDGMQSLR |
| ULK3 | ASVENLLTEIEILK |
| YES1 | AANILVGENLVCK |
| YES1 | EVLEQVER |

TABLE 16
Kinases and sequences of proteotypic peptides identified in the mix
experiment (beads mixed post coupling)

| KINASE NAME | PROTEOTYPIC PEPTIDE |
|---|---|
| AAK1 | ADIWALGCLLYK |
| AURKB | HFTIDDFEIGRPLGK |
| AURKB | IYLILEYAPR |
| AURKB | LPLAQVSAHPWVR |
| AURKB | SNVQPTAAPGQK |
| BMP2K | ADIWALGCLLYK |
| BMP2K | ITDTIGPTETSIAPR |
| CAMK2D | AGAYDFPSPEWDTVTPEAK |
| CAMK2D | FTDEYQLFEELGK |
| CAMK2D | FYFENALSK |
| CAMK2D | GAILTTMLATR |
| CAMK2G | AGAYDFPSPEWDTVTPEAK |
| CAMK2G | DLKPENLLLASK |
| CAMK2G | FTDDYQLFEELGK |
| CAMK2G | LTQYIDGQGRPR |
| CAMK2G | NLINQMLTINPAK |
| CDK2 | ALFPGDSEIDQLFR |
| CDK2 | FMDASALTGIPLPLIK |
| FER | HSIAGIIR |
| FER | THAEDLNSGPLHR |
| FRAP1 | GPTPAILESLISINNK |
| GSK3A | SQEVAYTDIK |
| GSK3A | VIGNGSFGVVYQAR |
| GSK3A | VTTVVATLGQGPER |
| GSK3A | YFFYSSGEK |
| GSK3B | DIKPQNLLLDPDTAVLK |
| GSK3B | LLEYTPTAR |
| GSK3B | LYMYQLFR |
| GSK3B | VIGNGSFGVVYQAK |
| GSK3B | YFFYSSGEK |
| JAK1 | LIMEFLPSGSLK |
| JAK1 | LSDPGIPITVLSR |
| MAPK8 | APEVILGMGYK |
| MAPK8 | NIIGLLNVFTPQK |
| MAPK9 | APEVILGMGYK |
| MAPK9 | ILDFGLAR |
| MAPK9 | NIISLLNVFTPQK |
| MAPK9 | VIEQLGTPSAEFMK |
| NEK9 | AGGGAAEQEELHYIPIR |
| NEK9 | LNPAVTCAGK |
| NEK9 | LQQENLQIFTQLQK |
| NEK9 | SSTVTEAPIAVVTSR |
| NEK9 | VLACGLNEFNK |
| PRKDC | AALSALESFLK |
| PRKDC | DFGLLVFVR |
| PRKDC | DILPCLDGYLK |
| PRKDC | DLLLNTMSQEEK |
| PRKDC | DQNILLGTTYR |
| PRKDC | FMNAVFFLLPK |
| PRKDC | FVPLLPGNR |
| PRKDC | GLSSLLCNFTK |
| PRKDC | HSSLITPLQAVAQR |
| PRKDC | INQVFHGSCITEGNELTK |
| PRKDC | LAGANPAVITCDELLLGHEK |
| PRKDC | LATTILQHWK |
| PRKDC | LSDFNDITNMLLLK |
| PRKDC | LYSLALHPNAFK |
| PRKDC | NELEIPGQYDGR |
| PRKDC | QCLPSLDLSCK |
| PRKDC | SDPGLLTNTMDVFVK |
| PRKDC | SIGEYDVLR |
| PRKDC | SLGPPQGEEDSVPR |
| PRKDC | VTELALTASDR |
| STK6 | QWALEDFEIGRPLGK |
| STK6 | SKQPLPSAPENNPEEELASK |
| STK6 | VEFTFPDFVTEGAR |
| TBK1 | IASTLLLYQELMR |
| TBK1 | LFAIEEETTTR |
| TBK1 | TTEENPIFVVSR |
| TBK1 | VIGEDGQSVYK |
| TEC | ELGSGLFGVVR |
| TEC | GQEYLILEK |
| TEC | HAFGSIPEIIEYHK |
| TNK1 | AAALSGGLLSDPELQR |
| TNK1 | ANLWDAPPAR |
| TNK1 | MLPEAGSLWLLK |
| TNK1 | VADFGLVRPLGGAR |
| TYK2 | AAALSFVSLVDGYFR |
| TYK2 | HGIPLEEVAK |
| TYK2 | LGLAEGTSPFIK |
| TYK2 | LSDPGVGLGALSR |
| TYK2 | MVVAQQLASALSYLENK |
| TYK2 | SLQLVMEYVPLGSLR |
| TYK2 | SQAPDGMQSLR |
| YES1 | EVLEQVER |

### Example 5

### Synthesis of Kinobead Ligand 5 (Indol Ligand 91)

### Synthesis indol ligand 91: 5-[5-Fluoro-2-oxo-1,2-dihydro-indol-(3Z)-ylidenemethyl]-2,4-dimethyl-1H-pyrrole-3-carboxylic acid (3-amino-propyl)-amide

### Step 1: 5-Fluoro-1,3-dihydro-indol-2-one

A solution of 5-Fluoroisatin (Ig) in Hydrazine hydrate (55%, 10 ml) was heated at 110° C. for 30 minutes. Once the suspension has gone into solution, the reaction was heated at 110° C. for 4 hours, then cooled at 0° C. The precipitate was filtered and washed with water. The solid was suspended in water (10 ml), the pH was lowered to pH2 by addition of HCl conc, and the solution stirred at room temperature for 5 hours. The precipitate was collected, the solid washed with water (2×15 ml) and dried in the vacuum oven at 40° C. (0.26 g, 30%). ¹H NMR (400 MHz, DMSO-d₆) δ 10.2 (s, 1H); 6.8-7.0 (dd, 2H); 6.6 (m, 1H); 3.2 (s, 2H);. LCMS: method D, RT=1.736 min, [MH⁺=152].

### Step 2: {3-[(5-Formyl-2,4-dimethyl-1H-pyrrole-3-carbonyl)-amino]-propyl}-carbamic acid tert-butyl ester

To a solution of 5-formyl-2,4-dimethyl-1H-pyrazol-3-carboxylic acid (0.300 g, 1.79 mmol), N-(3-Dimethylaminopropyl)-N′-ethylcarbodiimide (0.516 g, 2.69 mmol), 1-Hydroxybenzotriazole hydrate (0.364 g, 2.69 mmol), triethylamine (0.502 ml, 3.56 mmol) in dimethylformamide (3 ml) was added N-Boc-1,3-diaminopropane (0.375 ml, 2.15 mmol). The solution was stirred at room temperature for 15 hours. A mixture of brine (1.5 ml), water (1.5 ml) and saturated aqueous sodium bicarbonate (1.5 ml) was added and the pH of the solution adjusted to 12 by addition of ION sodium hydroxide. The solution was extracted 3 times with a mixture of dichloromethane:Methanol (9:1). The organic layer was dried with anhydrous magnesium sulfate. The solvent was removed and the residue purified by flash chromatography (Hexane:Ethyl acetate (50 to 100%)) to yield the desired compound as a yellow solid (0.30 g, 52%). ¹H NMR (400 MHz, DMSO-d₆) δ 11.90 (s, 1H); 9.50 (s, 1H); 7.50 (m, 1H); 6.90 (m, 1H); 3.20 (q, 2H); 3.00 (q, 2H); 2.30 (s, 3H)); 2.20 (s, 3H)); 1.60 (m, 2H)); 1.40 (s, 9H). LCMS: method D, RT=2.103 min, [M+Na⁺=346], and [M-Boc+Na⁺=246].

### Step 3: [3-({5-[5-Fluoro-2-oxo-1,2-dihydro-indol-(3Z)-ylidenemethyl]-2,4-dimethyl-1H-35 pyrrole-3-carbonyl}-amino)-propyl]-carbamic acid tert-butyl ester

A solution of {3-[(5-Formyl-2,4-dimethyl-1H-pyrrole-3-carbonyl)-amino]-propyl}-carbamic acid tert-butyl ester (0.200 g, 0.62 mmol) and 5-Fluoro-1,3-dihydro-indol-2-one (0.011 g, 0.62 mmol), pyrrolidine (0.003 ml) in ethanol (2 ml) was heated at 78° C. for 3 hours. The reaction was cooled to 0° C. and the resulting precipitate filtered, washed with cold ethanol. The product was suspended in ethanol (4 ml) and stirred at 72° C. for 30 minutes. The reaction was filtered, the precipitate dried in a vacuum oven at 40° C. to yield the desired compound as a solid (0.265 g, 94%). ¹H NMR (400 MHz, DMSO-d₆) δ 13.8 (s, 1H); 11.00 (s, 1H); 7.80 (m, 211); 7.70 (m, 1H); 7.0 (m, 1H); 6.9 (m, 211); 3.30 (q, 211); 3.10 (q, 2H); 2.50 (dd, 6H) 1.60 (m, 2H)); 1.40 (s, 9H). LCMS: method D, RT=2.86 min, [M+Na⁺=479], and [M-Boc+Na⁺=379].

### Step 4: 5-[5-Fluoro-2-oxo-1,2-dihydro-indol-(3Z)-ylidenemethyl]-2,4-dimethyl-1H-pyrrole-3-carboxylic acid (3-amino-propyl)-amide

[3-({5-[5-Fluoro-2-oxo-1,2-dihydro-indol-(3Z)-ylidenemethyl]-2,4-dimethyl-1H-pyrrole-3-carbonyl}-amino)-propyl]-carbamic acid tert-butyl ester was suspended in methanol. 2 ml of HCl (4N) in dioxane was added and the reaction stirred at room temperature overnight. The solvent was removed to yield the desired compound (0.098 g, 91%). ¹H NMR (400 MHz, DMSO-d₆) δ 13.8 (s, 1H); 11.00 (s, 1H); 7.90-7.70 (m, 5H); 6.9 (m, 2H); 3.30 (q, 2H); 2.80 (q, 2H); 2.50 (dd, 6H) 1.80 (m, 2H). LCMS: inconclusive due to fluorescence.

All reactions were carried out under inert atmosphere. NMR spectra were obtained on a Bruker dpx400. LCMS was carried out on an Agilent 1100 using a zorbax SBC-18, 4.6 mm×150 mm-5μ column or a Small column: ZORBAX® SB-C18, 4.6×75 mm, 3.5 microns (“short column”). Column flow was 1 ml/min and solvents used were water and acetonitrile (0.1% TFA) with an injection volume of 10 ul. Wavelengths were 254 and 210 nm. Methods are described below.

TABLE 17
Analytical methods

| | Easy Access | ChemStation | Flow | | Run |
|---|---|---|---|---|---|
| Method | Method Name | Method Name | Rate | Solvent | Time |
| A | Analytical positive | ANL_POS7.M | 1 ml/min | 0-2.5 min | 7 min |
| | 7 mn | | | 5-95% MeCN | |
| | | | | 2.5-6 min | |
| | | | | 95% MeCN | |
| B | Analytical positive | ANAL_POS.M | 1 ml/min | 0-11 min | 15 min |
| | Ion | | | 5-95% MeCN | |
| | | | | 11-13 min | |
| | | | | 95% MeCN | |
| C | Loop injection, | | 1 ml/min | 95% MeCN | 1 min |
| | Positive | | | | |
| D | Analytical positive | Short column | 1 ml/mn | 0-4.5 min | 5 min |
| | Ion | ANL Positive | | 30-95% MeCN | |
| E | Analytical High pH | Analytical High | 3 ml/min | 0 to 8 min | 10 min |
| | | pH | | 5-95% MeCN | |
| | | | | 8 to 9 min | |
| | | | | 95% MeCN | |

TABLE 18
Abbreviations used in chemistry protocols

| | |
|---|---|
| aq | aqueous |
| D | doublet |
| DMSO | dimethyl sulfoxide |
| G | gram |
| HCl | Hydrochloric acid |
| HPLC | high pressure liquid chromatography |
| LCMS | liquid chromatography —mass spectrometry |
| M | multiplet |
| mins | minute |
| mmol | millimole |
| N | Normal |
| NMR | nuclear magnetic resonance |
| Q | quartet |
| RT | retention time |
| S | singlet |
| sat | saturated |
| T | triplet |

### Example 6

### Synthesis of Kinobead Ligand 6 (Quinazoline Ligand 32)

### Synthesis of quinazoline ligand 32: 7-(4-amino)-butyloxy-4-(2-fluoro-4-bromo-phenyl)-amino-6-methoxyquinazoline

### Step 1: 7-benzyloxy-4-(2-fluoro-4-bromo-phenyl)-amino-6-methoxyquinazoline

To a solution of 7-benzyloxy-4-chloro-6-methoxyquinazoline (0.250 g, 0.83 mmol) and 4-bromo-2-fluoroaniline (190 mg, 1 mmol) in isopropanol (10 ml) was added hydrochloric acid (4M in dioxane, 0.230 ml, 0.92 mmol). The mixture was stirred at 90° C. for 2 hours. The reaction mixture was allowed to cool down to room temperature. The solid was filtered off, washed with cold isopropanol and ether and finally dried overnight at 50° C., affording the title compound (0.297 g-79%). ¹H NMR (400 MHz, CD₃OD-d₄) δ 8.65 (s, 1H); 7.96 (s, 1H); 7.55 (m, 5H); 7.40 (t, 3H); 7.30 (s, 1H); 5.37 (s, 2H); 4.89 (s, 1H); 4.08 (s, 1H). LCMS: method C, [M⁺=454].

### Step 2: 7-hydroxy-4-(2-fluoro-4-bromo-phenyl)-amino-6-methoxyquinazoline

7-benzyloxy-4-(2-fluoro-4-bromo-phenyl)-amino-6-methoxyquinazoline (297 mg, 0.654 mmol) was dissolved in trifluoroacetic acid (5 ml) and the solution was stirred at reflux for one hour. The reaction mixture was allowed to cool down to room temperature and poured onto ice. The solid was filtered off and taken up in methanol. The solution was basified using aqueous ammonia (to pH11) and reduced in vacuo. The solid was collected by filtration, washed with cold water and ether and finally dried overnight under vacuum at 50° C., affording the title compound. (0.165 g-69%). ¹H NMR (400 MHz, CD₃OD-d₄) δ 8.55 (s, 1H); 7.89 (s, 1H); 7.52 (m, 3H); 7.13 (s, 1H); 4.08 (s, 3H). LCMS: method C, [M⁺=364].

### Step 3: 7-(4-amino-phthalimide)-butyloxy-4-(2-fluoro-4-bromo-phenyl)-amino-6-methoxyquinazoline

The N(-4-bromobutyl)-phthalimide (0.355 g, 0.1.187 mmol) was added in one portion to a mixture of 7-hydroxy-4-(2-fluoro-4-bromo-phenyl)-amino-6-methoxyquinazoline (360 mg, 0.989 mmol) and potassium carbonate (410 mg, 2.967 mmol) in dimethylformamide (7 ml). The reaction mixture was stirred at 60° C. for 2 hours and then allowed to cool down to room temperature. Water (10 ml) was added and the precipitate was filtered off. The solid was washed with cold water and methanol and finally dried overnight at 50° C. under vacuum, affording the title compound (0.317 mg-57%). ¹H NMR (400 MHz, CD₃OD-d₄) δ 9.49 (s, 1H); 8.29 (s, 1H); 7.82 (m, 4H); 7.71 (s, 1H); 7.61 (m, 1H); 7.47 (t, 1H, J=8.3 Hz); 7.413 (m, 1H); 7.11 (s, 1H); 4.09 (m, 2H); 3.87 (s, 3H); 3.62 (m, 2H); 1.75 (broad s, 4H). LCMS: method B, RT=9.20 min, [MH⁺=410].

### Step 4: 7-(4-amino)-butyloxy-4-(2-fluoro-4-bromo-phenyl)-amino-6-methoxyquinazoline

A suspension of 7-(4-amino-phthalimide)-butyloxy-4-(2-fluoro-4-bromo-phenyl)-amino-6-methoxyquinazoline (150 mg, 0.265 mmol) in dimethylformamide (5 ml) was treated with hydrazine monohydrate. The reaction mixture was stirred at room temperature over 2 days (solubilisation occurred after few minutes and total consumption of starting material observed) and then reduced in vacuo. The thick yellow oil was purified using the “catch and release” method (Isolute SCX-2 cartridge with optimised realeasing method) affording the title compound (63 mg, 51%). ¹H NMR (400 MHz, CDCl₃-d₆) δ 8.68 (s, 1H); 8.48 (t, 1H, J=8.5 Hz); 7.36 (m, 1H); 7.33 (m, 1H); 7.32 (broad s, 114); 7.25 (s, 1H); 7.00 (s, 1H); 4.19 (m, 2H); 4.02 (s, 314); 2.80 (m, 2H); 1.98 (m, 2H); 1.67 (m, 2H); 1.49 (broad s, 2H). HPLC: method D, RT=3.52 min.

All reactions were carried out under inert atmosphere. NMR spectra were obtained on a Bruker dpx400. LCMS was carried out on an Agilent 1100 using a zorbax SBC-18, 4.6 mm×150 mm-5μ column or a Small column: ZORBAX® SB-C18, 4.6×75 mm, 3.5 microns (“short column”). Column flow was 1 ml/min and solvents used were water and acetonitrile (0.1% TFA) with an injection volume of 10 ul. Wavelengths were 254 and 210 nm. Methods are described below.

TABLE 19
Analytical methods

| | Easy Access | ChemStation | Flow | | Run |
|---|---|---|---|---|---|
| Method | Method Name | Method Name | Rate | Solvent | Time |
| A | Analytical positive | ANL_POS7.M | 1 ml/min | 0-2.5 min | 7 min |
| | 7 mn | | | 5-95% MeCN | |
| | | | | 2.5-6 min | |
| | | | | 95% MeCN | |
| B | Analytical positive | ANAL_POS.M | 1 ml/min | 0-11 min | 15 min |
| | Ion | | | 5-95% MeCN | |
| | | | | 11-13 min | |
| | | | | 95% MeCN | |
| C | Loop injection, | | 1 ml/min | 95% MeCN | 1 min |
| | Positive | | | | |
| D | Analytical positive | Short column | 1 ml/mn | 0-4.5 min | 5 min |
| | Ion | ANL Positive | | 30-95% MeCN | |
| E | Analytical High pH | Analytical High | 3 ml/min | 0 to 8 min | 10 min |
| | | pH | | 5-95% MeCN | |
| | | | | 8 to 9 min | |
| | | | | 95% MeCN | |

TABLE 20
Abbreviations used in chemistry protocols

| | |
|---|---|
| aq | aqueous |
| D | doublet |
| DMSO | dimethyl sulfoxide |
| G | gram |
| HCl | Hydrochloric acid |
| HPLC | high pressure liquid chromatography |
| LCMS | liquid chromatography - mass spectrometry |
| M | multiplet |
| mins | minute |
| mmol | millimole |
| N | Normal |
| NMR | nuclear magnetic resonance |
| Q | quartet |
| RT | retention time |
| S | singlet |
| sat | saturated |
| T | triplet |

### Example 7

### Synthesis of Kinobead Ligand 7 (Modified Staurosporine)

Kinobead ligand 7 based on Staurosporine was synthesized according to the following protocol.

Step 1: Modification of Staurosporine with Diglycolic Acid Anhydride

Dissolve Staurosporine (typically 10 mg, IRIS-Biotech, Germany) in 1 ml waterfree DMF, add 5 μL of TEA, cool to 0° C. From this solution, 5 μl are taken and mixed with 50 μl of ACN for determination of the relative starting amount using a LC-MS system (AGILENT, Germany). For the LC-MS analysis 5 μl of the reaction mixture is diluted into 50 μl 100% ACN. After thorough mixing 5 μl are applied to the HPLC analysis using an autosampler. Separation is carried out at a flow rate of 450 μl/min with 0.1% formic acid in water as solvent A and 0.1% formic acid in 100% ACN as solvent B using a 3×50 mm C18 column (ZORBAX-Extended C18, AGILENT, Germany). A gradient from 10% A to 95% B in 75 minutes is used. UV absorbance is observed at 254 nm, the molecular mass of the compound is determined online by a single stage quadrupol MS system (MSD, AGILENT, Germany). The recorded data are checked manually. This first analysis serves as the standard for the calculation of the yields of reaction products. The peak area of the UV signal is set to 100% as starting amount, based on the pure Staurosporine.

To the ice cold Staurosporine solution a 10 fold excess of diglycolic acid anhydride (Merck Germany) in DMF is added. The reaction with close to 100% yield is finished within 10 minutes, check by HPLC by mixing 5 μl of the reaction mixture with 50 μl ACN and analyse by LC-MS as described above. The molecular mass shifts from 467.5 Da (unmodified Staurosporine) to 545.6 Da (modified Staurosporine) for the singly charged molecule. If the reaction was not complete another tenfold excess of the diglycolic acid anhydride is added and the mixture kept for another 30 minutes at room temperature. The reaction mixture is analysed by LC-MS as described above.

After the reaction is completed, no unmodified Staurosporine can be detected. 5 mL water is added to the reaction mixture, first to quench the acid anhydride, secondly to dilute for solid phase extraction. Prepare a 500 mg C18 solid phase extraction cartridge (Phenomenex, Germany) by activation with 10 ml methanol and equilibration with 0.1% TFA in water. The solvents are drawn through the cartridge with a flow rate of approximately 2 to 3 ml/min by using a membrane vacuum pump. The aqueous reaction mixture is applied to the cartridge and drawn through it with the same flow rate as above. After the solution has completely passed through the cartridge and the modified staurosporine is bound to the C18 material, it is washed first with 5% ACN 0.1% TFA, then with 10% ACN, and finally with 20% ACN, all with 0.1% TFA in water. The modified Staurosporine is then eluted with 70% ACN, 0.1% TFA in water, followed by 80% ACN, 0.1% TFA in water. The eluates are combined and dried in vacuum.

Step 2: Coupling of the Modified Staurosporine to the Solid-Phase Bound Diamine using PyBroP-Chemistry

Dissolve the modified Staurosporine in waterfree DMF and add to pre-swollen Bis-(aminoethyl)ethylene glycol-trityl resin in DMF (IRIS Biotech, Germany). The solid phase bound diamine is added in 2 to 3 fold excess. To the slurry add 10 μl of diisopropyl ethylamine (DIEA, FLUKA, Germany) and a five fold excess of PyBroP over the modified Staurosporine. The reaction is carried out for 16 hours at room temperature under permanent mixing over an end-over-end mixer. Check the supernatant for unbound modified Staurosporine by HPLC using the LC-MS system as described above, This step is not quantitative, only the disappearance of unbound modified Staurosporine is measured. After the reaction is completed (no unbound modified Staurosporine can be detected anymore) the resin washed with 10 volumes of waterfree DMF and two times with 10 volumes of DCM.

### Step 3: Cleavage Reaction

The resin is then resuspended in 5 volumes DCM, cooled to 0° C. and 1 ml TFA is added. The former light yellow resin should now turn to dark red indicating the reaction. The cleavage is carried out for 20 minutes at 0° C. and 20 minutes at room temperature. The solution is collected and the resin washed two times with 10 volumes of DCM. All eluates are combined and dried using a rotary evaporator. The remaining oily film is dissolved in 0.5 ml DMF and 5 ml of water is added. The modified Staurosporine is purified by solid phase extraction as described above and dried under vacuum. Yield is checked by HPLC with UV absorbance at 254 nm against the original unmodified staurosporine solution as described above, relative to the solution of the unmodified Staurosporine after dissolving the reaction product in 1 ml DMF. From this solution 5 μl are mixed with 50 μl of ACN and 5 μl are analysed by LC-MS. The molecular mass of the expected product is 727.8 Da. The peak area at 254 nm is related to the peak area of the unmodified Staurosporine as 100% analysed as described.

The modified staurosporine is used for coupling to NHS-activated sepharose as usual via its amino group. Before coupling, 1 ml of beads is washed three times with 12 ml waterfree DMSO and then resuspended with 1 mL waterfree DMSO. To this suspension 20 μl of TEA is added and an equivalent of 1 μmole of the modified Staurosporine in DMF. Directly after adding and well mixing and short centrifugation for 1 minute at 1200 rpm, 20 μl of the supernatant is taken and 5 μl is analysed by LC-MS. After 16 hours under continuous mixing on a rotary mixer, the suspension is centrifuged again and 20 μl are taken to determine the remaining unbound modified Staurosporine by analysis of 5 μl by LC-MS as described above. Usually 100% of the modified Staurosporine is bound. The beads are washed afterwards three times with 12 ml DMSO, resuspended again with 1 ml DMSO and 50 μl of ethanolamine (MERCK, Germany) is added to block unreacted NHS-activated groups. The reaction is carried out for 16 h under continuous mixing. After the blocking reaction, the beads are washed three times with 12 ml iso-propanole (MERCK, Germany) and stored as 1:1 slurry at 4° C. until use.

The following reagents were used:

### DMF: N,N-Dimethylformamide (FLUKA, 40228);

### TEA: Triethylamine (SIGMA-Aldrich, T0886);

### ACN: Acetonitrile for HPLC (Merck, 1.00030);

TFA: Trifluoroacetic acid (FLuKA, 09653);
PyBroP: Bromo-tris-pyrrolidino-phosphonium hexafluorophosphate (Novabiochem, 01-62-0017);

### DCM: Dichlormethane (FLUKA, 66749);

### DMSO: Dimethylsulfoxide (FLUKA, 41648).

### Example 8

### In-Lysate Competition Binding and Quantitative Protein Affinity Profile SAP)

This example illustrates competition binding in cell lysates (see particularly the third aspect of the invention). A test compound, Bisindolylmaleimide VIII (Bis VIII, a well known kinase inhibitor; Davies et al., 2000. Specificity and mechanism of action of some commonly used protein kinase inhibitors. Biochemical Journal 351 (Pt 1): 95-105) was added to a cell lysate thereby allowing the test compound to bind to the target proteins in the lysate. Then the lysate was contacted with the kinobeads affinity matrix to capture remaining free target proteins. The proteins bound to the kinobeads matrix were then eluted with detergent-containing buffer, separated on a SDS-polyacryamide gel and analyzed by immunodetection (Western blots) or mass spectrometry detection.

For Western blot analysis proteins bound to the affinity matrix were eluted from the affinity matrix and subsequently separated by SDS-Polyacrylamide gel elecrophoresis and transferred to a blotting membrane. Individual kinases were detected with specific antibodies against GSK3alpha, GSK3beta and ITK (FIG. 4A). The result shows that preincubation of the cell lysate with Bis VIII prevented binding of the target proteins GSK3alpha and GSK3beta to the kinobeads matrix in a dose dependent manner. Increasing concentrations of the kinase inhibitor Bis VIII specifically prevented binding of GSK3 alpha and GSK3beta to the kinobeads but not binding of ITK. For GSK3beta the signal was quantified and plotted against the Bis VIII concentration added to the cell lysate (FIG. 4B).

For the quantitative detection of proteins by mass spectrometry proteins were eluted from the affinity matrix and subsequently separated by SDS-Polyacrylamide gel elecrophoresis. Suitable gel areas were cut out and subjected to in-gel proteolytic digestion with trypsin.

Four tryptic digest samples (corresponding to three different Bis VIII concentrations in the lysate and one DMSO control) were labeled with ITRAQ reagents and the combined samples were analyzed in a single LC-MS/MS mass spectrometry analysis followed by peak quantification in the MS/MS spectrum (Ross et al., 2004. Multiplexed protein quantitation in Saccharomyces cerevisiae using amine-reactive isobaric tagging reagents. Mol. Cell. Proteomics 3(12): 1154-1169). The result shows that different levels of individual proteins were detected and relative intensity values were calculated (Table 22). Binding curves for individual kinases are shown (FIG. 5). The relative intensity 50 (RI50) values representing the affinity of kinase-compound pairs are similar to IC₅₀ values reported by kinase enzyme assays (Davies et al., 2000. Specificity and mechanism of action of some commonly used protein kinase inhibitors. Biochemical Journal 351 (Pt 1): 95-105).

### 1. Cell Culture

Jurkat cells (clone E6-1 from ATCC, number TIB-152) were grown in 1 litre Spinner flasks (Integra Biosciences, #182101) in suspension in RPMI 1640 medium (Invitrogen, #21875-034) supplemented with 10% Fetal Bovine Serum (Invitrogen) at a density between 0.15×10⁶ and 1.2×10⁶ cells/ml and harvested by centrifugation. Cell pellets were frozen in liquid nitrogen and subsequently stored at −80° C.

### 2. Preparation of Cell Lysates

Jurkat cells were homogenized in a Potter S homogenizer in lysis buffer: 50 mM Tris-HCl, 0.8% NP40, 5% glycerol, 150 mM NaCl, 1.5 mM MgCl₂, 25 mM NaF, 1 mM sodium vanadate, 1 mM DTT, pH 7.5. One complete EDTA-free tablet (protease inhibitor cocktail, Roche Diagnostics, 1 873 580) per 25 ml buffer was added. The material was dounced 10 times using a mechanized POTTER S, transferred to 50 ml falcon tubes, incubated for 30 minutes on ice and spun down for 10 minutes at 20,000 g at 4° C. (10,000 rpm in Sorvall SLA600, precooled). The supernatant was transferred to an ultracentrifuge (UZ)-polycarbonate tube (Beckmann, 355654) and spun for 1 hour at 100.000 g at 4° C. (33.500 rpm in Ti50.2, precooled). The supernatant was transferred again to a fresh 50 ml falcon tube, the protein concentration was determined by a Bradford assay (BioRad) and samples containing 50 mg of protein per aliquot were prepared. The samples were immediately used for experiments or frozen in liquid nitrogen and stored frozen at −80° C.

3. Preincubation of Lysate with Test Compound

Aliqots of Jurkat lysate (10 mg protein) were incubated with different Bis VIII concentrations for one hour at 4° C. (0.025 μM, 0.074 μM, 0.22 μM, 0.67 μM, 2.0 μM and 6.0 μM final concentration of Bis VIII). To this end Bis VIII solutions were prepared in 100% DMSO as solvent that corresponded to 200 fold desired final Bis VIII concentration (Bis VIII from Alexis Biochemicals cat. number ALX 270-056). Five μl of these solutions were added to 1 ml of lysate resulting in the indicated final concentrations. For a control experiment 5 μl of DMSO without Bis VIII were used.

4. Protein Capturing with Kinobeads

To each preincubated lysate sample 40 μl of kinobeads (see example 1) were added and incubated for one hour at 4° C. During the incubation the tubes were rotated on an end-over-end shaker (Roto Shake Genie, Scientific Industries Inc.). Beads were collected by centrifugation, transfered to Mobicol-columns (MoBiTech 10055) and washed with 10 ml 1×DP buffer containing 0.4% NP40 detergent, followed by a wash with 5 ml 1×DP buffer with 0.2% NP40. To elute the bound proteins, 100 μl 2×SDS sample buffer was added, the column was heated for 30 minutes at 50° C. and the eluate was transferred to a microfuge tube by centrifugation. Proteins were then separated by SDS-Polyacrylamide electrophoresis (SDS-PAGE). The composition and preparation of buffers is described in example 2.

### 5. Protein Detection by Western Blot Analysis

Western blots were performed according to standard procedures and developed with the ECL Western blotting detection system according to the instructions of the manufacturer (Amersham Biosciences, #RPN2106). The ECL Western blotting system from Amersham is a light emitting non-radioactive method for the detection of specific antigens, directly or indirectly with Horseradish Peroxidase (HRP) labeled antibodies.

The anti-Glycogen Synthase Kinase 3 beta (GSK3beta) antibody was used at a dilution of 1:1000 (rabbit polyclonal anti-GSK3P, Stressgen Bioreagents, Victoria, Canada, product number KAP-ST002). This antibody also recognizes GSK3alpha. The anti-ITK antibody was also used at a dilution of 1:1000 (rabbit polyclonal anti-ITK antibody, Upstate Lake Placid, N.Y., catalog number 06-546).

### 6. Protein Detection by Mass Spectrometry

### 6.1 Protein Digestion Prior to Mass Spectrometric Analysis

Gel-separated proteins were reduced, alkylated and digested in gel essentially following the procedure described by Shevchenko et al., 1996, Anal. Chem. 68:850-858. Briefly, gel-separated proteins were excised from the gel using a clean scalpel, reduced using 10 mM DTT (in 5 mM ammonium bicarbonate, 54° C., 45 minutes) and subsequently alkylated with 55 mM iodoacetamid (in 5 mM ammonium bicarbonate) at room temperature in the dark for 30 minutes. Reduced and alkylated proteins were digested in gel with porcine trypsin (Promega) at a protease concentration of 10 ng/μl in 5 mM Triethylammonium hydrogencarbonate (TEAB). Digestion was allowed to proceed for 4 hours at 37° C. and the reaction was subsequently stopped using 5 μl 5% formic acid.

### 6.2 Sample Preparation Prior to Analysis by Mass Spectrometry

Gel plugs were extracted twice with 20 μl 1% formic acid in water and once with 20 μl 0.1% formic acid, 60% acetonitrile in water and pooled with acidified digest supernatants. Samples were dried in a a vaccuum.

6.3 iTRAQ Labeling of Peptide Extracts

The peptide extracts of samples treated with different concentrations of the test compound (0.074 μM, 0.22 μM and 0.67 μM Bis VIII) and the solvent control (0.5% DMSO) were treated with different isomers of the isobaric tagging reagent (iTRAQ Reagents Multiplex Kit, part number 4352135, Applied Biosystems, Foster City, Calif., USA). The iTRAQ reagents are a set of multiplexed, amine-specific, stable isotope reagents that can label all peptides in up to four different biological samples enabling simultaneous identification and quantitation of peptides. The iTRAQ reagents were used according to instructions provided by the manufacturer.

The samples were resuspended in 10 μl 50 mM TEAB solution, pH 8.5 and 10 Pt ethanol were added. The iTRAQ reagent was dissolved in 85 μl ethanol and 10 pt of reagent solution were added to the sample. The labeling reaction was performed at room temperature for one hour on a horizontal shaker and stopped by adding 10 μl of 10% formic acid in water. The four labeled sampled were then combined, dried in a vacuum centrifuge and resuspended in 10 μl of 0.1% formic acid in water.

### 6.4 Mass Spectrometric Data Acquisition

Peptide samples were injected into a nano LC system (CapLC, Waters or Ultimate, Dionex) which was directly coupled to a quadrupole TOF (QTOF2, QTOF Ultima, QTOF Micro, Micromass) or ion trap (LTQ Deca XP) mass spectrometer. Peptides were separated on the LC system using a gradient of aqueous and organic solvents (see below). Solvent A was 5% acetonitrile in 0.5% formic acid and solvent B was 70% acetonitrile in 0.5% formic acid.

TABLE 21
Peptides eluting off the LC system were partially sequenced
within the mass spectrometer.

| Time (min) | % solvent A | % solvent B |
|---|---|---|
| 0 | 95 | 5 |
| 5.33 | 92 | 8 |
| 35 | 50 | 50 |
| 36 | 20 | 80 |
| 40 | 20 | 80 |
| 41 | 95 | 5 |
| 50 | 95 | 5 |

### 6.5 Protein Identification

The peptide mass and fragmentation data generated in the LC-MS/MS experiments were used to query fasta formatted protein and nucleotide sequence databases maintained and updated regularly at the NCBI (for the NCBInr, dbEST and the human and mouse genomes) and European Bioinformatics Institute (EBI, for the human, mouse, D. melanogaster and C. elegans proteome databases). Proteins were identified by correlating the measured peptide mass and fragmentation data with the same data computed from the entries in the database using the software tool Mascot (Matrix Science; Perkins et al., 1999. Probability-based protein identification by searching sequence databases using mass spectrometry data. Electrophoresis 20, 3551-3567). Search criteria varied depending on which mass spectrometer was used for the analysis.

### 6.6 Protein Quantitation

Relative protein quantitation was performed using peak areas of iTRAQ reporter ion signals essentially as described by Ross and colleagues (Ross et al., 2004. Multiplexed protein quantitation in Saccharomyces cerevisiae using amine-reactive isobaric tagging reagents. Mol. Cell. Proteomics 3(12): 1154-1169).

### 6.7 Binding Curves and Determination of RI50 Values

The Relative Intensity (R1) values for the identified kinases are shown in Table 22. The test compound Bis VIII was used at three different concentrations in the cell lysate and the RI values were normalized to the DMSO control. For selected kinases the RI values were plotted against the concentration of Bis VIII and curve fitting was performed using the Xlfit program (ID Business Solutions Ltd.) using a hyperbolic equilibrium model (FIG. 5). The RI50 value corresponds to the test compound (Bis VIII) concentration at which the relative intensity of the MS signal for a kinase is 50% compared to the solvent (DMSO) control.

TABLE 22
Proteins identified by mass spectrometry analysis
The test compound Bis VIII was used at three different concentrations in the lysate and the
resulting RI values were normalized to the DMSO control which was set to 1.0. Relative
Intensity values are listed. The Representative refers to the database accession number of
the International Protein Index (IPI, European Bioinformatics Institute), a compilation of
protein sequences derived from several high-quality sequence databases (including
GenBank, EMBL, SwissProt, RefSeq, Ensembl). Kinase names are in accordance with the
human kinase nomenclature (http://kinase.com) and Manning et al., 2002, Science 298,
1912-1934 as specified in supplementary material).

| | | | Sample 1 | Sample 2 | Sample 3 | Sample 4 |
|---|---|---|---|---|---|---|
| Representative | Clustername | Kinase Name | DMSO | 0.074 uM | 0.22 uM | 0.67 uM |
| IPI00298977.4 | AAK1 | AAK1 | 1.00 | 0.82 | 0.90 | 0.75 |
| IPI00329488.4 | ABL2 | ARG | 1.00 | 0.96 | 0.97 | 0.91 |
| IPI00176642.3 | AURKB | AurB | 1.00 | 0.95 | 1.04 | 0.97 |
| CZB00000043.1 | ABI1 | Abl1 | 1.00 | 0.81 | 0.90 | 1.03 |
| IPI00306217.1 | BLK | BLK | 1.00 | 0.90 | 0.96 | 0.83 |
| IPI00430291.1 | CAMK2D | CaMK2d | 1.00 | 0.91 | 0.99 | 0.88 |
| IPI00169392.3 | CAMK2G | CaMK2g | 1.00 | 0.83 | 0.98 | 0.88 |
| IPI00031681.1 | CDK2 | CDK2 | 1.00 | 0.89 | 0.85 | 0.65 |
| IPI00023530.4 | CDK5 | CDK5 | 1.00 | 0.92 | 1.02 | 0.95 |
| IPI00000685.1 | CDK7 | CDK7 | 1.00 | 1.03 | 1.04 | 0.89 |
| IPI00013212.1 | CSK | CSK | 1.00 | 0.84 | 0.94 | 0.89 |
| IPI00183400.8 | CSNK1A1 | CK1a | 1.00 | 0.97 | 1.04 | 1.02 |
| IPI00465058.1 | CSNK1G1 | CK1g1 | 1.00 | 1.01 | 1.05 | 0.96 |
| IPI00219012.2 | FYN | FYN | 1.00 | 0.91 | 1.04 | 0.93 |
| IPI00298949.1 | GAK | GAK | 1.00 | 0.92 | 1.01 | 0.97 |
| IPI00292228.1 | GSK3A | GSK3A | 1.00 | 0.50 | 0.29 | 0.20 |
| IPI00216190.1 | GSK3B | GSK3B | 1.00 | 0.54 | 0.39 | 0.28 |
| IPI00004566.1 | ITK | ITK | 1.00 | 0.80 | 0.94 | 0.91 |
| IPI00515097.1 | LCK | LCK | 1.00 | 0.87 | 1.00 | 0.94 |
| IPI00003479.1 | MAPK1 | Erk2 | 1.00 | 0.89 | 1.04 | 0.98 |
| IPI00002857.1 | MAPK14 | p38a | 1.00 | 0.82 | 0.95 | 0.93 |
| IPI00024672.1 | MAPK8 | JNK1 | 1.00 | 0.91 | 1.02 | 0.83 |
| IPI00024673.1 | MAPK9 | JNK2 | 1.00 | 0.97 | 1.08 | 1.04 |
| IPI00012069.1 | NQO1 | | 1.00 | 0.84 | 0.99 | 0.87 |
| IPI00219129.7 | NQO2 | | 1.00 | 0.87 | 0.95 | 0.78 |
| IPI00410287.1 | PRKAA1 | AMPKa1 | 1.00 | 0.88 | 1.00 | 0.91 |
| IPI00220409.2 | PRKAB1 | | 1.00 | 0.91 | 0.94 | 0.79 |
| IPI00549328.2 | PRKAG1 | | 1.00 | 0.78 | 0.79 | 0.68 |
| IPI00385449.3 | PRKCA | PKCa | 1.00 | 0.25 | 0.19 | 0.18 |
| IPI00219628.1 | PRKCB1 | PKCb | 1.00 | 0.29 | 0.23 | 0.17 |
| IPI00029702.1 | PTK2B | PYK2 | 1.00 | 0.82 | 0.90 | 0.74 |
| IPI00465291.3 | SNF1LK2 | QIK | 1.00 | 0.93 | 1.11 | 0.98 |
| IPI00479211.2 | SRC | SRC | 1.00 | 0.93 | 0.98 | 0.94 |
| IPI00298940.2 | STK6 | AurA | 1.00 | 0.90 | 1.03 | 0.87 |
| IPI00293613.1 | TBK1 | TBK1 | 1.00 | 1.05 | 1.13 | 1.14 |
| IPI00411818.3 | ULK3 | ULK3 | 1.00 | 0.95 | 0.97 | 1.15 |
| IPI00025830.1 | WEE1 | Wee1 | 1.00 | 1.21 | 1.15 | 1.14 |
| IPI00477734.1 | YES1 | YES | 1.00 | 0.82 | 0.92 | 0.87 |

### Example 9

### A Quantitative Chemical Proteomics Approach Reveals Novel Modes of Action of Clinical ABL Kinase Inhibitors

Example 9 corresponds to the publication Bantscheff, M. et al., Nature Biotechnology, 25:1035-1044, herewith incorporated by reference.

### Abstract

We describe a novel chemical proteomics approach to profile the interaction of small molecules with hundreds of endogenously expressed protein kinases and purine-binding proteins. This sub-proteome is captured by immobilized non-selective kinase inhibitors (kinobeads) and bound proteins are quantified in parallel by mass spectrometry using isobaric tags for relative and absolute quantification (iTRAQ). By measuring the competition with the affinity matrix, we assess the binding of drugs to their targets in cell lysates and in cells. By mapping drug-induced changes in the phosphorylation state of the captured proteome, we also analyze signaling pathways downstream of target kinases. Quantitative profiling of the drugs imatinib, dasatinib and bosutinib in K562 cells confirms known targets including ABL and SRC family kinases and identifies the receptor tyrosine kinase DDR1 and the oxidoreductase NQO2 as novel targets of imatinib. The data indicate that our approach is a valuable tool for drug discovery.

### Introduction

Studies of drug action classically assess biochemical activity in settings which typically contain only the isolated target. Regularly, recombinant enzymes or protein fragments are used instead of the full-length endogenous proteins. To correlate accurately the activity of a compound determined in such assays with pharmacodynamic efficacy remains a challenge¹. One reason for this discrepancy is that an isolated recombinant protein may not reflect the native conformation and activity of the target in its physiological context, because of the lack of interacting regulatory proteins, expression of alternative splice variants, or incorrect protein folding or post-translational modifications. As a consequence, results from in-vitro experiments may not be predictive for the effects of a compound or drug in cell-based or in vivo systems. Moreover, although drugs are traditionally optimized against a single protein, many compounds act on multiple targets². These ‘off-targets’ may increase the therapeutic potential of a drug, but they might also cause toxic side effects.

Protein kinases represent an important class of drug targets particularly in oncology and inflammation³. However, kinase drug discovery epitomizes the shortcomings of the single-gene/single-protein/single-assay paradigm, as kinase inhibitors can be both conformation-specific and multi-targeted as demonstrated by recently launched multi-kinase drugs⁴⁻⁷. Evidently, compounds directed at the ATP-binding site of kinases are not likely to be specific for a single kinase, because there are around 500 protein kinases and more than 2000 other purine binding proteins in humans which share similar binding so pockets^{8, 9}. Conventional drug discovery mostly relies on panels of recombinant enzymes and cellular model systems to address compound potency, selectivity and potential off-target liabilities rather than attempting to determine the bona fide targets of a drug directly in an unbiased manner^{10, 11}.

Recent progress in affinity-based proteomic strategies has enabled the direct determination of protein-binding profiles of small molecule drugs under more physiological conditions¹². To date, methods rely on the attachment of labels to the compound (immobilization, fluorescent or affinity tags) or to the proteins^{10, 13, 14}, which may introduce artifacts driven by the altered properties of the compound or the protein. In the present study, we describe a chemical proteomics methodology which enables the capturing of a defined sub-proteome, consisting of a large portion of the expressed kinome and related proteins, on a mixed kinase inhibitor matrix (kinome beads or kinobeads), and subsequent analysis by quantitative protein mass spectrometry¹⁵. This approach allows the parallel quantitative determination of protein affinity profiles of kinase inhibitors in any cell type or primary tissue as well as the differential mapping of drug-induced changes of phosphorylation events on the captured sub-proteome. We apply the methodology to three drugs targeting the oncogenic BCR-ABL kinase, which induces chronic myelogenous leukemia (CML)¹⁶⁻¹⁸.

### Results

Target Profiling with Immobilized Kinase Inhibitors

Affinity purification strategies combined with mass spectrometry-based protein identification enable the identification of potential drug targets directly from cells or tissues^{12, 19}. We applied this strategy to a collection of more than 100 ATP-competitive kinase inhibitors including chemical scaffolds, research tool compounds, drug candidates in development, as well as approved drugs (FIG. 11). Most of these compounds do not contain functional groups suitable for covalent coupling to an affinity matrix while preserving activity. To overcome this limitation, analogues containing primary amino groups were synthesized (see

ary Table 1 for chemical structures). Following immobilization of the compounds, the beads were incubated with lysates of HeLa or K562 cells to allow protein binding. After separation of the beads from the lysate, bound proteins were eluted, digested with trypsin, and identified by mass spectrometry. FIG. 11 illustrates representative kinase binding profiles of 12 immobilized tool compounds and drugs. The data is represented as heat maps, using the number of spectrum-to-sequence matches as a measure for the amount of captured protein. In cases where the known targets of the compounds are expressed in HeLa or K562 cells, these targets were frequently identified (FIGS. 17 and 18). In addition, novel potential targets were found. Some of the compounds interacted rather selectively with few kinase targets whereas others displayed low apparent selectivity. For instance, the immobilized analogues of the EGF receptor inhibitors gefitinib and lapatinib, or the ABL inhibitor imatinib, bind few other kinases beyond their known targets. In contrast, all of the tool compounds, and the analogues of the clinical compounds sunitinib and vandetanib, bind to a much larger number of kinases.

### Kinobeads—a Mixed Kinase Inhibitor Affinity Resin

While the affinity profiles of immobilized compounds reveal novel target candidates, they are problematic for the validation of inhibitor selectivity for a number of reasons. First, the results obtained for the immobilized molecule may not relate directly to the original compound owing to altered potency and selectivity due to the attachment of the linker. Moreover, the resulting binding profiles are biased towards abundant proteins, which are often only weakly affected in subsequent activity-based assays^{13, 14, 21}.

To overcome these limitations, we developed a different approach, which uses the immobilized broad-selectivity inhibitors as kinase capturing tools to analyze the interaction of competing ‘free’ compound with their protein targets in solution. The method is based on measuring the degree of competition between the unmodified test compound and the immobilized ligands for ATP-binding and related sites on proteins. For an unbiased target profile, a capturing ligand binding to all members of a target class of interest would be required. A previously described method utilized immobilized ATP as the ligand²². However, in our experience this approach resulted in the capture of only a small number of kinases (<10) and instead was dominated by the binding of heat shock proteins (data not shown). Building on the observations from the immobilized kinase inhibitors described above, we selected from the set of immobilized compounds those ligands that displayed little selectivity and interacted with kinases located on different branches of the kinase phylogenetic tree. Following this rationale, a mixed inhibitor resin was created by immobilizing a combination of seven ligands. These mixed kinase inhibitor beads (kinobeads) captured a large portion of the expressed kinome. Using mass spectrometric analysis, a total of 174 and 183 protein kinases from HeLa and K562 cells respectively were identified in single pulldown experiments, with a confidence interval for the identification set at 99% (FIG. 6 and FIG. 19). When all the data obtained from kinobead pulldowns from 14 different human and mouse cell lines and tissues is accumulated, we identified a total of 307 non-redundant protein kinases (FIG. 7). While a slightly lower coverage was observed within the serine/threonine kinase branches compared to the tyrosine kinase branches, there were no major gaps.

Kinobeads do not only capture protein kinases, but bind a defined sub-proteome consisting also of other ATP- and purine-binding proteins such as chaperones, helicases, ATPases, motor proteins, transporters, and metabolic enzymes (Table 23 and FIG. 19). Based on the total mass spectrometric signal, we estimate that kinases account for almost 80% of the total captured protein amount (FIG. 12).

### Target Profiling of Drugs in Cell and Tissue Lysates

We applied kinobeads to the quantitative profiling of three inhibitors of the tyrosine kinase ABL; the drug candidate bosutinib (SKI-606) which is currently in clinical studies and the marketed drugs imatinib (Gleevec) and dasatinib (Sprycel)^{17, 23, 24}. All experiments were performed using K562 chronic myeloid leukemia cells, which express the constitutively active BCR-ABL oncogene²⁵. The drugs were added to cell lysates in concentrations ranging from 100 pM to 10 μM and the lysates were subsequently subjected to kinobeads precipitation. When the drug in the lysate binds its target and thus blocks the ATP binding site, a reduced amount of the free target is available for capturing on kinobeads, while the binding of non-targeted kinases and other proteins is unaffected (FIG. 8a). The kinobeads-bound material from each spiking experiment was subjected to tryptic digestion and peptides were labeled with the different forms of the iTRAQ reagent¹⁵. Subsequently, peptide mixtures were combined and subjected to mass spectrometric peptide sequencing. Relative protein quantification was achieved by measuring the signal of the iTRAQ reporter ions relative to vehicle-treated lysate. From this dataset, dose-response binding profiles were computed for >500 proteins in each sample, including ˜150 kinases (FIG. 20).

For imatinib, 13 proteins exhibited more than 50% binding reduction on kinobeads at 1 μM drug in the lysate. Among the competed proteins are ABL/BCR-ABL (IC50=250 nM), the ABL family kinase Arg (272 nM), and two novel target candidates, the receptor tyrosine kinase DDR1 (90 nM), and the quinone oxidoreductase NQO2 (43 nM) (FIG. 8b and FIG. 13a). Note that our mass spectrometry data do not reliably discriminate the normal ABL kinase expressed by the wild-type allele from the BCR-ABL fusion protein.

While imatinib affected only three out of the 142 kinases that were quantified in K562 lysate, dasatinib and bosutinib reveal broad target profiles (39 and 53 proteins respectively showed >50% competition at 1 μM), including the three imatinib targets BCR-ABL, ARG, and DDR1 (FIG. 8c and FIGS. 13b and c). The majority of the novel kinase targets were not available in commercial kinase panels, but for the tyrosine kinases Btk, EphB4, FAK/PTK2, FER, MER, and SYK, and the serine/threonine kinases GCK, KHS1 and p38a we determined IC₅₀ values in enzyme activity assays, which show a general trend supporting the kinobeads data (Table 24). A notable exception is the focal adhesion kinase FAK/PTK2, which was affected only by bosutinib, in line with published data reporting no binding of dasatinib or imatinib to FAK in phage display studies^{10, 26}. However, a fraction of FAK corresponding to an activated conformation (detected by spectra of a di-phosphorylated peptide representing the activation segment of the FAK kinase domain) was strongly affected by dasatinib, suggesting that the drug binds selectively to this activated conformation (FIG. 14). In agreement with this interpretation, dasatinib potently inhibited the activity of purified recombinant FAK.

In addition to kinases, several non-kinase targets were identified (FIG. 20), some of which do not contain obvious small molecule binding sites and hence are likely to bind indirectly to the drugs. Proteins which reside in a complex with the drug target are expected to exhibit similar competition behaviour. Indeed the BCR-ABL interacting proteins GRB2, SHC1, and SHIP2 displayed similar competition behaviour. STS-1, an adaptor protein described to inhibit the ubiquitin ligase CBL²⁷, also showed a similar dose-response for all three drugs. Consequently we propose it as a BCR-ABL kinase interacting protein (FIG. 8b; top row).

In addition to the ABL kinases, imatinib is also known to inhibit oncogenic mutants of the KIT and PDGF receptors, which is the basis of its therapeutic application in gastrointestinal stromal tumours²⁸. PDGF receptors are not expressed in K562 cells²⁹. Although K562 cells do express wild type KIT, no substantial competition of imatinib for kinobeads-captured KIT was detected by mass spectrometry. Likewise bosutinib did not substantially affect KIT, but dasatinib showed potent binding (IC_{50=0.30} μM). This observation was confirmed by western blot analysis of the kinobeads-captured material from imatinib-treated lysates using KIT antibodies (FIG. 9a). However, when the same blots were probed for activated KIT using a phospho-specific antibody directed against tyrosine 703, sub-micromolar competition by imatinib was seen. Hence, the kinobeads binding assay can differentiate between binding of a drug to distinct conformations of a target present in the same cell.

### DDR1 and NQO2 are Novel Targets of Imatinib

The discoidin domain receptor DDR1 represents a potential imatinib target. We confirmed the dose-response established by mass spectrometry by probing the same samples with DDR1 antibodies (FIG. 9a). Next, we tested whether imatinib inhibits the DDR1 kinase activity. DDR1 is a receptor tyrosine kinase which exhibits autophosphorylation and limited proteolysis in response to collagen binding or pervanadate treatment³⁰. Pre-incubation of the cells with imatinib reduced tyrosine phosphorylation and proteolytic processing of DDR1 (FIG. 9b). Finally, we confirmed DDR1 as potent imatinib target by measuring inhibition of the purified catalytic domain by means of autophosphorylation (IC50=22 nM) and the phosphorylation of a peptide substrate (IC50=31 nM, FIG. 9c). Consistent with the kinobeads data, DDR1 is inhibited by all three drugs. The only paralogue, DDR2, is likewise inhibited by imatinib and dasatinib (Table 24).

The oxidoreductase NQO2 represents the first potential non-kinase target of imatinib. Although NQO2 represents the most prominent non-kinase protein captured from K562 cells, the binding to imatinib is specific, as dasatinib and bosutinib did not efficiently compete. None of the three drugs did bind to the closely related NQO1 isoenzyme. NQO2 is a cytosolic flavoprotein which catalyzes the metabolic reduction of quinones and related xenobiotics³¹. We tested the ability of imatinib to inhibit recombinant NQO2 in an enzyme assay measuring the reduction of menadione³². Imatinib displayed potent competitive inhibition with a Kᵢ of 39 nM (FIG. 9d), in good agreement with the IC₅₀ of 43 nM determined by kinobeads binding, but itself was not modified by NQO2 (data not shown). Consistent with the kinobeads data, dasatinib and bosutinib did not inhibit NQO2.

### Profiling of Drug Effects on Signaling Pathways

Potent kinase inhibitors typically exhibit slow off-rates, which permits a variation of the previous experimental strategy. Instead of adding the drugs to the lysate, we applied them over a range of concentrations to cultured cells 5 hours prior to lysis and kinobeads precipitation. The results confirmed almost all of the targets obtained with the previous lysate competition experiments (FIG. 21). In a few cases, we observed competition when the compound was applied to cells, but not in the lysate competition experiment, for instance in the case of imatinib and KIT (FIG. 15).

To explore not only the direct targets of the drugs but also their downstream effects on signaling pathways, aliquots of the iTRAQ-labeled peptide mixtures from kinobeads precipitates were subjected to phosphopeptide enrichment and subsequent identification and quantification by mass spectrometry. For imatinib-treated cells, 379 tyrosine and serine/threonine phosphorylation sites on 136 different proteins were identified. Eight of these sites on 5 different proteins exhibited substantial down-regulation of their phosphorylation status in response to the drug (FIG. 10a). Indeed, most of these proteins have been implicated in signaling events downstream of ABL (FIG. 10b)^{16, 18}. For all three drugs together, we found down-regulation of 20 sites on 13 proteins (FIGS. 22 and 23).

### Discussion

The catalytic domains of kinases display high structural homology. Therefore, an early understanding of inhibitor selectivity in relevant tissues should increase the predictability of drug discovery, particularly for the application of kinase drugs in chronic conditions. Recently, techniques have been described to assess target profiles across the target class, ranging from affinity capturing of proteins in lysates using immobilized compounds to reactive ATP analogues^{12, 13, 22, 33}. While these methods enable the mapping of binding proteins directly in tissue lysates, they have limited potential for drug discovery since they do not generate quantitative data. Therefore, the validation of inhibitor selectivity mostly relies on data from large assay panels using recombinant enzymes and enzyme fragments, which are then correlated with results from cell-based assays^{10, 26, 34}. The kinobeads methodology described in this study enables, for the first time, the quantitative parallel profiling of the targets of ATP-site directed drugs directly in cells or in tissue lysate, without the need to modify either the compound or the proteins. The kinobeads matrix specifically captures around 200 protein kinases from any given cell type—estimated to represent at least two thirds of the expressed kinome²⁹- and >600 additional chemically tractable proteins. Hence, a pharmacologically relevant sub-proteome becomes available for the study of drug binding and drug-induced changes such as post-translational modifications under close to physiological conditions. Single target binding data can be recorded from the kinobeads with antibody reagents, enabling the screening of compounds against defined targets directly in tissue lysate, and in addition, a comprehensive readout is provided by the recently developed multiplexed quantitative mass spectrometry techniques¹⁵.

We validated this approach by profiling three ABL inhibitors and determined IC50 values in K562 lysate for several of their known targets, which are in line with reported cellular activities^{23, 35, 36}. The obtained IC50 binding data are largely independent of the affinity of the targets for the immobilized ligands, because the effective concentration of capturing molecules is typically below the range of affinities of the competing compound for its targets³⁷. Hence data obtained for all proteins in the same samples can be directly compared, which is an advantage compared to IC50 values determined in enzyme assays, which depend to a considerable degree on the assay conditions

Our data propose novel kinase and non-kinase targets for all three drugs. We confirm the unusually high selectivity of imatinib as just one novel kinase target was identified, the receptor tyrosine kinase DDR1, which is thought to play a role in various diseases including tumor progression and metastasis, atherosclerosis, lung inflammation and fibrosis³⁸. Activation of DDR1 inhibits p53-mediated apoptosis³⁹, possibly contributing to the synergetic effect of irradiation and imatinib treatment on tumor cell lines⁴⁰. Additional testing of imatinib in relevant disease models should further validate the role of DDR1 as a target but one interesting link is provided by our data. DDR1 knockout mice are resistant to bleomycin-induced lung fibrosis, a model in which imatinib shows efficacy^{41, 42}. A second unexpected target is the oxidoreductase NQO2, which protects cells against oxidative stress caused by xenobiotics³¹. High expression of NQO2 is found in myeloid cells, which are also the target of imatinib in chronic myelogenous leukemia. The potential consequences of NQO2 inhibition in patients treated with imatinib is beyond the scope of this study, but it is intriguing that the deletion of NQO2 in mice was reported to cause myeloid hyperplasia, and this may be exacerbated in the human population where NQO1 deficiency is a relatively common occurrence³¹. The second generation ABL inhibitors dasatinib and bosutinib were developed as dual Src/ABL kinase inhibitors and show overlapping but distinct target profiles. Bosutinib appears to be the first ABL inhibitor not to inhibit KIT. Since BCR-ABL up-regulates KIT expression and stem cell factor responsiveness is associated with proliferation of leukemic stem cells²⁷, lack of KIT inhibition might limit efficacy in CML. We identified many novel target candidates, and, where amenable, they were confirmed in biochemical activity assays. The potent inhibition of Btk and Syk, which signal downstream of immune receptors on granulocytes and B-cells, predicts immunomodulatory potential, which indeed has recently been demonstrated for dasatinib⁴³. The physiological role of several other target candidates—for example Ack/Tnk2, GAK, QIK, and QSK—is hitherto poorly understood. All of these kinases potently bind to dasatinib and bosutinib at therapeutically relevant drug concentrations, therefore they are likely affected during treatment. However, because these drugs appear to be relatively well tolerated, it can be inferred that the inhibition of these targets has no severe adverse consequences or may even contribute to efficacy.

For most targets similar potencies were obtained by applying the compounds directly to cells in culture compared to adding them to the lysate. A notable exception is the binding of imatinib to KIT, which was detected only when applied to cells in culture. This may be explained by two conformations existing in equilibrium in K562 cells, one of which binds imatinib. In the lysate, no competition was observed for the bulk of KIT, suggesting that the imatinib-binding form may amount to only a small fraction of total KIT. By contrast, when adding the drug to cultured cells, competition was observed for a substantial fraction of KIT. This suggests that imatinib effectively removes the high affinity form from the equilibrium, leading to depletion of the non imatinib-binding form. The high affinity conformation is presumably represented by KIT phosphorylated at Y703 since this form, while present only in low amounts, was competed also in the lysate (FIG. 9a). This observation is consistent with previous findings and with our enzyme assay data (Table 24) showing that imatinib does not effectively inhibit recombinant KIT⁴⁴. The imatinib-binding phospho-form possibly mimics the conformation of the oncogenic KIT mutants, which represent the target of imatinib in gastrointestinal stromal tumors¹⁷.

For a better prediction of drug effects it is useful to analyze the impact on the underlying signaling pathways. The mapping of drug-induced post-translational changes in the cellular kinome and its associated proteins can reveal effects of a drug beyond its direct targets. We find that a large portion of the BCR-ABL signaling pathway¹⁶ is recapitulated in the kinobeads data (FIG. 10b): Imatinib binding to its direct target BCR-ABL results not only in the loss of BCR-ABL from kinobeads, but similarly reduces the amount of the associated signaling proteins GRB2, SHC, and SHIP2. Since STS-1 is reduced with similar dose-response characteristics, it may represent another member of the signaling complex. The inhibition of BCR-ABL leads to decreased tyrosine phosphorylation of the adaptors SHC (at Y427) and DOK1 (at several sites), and of the GTPase activating protein RasGAP (at Y460). In turn, MAP kinase is down-regulated (at T184/Y186) leading to reduced phosphorylation of RSK kinases (at S360/377), preventing nuclear translocation and the induction of transcription⁴⁵.

In conclusion, our quantitative chemical proteomic approach enables, for the first time, the determination of the binding of small molecule inhibitors to their targets directly in cells or lysates of relevant tissues, as well as future applications to patient specimens such as tumor biopsies. The mixed affinity matrix in combination with quantitative mass spectrometry provides a versatile tool to map a drug's direct and indirect targets in a single set of experiments. We anticipate that this approach will prove valuable at various stages of drug discovery as well as in translational studies of drug action in patient tissues.

### Methods

Kinobeads and competition assays. Reagents were purchased from Sigma unless otherwise noted. Compounds for immobilization to beads (FIG. 17) were synthesized as described⁴⁶. Kinobeads were prepared by immobilizing Bis-(III) indolyl-maleimide, purvalanol B, staurosporine, and CZC8004, and the analogues of PD173955, sunitinib, and vandetanib on NHS-activated Sepharose 4 beads (Amersham) as described⁴⁶. Imatinib was purified from Gleevec tablets (Novartis) by HPLC. Dasatinib and bosutinib were synthesized following published procedures^{47, 48}. HeLa and K562 cells were obtained from ATCC and were cultured following ATCC protocols. Antibodies were purchased from Cell Signaling Technology (KIT, Y703P-KIT) and Santa Cruz (DDR1) and western blots were performed using a LI-COR Odyssey System.

Kinobeads profiling was performed essentially as described⁴⁶. Additional details are also provided below. Briefly, cells were homogenized in lysis buffer (50 mM Tris/HCl pH 7.5, 5% glycerol, 1.5 mM MgCl₂, 150 mM NaCl, 20 mM NaF, 1 mM Na₃VO₄, 1 mM DTT, 5 μM Calyculin A, 0.8% Igepal-CA630, and a protease inhibitor cocktail) using a Dounce homogenizer on ice. Lysates were cleared by centrifugation and adjusted to 5 mg/mL protein concentration using the Bradford assay. Compounds were dissolved in DMSO and added to 5 mL lysate samples, and 50 μL of a kinobeads suspension was added and agitated for 30 minutes at 4° C. Subsequently, the beads were washed, collected by centrifugation, and bound material was eluted with SDS sample buffer and fractionated by SDS gel electrophoresis. For profiling of signaling pathways, compounds were added to 10⁸ K562 cells per data point, grown at 10⁶ cells/mL in RPMI/10% FCS. Beads were eluted with NuPAGE buffer, eluates were reduced, alkylated, separated on 4-12% NuPAGE gels (Invitrogen), and stained with colloidal Coomassie.

Mass Spectrometry and Data analysis. Procedures were essentially as described⁴⁶ and are detailed below. Briefly, gel lanes were cut into slices across the separation range and subjected to in-gel tryptic digestion⁴⁹, followed by labeling with iTRAQ™ reagents (Applied Biosystems) as described¹⁵. Labeled peptide samples were combined and phosphopeptides were enriched using immobilized metal affinity chromatography (PhosSelect, Sigma)⁵⁰. Sequencing was performed by LC-MS/MS on an Eksigent 1D+ HPLC system coupled to a LTQ-Orbitrap mass spectrometer (Thermo Scientific). Peptide extracts of vehicle controls were labeled with iTRAQ reagent 117 and combined with extracts from compound-treated samples labeled with iTRAQ reagents 114-116 as detailed in FIG. 24. Tandem mass spectra were generated using pulsed-Q dissociation, enabling detection of iTRAQ reporter ions (see FIG. 25 and Supplementary Tables 1 and 2 submitted in electronic format). Peptide mass and fragmentation data were used to query an in-house curated version of the IPI database using Mascot (Matrix Science). Protein identifications were validated using a decoy data base. iTRAQ reporter ion-based quantification was performed with in-house developed software. Curve fitting was performed using R software (www.r-project.org).

Enzyme assays. DDR1 activation was assayed in K562 cells as described³⁰. NQO2 activity was determined using purified recombinant human NQO2 with menadione as substrate and CMCDP (1-Carbamoylmethyl-3-carbamoyl-1,4-dihydropyrimidine) as cofactor³². Kinase enzyme assays were performed as detailed below.

Accession numbers. PRIDE database (http://www.ebi.ac.uk/pride): complete mass spectrometry data set accession numbers 2445-3178. IntAct molecular interaction database (http:/www.ebi.ac.uk/intact): EBI-1379264, EBI-130386, EBI-1380809, EBI-1380831 and EBI-1380874.

### REFERENCES

1. Hall, S. E. Chemoproteomics-driven drug discovery: addressing high attrition rates. Drug Discov. Today 11, 495-502 (2006).
2. Morphy, R., Kay, C., & Rankovic, Z. From magic bullets to designed multiple ligands. Drug Discov. Today 9, 641-651 (2004).
3. Cohen, P. Protein kinases—the major drug targets of the twenty-first century? Nat. Rev. Drug Discov. 1, 309-315 (2002).
4. Liu, Y. & Gray, N. S. Rational design of inhibitors that bind to inactive kinase conformations. Nat. Chem. Biol. 2, 358-364 (2006).
5. Daub, H., Specht, K., & Ullrich, A. Strategies to overcome resistance to targeted protein kinase inhibitors. Nat. Rev. Drug Discov. 3, 1001-1010 (2004).
6. Mol, C. D., Fabbro, D., & Hosfield, D. J. Structural insights into the conformational selectivity of STI-571 and related kinase inhibitors. Curr. Opin. Drug Discov. DeveL 7, 639-648 (2004).
7. Fabbro, D. & Garcia-Echeverria, C. Targeting protein kinases in cancer therapy. Curr. Opin. Drug Discov. Devel. 5, 701-712 (2002).
8. Manning, G., Whyte, D. B., Martinez, R., Hunter, T., & Sudarsanam, S. The protein kinase complement of the human genome. Science 298, 1912-1934 (2002).
9. Haystead, T. A. The purinome, a complex mix of drug and toxicity targets. Curr. Top. Med. Chem. 6, 1117-1127 (2006).
10. Fabian, M. A. et al. A small molecule-kinase interaction map for clinical kinase inhibitors. Nat. Biotechnol. 23, 329-336 (2005).
11. Fliri, A. F., Loging, W. T., Thadeio, P. F., & Volkmann, R. A. Analysis of drug-induced effect patterns to link structure and side effects of medicines. Nat. Chem. Biol. 1, 389-397 (2005).
12. Szardenings, K., Li, B., Ma, L., & Wu, M. Fishing for targets: novel approaches using small molecule baits. Drug Discovery Today: Technologies 1, 9-15 (2004).
13. Godl, K. et al. An efficient proteomics method to identify the cellular targets of protein kinase inhibitors. Proc. Natl. Acad. Sci. U. S. A 100, 15434-15439 (2003).
14. Knockaert, M. et al. Intracellular targets of cyclin-dependent kinase inhibitors: identification by affinity chromatography using immobilised inhibitors. Chem. Biol. 7, 411-422 (2000).
15. Ross, P. L. et al. Multiplexed protein quantitation in Saccharomyces cerevisiae using amine-reactive isobaric tagging reagents. Mol. Cell. Proteomics. 3, 1154-1169 (2004).
16. Hantschel, O. & Superti-Furga, G. Regulation of the c-Abl and Bcr-Abl tyrosine kinases. Nat. Rev. Mol. Cell. Biol. 5, 33-44 (2004).
17. Capdeville, R., Buchdunger, E., Zimmermann, J., & Matter, A. Glivec (ST1571, imatinib), a rationally developed, targeted anticancer drug. Nat. Rev. Drug Discov. 1, 493-502 (2002).
18. Weisberg, E., Manley, P. W., Cowan-Jacob, S. W., Hochhaus, A., & Griffin, J. D. Second generation inhibitors of BCR-ABL for the treatment of imatinib-resistant chronic myeloid leukaemia. Nat. Rev. Cancer 7, 345-356 (2007).
19. Ding, S. et al. Synthetic small molecules that control stem cell fate. Proc. Natl. Acad. Sci. U S. A 100, 7632-7637 (2003).
20. Rappsilber, J., Ryder, U., Lamond, A. I., & Mann, M. Large-scale proteomic analysis of the human spliceosome. Genome Res. 12, 1231-1245 (2002).
21. Wissing, J. et al. Chemical proteomic analysis reveals alternative modes of action for pyrido[2,3-d]pyrimidine kinase inhibitors. Mol. Cell. Proteomics. 3, 1181-1193 (2004).
22. Graves, P. R. et al. Discovery of novel targets of quinoline drugs in the human purine binding proteome. Mol. Pharmacol. 62, 1364-1372 (2002).
23. Golas, J. M. et al. SKI-606, a 4-anilino-3-quinolinecarbonitrile dual inhibitor of Src and Abl kinases, is a potent antiproliferative agent against chronic myelogenous leukemia cells in culture and causes regression of K562 xenografts in nude mice. Cancer Res. 63, 375-381 (2003).
24. Kantarjian, H., Jabbour, E., Grimley, J., & Kirkpatrick, P. Dasatinib. Nat. Rev. Drug Discov. 5, 717-718 (2006).
25. Druker, B. J. et al. Effects of a selective inhibitor of the Abl tyrosine kinase on the growth of Bcr-Abl positive cells. Nat. Med. 2, 561-566 (1996).
26. Carter, T. A. et al. Inhibition of drug-resistant mutants of ABL, KIT, and EGF receptor kinases. Proc. Natl. Acad. Sci. U. S. A 102, 11011-11016 (2005).
27. Kowanetz, K. et al. Suppressors of T-cell receptor signaling Sts-1 and Sts-2 bind to Cbl and inhibit endocytosis of receptor tyrosine kinases. J. Biol. Chem. 279, 32786-32795 (2004).
28. Tuveson, D. A. et al. ST1571 inactivation of the gastrointestinal stromal tumor c-KIT oncoprotein: biological and clinical implications. Oncogene 20, 5054-5058 (2001).
29. Su, A. I. et al. Large-scale analysis of the human and mouse transcriptomes. Proc. Natl. Acad. Sci. U S. A 99, 4465-4470 (2002).
30. L'hote, C. G., Thomas, P. H., & Ganesan, T. S. Functional analysis of discoidin domain receptor 1: effect of adhesion on DDR1 phosphorylation. FASEB J. 16, 234-236 (2002).
31. Vella, F., Ferry, G., Delagrange, P., & Boutin, J. A. NRH: quinone reductase 2: an enzyme of surprises and mysteries. Biochem. Pharmacol. 71, 1-12 (2005).
32. Knox, R. J. et al. Bioactivation of 5-(aziridin-1-yl)-2,4-dinitrobenzamide (CB 1954) by human NAD(P)H quinone oxidoreductase 2: a novel co-substrate-mediated antitumor prodrug therapy. Cancer Res. 60, 41794186 (2000).
33. Patricelli, M. P. et al. Functional interrogation of the kinome using nucleotide acyl phosphates. Biochemistry 46, 350-358 (2007).
34. Knight, Z. A. & Shokat, K. M. Features of selective kinase inhibitors. Chem. Biol. 12, 621-637 (2005).
35. Buchdunger, E. et al. Inhibition of the Abl protein-tyrosine kinase in vitro and in vivo by a 2-phenylaminopyrimidine derivative. Cancer Res. 56, 100-104 (1996).
36. Lombardo, L. J. et al. Discovery of N-(2-chloro-6-methyl-phenyl)-2-(6-(4-(2-hydroxyethyl)-piperazin-1-yl)-2-methylpyrimidin-4-ylamino)thiazole-5-carboxamide (BMS-354825), a dual Src/Abl kinase inhibitor with potent antitumor activity in preclinical assays. J. Med. Chem. 47, 6658-6661 (2004).
37. Lowe, C. R., Harvey, M. J., Craven, D. B., & Dean, P. D. Some parameters relevant to affinity chromatography on immobilized nucleotides. Biochem. J. 133, 499-506 (1973).
38. Vogel, W. F., Abdulhussein, R., & Ford, C. E. Sensing extracellular matrix: an update on discoidin domain receptor function. Cell Signal. 18, 1108-1116 (2006).
39. Ongusaha, P. P. et al. p53 induction and activation of DDR1 kinase counteract p53-mediated apoptosis and influence p53 regulation through a positive feedback loop. EMBO J. 22, 1289-1301 (2003).
40. Rossler, J., Zambrzycka, I., Lagodny, J., Kontny, U., & Niemeyer, C. M. Effect of STI-571 (imatinib mesylate) in combination with retinoic acid and gamma-irradiation on viability of neuroblastoma cells. Biochem. Biophys. Res. Commun. 342, 1405-1412 (2006).
41. Avivi-Green, C., Singal, M., & Vogel, W. F. Discoidin domain receptor 1-deficient mice are resistant to bleomycin-induced lung fibrosis. Am. J. Respir. Crit. Care Med. 174, 420-427 (2006).
42. Daniels, C. E. et al. Imatinib mesylate inhibits the profibrogenic activity of TGF-beta and prevents bleomycin-mediated lung fibrosis. J. Clin. Invest 114, 1308-1316 (2004).
43. Kneidinger, M. et al. Dasatinib (BMS354825) inhibits IgE-dependent activation and histamine release in human blood basophils. Blood 108, Abs. 1365 (2006).
44. Shah, N. P. et al. Dasatinib (BMS-354825) inhibits KITD816V, an imatinib-resistant activating mutation that triggers neoplastic growth in most patients with systemic mastocytosis. Blood 108, 286-291 (2006).
45. Yang, T. T., Xiong, Q., Graef, l. A., Crabtree, G. R., & Chow, C. W. Recruitment of the extracellular signal-regulated kinase/ribosomal S6 kinase signaling pathway to the NFATc4 transcription activation complex. Mol. Cell. Biol. 25, 907-920 (2005).
46. Drewes, G. et al. Process for the identification of novel enzyme interacting compounds. Patent WO 2006/134056 A1 (2006).
47. Boschelli, D. H. et al. 7-Alkoxy-4-phenylamino-3-quinolinecar-bonitriles as dual inhibitors of Src and AbI kinases. J. Med. Chem. 47, 1599-1601 (2004).
48. Das, J. et al. 2-aminothiazole as a novel kinase inhibitor template. Structure-activity relationship studies toward the discovery of N-(2-chloro-6-methylphenyl)-2-[[6-[4-(2-hydroxyethyl)-1-piperazinyl)]-2-methyl-4-pyrimidinyl]amino)]-1,3-thiazole-5-carboxamide (dasatinib, BMS-354825) as a potent pan-Src kinase inhibitor. J. Med. Chem. 49, 6819-6832 (2006).
49. Shevchenko, A., Wilm, M., Vorm, O., & Mann, M. Mass spectrometric sequencing of proteins silver-stained polyacrylamide gels. Anal. Chem. 68, 850-858 (1996).
50. Pozuelo, R. M., Campbell, D. G., Morrice, N. A., & Mackintosh, C. Phosphodiesterase 3A binds to 14-3-3 proteins in response to PMA-induced phosphorylation of Ser428. Biochem. J. 392, 163-172 (2005).

### Additional Information on Example 9

### Preparation of Kinobeads

Broad spectrum capturing ligands were immobilized on Sepharose beads through covalent linkage using amino and carboxyl groups. Compounds that do not contain a suitable functional group were modified in order to introduce such a group (see FIG. 17). Details of ligand syntheses are reported elsewhere¹. For the immobilization via amino groups, 1 mL of NHS-activated Sepharose (Amersham) was equilibrated in DMSO and the ligand (0.1 μmol/mL of beads in DMSO) and 15 μL of triethylamine were added and the reaction allowed to proceed on an end-over-end shaker for 16 hours. The coupling reaction was monitored by HPLC. Free NHS-groups were blocked with aminoethanol and washed beads were stored in isopropanol at −20° C. For the immobilization of compounds via carboxyl groups, NHS-activated Sepharose 4 equilibrated in DMSO is added to a 4:4:1 mixture of aminoethanol, triethylamine and ethylenediamine and the reaction was allowed to proceed for 16 hours on a shaker. After the reaction, the beads were washed with DMSO, and equilibrated in DMF. 100 μL of diisopropylethylamine and the ligand (0.1 μmol/mL of beads) were added, followed by 100 μL of a 100 mM solution of bromo-tris-pyrrolidino-phosphonium hexafluorophosphate in DMF. After incubation over night on a shaker, the beads were blocked by 100 μL of 100 mM NHS-acetate as blocking reagent for 16 hours. Coupling was monitored by HPLC. Beads were washed in DMSO and stored in isopropanol and stored at −20° C.

### Kinobeads Competition Binding Assay

Cells were harvested by centrifugation and homogenized in lysis buffer (50 mM Tris/HCl pH 7.5, 5% glycerol, 1.5 mM MgCl2, 150 mM NaCl, 20 mM NaF, 1 mM Na₃VO₄, 1 mM DTT, 5 M Calyculin A, 0.8% Igepal-CA630, and a protease inhibitor cocktail) using a Dounce homogenizer on ice. Lysates were cleared by centrifugation at 50,000 g for 30 min. at 4° C., and adjusted to 5 mg/mL total protein concentration using the Bradford assay. Compounds were dissolved in DMSO and added to 1 mL lysate samples, and 35 μL of a kinobeads suspension was added and agitated for 30 minutes at 4° C. This results in sufficient material for at least 10 LC-MS/MS samples for protein identification analysis, and in addition duplicate IMAC phospho-peptide samples (see below). For profiling of signaling pathways, compounds were added to 10⁸ K562 cells per data point, grown at 10⁶ cells/mL in RPMI/10% FCS.

After the incubation step, the beads were collected by centrifugation in a benchtop centrifuge for 1 minute at 800 rpm at 4° C., and washed once with 1 mL of ice-cold buffer (50 mM Tris/HCl pH 7.5, 5% (v/v) glycerol, 1.5 mM MgCl₂, 150 mM NaCl, 20 mM NaF, 1 mM DTT, 0.4% Igepal-CA630). Beads were eluted with NuPAGE LDS buffer (Invitrogen), eluates were reduced, alkylated, separated on 4 12% NuPAGE gels (Invitrogen), and stained with colloidal Coomassie blue.

ITRAQ labeling of Peptides

For quantitative experiments, reduced and carbamidomethylated kinobead eluates were concentrated on 4 12% NuPAGE gels (Invitrogen) by running sample approximately 1 cm into the gel. After staining with colloidal Coomassie, gels were cut into three slices and subjected to in-gel digestion as described². Subsequently, peptide extracts were labeled with iTRAQ reagents (Applied Biosystems) by adding 10 μL reagent in ethanol and incubation for 1 hr at 20° C. in 60% ethanol, 40 mM triethylammoniumbicarbonate (TEAB), pH 8.5³ After quenching of the reaction with glycin all labeled extracts of one gel lane were combined and mixed with differently labeled extracts from other competition experiments according to FIG. 24.

### Enrichment of Phospho-Peptides by Immobilized Metal Affinity Chromatography

As indicated in FIG. 24, selected samples were subjected to enrichment of phosphorylated peptides by immobilized metal affinity chromatography (IMAC; PhosSelect, Sigma) prior to mass spectrometric analysis as described⁴. LC-MS/MS for IMAC samples were performed twice.

LC-MS/MS Analysis

IMAC-binding and non-binding fractions were collected separately, acidified and dried in vacuo. Samples were then re-suspended in 0.1% formic acid in water (non-binding fraction) or 4 mM EDTA, 10 mM TEAB, pH 8.5 in water (phospho-peptide enriched fraction) and aliquots of the sample were injected into a nano-LC system (Eksigent 1D+) which was directly coupled to a LTQ-Orbitrap mass spectrometer (Thermo-Finnigan). Peptides were separated on a custom made 20 cm×75 uM (ID) reversed phase column (Reprosil). Gradient elution was performed from 2% acetonitrile to 40% acetonitrile in 0.1% formic acid within 4 hrs. The LTQ-Orbitrap was operated under the control of XCalibur Developers kit 2.0. Intact peptides were detected in the Orbitrap at 60.000 resolution. Internal calibration was performed using the ion signal from (Si(CH₃)₂O)₆H+ at m/z 445.120025⁵. Data dependent tandem mass spectra were generated for up to six peptide precursors in the linear ion trap using pulsed-Q dissociation (PQD) to enable detection of iTRAQ reporter ions⁶. For PQD, the Q-value was set to 0.55, activation time was set to 0.32 ms and collision energy of 26 was used. Up to 1E5 ions were accumulated in the ion trap within a maximum ion accumulation time of 1 sec and two spectra were averaged per peptide precursor. Further details on PQD/iTRAQ procedures will be published elsewhere (Bantscheff et al, manuscript in preparation).

### Peptide and Protein Identification

Mascot™ 2.0 software (Matrix Science) was used for protein identification using 5 ppm mass tolerance for peptide precursors and 0.8 Da tolerance for fragment ions. Carbamidomethylation of cysteine residues and iTRAQ modification of lysine residues were set as fixed modifications and S,T,Y phosphorylation, methionine oxidation, Nterminal acetylation of proteins and iTRAQ modification of peptide N-termini were set as variable modifications. The search data base consisted of an in-house curated version of the IPI protein sequence database combined with a decoy version of this database⁷. The decoy data base was created using a script supplied by Matrix Science. The Mascot ion score threshold for this database was 38 (indicating <5% random spectrum to sequence assignments). Unless stated otherwise, we accepted protein identifications as follows:

(i) for single spectrum to sequence assignments, we required this assignment to be the best match and a minimum Mascot score of 37 and a 10× difference of this assignment over the next best assignment. Based on these criteria, the decoy search results indicate <1% false positive identification rate;
(ii) for multiple spectrum to sequence assignments and using the same parameters, the decoy search results indicate <0.1% false positive identification rate;
(iii) for phospho-peptides <3% false positive identification rate was achieved either by a decoy analysis at a minimum Mascot score of 31 or by requiring the identification of a phospho-peptide in at least 12 of the 24 IMAC experiments.

### Functional Annotation

The functional annotation provided in Table 23 and FIG. 19 was performed by matching each identified protein to the Sugen kinase lists, to Gene Ontology (GO) terms⁹ and to Interpro domains¹⁰.

### Heat Map Generation

For generation of heat maps (FIG. 6 and FIG. 11), a semi-quantitative estimation of relative protein abundance was achieved using the total number of spectrum to sequence matches (SSMs) obtained for individual proteins in cell lines¹¹. The cell line for which the highest number of SSMs was obtained is indicated in dark blue. Lighter levels of blue indicate lower numbers of SSMs using a total of 15 levels of blue.

### Peptide and Protein Quantification

Centroided iTRAQ reporter ion signals were computed by the XCalibur software operating the mass spectrometer and extracted from MS data files using in-house developed software. Only peptides unique for identified proteins were used for relative protein quantification. iTRAQ reporter ion intensities were multiplied with the ion accumulation time yielding an area value proportional to the number of reporter ions present in the ion trap. Fold changes are reported based on iTRAQ reporter ion areas in comparison to vehicle control and were calculated using a linear model. For quantification of phosphorylated peptides, only those were considered for which the sum of iTRAQ areas was greater than 100.000. For more details see FIGS. 17-25 and Supplementary Tables 1 and 2 submitted in electronic format.

### Dose Response Binding Curves and IC₅₀ Calculation

Dose-response curves were fitted using R (www.r-project.org)¹² and the drc package (www.bioassay.dk)¹³. For each protein, relative displacement values to the vehicle control were fitted to concentrations of compound using a 4-parameter, unconstrained log-logistic equation. In some cases, the upper limit had to be fixed to 1 (vehicle control) to allow proper fitting. Inflection point and IC₅₀ (corresponding the 50% of the vehicle control) were reported for any protein that was displaced at least 40% compared to the vehicle control.

Quality Control and Robustness of Kinobead Profiling Kinobeads were generated in batches from 1 mL up to 100 mL. Quality controls of different batches were performed by monitoring the coupling reaction by HPLC and by testing each batch in a compound competition binding assay where IC50 binding values for a number of kinases are generated using western blot-based quantification on a LICOR Odyssey instrument, as shown in FIG. 9a. The observed reproducibility of the resulting IC₅₀ values between batches is typically better than twofold. In six different experiments using different batches of cell lysate, different batches of kinobeads, and different mass spectrometers, IC₅₀ binding values obtained were typically also within twofold. For instance, the IC₅₀ binding values for imatinib to BCR-ABL and NQO2 in K562 lysate determined in these six independent experiments were 128+/−93 nM and 42+/−14 nM, respectively. These values show only slightly higher variability than the values determined using the same batch of kinobeads and lysate (see FIG. 13). For the profiling experiments shown in FIG. 6 and FIG. 8, one single batch of K562 cell lysate was used.

### Biochemical Kinase Activity Assays

Biochemical kinase activity assays were performed by the Invitrogen SelectScreen service, for the following kinases: BTK, EphB4, FAK/PTK2, FER, GCK, KHS1, KIT, MER, p38, and SYK. The concentration of ATP was selected to equal Kₘ, except for p38, where 100 μM ATP was used. Inhibition data for DDR2 were generated by the Upstate IC₅₀Profiler Express-m service, using an ATP concentration of 200 μm. Inhibition data for DDR1 were generated in-house, using a purified recombinant fragment of human DDR1 containing the catalytic domain, purchased from Carna Biosciences. Inhibition was assayed in kinase buffer (20 mM Tris pH 7.5, 2 mM MgCl₂, 2 mM MnCl₂, 0.1 mM Na₃VO₄, 0.05% Brij-35) supplemented with 10 μM (gama-31P)ATP (20 Ci/mmol) and 25 μM IRS1 peptide-F (a generous gift of Dr. Takashi Hara, Cama Biosciences) following published procedures¹⁴. Inhibition of DDR1 was also assayed by autophosphorylation using (per data point) 100 ng DDR1 in kinase buffer supplemented with 2 μM (gamma-³²P)ATP (100 Ci/mmol). Radioactive phosphate incorporated in DDR1 was quantified by SDS-PAGE and autoradiography using a Typhoon 9200 (Amersham Biosciences).

### Appendix: Factors Influencing the Competition Binding Assay

There are a number of variables which in theory should affect the degree of competition of a protein binding to the capturing ligands on the kinobeads: (1) the affinity of a givenprotein for the capturing ligand, (2) the concentration of the capturing ligand, (3) the expression level of the kinase, or more directly, the concentration of the kinase in the lysate, and (4) the concentration and affinity of the non-immobilized compound in competition with the capturing ligand. It is advantageous to minimize the impact of the first three factors, so that, under conditions of competition with free compound, the determined IC50 competition values are close to true dissociation constants, and are not influenced markedly by the other variables which tend to differ between individual kinases, ligands, and lysates. Therefore, we have selected conditions under which we do observe little (<10%) or no depletion of proteins from the lysate. This is achieved by (1) keeping the concentration of capturing ligands during the incubation at sub-micromolar levels, and (ii) by using a large access of lysate to become independent of expression levels. Under these conditions, the data obtained for all proteins in the same sample can be directly compared. A more quantitative discussion of these factors is given below, and can also be found in the literature^{15,16}.

However, in some cases when one or more of the immobilized inhibitors exhibit very high affinity of for a given protein, the binding results for the non-immobilized test compounds binding to this protein could be skewed. The binding results would show a systematic shift towards higher IC50 values if the dissociation constant (Kd) of a given protein for the immobilized ligand would be substantially lower (by one or more orders of magnitude) than the concentration of the capturing ligand during the kinobeads binding step. In practical terms, this would be the case for proteins where the capturing ligand exhibits low nanomolar or even picomolar Kd values, which is not expected to be the case for the immobilized broad-selectivity ligands used in this study. Such very high affinity capturing ligands may lead to substantial depletion of binding proteins from the lysate. We have tested this for a number of kinases and in no case observed more than 10% depletion. However, it should be noted that the relative order of binding for a number of free test compounds to such a protein would still be correct.

The above arguments can be derived from a set of binding equations. If a compound C binds to a protein P:

C+P [symbol] PC, Equation 1

the equilibrium is defined by:

$$\begin{matrix}{K}_{D} = \frac{[ C ] ⋆ [ P ]}{[ PC ]} , & Equation 2 \\ resulting in & \\ \begin{matrix} ∀ [ C ] = {[ C ]}_{1 / 2} , \\ [ P ] = [ PC ]\end{matrix} , & Equation 3 \\ and & \\ {K}_{D} = {[ C ]}_{1 / 2} . & Equation 4\end{matrix}$$

Upon the addition of a capturing ligand, this equilibrium is affected by a second process, namely, the binding of free protein to the immobilized ligand B:

B+P [symbol] PB Equation 5

Note that in the following equations, this does not necessarily implicate that an equilibrium state is reached. PB could also be a function of time.

This reaction influences the half-binding concentration of C as follows:

$$\begin{matrix}∀ [ C ] = {[ C ]}_{1 / 2} , \\ [ P ] + [ PB ] = [ PC ] .\end{matrix} \qquad Equation 6$$

Furthermore, the initial protein concentration Po is:

[P]₀=[P]+[PC]+[PB], Equation 7

Thus resulting in

$$\begin{matrix}\begin{matrix}{[ P ]}_{0} = [ P ] + [ P ] + [ PB ] + [ PB ] \\ = 2 [ P ] + 2 [ PB ] \\ = 2 [ PC ] .\end{matrix} & Equation 8\end{matrix}$$

Therefore, the initial concentration of free compound is:

$$\begin{matrix}\begin{matrix}{[ C ]}_{1 / 2 , 0} = {[ C ]}_{1 / 2} + [ PC ] \\ = {[ C ]}_{1 / 2} + \frac{{[ P ]}_{0}}{2} ,\end{matrix} & Equation 9\end{matrix}$$

And using equation 6, equation 2 is transformed into:

$$\begin{matrix}{[ C ]}_{1 / 2 , 0} = {K}_{D} ⋆ \frac{[ PC ]}{[ P ]} + \frac{{[ P ]}_{0}}{2} \\ = {K}_{D} + {K}_{D} ⋆ \frac{[ PB ]}{[ P ]} + \frac{[ P ]}{2} .\end{matrix} \qquad Equation 10$$

Assuming that B+P [symbol] PB are in equilibrium, this results in

$$\frac{[ B ]}{{K}_{DB}} = \frac{[ PB ]}{[ P ]} . \\ Assuming that \frac{{[ P ]}_{0}}{2} << {K}_{D} , \qquad Equation 11$$

Equations 10 and 11 can be combined, yielding:

$${[ C ]}_{1 / 2 , 0} = {K}_{D} ⋆ ( 1 + \frac{[ B ]}{{K}_{DB}} ) + \frac{{[ P ]}_{0}}{2} . \qquad Equation 12$$

However, in kinobeads competition experiments B+P [symbol] PB are not necessarily in equilibrium. Hence, using equations 6-8, equation 2 can then be expressed as:

$${K}_{D} = \frac{{[ C ]}_{1 / 2} ⋆ ( \frac{{[ P ]}_{0}}{2} - [ PB ] )}{\frac{{[ P ]}_{0}}{2}} . \qquad Equation 13$$

Or, using equation 9:

$${[ C ]}_{1 / 2 , 0} = \frac{{K}_{D}}{1 - 2 \frac{[ PB ]}{{[ P ]}_{0}}} + \frac{{[ P ]}_{0}}{2} . \qquad Equation 14$$

where PB is a function of time, until an equilibrium is reached.
Now, the influence of depletion (the fraction of a given protein bound to the capturing ligands) can be calculated. Based on equation 14, FIG. 16 shows how the fraction of protein bound to the capturing ligand affects the deviation of the competition binding IC50 value from the Kd:

Thus, as long as the fraction of protein depleted is below 25%, the IC50 will be less than two times the Kd. As shown this is governed by an asymptotic function with a non-defined point at 50%. Thus, in regions >40% depletion one is unlikely to measure any competition.

### Since

$$\frac{d ( [ PB ] )}{d t} = {k}_{on} ⋆ [ P ] ⋆ [ B ] , \qquad Equation 15$$

the rate of protein binding to the capturing ligands on the beads is a function of the capturing ligand concentration, the protein concentration and time. As noted, the influence of IC50/Kd can be minimized by using a low concentration of capturing ligands and/or a ligand with low affinity. As long as the initial protein concentration is below Kd, using a lower protein concentration is not favorable since in equation 14, the ratio of concentration of protein on beads to initial protein concentration is the relevant term.

### REFERENCES

1. Drewes, G. et al. Process for the identification of novel enzyme interacting compounds. Patent WO 2006/134056 A1 (2006).
2. Rosenfeld, J., Capdevielle, J., Guillemot, J. C., & Ferrara, P. In-gel digestion of proteins for internal sequence analysis after one- or two-dimensional gel electrophoresis. Anal. Biochem. 203, 173-179 (1992).
3. Ross, P. L. et al. Multiplexed protein quantitation in Saccharomyces cerevisiae using aminereactive isobaric tagging reagents. Mol. Cell. Proteomics. 3, 1154-1169 (2004).
4. Pozuelo, R. M., Campbell, D. G., Morrice, N. A., & Mackintosh, C. Phosphodiesterase 3A binds to 14-3-3 proteins in response to PMA-induced phosphorylation of Ser428. Biochem. J. 392, 163-172 (2005).
5. Olsen, J. V. et al. Parts per million mass accuracy on an Orbitrap mass spectrometer via lock mass injection into a C-trap. Mol. Cell. Proteomics. 4, 2010-2021 (2005).
6. Meany, D. L., Xie, H., Thompson, L. V., Arriaga, E. A., & Griffin, T. J. Identification of carbonylated proteins from enriched rat skeletal muscle mitochondria using affinity chromatography-stable isotope labeling and tandem mass spectrometry. Proteomics. 7, 1150-1163 (2007).
7. Elias, J. E., Haas, W., Faherty, B. K., & Gygi, S. P. Comparative evaluation of mass spectrometry platforms used in large-scale proteomics investigations. Nat. Methods 2, 667-675 (2005).
8. Manning, G., Whyte, D. B., Martinez, R., Hunter, T., & Sudarsanam, S. The protein kinase complement of the human genome. Science 298, 1912-1934 (2002).
9. Ashbumer, M. et al. Gene ontology: tool for the unification of biology. The Gene Ontology Consortium. Nat. Genet. 25, 25-29 (2000).
10. Mulder, N. J. et al. New developments in the InterPro database. Nucleic Acids Res. 35, D224-D228 (2007).
11. Allet, N. et al. In vitro and in silico processes to identify differentially expressed proteins. Proteomics. 4, 2333-2351 (2004).
12. R Development Core Team. R: A language and environment for statistical computing. Vienna, Austria (2007).
13. Ritz, C. & Streibig, J. C. Bioassay Analysis using R. J. Statist. Software 12, (2007).
14. Casnellie, J. E. Assay of protein kinases using peptides with basic residues for phosphocellulose binding. Methods Enzymol. 200, 115-120 (1991).
15. Lowe, C. R., Harvey, M. J., Craven, D. B., & Dean, P. D. Some parameters relevant to affinity chromatography on immobilized nucleotides. Biochem. J. 133, 499-506 (1973).
16. Fabian, M. A. et al. A small molecule-kinase interaction map for clinical kinase inhibitors. Nat. Biotechnol. 23, 329-336 (2005).

TABLE 23
Number and functional categorization of proteins captured on kinobeads from
human cell lines and tissue.

| Family | Subfamilies | HeLa | Placenta | Jurkat | K562 | Ramos |
|---|---|---|---|---|---|---|
| Protein Kinases | TK | 37 | 52 | 36 | 38 | 29 |
| | TKL | 21 | 21 | 20 | 23 | 23 |
| | STE | 23 | 20 | 17 | 21 | 21 |
| | CK1 | 4 | 4 | 6 | 6 | 6 |
| | AGC | 9 | 13 | 15 | 11 | 10 |
| | CAMK | 16 | 13 | 20 | 18 | 20 |
| | CMGC | 20 | 16 | 26 | 21 | 23 |
| | Atypical | 13 | 8 | 11 | 13 | 13 |
| | TIF1 | 1 | | 1 | 1 | 1 |
| | Other | 29 | 18 | 29 | 29 | 37 |
| | Total | 173 | 165 | 181 | 181 | 183 |
| Lipid Kinases | — | 4 | 5 | 2 | 6 | 4 |
| Sugar kinases | — | 5 | 2 | 4 | 4 | 3 |
| Nucleotide kinases | — | 3 | 4 | 2 | 6 | 4 |
| Other kinases | — | 3 | 4 | 1 | 3 | 2 |
| Enzymes | GTPases | 2 | 4 | 4 | 3 | 4 |
| | Helicases | 20 | 5 | 14 | 16 | 20 |
| | Hydrolases | 76 | 105 | 81 | 108 | 128 |
| | Isomerases | 19 | 18 | 7 | 24 | 19 |
| | Ligases | 40 | 19 | 33 | 45 | 52 |
| | Lyases | 10 | 13 | 8 | 15 | 12 |
| | Motor proteins | 8 | 6 | 7 | 10 | 13 |
| | Oxidoreductases | 48 | 66 | 43 | 62 | 66 |
| | Proteases | 1 | 7 | 0 | 0 | 0 |
| | Peroxidases | 7 | 11 | 6 | 11 | 8 |
| | Phospholipases | 0 | 1 | 0 | 1 | 1 |
| | Transferases | 72 | 57 | 58 | 69 | 92 |
| | Glucosidases | 1 | 1 | 1 | 1 | 0 |
| | Phosphatases | 1 | 2 | 1 | 1 | 2 |
| | Phosphodiesterases | 4 | 6 | 4 | 2 | 4 |
| | Ubiquitination | 6 | 2 | 4 | 7 | 7 |
| | Total | 315 | 323 | 271 | 375 | 428 |
| Heat Shock proteins | — | 14 | 17 | 16 | 21 | 21 |
| Nucleic acid binding | Purine metabolism | 14 | 6 | 15 | 12 | 12 |
| | Pyrimidine metabolism | 3 | 3 | 3 | 4 | 3 |
| | Ribosomal proteins | 24 | 24 | 21 | 47 | 40 |
| | Transcription factors | 39 | 25 | 41 | 49 | 61 |
| | Translation factors | 31 | 18 | 22 | 29 | 35 |
| | Others | 55 | 47 | 48 | 53 | 77 |
| | Total | 166 | 123 | 150 | 194 | 228 |
| Protein binding | Cytoskeleton | 27 | 33 | 19 | 25 | 30 |
| | Enzyme inhibitors | 16 | 29 | 14 | 14 | 22 |
| | GTPase regulators | 17 | 16 | 21 | 22 | 30 |
| | Kinase regulatory | 17 | 10 | 15 | 15 | 23 |
| | Receptor binding | 18 | 31 | 19 | 25 | 20 |
| | Others | 192 | 200 | 200 | 218 | 277 |
| | Total | 287 | 319 | 288 | 319 | 402 |
| Transporters | — | 82 | 108 | 87 | 89 | 130 |
| Receptors | — | 24 | 48 | 35 | 24 | 50 |
| Lipid binding | — | 9 | 14 | 10 | 13 | 15 |
| Vesicle Trafficking | — | 8 | 19 | 8 | 7 | 14 |
| Others or unknown | — | 160 | 140 | 143 | 162 | 281 |

| | | | | | |
|---|---|---|---|---|---|
| Total | 1253 | 1291 | 1198 | 1404 | 1765 |

TABLE 24
IC50 values determined in biochemical enzyme assays.

| Purified | compound: | | |
|---|---|---|---|
| kinase | imatinib | dasatinib | bosutinib |
| assayed: | IC50 (nM) or % inhibition3 | | |
| ARG³ | 500 | NA | NA |
| BCR- | 250 | 3.0 | 1.0 |
| ABL³ | | | |
| BTK | 4% at 5 μM | 1.1 | 2.5 |
| CSK³ | NA | NA | 310 |
| DDR1² | 31 (22) | ND (7) | ND (12) |
| DDR2¹ | 112 | 133 | 4900 |
| EphB4 | 5% at 5 μM | 3.7 | 5.5 |
| FAK | 2% at 5 μM | 0.2 | 1.0 |
| FER | 14% at 5 μM | 36% at 5 μM | 129 |
| FYN³ | NA | 0.2 | NA |
| GCK | 10% at 5 μM | 60% at 5 μM | 9.9 |
| KHS1 | 14% at 5 μM | 22 | 0.3 |
| KIT | 13% at 1 μM | 13 | NA |
| LYN³ | NA | 3.0 | 8.0 |
| MER | 11% at 5 μM | 40% at 5 μM | 15.7 |
| p38a | 31% at 5 μM | 867 | 1400 |
| SRC³ | NA | 0.5 | 1.0 |
| SYK | 38% at 5 μM | 440 | 52 |
| YES³ | NA | 0.4 | NA |

All values were determined by the Invitrogen “SelectScreen” service with the exception of:
¹Data generated by the “Upstate IC50Profiler Express” service
²Inhibition of autophosphorylation given in parenthesis (see paragraph “Biochemical kinase activity assays”)
³Published data (from Investigational Drugs Database, copyright Current Drugs 2007)
NA; not available,
ND; not determined

### Acknowledgments

This work was partially supported by a Grant from the German Bundesministerium für Bildung und Forschung (BMBF BioChancePLUS grant 0313335A).

1. A method for the characterization of at least one enzyme, comprising the steps of
a) providing a protein preparation containing the enzyme,
b) contacting the protein preparation under essentially physiological conditions with at least one broad spectrum enzyme ligand immobilized on a solid support under conditions allowing the binding of the enzyme to said broad spectrum enzyme ligand,
c) eluting the enzyme, and
d) characterizing the eluted enzyme by mass spectrometry.

2. The method of claim 1, wherein the provision of a protein preparation in step a) includes the steps of harvesting at least one cell containing the enzyme and lysing the cell.

3. A method for the characterization of at least one enzyme, comprising the steps of:
a) providing two aliquots comprising each at least one cell containing the enzyme,
b) incubating one aliquot with a given compound,
c) harvesting the cells,
d) lysing the cells,
e) contacting the cell lysates under essentially physiological conditions with at least one broad spectrum enzyme ligand immobilized on a solid support under conditions allowing the binding of the enzyme to said broad spectrum enzyme ligand,
f) eluting the enzyme or enzymes, and
g) characterizing the eluted enzyme or enzymes by mass spectrometry.

4. The method of claim 3, wherein by characterizing the enzyme it is determined whether the administration of the compound results in a differential expression or activation state of the enzyme.

5. A method for the characterization of at least one enzyme, comprising the steps of:
a) providing two aliquots of a protein preparation containing the enzyme,
b) contacting one aliquot under essentially physiological conditions with at least one broad spectrum enzyme ligand immobilized on a solid support under conditions allowing the binding of the enzyme to said broad spectrum enzyme ligand,
c) contacting the other aliquot under essentially physiological conditions with at least one broad spectrum enzyme ligand immobilized on a solid support under conditions allowing the binding of the enzyme to said broad spectrum enzyme ligand and with a given compound,
d) eluting the enzyme or enzymes, and
e) characterizing the eluted enzyme or enzymes by mass spectrometry.

6. The method of claim 5, wherein the provision of a protein preparation in step a) includes the steps of harvesting at least one cell containing the enzyme and lysing the cell.

7. The method of any of claims 5 or 6, wherein a reduced detection of the enzyme in the aliquot incubated with the compound indicates that the enzyme is a direct target of the compound.

8. A method for the characterization of at least one enzyme-compound complex, comprising the steps of:
a) providing a protein preparation containing the enzyme,
b) contacting the protein preparation under essentially physiological conditions with at least one broad spectrum enzyme ligand immobilized on a solid support under conditions allowing the binding of the enzyme to said broad spectrum enzyme ligand,
c) contacting the bound enzymes with a compound to release at least one bound enzyme, and
d) characterizing the released enzyme or enzymes by mass spectrometry, or
e) eluting the enzyme or enzymes from the ligand and characterizing the enzyme or enzymes by mass spectrometry, thereby identifying one or more binding partners of the compound.

9. The method of claim 8, wherein the provision of a protein preparation in step a) includes the steps of harvesting at least one cell containing the enzyme and lysing the cell.

10. The method of claim 9, performed as a medium or high throughput screening.

11. The method of any of claims 3 to 10, wherein said compound is selected from the group consisting of synthetic compounds, or organic synthetic drugs, more preferably small molecule organic drugs, and natural small molecule compounds.

12. The method any of claims 1 to 11, wherein the enzyme is selected from the group consisting of a kinase, a phosphatase, a protease, a phophodiesterase, a hydrogenase, a dehydrogenase, a ligase, an isomerase, a transferase, an acetylase, a deacetylase, a GTPase, a polymerase, a nuclease, and a helicase.

13. The method of any of claims 1 to 12, wherein the ligand binds to 10% to 50%, preferably 30% to 50% of the enzymes of a given class of enzymes.

14. The method of any of claims 1 to 13, wherein the ligand is an inhibitor.

15. The method of claim 14, wherein the enzyme is a kinase and the ligand is selected from the group consisting of Bisindolylmaleimide VIII, Purvalanol B, CZC00007324 (linkable PD173955), and CZC00008004.

16. The method of any of claims 1 to 15, wherein the characterization of the enzyme is performed by characterizing coeluated binding partners of the enzyme, enzyme subunits or posttranslational modifications of the enzyme.

17. The method of any of claims 1 to 16, wherein the characterization is performed by the identification of proteotypic peptides of the enzyme or of the binding partner of the enzyme.

18. The method of claim 17, wherein the characterization is performed by comparing the proteotypic peptides obtained for the enzyme or the binding partner with known proteotypic peptides.

19. The method of any of claims 1 to 18, wherein the solid support is selected from the group consisting of agarose, modified agarose, sepharose beads (e.g. NHS-activated sepharose), latex, cellulose, and ferro- or ferrimagnetic particles.

20. The method of any of claims 1 to 19, wherein the broad spectrum enzyme ligand is covalently coupled to the solid support.

21. The method of any of claims 1 to 20, wherein 1 to 10 different ligands, preferably 1 to 6, more preferably 1 to 4 are used.

22. The method of any of claims 1 to 21, wherein, when more than one ligand is used, each ligand is present on a different solid support.

23. The method of any of claims 1 to 21, wherein, when more than one ligand is used, at least two different ligands are present on one solid support.

24. The method of any of claims 1 to 23, wherein by characterizing the enzyme or compound-enzyme complex the identity of all or parts of the members of an enzyme class in the cell is determined.

25. The method of any of claims 3 to 24, wherein the compound is different from the ligand.

26. The method of any of claims 1 to 25, wherein the binding between ligand and enzyme is a non-covalent binding.

27. A method for the production of a pharmaceutical composition, comprising the steps of:
a) identifying an enzyme-compound comples according to any of claims 6 to 17, and
b) formulating the compound to a pharmaceutical composition.

28. The method of claim 27, further comprising the step of modulating the binding affinity of the compound to the enzyme.

29. Use of at least one broad spectrum enzyme ligand immobilized on a solid support for the characterization of at least one enzyme or for the characterization of at least on enzyme-compound complex.
